(19) United States
(12) Patent Application Publication
(10) Pub. No.: US 2022/0135601 A1
(43) Pub. Date: May 5, 2022

(54) HETEROCYCLIC COMPOUND, LIGHT-EMITTING DEVICE INCLUDING THE HETEROCYCLIC COMPOUND, AND ELECTRONIC APPARATUS INCLUDING THE LIGHT-EMITTING DEVICE

(71) Applicant: Samsung Display Co., Ltd., Yongin-si (KR)

(72) Inventors: Yeseul LEE, Yongin-si (KR); Hyeongmin KIM, Yongin-si (KR); Heechoon AHN, Yongin-si (KR); Hyunah UM, Yongin-si (KR); Hyoyoung LEE, Yongin-si (KR); Yirang IM, Yongin-si (KR); Seowon CHO, Yongin-si (KR)

(21) Appl. No.: 17/446,810

(22) Filed: Sep. 2, 2021

(30) Foreign Application Priority Data
Nov. 5, 2020 (KR) 10-2020-0147080

(51) Int. Cl. C07F 7/08; C07D 209/86; C07D 405/14; C07D 403/12; C07D 417/14; C07D 417/12; H01L 51/00

(52) U.S. Cl. CPC C07F 7/0814; C07D 209/86; C07D 405/14; C07D 403/12; C07D 417/14; H01L 51/5016; C07F 7/0816; H01L 51/0094; H01L 51/0074; H01L 51/0072; H01L 51/0073; C07D 417/12

(57) ABSTRACT

Provided are a heterocyclic compound represented by Formula 1, a light-emitting device including the heterocyclic compound, and an electronic apparatus including the light-emitting device:

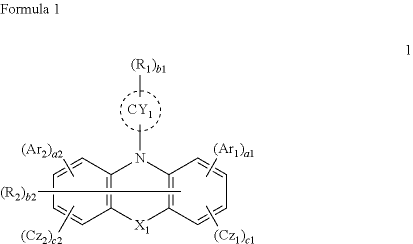

wherein, the detailed description of Formula 1 is the same as described in the present specification.

### CROSS-REFERENCE TO RELATED APPLICATION

This application claims priority to and the benefit of Korean Patent Application No. 10-2020-0147080, filed on Nov. 5, 2020, in the Korean Intellectual Property Office, the disclosure of which is incorporated by reference herein in its entirety.

### BACKGROUND

### 1. Field

One or more embodiments relate to a heterocyclic compound, a light-emitting device including the heterocyclic compound, and an electronic apparatus including the light-emitting device.

### 2. Description of Related Art

From among light-emitting devices, organic light-emitting devices (OLEDs) are self-emission devices that have wide viewing angles, high contrast ratios, short response times, and/or excellent characteristics in terms of luminance, driving voltage, and/or response speed, compared to devices in the related art.

OLEDs may include a first electrode located on a substrate, and a hole transport region, an emission layer, an electron transport region, and a second electrode sequentially stacked on the first electrode. Holes provided from the first electrode may move toward the emission layer through the hole transport region, and electrons provided from the second electrode may move toward the emission layer through the electron transport region. Carriers, such as holes and electrons, recombine in the emission layer to produce excitons. These excitons then transition from an excited state to a ground state to thereby generate light.

### SUMMARY

Aspects according to one or more embodiments are directed toward a light-emitting device including a compound having excellent (e.g., relatively high) luminescence efficiency and an electronic apparatus including the light-emitting device.

Additional aspects will be set forth in part in the description, which follows and, in part, will be apparent from the description, or may be learned by practice of the presented embodiments of the disclosure.

According to one or more embodiments, a heterocylic compound may be represented by Formula 1.

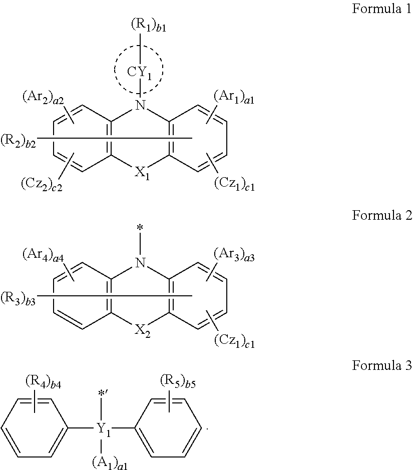

In Formula 1, X₁ may be a single bond, O, S, N(Z₁₁), C(Z₁₁)(Z₁₂), or Si(Z₁₁)(Z₁₂),

ring CY₁ may be a C₃-C₆₀carbocyclic group or a C₁-C₆₀ heterocyclic group,

Cz₁ and Cz₂ may each independently be a group represented by Formula 2,

c1 and c2 may each independently be an integer selected from 0 to 4, and a sum of c1 and c2 may be 1 or more.

In Formula 2, X₂ may be a single bond, O, S, N(Z₂₁), C(Z₂₁)(Z₂₂), or Si(Z₂₁)(Z₂₂).

In Formulae 1 and 2, Ar₁ to Ar₄ may each independently be a group represented by Formula 3,

a1 and a2 may each independently be an integer selected from 0 to 3, a3 and a4 may each independently be an integer selected from 0 to 4, and a sum of a1 to a4 may be 1 or more.

In Formula 3, Y₁ may be C, Si, or a phenylene group,

A₁ may be hydrogen, deuterium (-D), —F, —Cl, —Br, —I, a hydroxyl group, a cyano group, a nitro group, a C₁-C₆₀ alkyl group unsubstituted or substituted with at least one R₁₀ₐ, or a C₆-C₆₀ aryl group unsubstituted or substituted with at least one R₁₀ₐ.

In Formulae 1 and 2, when a2 and c1 are each 1, and a1, a3, a4 and c2 are each 0, Ar₂ may not be a triphenylsilane.

In Formula 3, d1 may be an integer selected from 1 to 3.

In Formulae 1 to 3, R₁ to R₅, Z₁₁, Z₁₂, Z₂₁, and Z₂₂ may each independently be hydrogen, deuterium (-D), —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —C(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂).

In Formula 1 and 2, b1 may be an integer selected from 0 to 10, b2 and b3 may each independently be an integer selected from 0 to 7.

In Formula 3, b4 and b5 may each independently be an integer selected from 0 to 5.

R₁₀ₐ may be

deuterium (-D), —F, —Cl, —Br, —I, a hydroxyl group, a cyano group, or a nitro group;

a C₁-C₆₀ alkyl group, a C₂-C₆₀ alkenyl group, a C₂-C₆₀ alkynyl group, or a C₁-C₆₀ group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —C(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;

a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —C(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or

—Si(Q₃₁)(Q₃₂)(Q₃₃), —C(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),

wherein Q₁ to Q₃, Q₁₁ to Q₁₃, Q₂₁ to Q₂₃, and Q₃₁ to Q₃₃ may each independently be hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

In Formula 2, * indicates a binding site to a neighboring atom of Formula 1, and

in Formula 3, *′ indicates a binding site to a neighboring atom of Formula 2 or 3.

According to another embodiment, a light-emitting device may include

a first electrode, a second electrode facing the first electrode, an interlayer located between the first electrode and the second electrode and including an emission layer, and a heterocyclic compound as described above.

According to another embodiment, an electronic apparatus may include the light-emitting device.

### BRIEF DESCRIPTION OF THE DRAWINGS

The above and other aspects, features, and enhancements of certain embodiments of the disclosure will be more apparent from the following description taken in conjunction with the accompanying drawings, in which:

FIG. 1 is a schematic cross-sectional view of a light-emitting device according to an embodiment;

FIG. 2 is a schematic cross-sectional view of a light-emitting apparatus according to an embodiment.

FIG. 3 is a schematic cross-sectional view of a light-emitting apparatus according to an embodiment.

### DETAILED DESCRIPTION

Reference will now be made in detail to embodiments, non-limiting examples of which are illustrated in the accompanying drawings, wherein like reference numerals refer to like elements throughout the specification. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Throughout the disclosure, the expression “at least one of a, b or c”, “at least one selected from a, b and c”, or “at least one of a to c” indicates only a, only b, only c, both a and b, both a and c, both b and c, all of a, b, and c, or variations thereof.

A heterocyclic compound represented by Formula 1 may be provided:

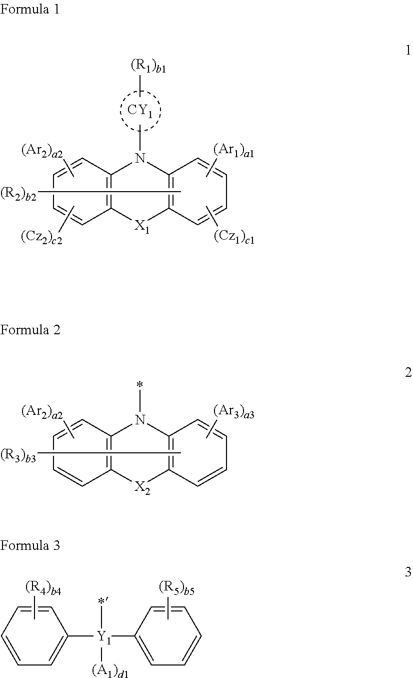

In Formula 1, X₁ may be a single bond, O, S, N(Z₁₁), C(Z₁₁)(Z₁₂), or Si(Z₁₁)(Z₁₂).

In an embodiment, in Formula 1, X₁ may be a single bond.

In Formula 1, ring CY₁ may be a C₃-C₆₀ carbocyclic group or a C₁-C₆₀ heterocyclic group.

In an embodiment, in Formula 1, ring CY₁ may be a benzene group, a naphthalene group, a fluorene group, a dibenzofuran group, or a dibenzothiophene group.

In one or more embodiments, in Formula 1, when ring CY₁ is a benzene group,

R₁ may be hydrogen, deuterium, —F, —Cl, —Br, —I, a hydroxyl group, a cyano group, or a nitro group;

a C₁-C₂₀ alkyl group, a C₂-C₂₀ alkenyl group, a C₂-C₂₀ alkynyl group, a C₁-C₂₀ alkoxy group, or a phenyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a C₂-C₂₀ alkenyl group, a C₂-C₂₀ alkynyl group, a C₁-C₂₀ alkoxy group, or any combination thereof; or

a naphthalene group, a fluorenyl group, a dibenzofuran group, or a dibenzothiophene group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, C₂-C₂₀ alkenyl group, C₂-C₂₀ alkynyl group, C₁-C₂₀ alkoxy group, a phenyl group, or any combination thereof; and

when R₁ is a phenyl group, b1 may be 1. R₁ and b1 are each the same as respectively described in the present specification.

In one or more embodiments, in Formula 1, when ring CY₁ is a benzene group,

R₁ may be hydrogen, deuterium, —F, —Cl, —Br, —I, a hydroxyl group, a cyano group, or a nitro group; or

a C₁-C₂₀ alkyl group, a C₂-C₂₀ alkenyl group, a C₂-C₂₀ alkynyl group, a C₁-C₂₀ alkoxy group, or a phenyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a C₂-C₂₀ alkenyl group, a C₂-C₂₀ alkynyl group, a C₁-C₂₀ alkoxy group, or any combination thereof; or

when R₁ is a phenyl group, b1 may be 1.

In an embodiment, in Formula 1, ring CY₁ may be a benzene group, a naphthalene group, a fluorene group, a dibenzofuran group, or a dibenzothiophene group,

and when CY₁ is a benzene group,

R₁ may be hydrogen, deuterium, —F, —Cl, —Br, —I, a hydroxyl group, a cyano group, or a nitro group;

a C₁-C₂₀ alkyl group, a C₂-C₂₀ alkenyl group, a C₂-C₂₀ alkynyl group, a C₁-C₂₀ alkoxy group, or a phenyl group each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a C₂-C₂₀ alkenyl group, a C₂-C₂₀ alkynyl group, a C₁-C₂₀ alkoxy group, or any combination thereof; or

a naphthalene group, a fluorenyl group, a dibenzofuran group, or a dibenzothiophene group each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, C₂-C₂₀ alkenyl group, C₂-C₂₀ alkynyl group, C₁-C₂₀ alkoxy group, a phenyl group, or any combination thereof; and

when R₁ is a phenyl group, b1 may be 1.

In Formula 1, Cz₁ and Cz₂ may each independently be a group represented by Formula 2. Formula 2 is the same as described in the present specification.

In Formula 1, c1 and c2 may each independently be an integer selected from 0 to 4, and the sum of c1 and c2 may be 1 or more.

In an embodiment, c1 and c2 may each independently be 0 or 1.

In Formula 2, X₂ may be a single bond, O, S, N(Z₂₁), C(Z₂₁)(Z₂₂), or Si(Z₂₁)(Z₂₂).

In an embodiment, in Formula 2, X₂ may be a single bond.

In Formulae 1 and 2, Ar₁ to Ar₄ may each independently be a group represented by Formula 3. Formula 3 is the same as described in the present specification.

In Formulae 1 and 2, a1 and a2 may each independently be an integer selected from 0 to 3, a3 and a4 may each independently be an integer selected from 0 to 4, and the sum of a1 to a4 may be 1 or more.

In an embodiment, a1 to a4 may each independently be 0 or 1.

In an embodiment, in Formulae 1 and 2, when c1 is 1 and c2 is 0, i) a1 and a2 may each independently be 0 or 1, or ii) a3 and a4 may each independently be 0 or 1.

In Formula 3, Y₁ may be C, Si, or a phenylene group.

In Formula 3, A₁ may be hydrogen, deuterium (-D), —F, —Cl, —Br, —I, a hydroxyl group, a cyano group, a nitro group, a C₁-C₆₀ alkyl group unsubstituted or substituted with at least one R₁₀ₐ, or a C₆-C₆₀ aryl group unsubstituted or substituted with at least one R₁₀ₐ.

In Formulae 1 and 2, when a2 and c1 are each 1, and a1, a3, a4 and c2 are each 0, Ar₂ may not be a triphenylsilane. Here, a triphenylsilane group refers to an unsubstituted triphenylsilane group.

In Formula 3, d1 may be an integer selected from 1 to 3.

In an embodiment, d1 may be 1, but embodiments of the present disclosure are not limited thereto.

In Formulae 1 to 3, R₁ to R₅, Z₁₁, Z₁₂, Z₂₁, and Z₂₂ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —C(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂).

In an embodiment, in Formula 1, R₁ may be: hydrogen, deuterium, —F, —Cl, —Br, —I, a hydroxyl group, a cyano group, or a nitro group;

a C₁-C₂₀ alkyl group, a C₂-C₂₀ alkenyl group, a C₂-C₂₀ alkynyl group, a C₁-C₂₀ alkoxy group, or a phenyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a C₂-C₂₀ alkenyl group, a C₂-C₂₀ alkynyl group, a C₁-C₂₀ alkoxy group, or any combination thereof; or

a naphthalene group, a fluorenyl group, a dibenzofuran group, or a dibenzothiophene group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, C₂-C₂₀ alkenyl group, C₂-C₂₀ alkynyl group, C₁-C₂₀ alkoxy group, a phenyl group, or any combination thereof.

In an embodiment, in Formulae 1 and 2, R₂, R₃, Z₁₁, Z₁₂, Z₂₁, and Z₂₂ may each independently be:

hydrogen, deuterium (-D), —F, —Cl, —Br, —I, a hydroxyl group, a cyano group, or a nitro group; or

a C₁-C₂₀ alkyl group, a C₂-C₂₀ alkenyl group, a C₂-C₂₀ alkynyl group, a C₁-C₂₀ alkoxy group, or a phenyl group, each unsubstituted or substituted with deuterium (-D), —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a C₂-C₂₀ alkenyl group, a C₂-C₂₀ alkynyl group, a C₁-C₂₀ alkoxy group, a phenyl group, or any combination thereof.

In an embodiment, in Formula 3, R₄ and R₅ may each independently be selected from:

hydrogen, deuterium (-D), —F, —Cl, —Br, —I, a hydroxyl group, a cyano group, or a nitro group; or

a C₁-C₂₀ alkyl group, a C₂-C₂₀ alkenyl group, a C₂-C₂₀ alkynyl group or a C₁-C₂₀ alkoxy group, each unsubstituted or substituted with deuterium (-D), —F, —Cl, —Br, —I, —CD₃, —CD₂H, —CDH₂, —CF₃, —CF₂H, —CFH₂, a hydroxyl group, a cyano group, a nitro group, —Si(Q₃₁)(Q₃₂)(Q₃₃), —C(Q₃₁)(Q₃₂)(Q₃₃), or any combination thereof;

a phenyl group, a fluorenyl group, a dibenzofuranyl group, or a dibenzothiophenyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a C₂-C₂₀ alkenyl group, a C₂-C₂₀ alkynyl group, a C₁-C₂₀ alkoxy group, a phenyl group, a fluorenyl group, a dibenzofuranyl group, a dibenzothiophenyl group, —Si(Q₃₁)(Q₃₂)(Q₃₃), —C(Q₃₁)(Q₃₂)(Q₃₃), or any combination thereof; or

Si(Q₁)(Q₂)(Q₃) or —C(Q₁)(Q₂)(Q₃).

In Formula 1, b1 may be an integer selected from 0 to 10.

In an embodiment, b1 may be an integer selected from 0 to 8.

In an embodiment, b1 may be an integer selected from 0 to 5.

In Formulae 1 and 2, b2 and b3 may each independently be an integer selected from 0 to 7.

In Formula 3, b4 and b5 may each independently be an integer selected from 0 to 5.

In Formula 2, * may indicate a binding site to a neighboring atom of Formula 1.

in Formula 3, *′ may indicate a binding site to a neighboring atom of Formula 2 or 3.

In an embodiment, the heterocyclic compound represented by Formula 1 may be represented by one of Formulae 1-1 to 1-6:

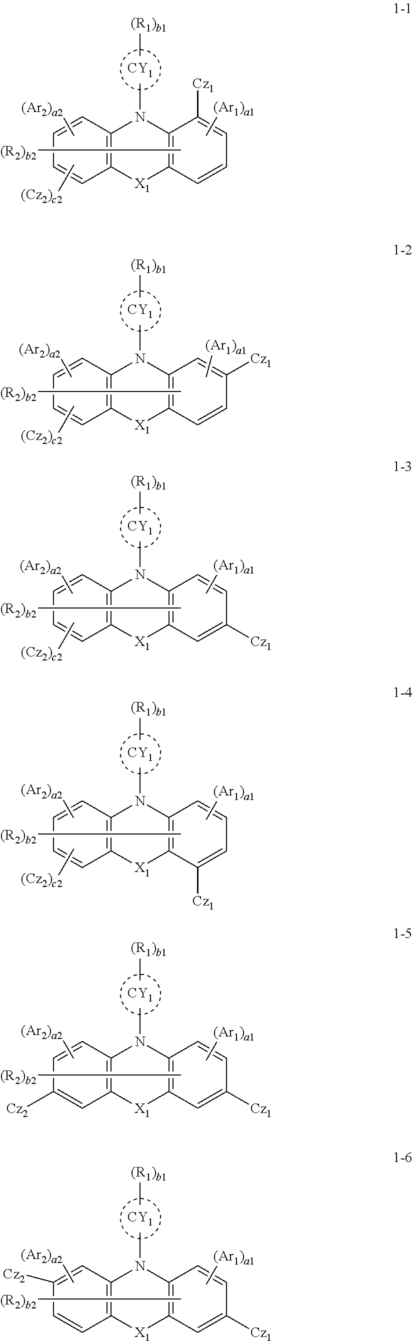

wherein, in Formulae 1-1 to 1-6,

X₁, CY₁, Ar₁, Ar₂, a1, a2, Cz₁, Cz₂, R₁, R₂, b1 and b2 are the same as respectively described in the present specification.

In an embodiment, the heterocyclic compound represented by Formula 1 may be represented by one of Formulae 1(1) to 1(4):

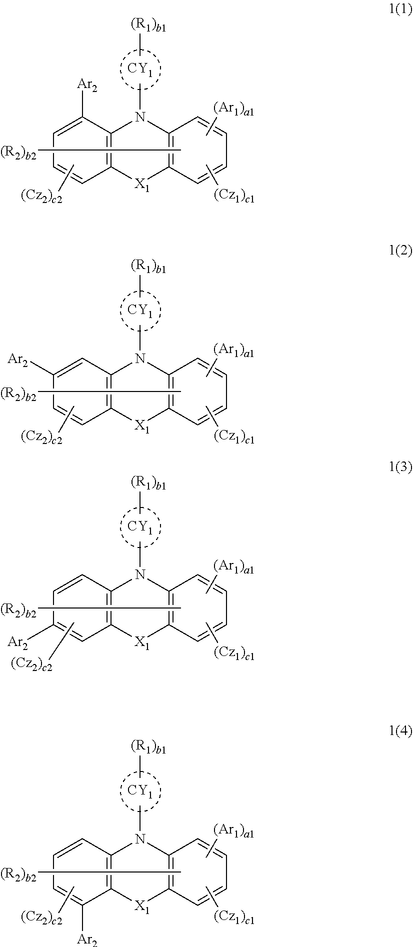

wherein, in Formulae 1(1) to 1(4),

X₁, CY₁, Ar₁, Ar₂, a1, Cz₁, Cz₂, c1, c2 R₁, R₂, b1 and b2 are each the same as respectively described in the present specification.

In an embodiment, the group represented by Formula 2 may be represented by one of Formulae 2-1 to 2-4:

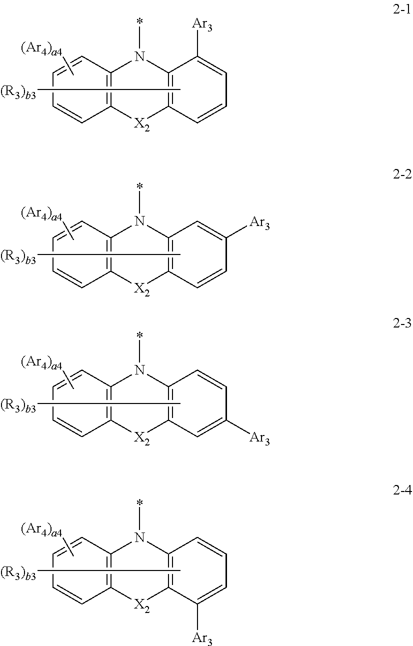

wherein, in Formulae 2-1 to 2-4,

X₂, Ar₃, Ar₄, a4, R₃ and b3 are each the same as respectively described in the present specification, and

* is a binding site to a neighboring atom in Formula 1.

In an embodiment, the group represented by Formula 2 may be represented by one of Formulae 2(1) to 2(5).

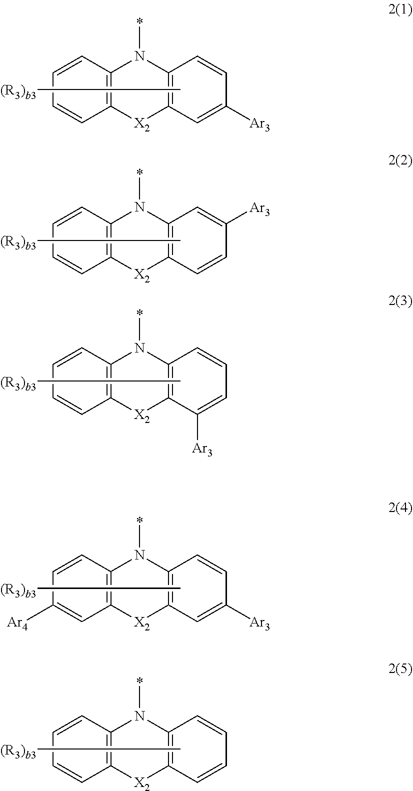

wherein, in Formulae 2(1) to 2(5),

X₂, Ar₃, Ar₄, R₃ and b3 each are the same as respectively described in the present specification, and

* is a binding site to a neighboring atom in Formula 1.

In an embodiment, heterocyclic compound represented by Formula 1 may be represented by one selected from Formulae 1-2a, 1-2b, 1-3a to 1-3e, and 1-4a to 1-4d:

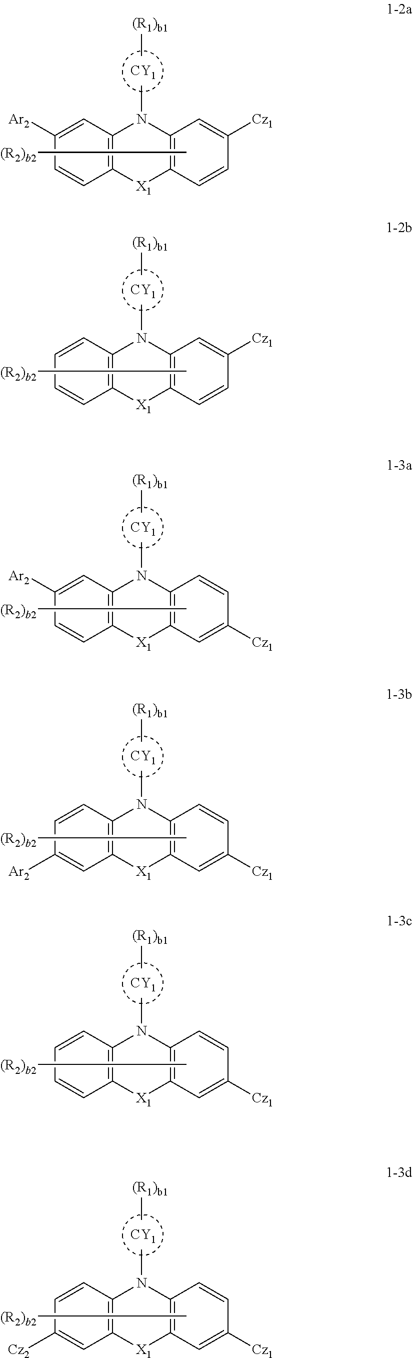

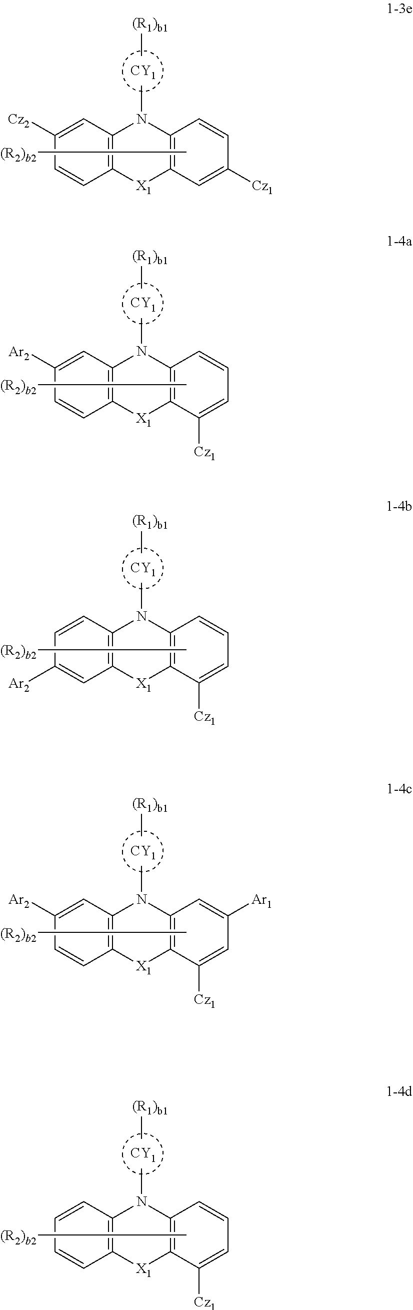

wherein, in Formulae 1-2a, 1-2b, 1-3a to 1-3e, and 1-4a to 1-4d,

X₁, CY₁, Ar₁, Ar₂, Cz₁, Cz₂, R₁, R₂, b1 and b2 are each the same as respectively described in the present specification.

In an embodiment, the heterocyclic compound may satisfy Condition 1 or Condition 2:

### Condition 1

Formula 1 is a group represented by one of Formulae 1-2a, 1-3a, 1-3b, and 1-4a to 1-4c, and

Formula 2 is a group represented by one of Formulae 2(1) to 2(5).

### Condition 2

Formula 1 is a group represented by one of Formulae 1-2b, 1-3c to 1-3e, and 1-4d, and

Formula 2 is a group represented by one of Formulae 2(1) to 2(4).

In an embodiment, the heterocyclic compound may be selected from Compounds 1 to 189:

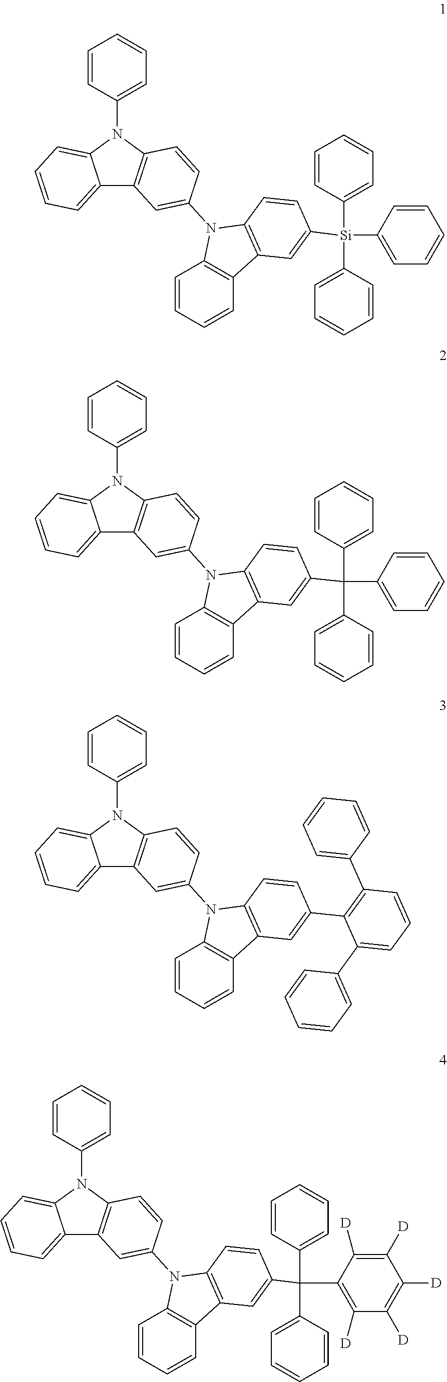 [SMILES: c1ccc(-n2c3ccccc3c3cc(-n4c5ccccc5c5cc([Si](c6ccccc6)(c6ccccc6)c6ccccc6)ccc54)ccc32)cc1 ; c1ccc(-n2c3ccccc3c3cc(-n4c5ccccc5c5cc(C(c6ccccc6)(c6ccccc6)c6ccccc6)ccc54)ccc32)cc1 ; c1ccc(-c2cccc(-c3ccccc3)c2-c2ccc3c(c2)c2ccccc2n3-c2ccc3c(c2)c2ccccc2n3-c2ccccc2)cc1 ; [2H]c1c([2H])c([2H])c(C(c2ccccc2)(c2ccccc2)c2ccc3c(c2)c2ccccc2n3-c2ccc3c(c2)c2ccccc2n3-c2ccccc2)c([2H])c1[2H]]

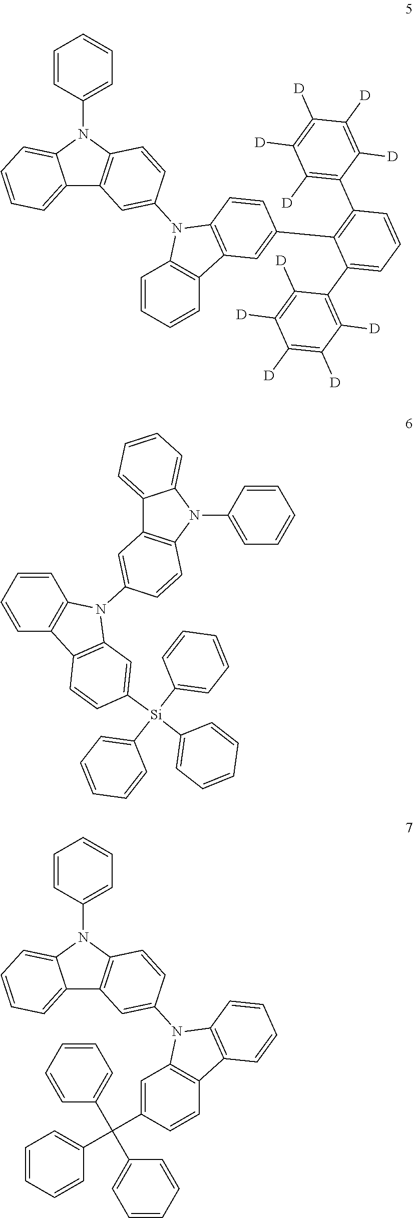

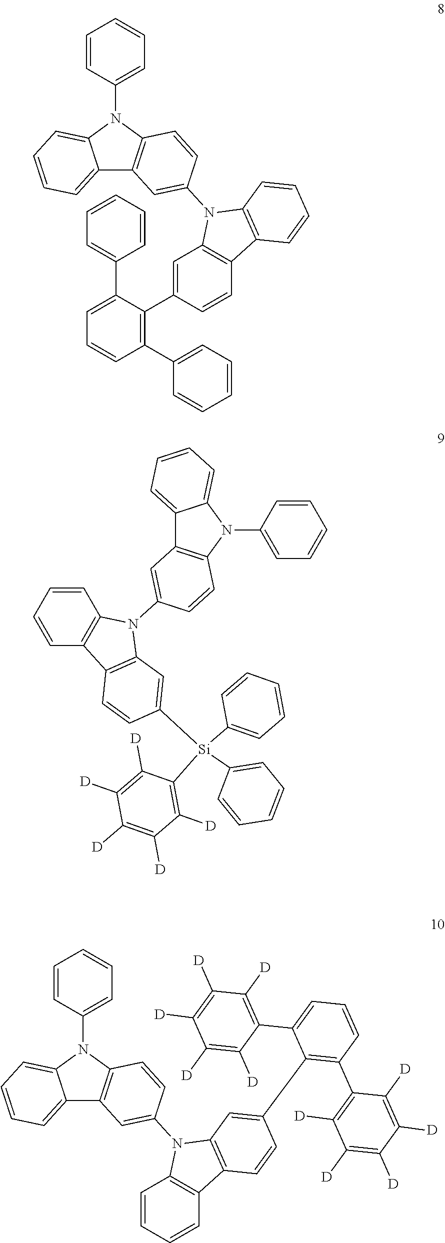

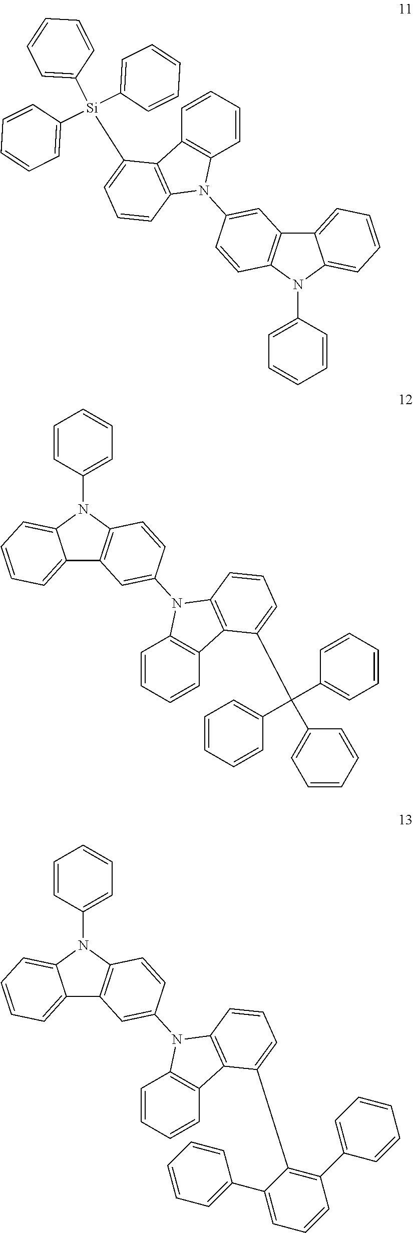 [SMILES: c1ccc(-n2c3ccccc3c3cc(-n4c5ccccc5c5c([Si](c6ccccc6)(c6ccccc6)c6ccccc6)cccc54)ccc32)cc1 ; c1ccc(-n2c3ccccc3c3cc(-n4c5ccccc5c5c(C(c6ccccc6)(c6ccccc6)c6ccccc6)cccc54)ccc32)cc1 ; c1ccc(-c2cccc(-c3ccccc3)c2-c2cccc3c2c2ccccc2n3-c2ccc3c(c2)c2ccccc2n3-c2ccccc2)cc1]

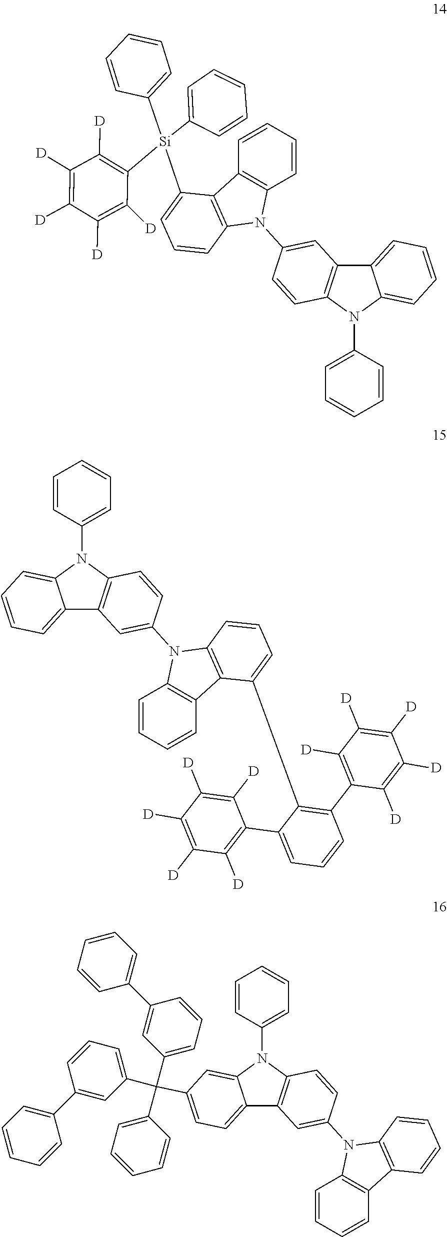 [SMILES: [2H]c1c([2H])c([2H])c([Si](c2ccccc2)(c2ccccc2)c2cccc3c2c2ccccc2n3-c2ccc3c(c2)c2ccccc2n3-c2ccccc2)c([2H])c1[2H] ; [2H]c1c([2H])c([2H])c(-c2cccc(-c3c([2H])c([2H])c([2H])c([2H])c3[2H])c2-c2cccc3c2c2ccccc2n3-c2ccc3c(c2)c2ccccc2n3-c2ccccc2)c([2H])c1[2H] ; c1ccc(-c2cccc(C(c3ccccc3)(c3cccc(-c4ccccc4)c3)c3ccc4c5cc(-n6c7ccccc7c7ccccc76)ccc5n(-c5ccccc5)c4c3)c2)cc1]

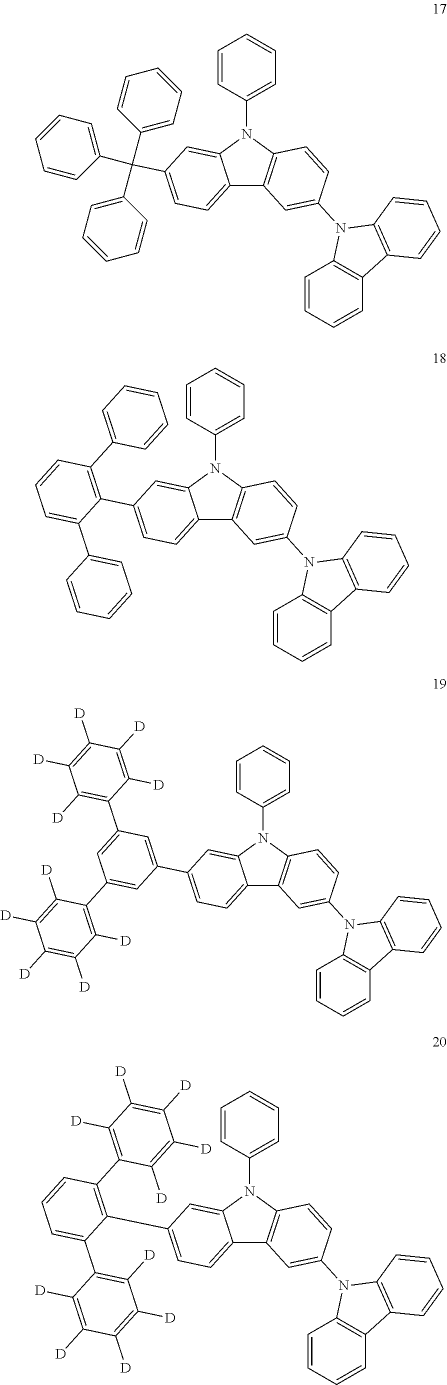 [SMILES: c1ccc(-n2c3ccc(-n4c5ccccc5c5ccccc54)cc3c3ccc(C(c4ccccc4)(c4ccccc4)c4ccccc4)cc32)cc1 ; c1ccc(-c2cccc(-c3ccccc3)c2-c2ccc3c4cc(-n5c6ccccc6c6ccccc65)ccc4n(-c4ccccc4)c3c2)cc1 ; [2H]c1c([2H])c([2H])c(-c2cc(-c3ccc4c5cc(-n6c7ccccc7c7ccccc76)ccc5n(-c5ccccc5)c4c3)cc(-c3c([2H])c([2H])c([2H])c([2H])c3[2H])c2)c([2H])c1[2H] ; [2H]c1c([2H])c([2H])c(-c2cccc(-c3c([2H])c([2H])c([2H])c([2H])c3[2H])c2-c2ccc3c4cc(-n5c6ccccc6c6ccccc65)ccc4n(-c4ccccc4)c3c2)c([2H])c1[2H]]

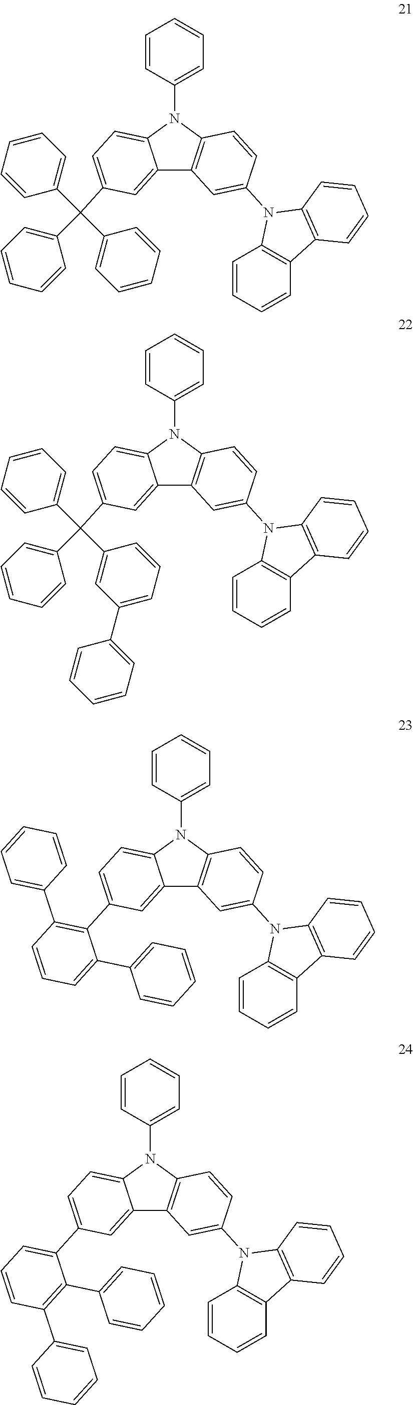 [SMILES: c1ccc(-n2c3ccc(-n4c5ccccc5c5ccccc54)cc3c3cc(C(c4ccccc4)(c4ccccc4)c4ccccc4)ccc32)cc1 ; c1ccc(-c2cccc(C(c3ccccc3)(c3ccccc3)c3ccc4c(c3)c3cc(-n5c6ccccc6c6ccccc65)ccc3n4-c3ccccc3)c2)cc1 ; c1ccc(-c2cccc(-c3ccccc3)c2-c2ccc3c(c2)c2cc(-n4c5ccccc5c5ccccc54)ccc2n3-c2ccccc2)cc1 ; c1ccc(-c2cccc(-c3ccc4c(c3)c3cc(-n5c6ccccc6c6ccccc65)ccc3n4-c3ccccc3)c2-c2ccccc2)cc1]

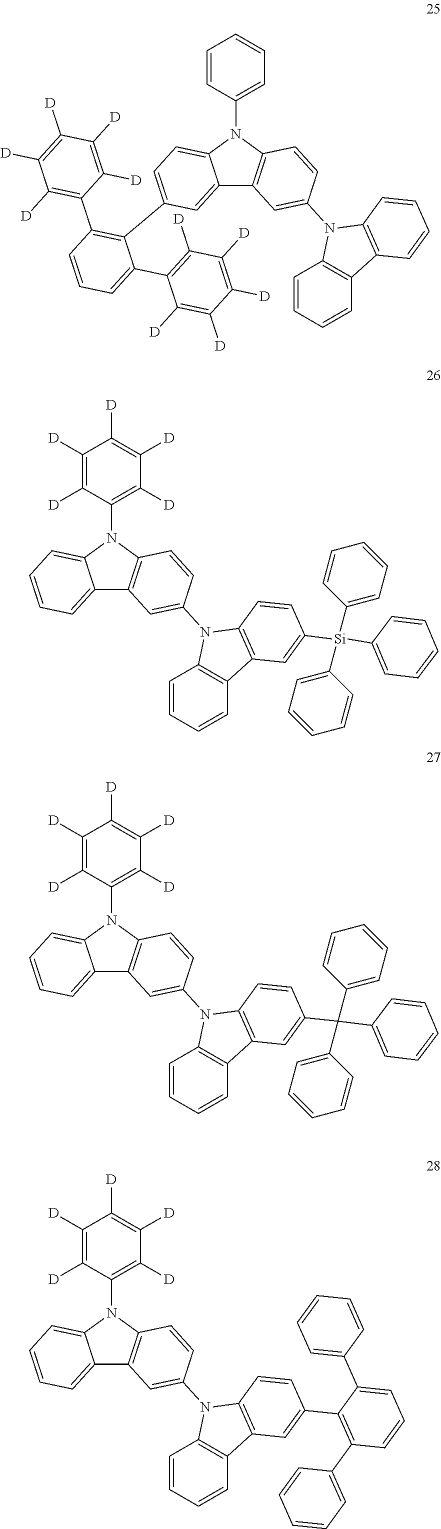 [SMILES: [2H]c1c([2H])c([2H])c(-c2cccc(-c3c([2H])c([2H])c([2H])c([2H])c3[2H])c2-c2ccc3c(c2)c2cc(-n4c5ccccc5c5ccccc54)ccc2n3-c2ccccc2)c([2H])c1[2H] ; [2H]c1c([2H])c([2H])c(-n2c3ccccc3c3cc(-n4c5ccccc5c5cc([Si](c6ccccc6)(c6ccccc6)c6ccccc6)ccc54)ccc32)c([2H])c1[2H] ; [2H]c1c([2H])c([2H])c(-n2c3ccccc3c3cc(-n4c5ccccc5c5cc(C(c6ccccc6)(c6ccccc6)c6ccccc6)ccc54)ccc32)c([2H])c1[2H] ; [2H]c1c([2H])c([2H])c(-n2c3ccccc3c3cc(-n4c5ccccc5c5cc(-c6c(-c7ccccc7)cccc6-c6ccccc6)ccc54)ccc32)c([2H])c1[2H]]

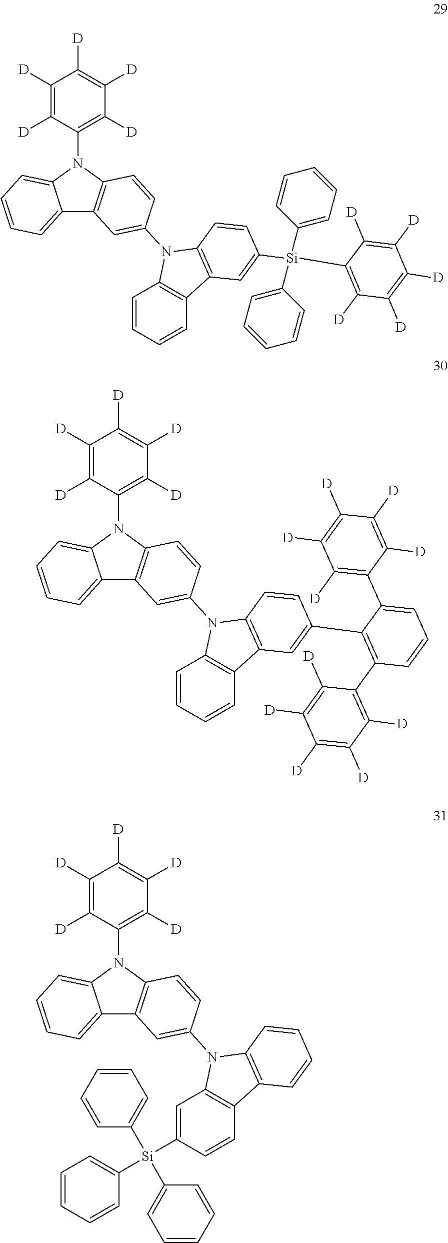 [SMILES: [2H]c1c([2H])c([2H])c(-n2c3ccccc3c3cc(-n4c5ccccc5c5cc([Si](c6ccccc6)(c6ccccc6)c6c([2H])c([2H])c([2H])c([2H])c6[2H])ccc54)ccc32)c([2H])c1[2H] ; [2H]c1c([2H])c([2H])c(-c2cccc(-c3c([2H])c([2H])c([2H])c([2H])c3[2H])c2-c2ccc3c(c2)c2ccccc2n3-c2ccc3c(c2)c2ccccc2n3-c2c([2H])c([2H])c([2H])c([2H])c2[2H])c([2H])c1[2H] ; [2H]c1c([2H])c([2H])c(-n2c3ccccc3c3cc(-n4c5ccccc5c5ccc([Si](c6ccccc6)(c6ccccc6)c6ccccc6)cc54)ccc32)c([2H])c1[2H]]

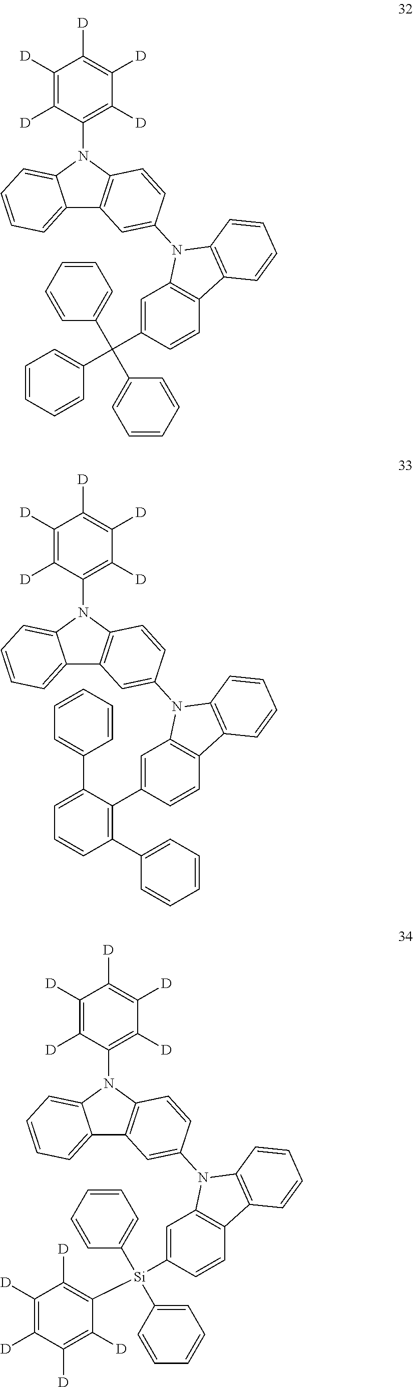 [SMILES: [2H]c1c([2H])c([2H])c(-n2c3ccccc3c3cc(-n4c5ccccc5c5ccc(C(c6ccccc6)(c6ccccc6)c6ccccc6)cc54)ccc32)c([2H])c1[2H] ; [2H]c1c([2H])c([2H])c(-n2c3ccccc3c3cc(-n4c5ccccc5c5ccc(-c6c(-c7ccccc7)cccc6-c6ccccc6)cc54)ccc32)c([2H])c1[2H] ; [2H]c1c([2H])c([2H])c(-n2c3ccccc3c3cc(-n4c5ccccc5c5ccc([Si](c6ccccc6)(c6ccccc6)c6c([2H])c([2H])c([2H])c([2H])c6[2H])cc54)ccc32)c([2H])c1[2H]]

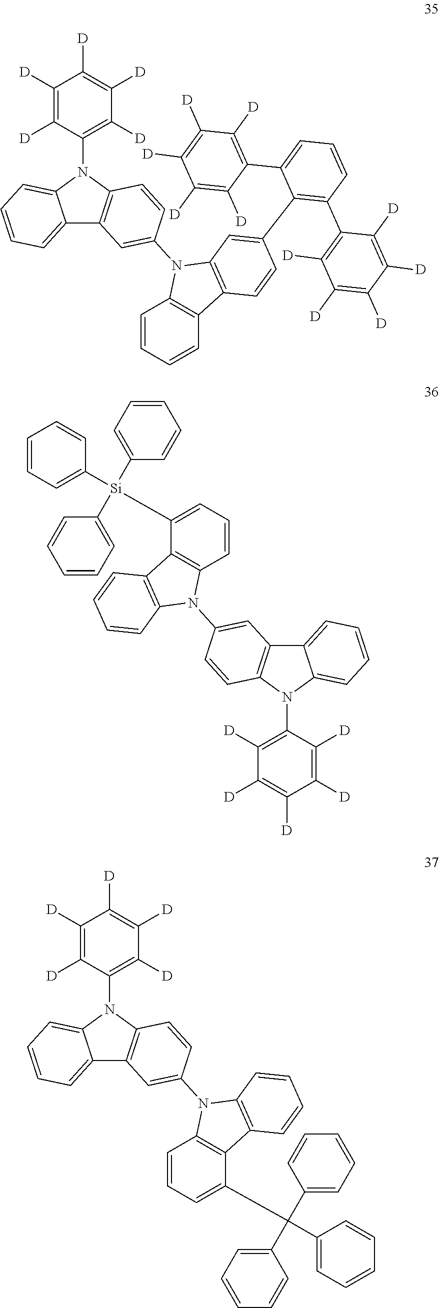

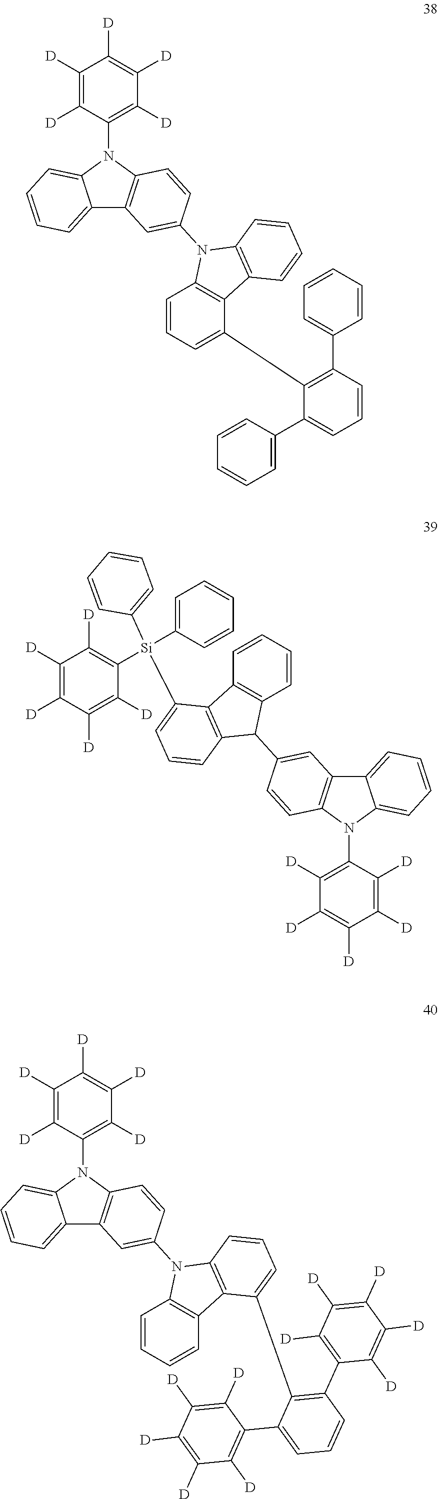 [SMILES: [2H]c1c([2H])c([2H])c(-n2c3ccccc3c3cc(-n4c5ccccc5c5c(-c6c(-c7ccccc7)cccc6-c6ccccc6)cccc54)ccc32)c([2H])c1[2H] ; [2H]c1c([2H])c([2H])c(-n2c3ccccc3c3cc(C4c5ccccc5-c5c4cccc5[Si](c4ccccc4)(c4ccccc4)c4c([2H])c([2H])c([2H])c([2H])c4[2H])ccc32)c([2H])c1[2H] ; [2H]c1c([2H])c([2H])c(-c2cccc(-c3c([2H])c([2H])c([2H])c([2H])c3[2H])c2-c2cccc3c2c2ccccc2n3-c2ccc3c(c2)c2ccccc2n3-c2c([2H])c([2H])c([2H])c([2H])c2[2H])c([2H])c1[2H]]

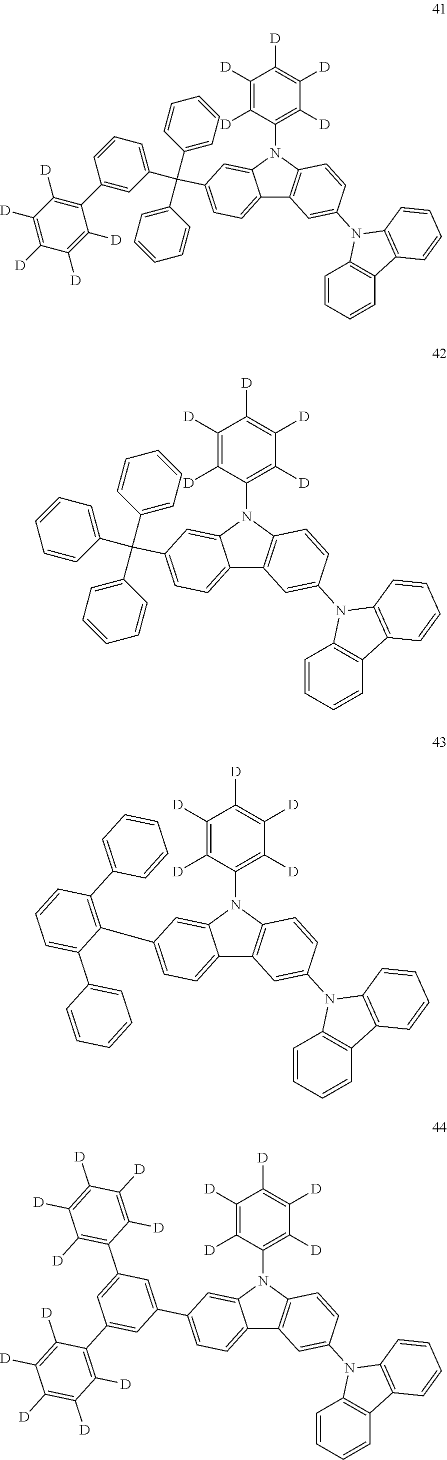

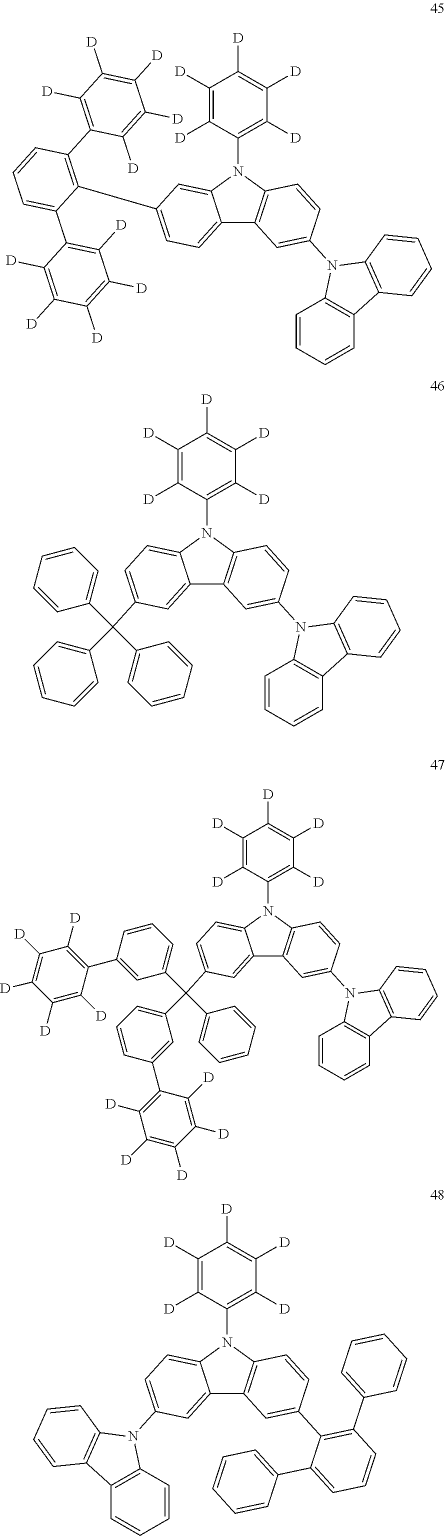

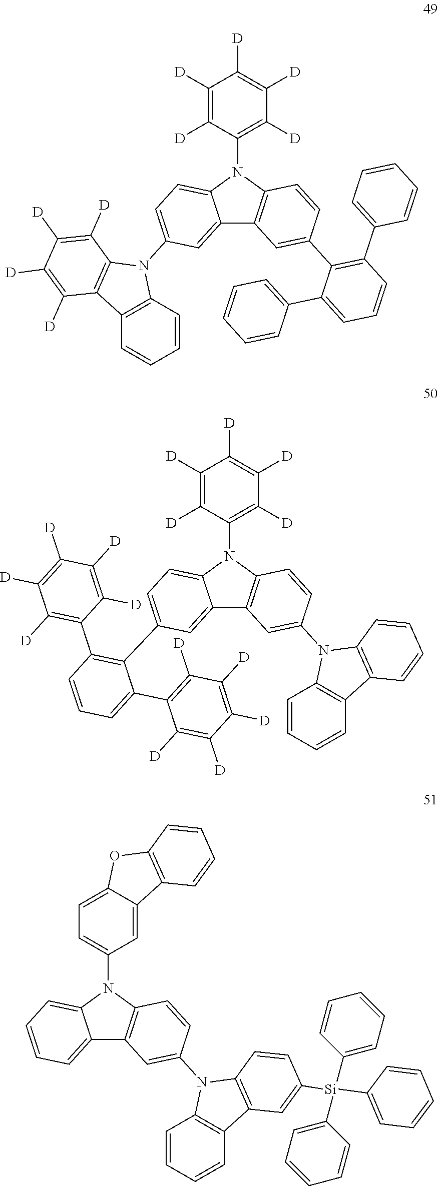 [SMILES: [2H]c1c([2H])c([2H])c(-n2c3ccc(-c4c(-c5ccccc5)cccc4-c4ccccc4)cc3c3cc(-n4c5ccccc5c5c([2H])c([2H])c([2H])c([2H])c54)ccc32)c([2H])c1[2H] ; [2H]c1c([2H])c([2H])c(-c2cccc(-c3c([2H])c([2H])c([2H])c([2H])c3[2H])c2-c2ccc3c(c2)c2cc(-n4c5ccccc5c5ccccc54)ccc2n3-c2c([2H])c([2H])c([2H])c([2H])c2[2H])c([2H])c1[2H] ; c1ccc([Si](c2ccccc2)(c2ccccc2)c2ccc3c(c2)c2ccccc2n3-c2ccc3c(c2)c2ccccc2n3-c2ccc3oc4ccccc4c3c2)cc1]

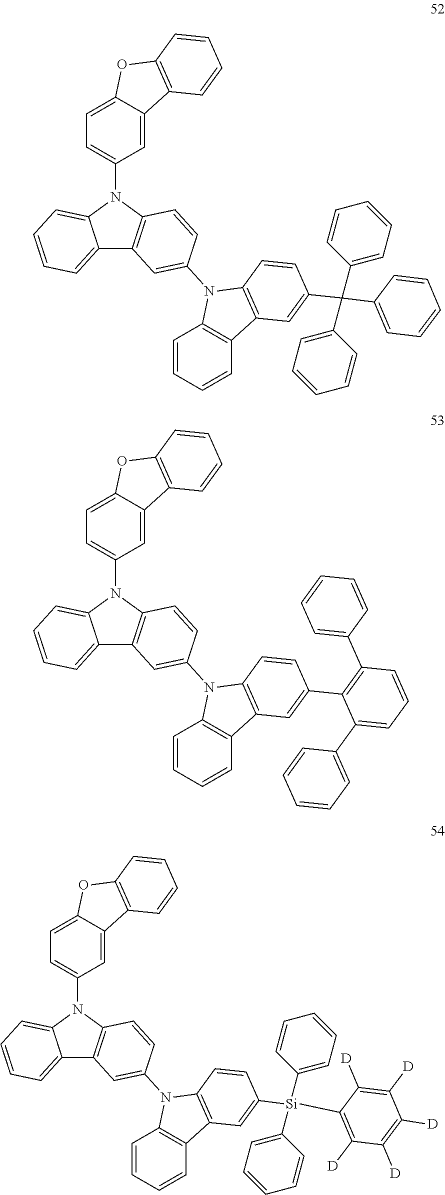

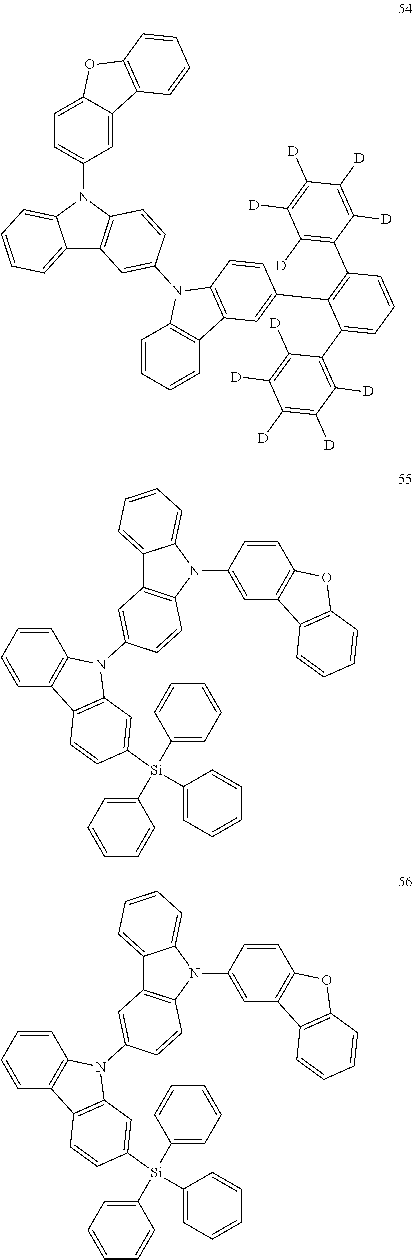

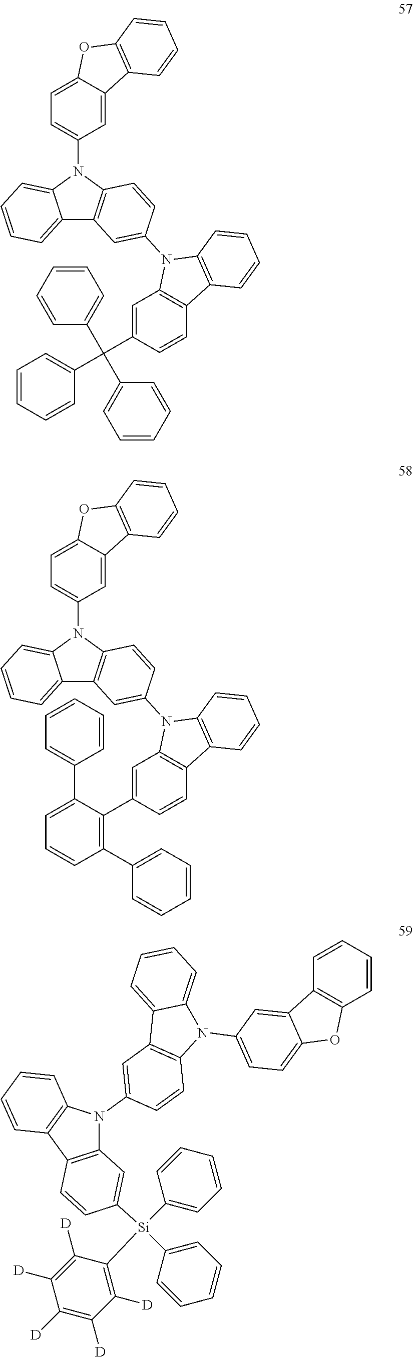

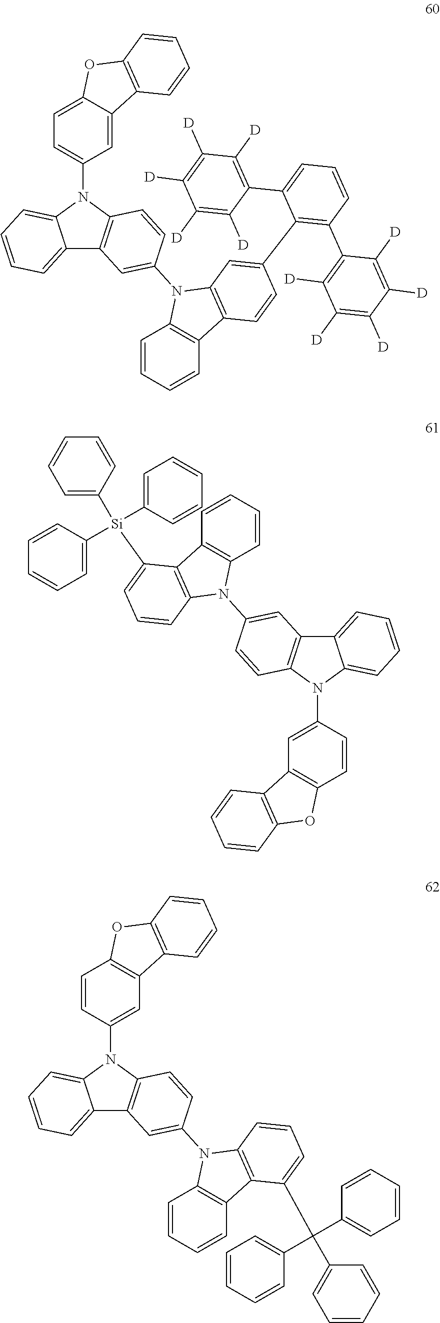

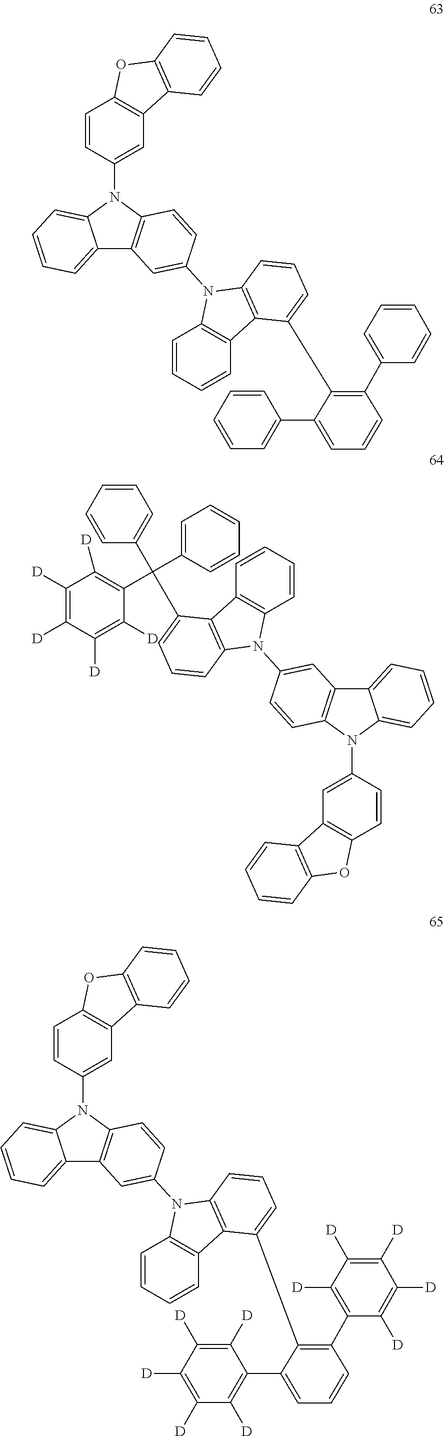 [SMILES: c1ccc(-c2cccc(-c3ccccc3)c2-c2cccc3c2c2ccccc2n3-c2ccc3c(c2)c2ccccc2n3-c2ccc3oc4ccccc4c3c2)cc1 ; [2H]c1c([2H])c([2H])c(C(c2ccccc2)(c2ccccc2)c2cccc3c2c2ccccc2n3-c2ccc3c(c2)c2ccccc2n3-c2ccc3oc4ccccc4c3c2)c([2H])c1[2H] ; [2H]c1c([2H])c([2H])c(-c2cccc(-c3c([2H])c([2H])c([2H])c([2H])c3[2H])c2-c2cccc3c2c2ccccc2n3-c2ccc3c(c2)c2ccccc2n3-c2ccc3oc4ccccc4c3c2)c([2H])c1[2H]]

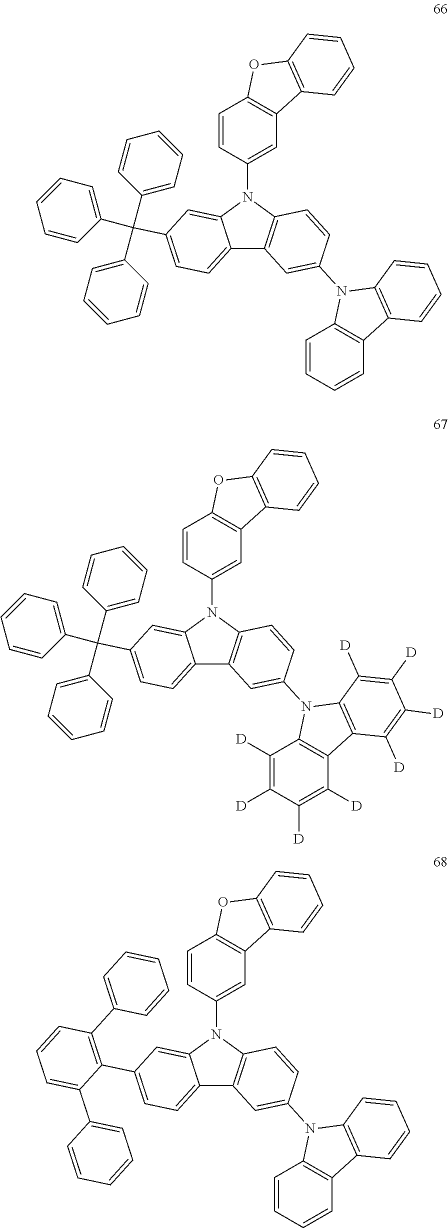

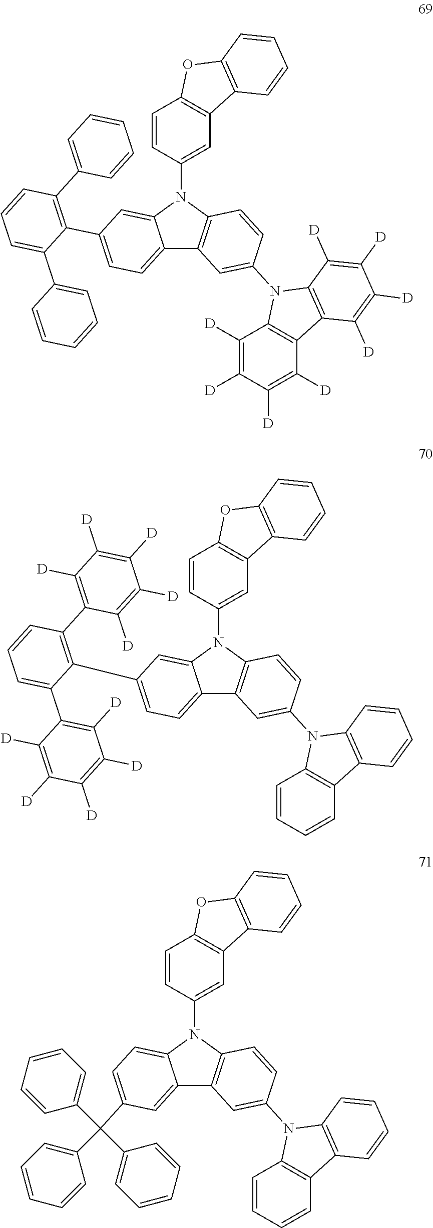

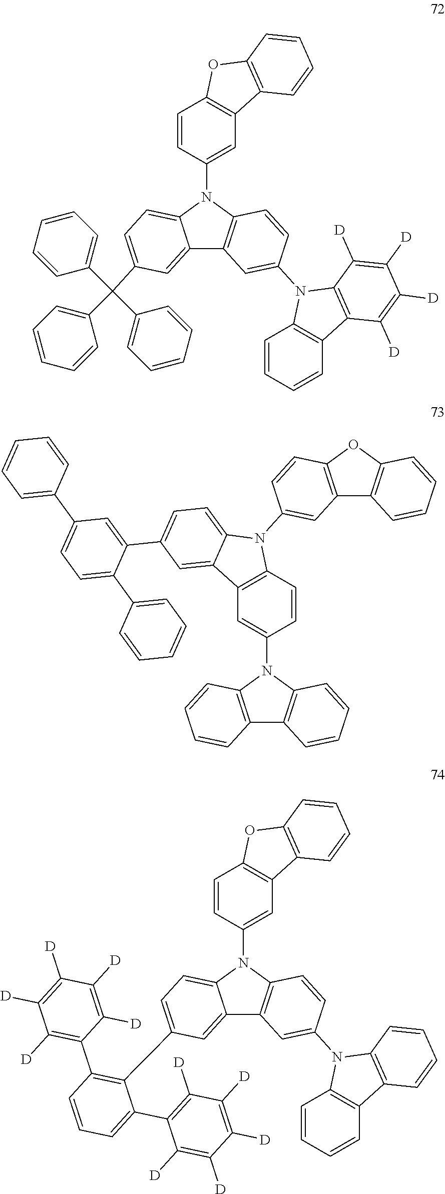 [SMILES: [2H]c1c([2H])c([2H])c2c(c1[2H])c1ccccc1n2-c1ccc2c(c1)c1cc(C(c3ccccc3)(c3ccccc3)c3ccccc3)ccc1n2-c1ccc2oc3ccccc3c2c1 ; c1ccc(-c2ccc(-c3ccccc3)c(-c3ccc4c(c3)c3cc(-n5c6ccccc6c6ccccc65)ccc3n4-c3ccc4oc5ccccc5c4c3)c2)cc1 ; [2H]c1c([2H])c([2H])c(-c2cccc(-c3c([2H])c([2H])c([2H])c([2H])c3[2H])c2-c2ccc3c(c2)c2cc(-n4c5ccccc5c5ccccc54)ccc2n3-c2ccc3oc4ccccc4c3c2)c([2H])c1[2H]]

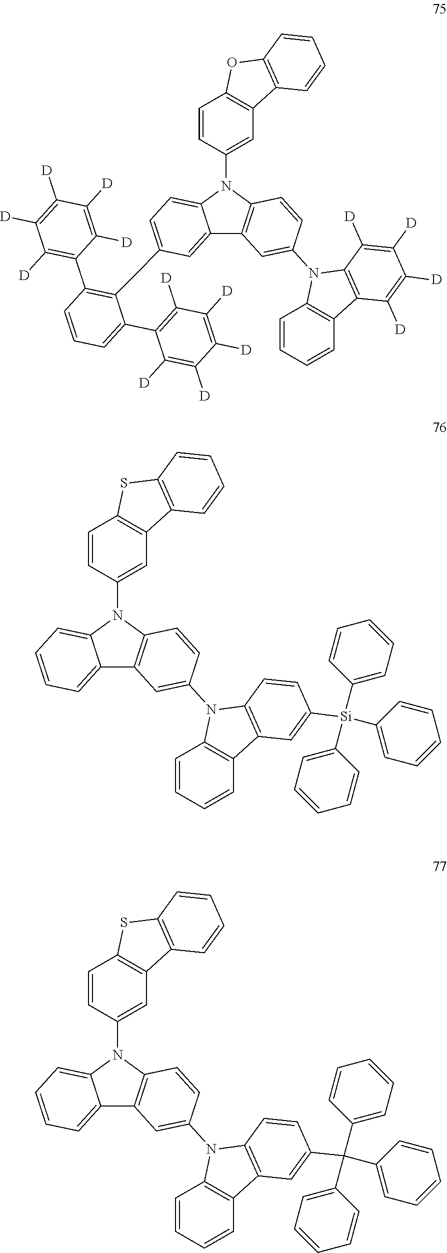 [SMILES: [2H]c1c([2H])c([2H])c(-c2cccc(-c3c([2H])c([2H])c([2H])c([2H])c3[2H])c2-c2ccc3c(c2)c2cc(-n4c5ccccc5c5c([2H])c([2H])c([2H])c([2H])c54)ccc2n3-c2ccc3oc4ccccc4c3c2)c([2H])c1[2H] ; c1ccc([Si](c2ccccc2)(c2ccccc2)c2ccc3c(c2)c2ccccc2n3-c2ccc3c(c2)c2ccccc2n3-c2ccc3sc4ccccc4c3c2)cc1 ; c1ccc(C(c2ccccc2)(c2ccccc2)c2ccc3c(c2)c2ccccc2n3-c2ccc3c(c2)c2ccccc2n3-c2ccc3sc4ccccc4c3c2)cc1]

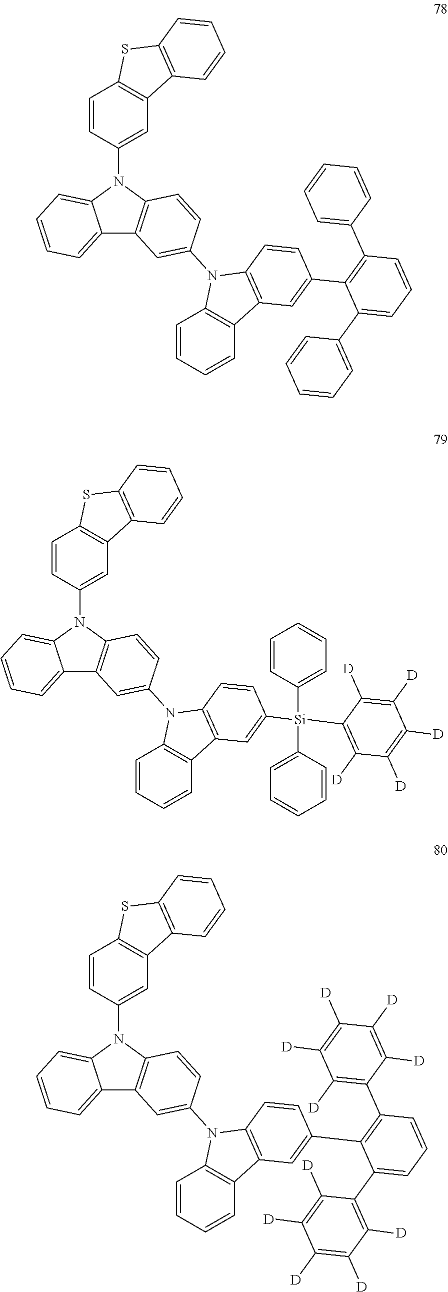 [SMILES: c1ccc(-c2cccc(-c3ccccc3)c2-c2ccc3c(c2)c2ccccc2n3-c2ccc3c(c2)c2ccccc2n3-c2ccc3sc4ccccc4c3c2)cc1 ; [2H]c1c([2H])c([2H])c([Si](c2ccccc2)(c2ccccc2)c2ccc3c(c2)c2ccccc2n3-c2ccc3c(c2)c2ccccc2n3-c2ccc3sc4ccccc4c3c2)c([2H])c1[2H] ; [2H]c1c([2H])c([2H])c(-c2cccc(-c3c([2H])c([2H])c([2H])c([2H])c3[2H])c2-c2ccc3c(c2)c2ccccc2n3-c2ccc3c(c2)c2ccccc2n3-c2ccc3sc4ccccc4c3c2)c([2H])c1[2H]]

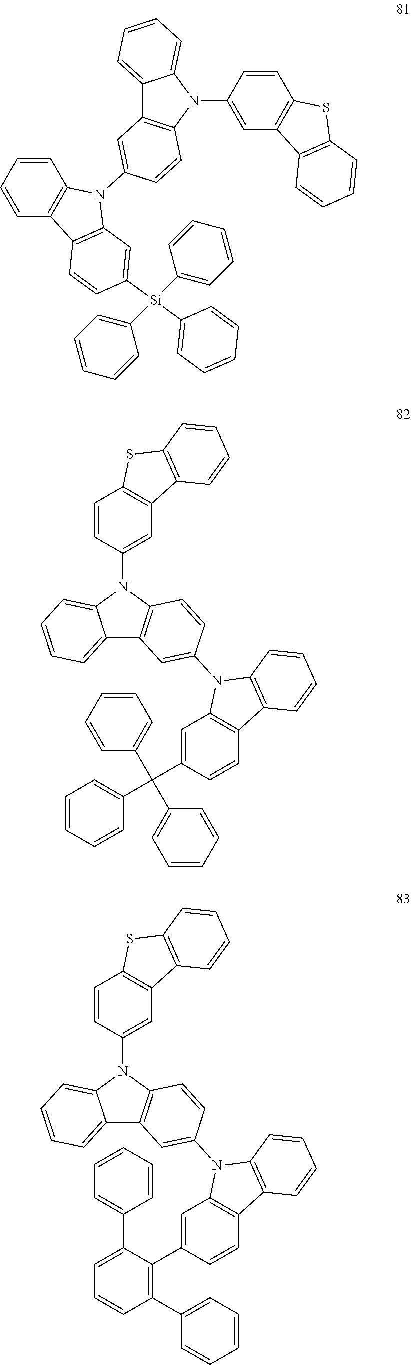

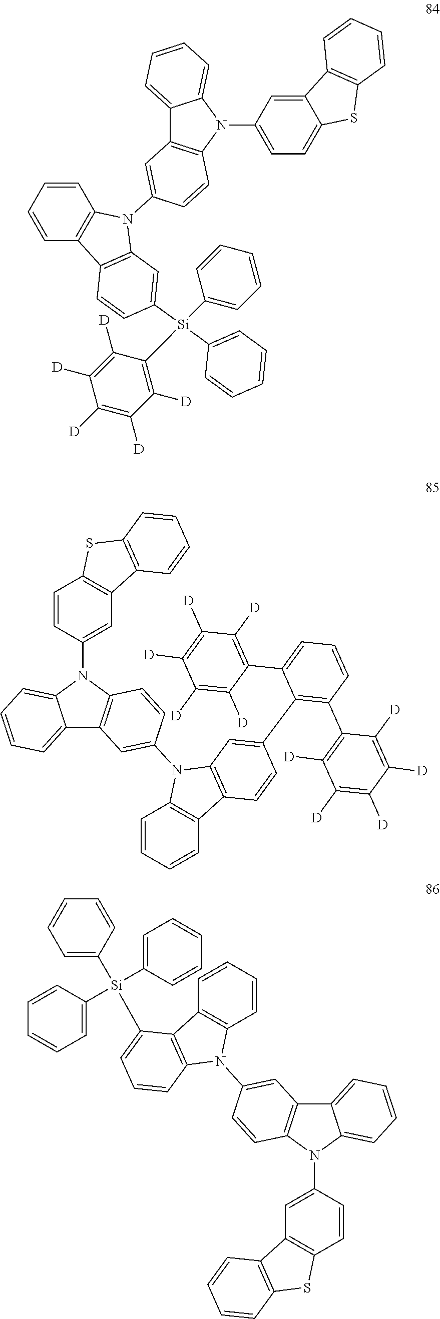 [SMILES: [2H]c1c([2H])c([2H])c([Si](c2ccccc2)(c2ccccc2)c2ccc3c4ccccc4n(-c4ccc5c(c4)c4ccccc4n5-c4ccc5sc6ccccc6c5c4)c3c2)c([2H])c1[2H] ; [2H]c1c([2H])c([2H])c(-c2cccc(-c3c([2H])c([2H])c([2H])c([2H])c3[2H])c2-c2ccc3c4ccccc4n(-c4ccc5c(c4)c4ccccc4n5-c4ccc5sc6ccccc6c5c4)c3c2)c([2H])c1[2H] ; c1ccc([Si](c2ccccc2)(c2ccccc2)c2cccc3c2c2ccccc2n3-c2ccc3c(c2)c2ccccc2n3-c2ccc3sc4ccccc4c3c2)cc1]

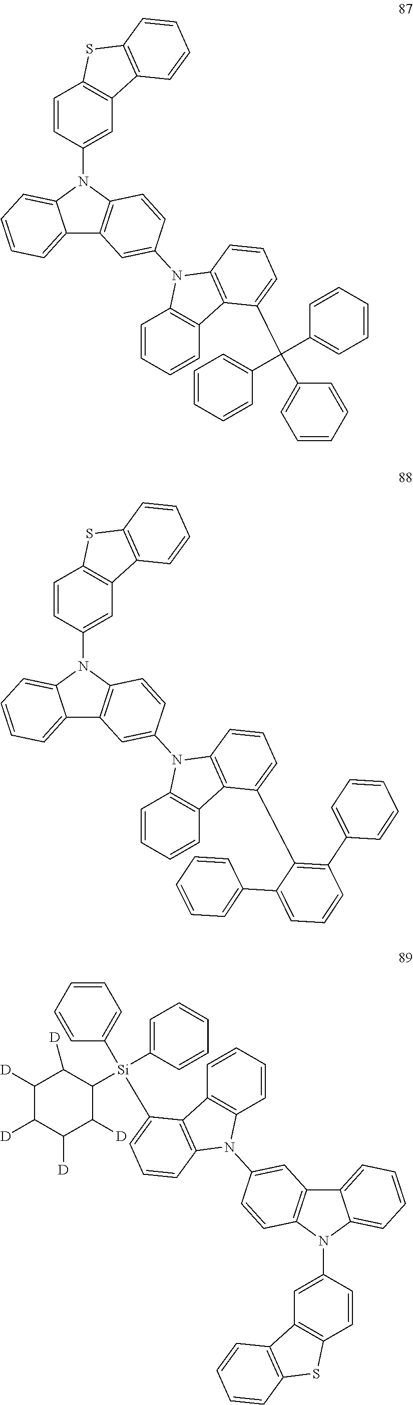 [SMILES: c1ccc(C(c2ccccc2)(c2ccccc2)c2cccc3c2c2ccccc2n3-c2ccc3c(c2)c2ccccc2n3-c2ccc3sc4ccccc4c3c2)cc1 ; c1ccc(-c2cccc(-c3ccccc3)c2-c2cccc3c2c2ccccc2n3-c2ccc3c(c2)c2ccccc2n3-c2ccc3sc4ccccc4c3c2)cc1 ; [2H]C1C([2H])C([2H])C([Si](c2ccccc2)(c2ccccc2)c2cccc3c2c2ccccc2n3-c2ccc3c(c2)c2ccccc2n3-c2ccc3sc4ccccc4c3c2)C([2H])C1[2H]]

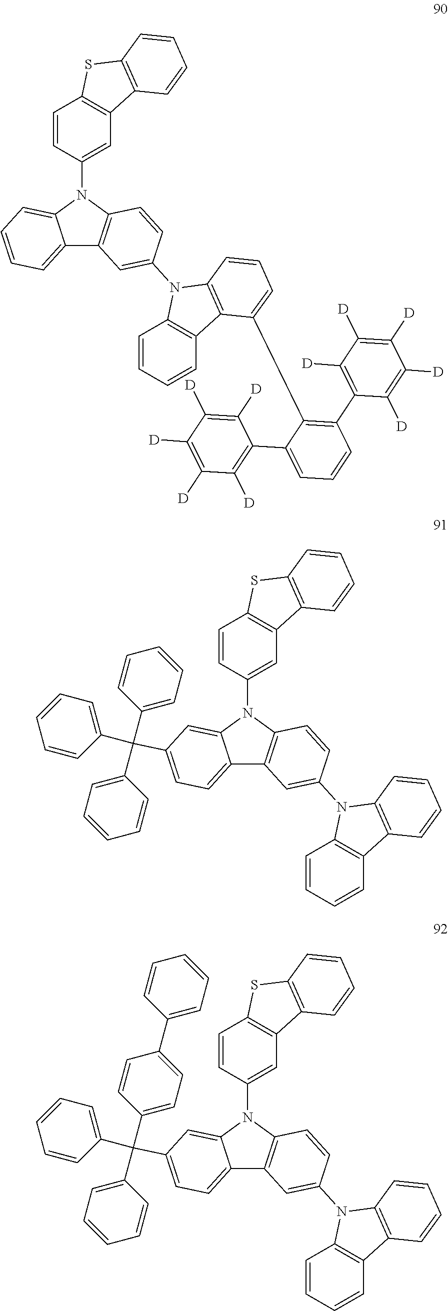 [SMILES: [2H]c1c([2H])c([2H])c(-c2cccc(-c3c([2H])c([2H])c([2H])c([2H])c3[2H])c2-c2cccc3c2c2ccccc2n3-c2ccc3c(c2)c2ccccc2n3-c2ccc3sc4ccccc4c3c2)c([2H])c1[2H] ; c1ccc(C(c2ccccc2)(c2ccccc2)c2ccc3c4cc(-n5c6ccccc6c6ccccc65)ccc4n(-c4ccc5sc6ccccc6c5c4)c3c2)cc1 ; c1ccc(-c2ccc(C(c3ccccc3)(c3ccccc3)c3ccc4c5cc(-n6c7ccccc7c7ccccc76)ccc5n(-c5ccc6sc7ccccc7c6c5)c4c3)cc2)cc1]

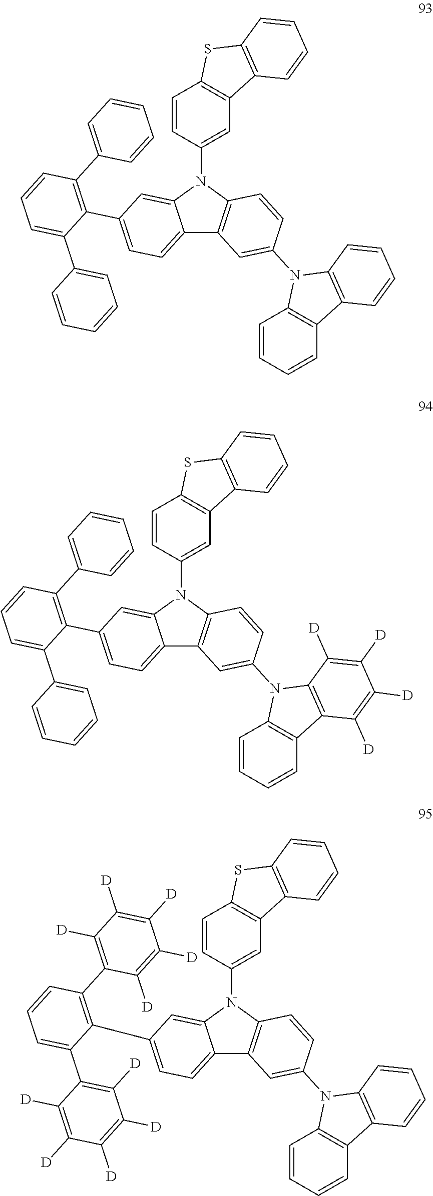 [SMILES: c1ccc(-c2cccc(-c3ccccc3)c2-c2ccc3c4cc(-n5c6ccccc6c6ccccc65)ccc4n(-c4ccc5sc6ccccc6c5c4)c3c2)cc1 ; [2H]c1c([2H])c([2H])c2c(c1[2H])c1ccccc1n2-c1ccc2c(c1)c1ccc(-c3c(-c4ccccc4)cccc3-c3ccccc3)cc1n2-c1ccc2sc3ccccc3c2c1 ; [2H]c1c([2H])c([2H])c(-c2cccc(-c3c([2H])c([2H])c([2H])c([2H])c3[2H])c2-c2ccc3c4cc(-n5c6ccccc6c6ccccc65)ccc4n(-c4ccc5sc6ccccc6c5c4)c3c2)c([2H])c1[2H]]

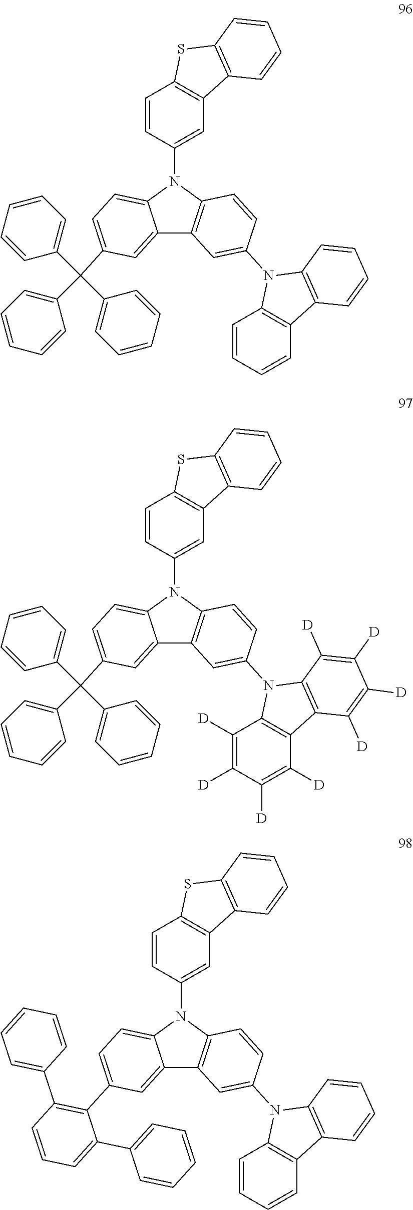

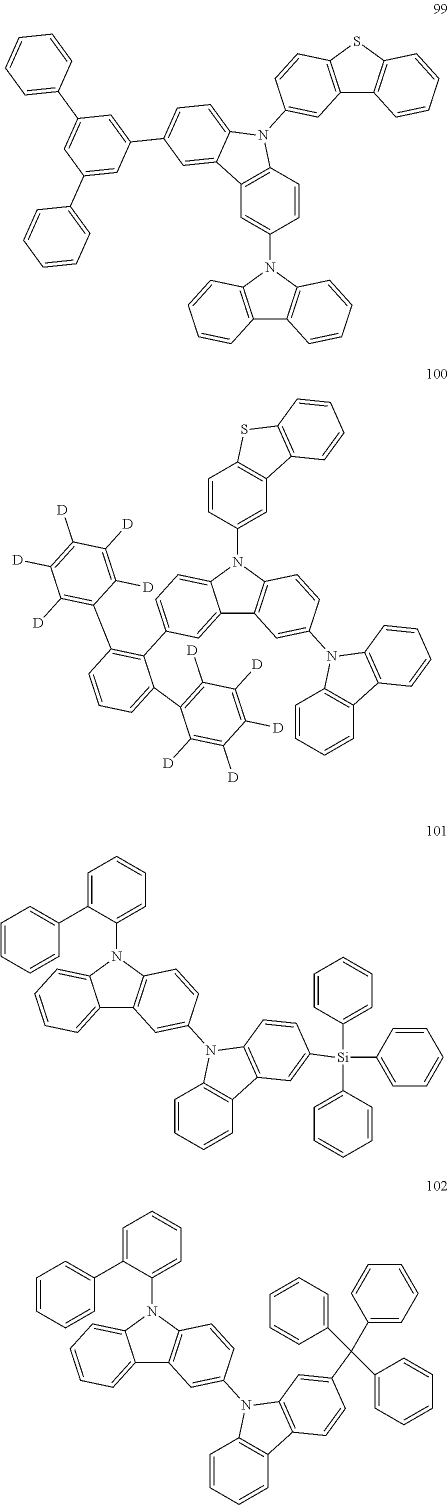 [SMILES: c1ccc(-c2cc(-c3ccccc3)cc(-c3ccc4c(c3)c3cc(-n5c6ccccc6c6ccccc65)ccc3n4-c3ccc4sc5ccccc5c4c3)c2)cc1 ; [2H]c1c([2H])c([2H])c(-c2cccc(-c3c([2H])c([2H])c([2H])c([2H])c3[2H])c2-c2ccc3c(c2)c2cc(-n4c5ccccc5c5ccccc54)ccc2n3-c2ccc3sc4ccccc4c3c2)c([2H])c1[2H] ; c1ccc(-c2ccccc2-n2c3ccccc3c3cc(-n4c5ccccc5c5cc([Si](c6ccccc6)(c6ccccc6)c6ccccc6)ccc54)ccc32)cc1 ; c1ccc(-c2ccccc2-n2c3ccccc3c3cc(-n4c5ccccc5c5ccc(C(c6ccccc6)(c6ccccc6)c6ccccc6)cc54)ccc32)cc1]

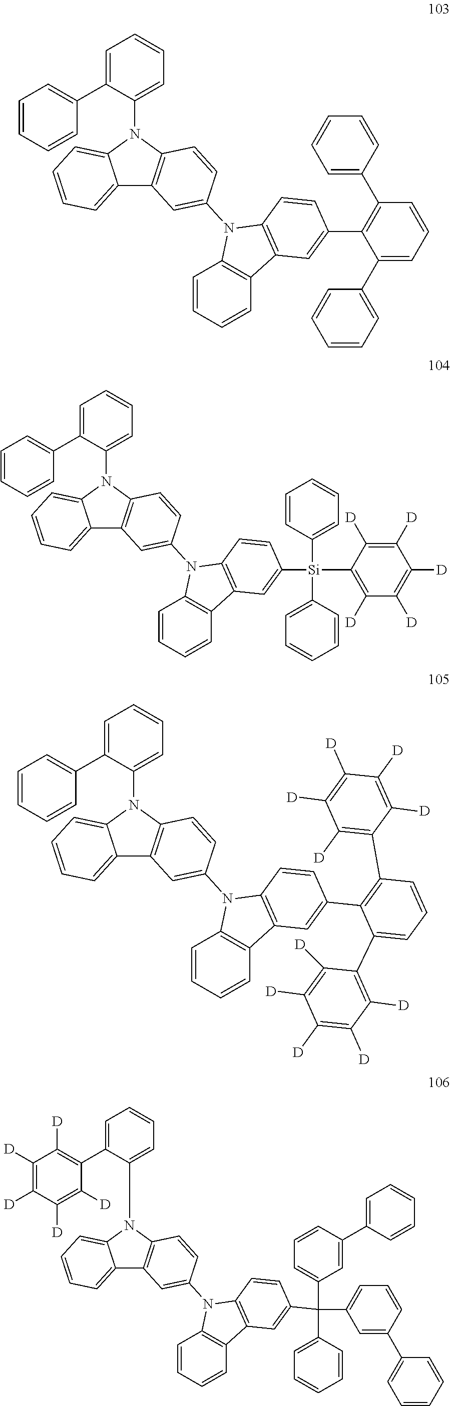

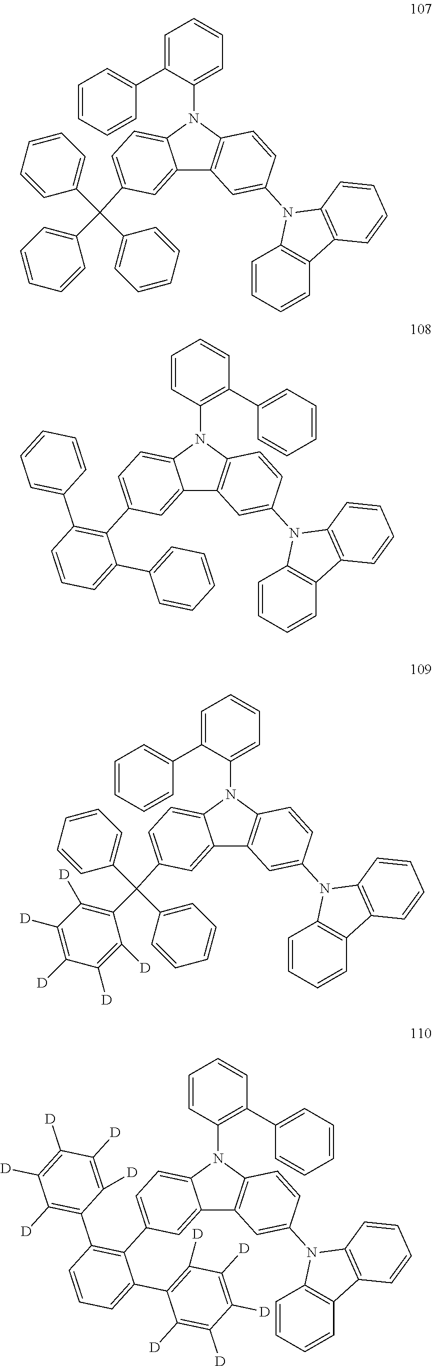 [SMILES: c1ccc(-c2ccccc2-n2c3ccc(-n4c5ccccc5c5ccccc54)cc3c3cc(C(c4ccccc4)(c4ccccc4)c4ccccc4)ccc32)cc1 ; c1ccc(-c2ccccc2-n2c3ccc(-c4c(-c5ccccc5)cccc4-c4ccccc4)cc3c3cc(-n4c5ccccc5c5ccccc54)ccc32)cc1 ; [2H]c1c([2H])c([2H])c(C(c2ccccc2)(c2ccccc2)c2ccc3c(c2)c2cc(-n4c5ccccc5c5ccccc54)ccc2n3-c2ccccc2-c2ccccc2)c([2H])c1[2H] ; [2H]c1c([2H])c([2H])c(-c2cccc(-c3c([2H])c([2H])c([2H])c([2H])c3[2H])c2-c2ccc3c(c2)c2cc(-n4c5ccccc5c5ccccc54)ccc2n3-c2ccccc2-c2ccccc2)c([2H])c1[2H]]

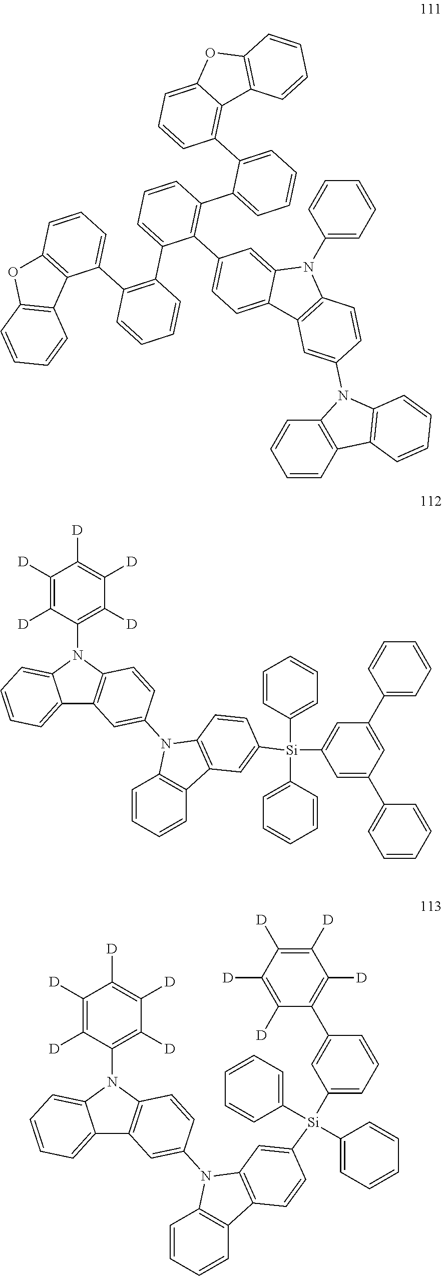 [SMILES: c1ccc(-n2c3ccc(-n4c5ccccc5c5ccccc54)cc3c3ccc(-c4c(-c5ccccc5-c5cccc6oc7ccccc7c56)cccc4-c4ccccc4-c4cccc5oc6ccccc6c45)cc32)cc1 ; [2H]c1c([2H])c([2H])c(-n2c3ccccc3c3cc(-n4c5ccccc5c5cc([Si](c6ccccc6)(c6ccccc6)c6cc(-c7ccccc7)cc(-c7ccccc7)c6)ccc54)ccc32)c([2H])c1[2H] ; [2H]c1c([2H])c([2H])c(-c2cccc([Si](c3ccccc3)(c3ccccc3)c3ccc4c5ccccc5n(-c5ccc6c(c5)c5ccccc5n6-c5c([2H])c([2H])c([2H])c([2H])c5[2H])c4c3)c2)c([2H])c1[2H]]

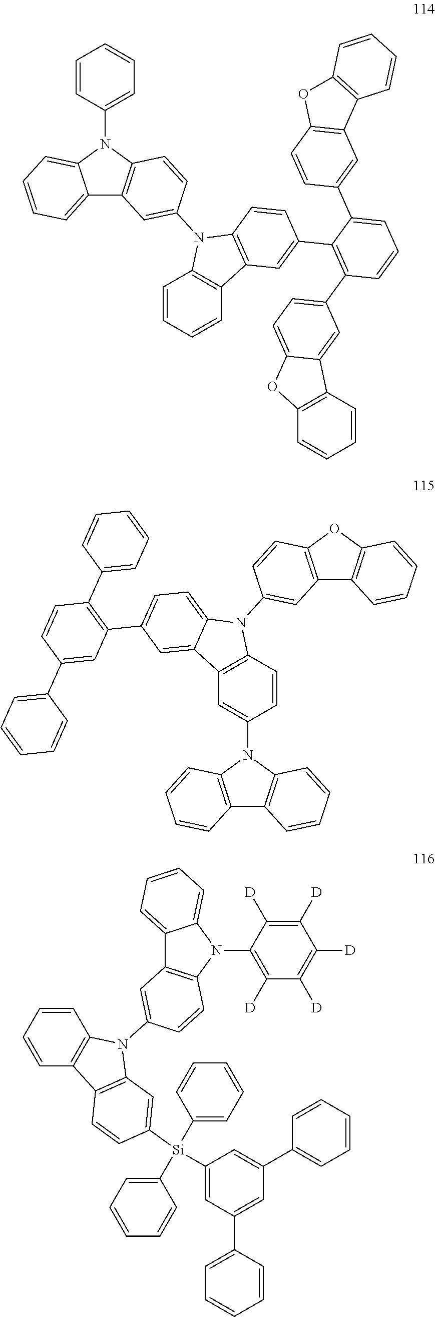

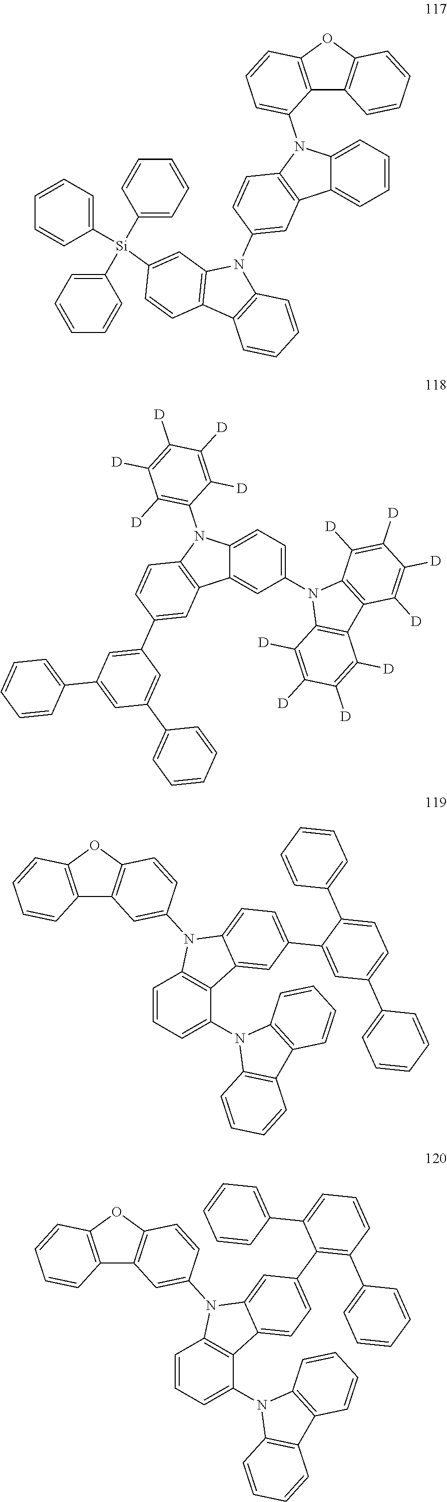 [SMILES: c1ccc([Si](c2ccccc2)(c2ccccc2)c2ccc3c4ccccc4n(-c4ccc5c(c4)c4ccccc4n5-c4cccc5oc6ccccc6c45)c3c2)cc1 ; [2H]c1c([2H])c([2H])c(-n2c3ccc(-c4cc(-c5ccccc5)cc(-c5ccccc5)c4)cc3c3cc(-n4c5c([2H])c([2H])c([2H])c([2H])c5c5c([2H])c([2H])c([2H])c([2H])c54)ccc32)c([2H])c1[2H] ; c1ccc(-c2ccc(-c3ccccc3)c(-c3ccc4c(c3)c3c(-n5c6ccccc6c6ccccc65)cccc3n4-c3ccc4oc5ccccc5c4c3)c2)cc1 ; c1ccc(-c2cccc(-c3ccccc3)c2-c2ccc3c4c(-n5c6ccccc6c6ccccc65)cccc4n(-c4ccc5oc6ccccc6c5c4)c3c2)cc1]

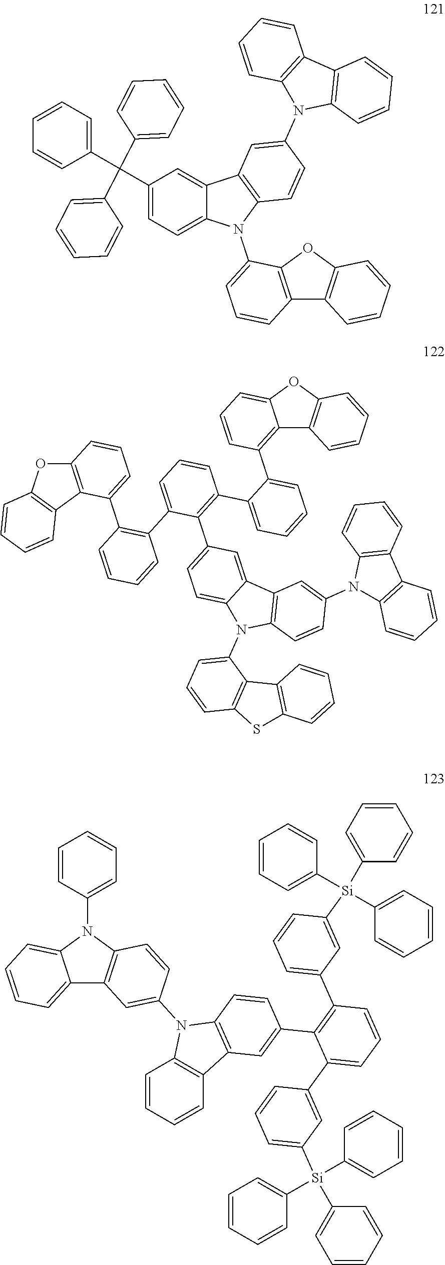 [SMILES: c1ccc(C(c2ccccc2)(c2ccccc2)c2ccc3c(c2)c2cc(-n4c5ccccc5c5ccccc54)ccc2n3-c2cccc3c2oc2ccccc23)cc1 ; c1ccc(-c2cccc3oc4ccccc4c23)c(-c2cccc(-c3ccccc3-c3cccc4oc5ccccc5c34)c2-c2ccc3c(c2)c2cc(-n4c5ccccc5c5ccccc54)ccc2n3-c2cccc3sc4ccccc4c23)c1 ; c1ccc(-n2c3ccccc3c3cc(-n4c5ccccc5c5cc(-c6c(-c7cccc([Si](c8ccccc8)(c8ccccc8)c8ccccc8)c7)cccc6-c6cccc([Si](c7ccccc7)(c7ccccc7)c7ccccc7)c6)ccc54)ccc32)cc1]

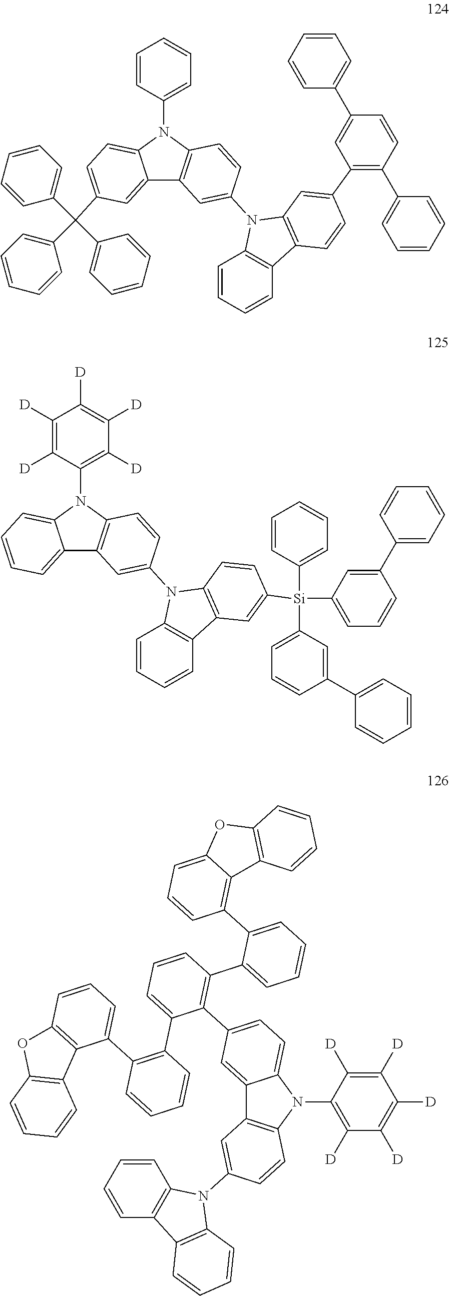 [SMILES: c1ccc(-c2ccc(-c3ccccc3)c(-c3ccc4c5ccccc5n(-c5ccc6c(c5)c5cc(C(c7ccccc7)(c7ccccc7)c7ccccc7)ccc5n6-c5ccccc5)c4c3)c2)cc1 ; [2H]c1c([2H])c([2H])c(-n2c3ccccc3c3cc(-n4c5ccccc5c5cc([Si](c6ccccc6)(c6cccc(-c7ccccc7)c6)c6cccc(-c7ccccc7)c6)ccc54)ccc32)c([2H])c1[2H] ; [2H]c1c([2H])c([2H])c(-n2c3ccc(-c4c(-c5ccccc5-c5cccc6oc7ccccc7c56)cccc4-c4ccccc4-c4cccc5oc6ccccc6c45)cc3c3cc(-n4c5ccccc5c5ccccc54)ccc32)c([2H])c1[2H]]

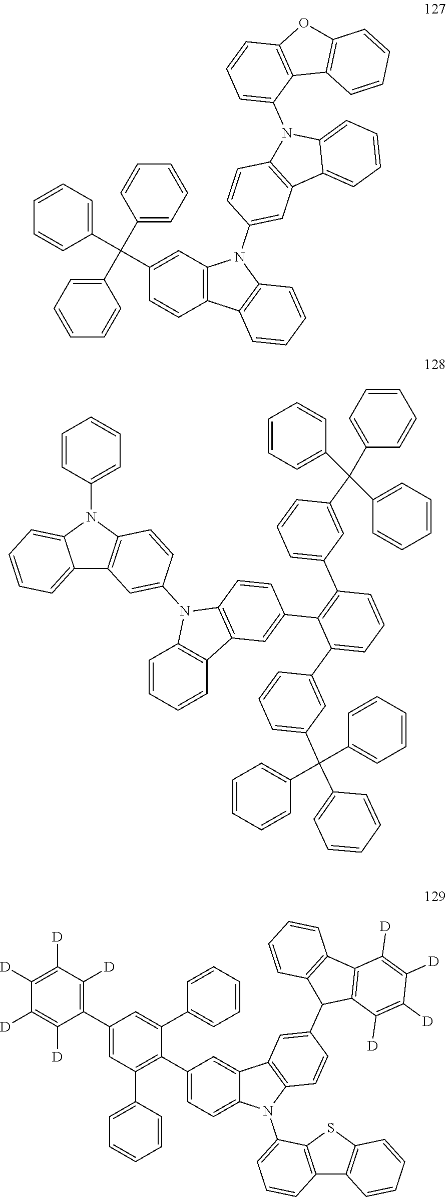 [SMILES: c1ccc(C(c2ccccc2)(c2ccccc2)c2ccc3c4ccccc4n(-c4ccc5c(c4)c4ccccc4n5-c4cccc5oc6ccccc6c45)c3c2)cc1 ; c1ccc(-n2c3ccccc3c3cc(-n4c5ccccc5c5cc(-c6c(-c7cccc(C(c8ccccc8)(c8ccccc8)c8ccccc8)c7)cccc6-c6cccc(C(c7ccccc7)(c7ccccc7)c7ccccc7)c6)ccc54)ccc32)cc1 ; [2H]c1c([2H])c([2H])c(-c2cc(-c3ccccc3)c(-c3ccc4c(c3)c3cc(C5c6ccccc6-c6c([2H])c([2H])c([2H])c([2H])c65)ccc3n4-c3cccc4c3sc3ccccc34)c(-c3ccccc3)c2)c([2H])c1[2H]]

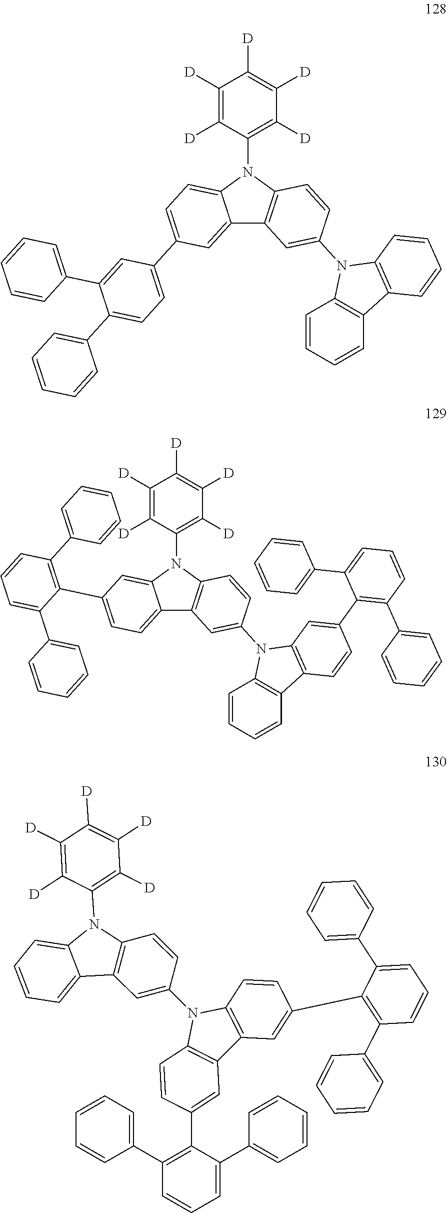 [SMILES: [2H]c1c([2H])c([2H])c(-n2c3ccc(-c4ccc(-c5ccccc5)c(-c5ccccc5)c4)cc3c3cc(-n4c5ccccc5c5ccccc54)ccc32)c([2H])c1[2H] ; [2H]c1c([2H])c([2H])c(-n2c3ccc(-n4c5ccccc5c5ccc(-c6c(-c7ccccc7)cccc6-c6ccccc6)cc54)cc3c3ccc(-c4c(-c5ccccc5)cccc4-c4ccccc4)cc32)c([2H])c1[2H] ; [2H]c1c([2H])c([2H])c(-n2c3ccccc3c3cc(-n4c5ccc(-c6c(-c7ccccc7)cccc6-c6ccccc6)cc5c5cc(-c6c(-c7ccccc7)cccc6-c6ccccc6)ccc54)ccc32)c([2H])c1[2H]]

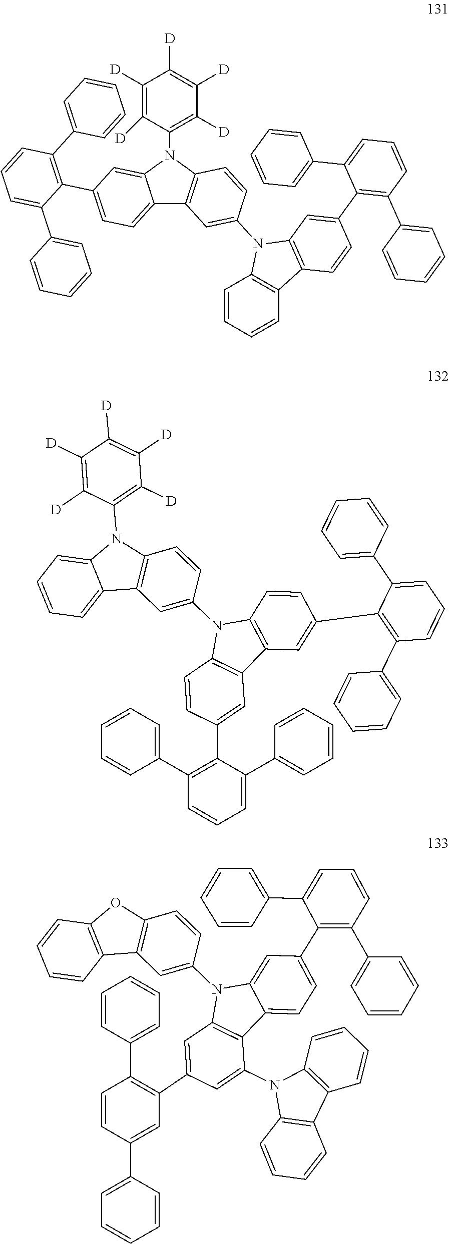

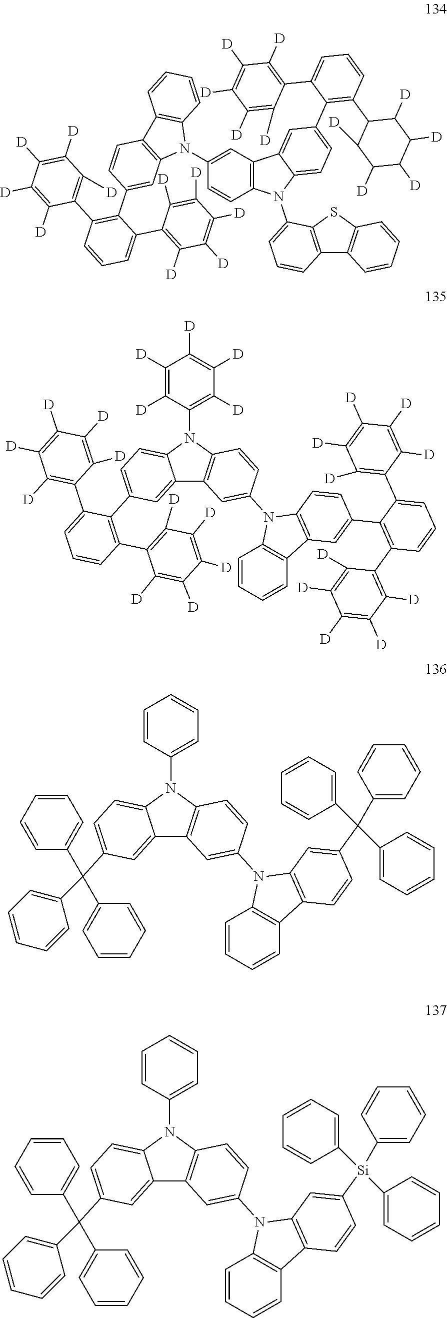

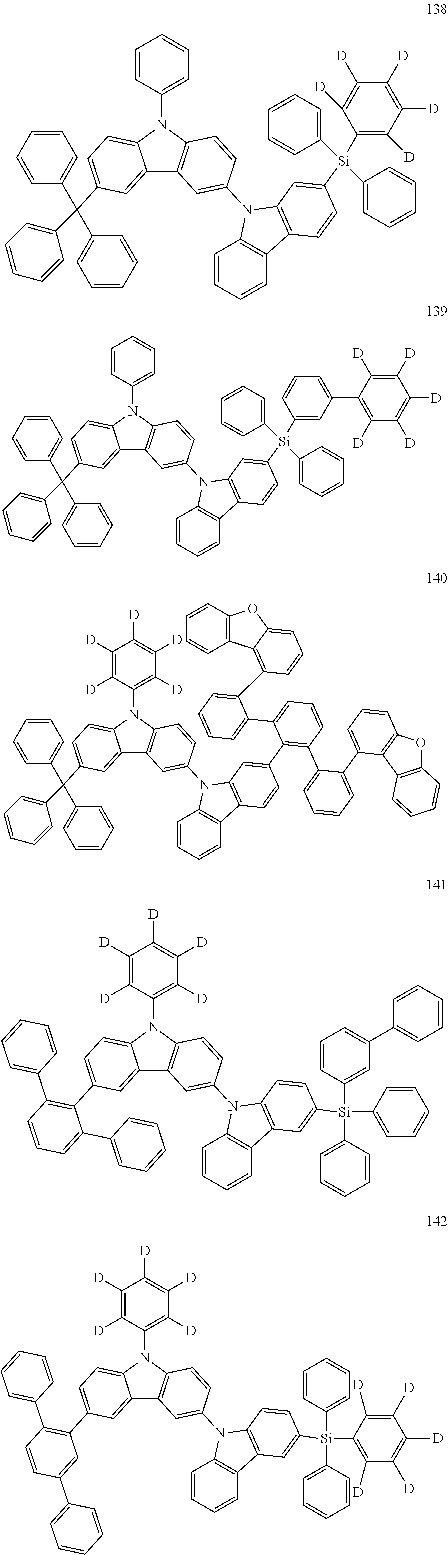

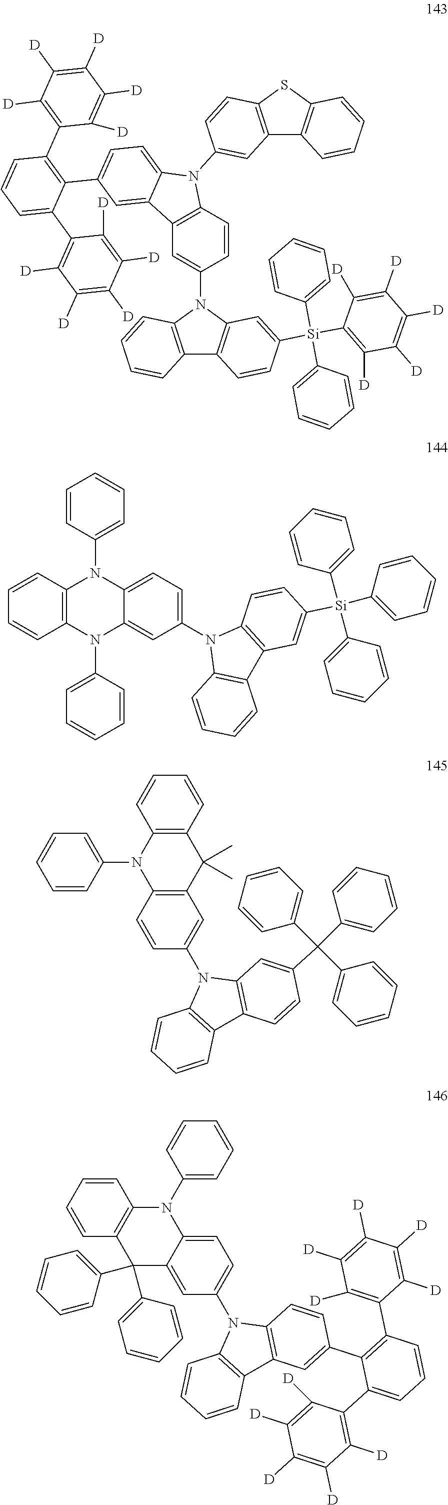

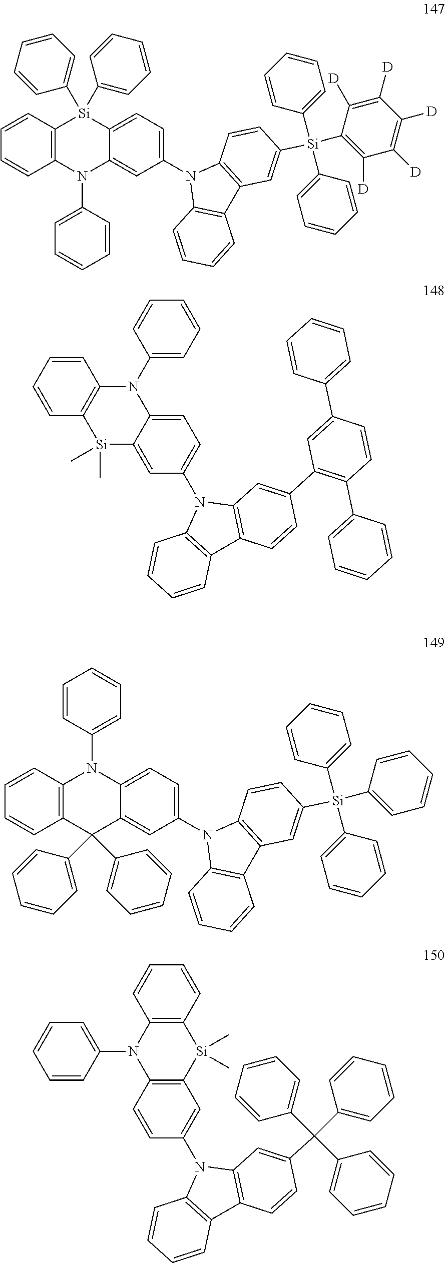

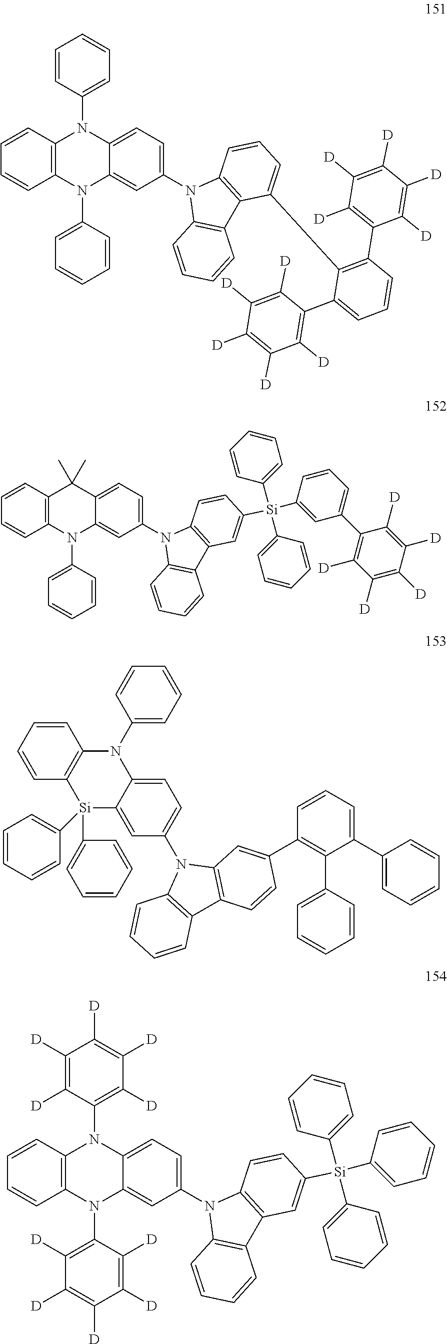

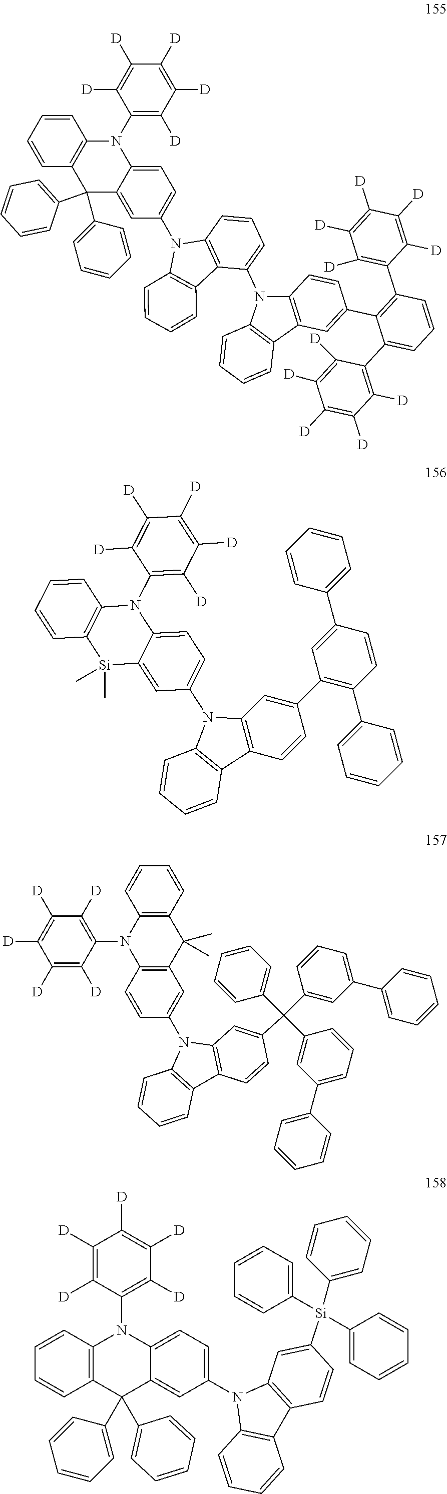

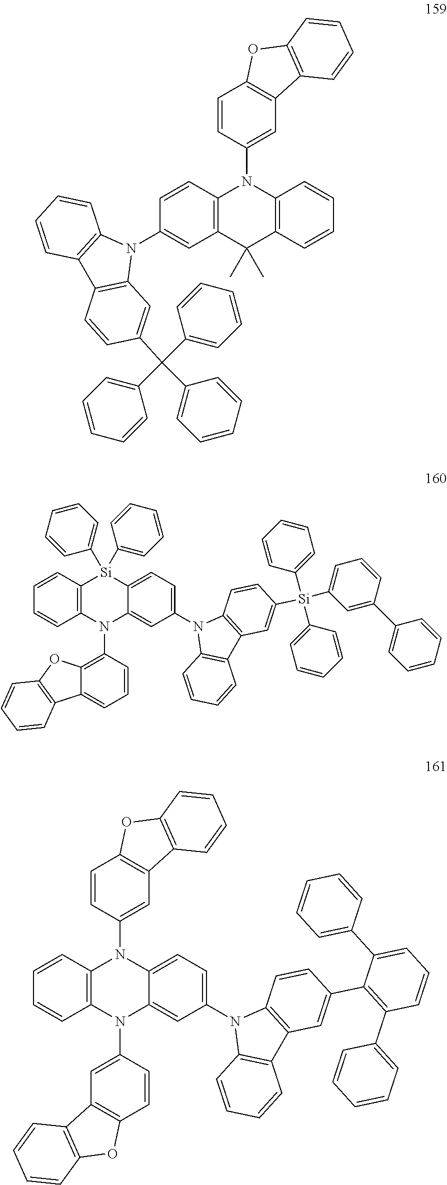 [SMILES: CC1(C)c2ccccc2N(c2ccc3oc4ccccc4c3c2)c2ccc(-n3c4ccccc4c4ccc(C(c5ccccc5)(c5ccccc5)c5ccccc5)cc43)cc21 ; c1ccc(-c2cccc([Si](c3ccccc3)(c3ccccc3)c3ccc4c(c3)c3ccccc3n4-c3ccc4c(c3)N(c3cccc5c3oc3ccccc35)c3ccccc3[Si]4(c3ccccc3)c3ccccc3)c2)cc1 ; c1ccc(-c2cccc(-c3ccccc3)c2-c2ccc3c(c2)c2ccccc2n3-c2ccc3c(c2)N(c2ccc4oc5ccccc5c4c2)c2ccccc2N3c2ccc3oc4ccccc4c3c2)cc1]

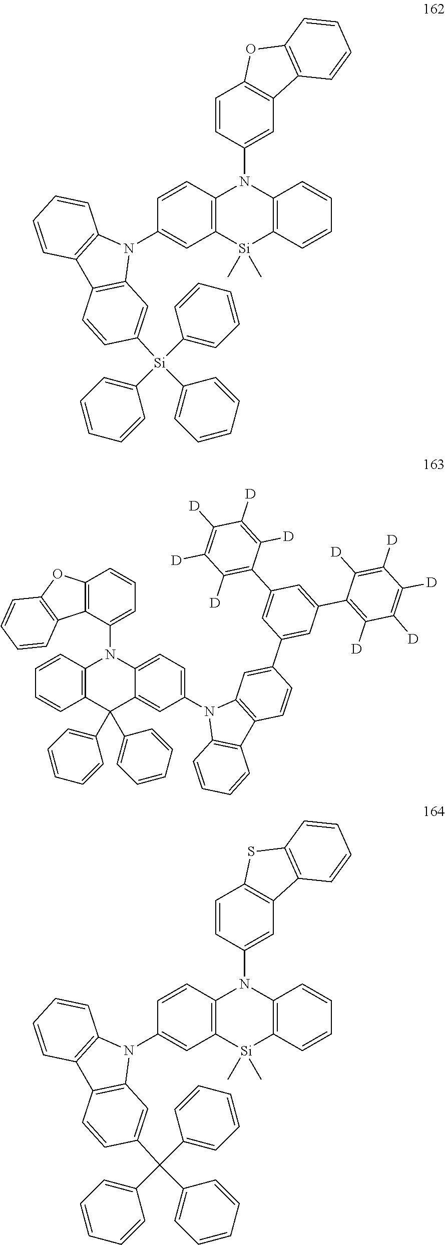 [SMILES: C[Si]1(C)c2ccccc2N(c2ccc3oc4ccccc4c3c2)c2ccc(-n3c4ccccc4c4ccc([Si](c5ccccc5)(c5ccccc5)c5ccccc5)cc43)cc21 ; [2H]c1c([2H])c([2H])c(-c2cc(-c3ccc4c5ccccc5n(-c5ccc6c(c5)C(c5ccccc5)(c5ccccc5)c5ccccc5N6c5cccc6oc7ccccc7c56)c4c3)cc(-c3c([2H])c([2H])c([2H])c([2H])c3[2H])c2)c([2H])c1[2H] ; C[Si]1(C)c2ccccc2N(c2ccc3sc4ccccc4c3c2)c2ccc(-n3c4ccccc4c4ccc(C(c5ccccc5)(c5ccccc5)c5ccccc5)cc43)cc21]

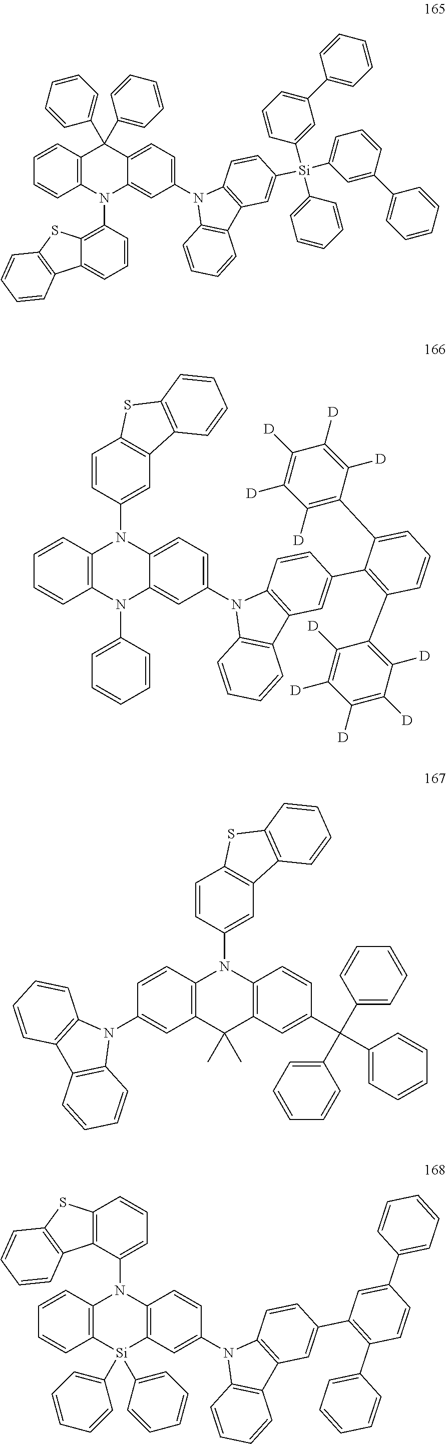

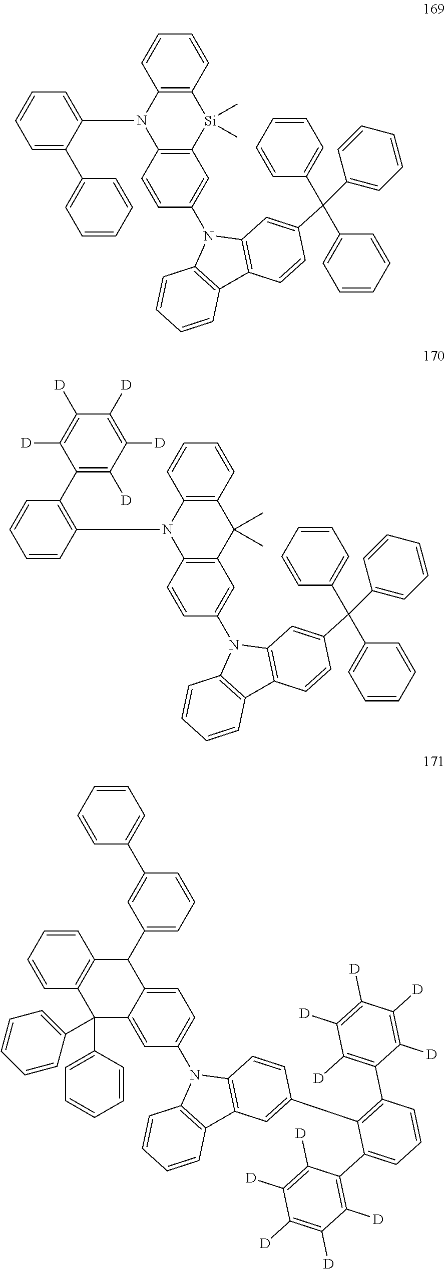 [SMILES: C[Si]1(C)c2ccccc2N(c2ccccc2-c2ccccc2)c2ccc(-n3c4ccccc4c4ccc(C(c5ccccc5)(c5ccccc5)c5ccccc5)cc43)cc21 ; [2H]c1c([2H])c([2H])c(-c2ccccc2N2c3ccccc3C(C)(C)c3cc(-n4c5ccccc5c5ccc(C(c6ccccc6)(c6ccccc6)c6ccccc6)cc54)ccc32)c([2H])c1[2H] ; [2H]c1c([2H])c([2H])c(-c2cccc(-c3c([2H])c([2H])c([2H])c([2H])c3[2H])c2-c2ccc3c(c2)c2ccccc2n3-c2ccc3c(c2)C(c2ccccc2)(c2ccccc2)c2ccccc2C3c2cccc(-c3ccccc3)c2)c([2H])c1[2H]]

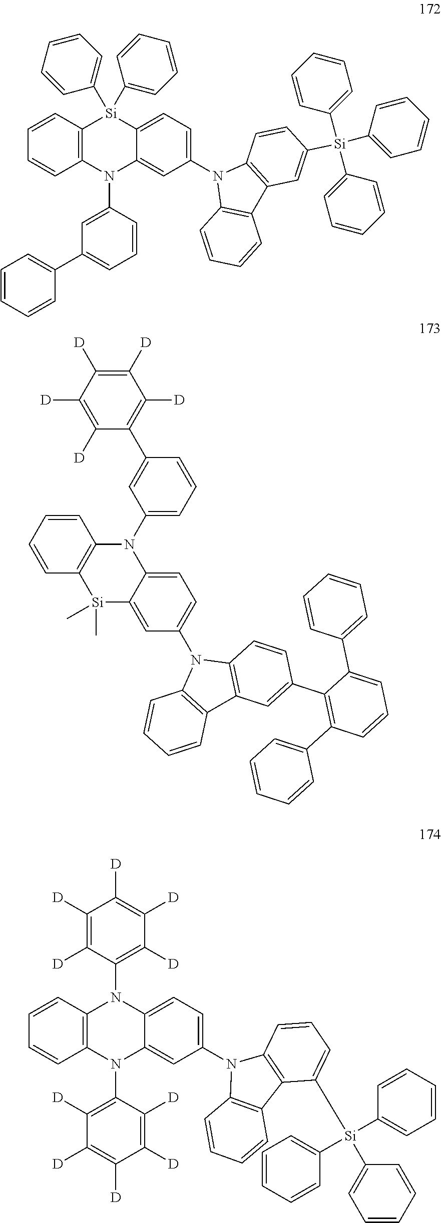 [SMILES: c1ccc(-c2cccc(N3c4ccccc4[Si](c4ccccc4)(c4ccccc4)c4ccc(-n5c6ccccc6c6cc([Si](c7ccccc7)(c7ccccc7)c7ccccc7)ccc65)cc43)c2)cc1 ; [2H]c1c([2H])c([2H])c(-c2cccc(N3c4ccccc4[Si](C)(C)c4cc(-n5c6ccccc6c6cc(-c7c(-c8ccccc8)cccc7-c7ccccc7)ccc65)ccc43)c2)c([2H])c1[2H] ; [2H]c1c([2H])c([2H])c(N2c3ccccc3N(c3c([2H])c([2H])c([2H])c([2H])c3[2H])c3cc(-n4c5ccccc5c5c([Si](c6ccccc6)(c6ccccc6)c6ccccc6)cccc54)ccc32)c([2H])c1[2H]]

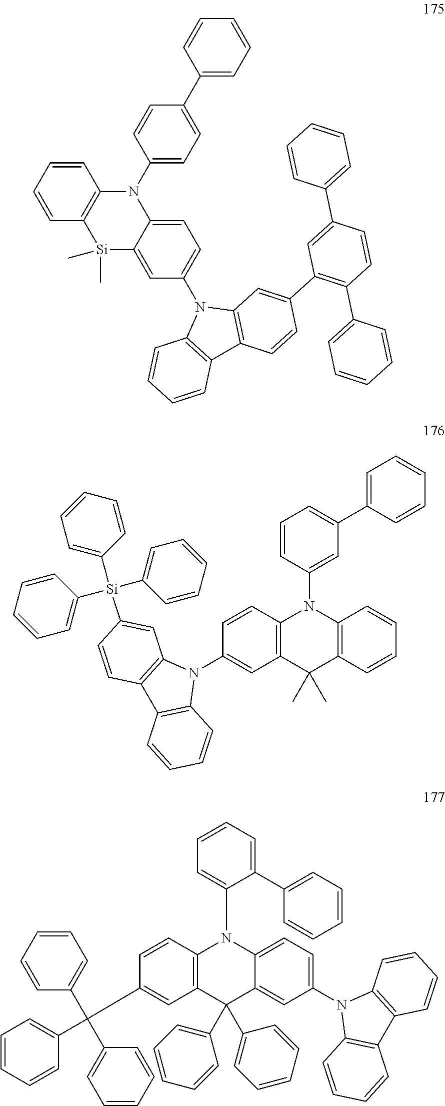 [SMILES: C[Si]1(C)c2ccccc2N(c2ccc(-c3ccccc3)cc2)c2ccc(-n3c4ccccc4c4ccc(-c5cc(-c6ccccc6)ccc5-c5ccccc5)cc43)cc21 ; CC1(C)c2ccccc2N(c2cccc(-c3ccccc3)c2)c2ccc(-n3c4ccccc4c4ccc([Si](c5ccccc5)(c5ccccc5)c5ccccc5)cc43)cc21 ; c1ccc(-c2ccccc2N2c3ccc(-n4c5ccccc5c5ccccc54)cc3C(c3ccccc3)(c3ccccc3)c3cc(C(c4ccccc4)(c4ccccc4)c4ccccc4)ccc32)cc1]

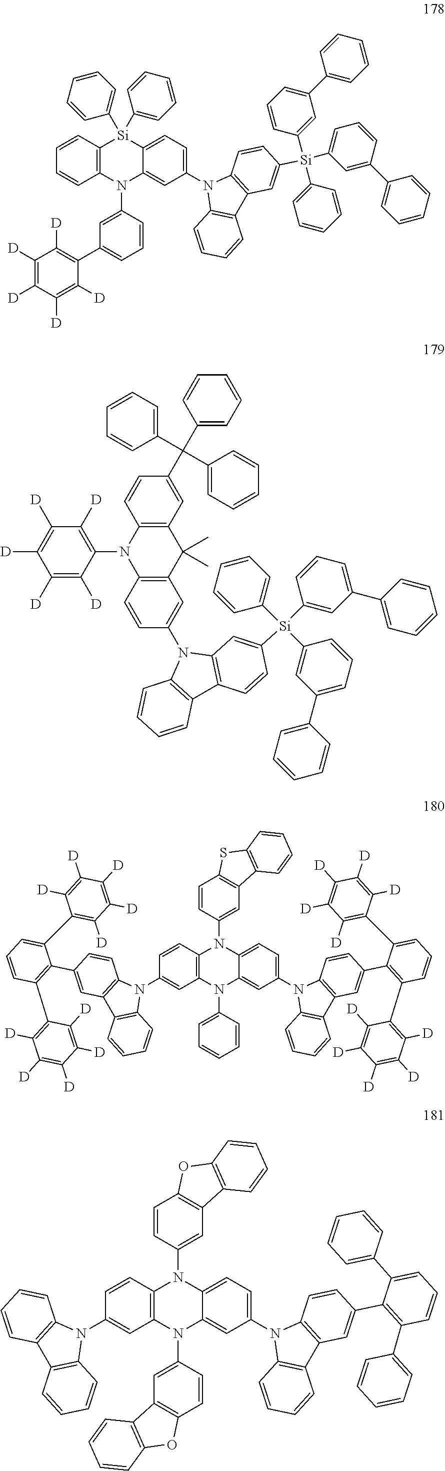

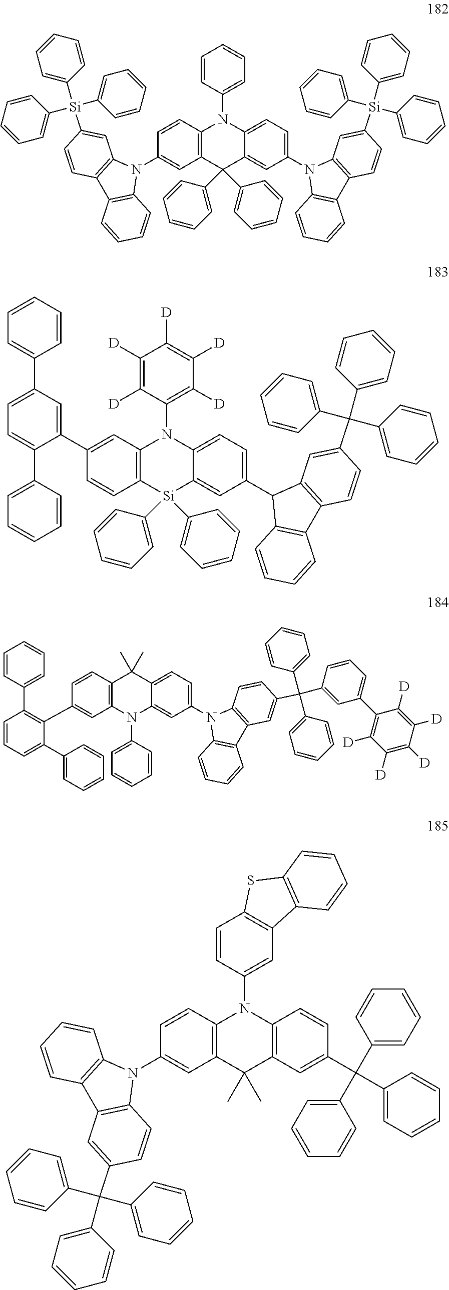

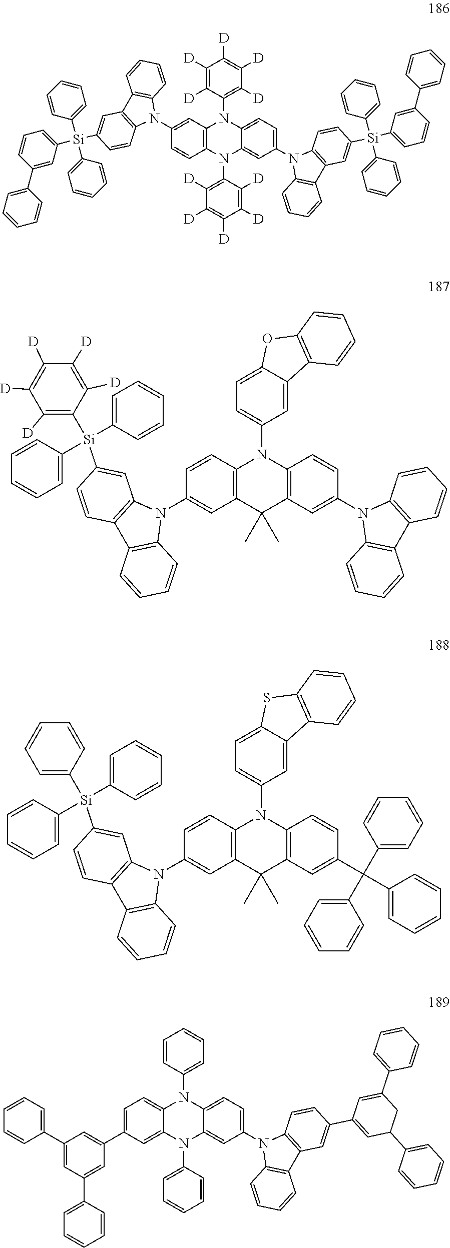

Regarding the heterocyclic compound, i) when a2 and c1 are each 1 and a1, a3, a4, and c2 are each 0, in Formulae 1 to 3, Ar₂ is not a triphenylsilane group. Accordingly, hole transportability and charge transportability may be improved, and hole mobility and charge mobility may be improved; ii) because the sum of a1 to a4 is 1 or more, the intermolecular attraction may be lowered through steric hindrance and thus relatively high triplet energy may be obtained. Accordingly, the heterocylic compound may be suitable as a phosphorescent host; iii) because the sum of c1 and c2 is 1 or more, the electron-donating group is substituted and the hole transportability may be improved. Accordingly, the driving voltage is lowered and the charge balance characteristic is improved, and thus luminescence efficiency may be improved. In addition, in the case of the heterocyclic compound according to an embodiment, the energy level of a compound and the polarity of a molecule may be easily controlled by introducing various substituents. Thus, an electronic device, for example, an organic light-emitting device, including the heterocyclic compound may have low driving voltage, excellent (e.g., high) luminescence efficiency and/or long lifetime.

Synthesis methods of the heterocyclic compound represented by Formula 1 may be recognizable by one of ordinary skill in the art by referring to Synthesis Examples and/or Examples provided below.

At least one heterocyclic compound represented by Formula 1 may be utilized in a light-emitting device (for example, an organic light-emitting device). Accordingly, a light-emitting device may include: a first electrode; a second electrode facing the first electrode; and an interlayer located between the first electrode and the second electrode and including an emission layer, wherein the interlayer includes the heterocyclic compound represented by Formula 1.

In some embodiments,

the first electrode of the light-emitting device may be an anode,

the second electrode of the light-emitting device may be a cathode,

the interlayer further includes a hole transport region located between the first electrode and the emission layer and an electron transport region located between the emission layer and the second electrode,

the hole transport region includes a hole injection layer, a hole transport layer, an emission auxiliary layer, an electron blocking layer, or any combination thereof, and

the electron transport region may include a buffer layer, a hole blocking layer, an electron transport layer, an electron injection layer, or any combination thereof.

In an embodiment, the heterocyclic compound may be included between a pair of electrodes of the light-emitting device. Accordingly, the heterocyclic compound may be included in the interlayer of the light-emitting device, for example, in the emission layer of the interlayer.

In one or more embodiments, the emission layer in the interlayer of the light-emitting device may include a dopant and a host, and the heterocyclic compound may be included in the host. That is, the heterocyclic cyclic compound may act as a host. The emission layer may emit red light, green light, blue light, and/or white light. For example, the emission layer may emit blue light. In an embodiment, the heterocyclic compound may emit blue light having a maximum emission wavelength of 390 nm or more and 440 nm or less, and may have a difference between a singlet (S₁) energy level and a triplet (T₁) energy level of 0.6 eV or less.

In an embodiment, the light-emitting device may include a capping layer located outside the first electrode (e.g., located on the side opposite to the side facing the second electrode) or outside the second electrode (e.g., located on the side opposite to the side facing the first electrode).

In an embodiment, the light-emitting device may further include at least one of a first capping layer located outside the first electrode or a second capping layer located outside the second electrode, and the heterocyclic compound represented by Formula 1 may be included in at least one of the first capping layer or the second capping layer. More details for the first capping layer and/or second capping layer are the same as described in the present specification.

In one or more embodiments, the light-emitting device may further include:

a first capping layer located outside the first electrode and containing the heterocyclic compound represented by Formula 1;

a second capping layer located outside the second electrode and containing the heterocyclic compound represented by Formula 1; or

the first capping layer and the second capping layer.

The expression “(an interlayer and/or a capping layer) includes at least one heterocyclic compound” as used herein may include a case in which “(an interlayer and/or a capping layer) includes identical heterocyclic compounds represented by Formula 1” and a case in which “(an organic layer) includes two or more different heterocyclic compounds represented by Formula 1”.

For example, the interlayer and/or capping layer may include only Compound 1 as the heterocyclic compound. In this regard, Compound 1 may exist in the emission layer of the light-emitting device. In some embodiments, the interlayer may include, as the heterocyclic compounds, Compounds 1 and 2. In this regard, Compound 1 and Compound 2 may exist in an identical layer (for example, Compound 1 and Compound 2 may both exist in an emission layer), or different layers (for example, Compound 1 may exist in an emission layer and Compound 2 may exist in an electron transport region).

The term “interlayer” as used herein refers to a single layer and/or all of a plurality of layers located between a first electrode and a second electrode of a light-emitting device.

According to another embodiment, an electronic apparatus may include the light-emitting device. The electronic apparatus may further include a thin-film transistor. In one or more embodiments, the electronic apparatus may further include a thin-film transistor including a source electrode and a drain electrode, and the first electrode of the light-emitting device may be electrically connected to the source electrode or the drain electrode. In an embodiment, the electronic apparatus may further include a color filter, a color conversion layer, a touch screen layer, a polarizing layer, or any combination thereof. More details on the electronic apparatus are the same as described in the present specification.

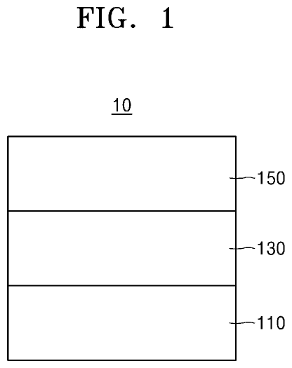

### Description of FIG. 1

FIG. 1 is a schematic cross-sectional view of a light-emitting device 10 according to an embodiment. The light-emitting device 10 includes a first electrode 110, an interlayer 130, and a second electrode 150.

Hereinafter, the structure of the light-emitting device 10 according to an embodiment and a method of manufacturing the light-emitting device 10 will be described in connection with FIG. 1.

### First Electrode 110

In FIG. 1, a substrate may be additionally located under the first electrode 110 or above the second electrode 150. As the substrate, a glass substrate or a plastic substrate may be utilized. In an embodiment, the substrate may be a flexible substrate, and may include plastics with excellent heat resistance and durability, such as polyimide, polyethylene terephthalate (PET), polycarbonate, polyethylene napthalate, polyarylate (PAR), polyetherimide, or any combination thereof.

The first electrode 110 may be formed by, for example, depositing or sputtering a material for forming the first electrode 110 on the substrate. When the first electrode 110 is an anode, a material for forming the first electrode 110 may be a high work function material that facilitates injection of holes.

The first electrode 110 may be a reflective electrode, a semi-transmissive electrode, or a transmissive electrode. When the first electrode 110 is a transmissive electrode, a material for forming the first electrode 110 may include indium tin oxide (ITO), indium zinc oxide (IZO), tin oxide (SnO₂), zinc oxide (ZnO), or any combinations thereof. In one or more embodiments, when the first electrode 110 is a semi-transmissive electrode or a reflective electrode, magnesium (Mg), silver (Ag), aluminum (Al), aluminum-lithium (Al—Li), calcium (Ca), magnesium-indium (Mg—In), magnesium-silver (Mg—Ag), or any combinations thereof may be utilized as a material for forming a first electrode.

The first electrode 110 may have a single layered structure consisting of a single-layer or a multilayer structure including a plurality of layers. For example, the first electrode 110 may have a three-layered structure of ITO/Ag/ITO.

### Interlayer 130

The interlayer 130 may be located on the first electrode 110. The interlayer 130 may include an emission layer.

The interlayer 130 may further include a hole transport region placed between the first electrode 110 and the emission layer and an electron transport region placed between the emission layer and the second electrode 150.

The interlayer 130 may further include metal-containing compounds (such as organometallic compounds), inorganic materials (such as quantum dots), and/or the like, in addition to various suitable organic materials.

In one or more embodiments, the interlayer 130 may include, i) two or more emitting units sequentially stacked between the first electrode 110 and the second electrode 150 and ii) a charge generation layer located between two adjacent emitting units. When the interlayer 130 includes the two or more emitting units and the charge generation layer as described above, the light-emitting device 10 may be a tandem light-emitting device.

### Hole Transport Region in Interlayer 130

The hole transport region may have: i) a single-layered structure consisting of a single layer consisting of a single material, ii) a single-layered structure consisting of a single layer consisting of a plurality of different materials, or iii) a multi-layered structure including a plurality of layers including different materials.

The hole transport region may include a hole injection layer, a hole transport layer, an emission auxiliary layer, an electron blocking layer, or any combination thereof.

For example, the hole transport region may have a multi-layered structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron blocking layer structure, wherein, in each structure, constituting layers are stacked sequentially from the first electrode 110 in the respective stated order.

The hole transport region may include a compound represented by Formula 201, a compound represented by Formula 202, or any combination thereof:

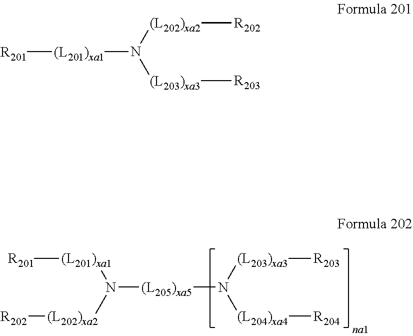

wherein, in Formulae 201 and 202,

L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

L₂₀₅ may be *—O—*′, *—S—*′, *—N(Q₂₀₁)-*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,

xa1 to xa4 may each independently an integer selected from 0 to 5,

xa5 may be an integer selected from 1 to 10,

R₂₀₁ to R₂₀₄ and Q₂₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

R₂₀₁ and R₂₀₂ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ to form a C₅-C_{60 polycyclic group (for example, a carbazole group and/or the like) unsubstituted or substituted with at least one R}₁₀ₐ (see Compound HT16 and/or the like),

R₂₀₃ and R₂₀₄ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group unsubstituted or substituted with at least one R₁₀ₐ, and

na1 may be an integer selected from 1 to 4.

In one or more embodiments, each of Formulae 201 and 202 may include at least one of groups represented by Formulae CY201 to CY217.

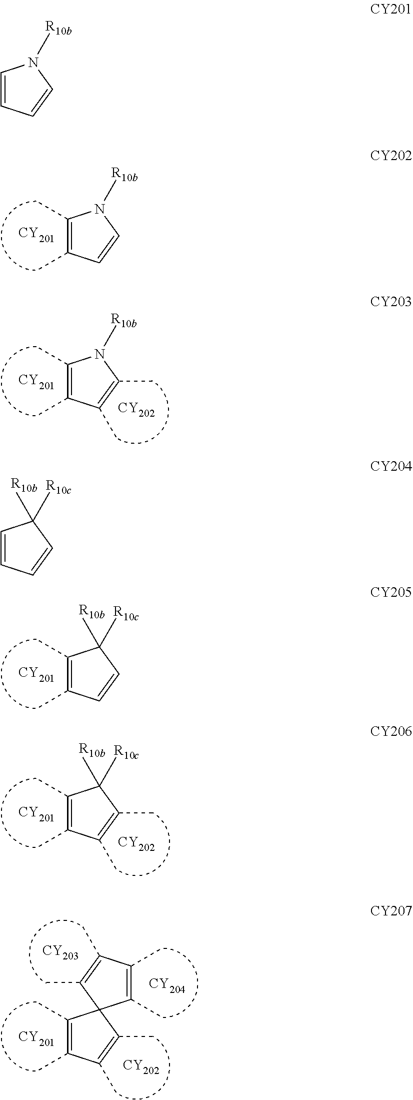

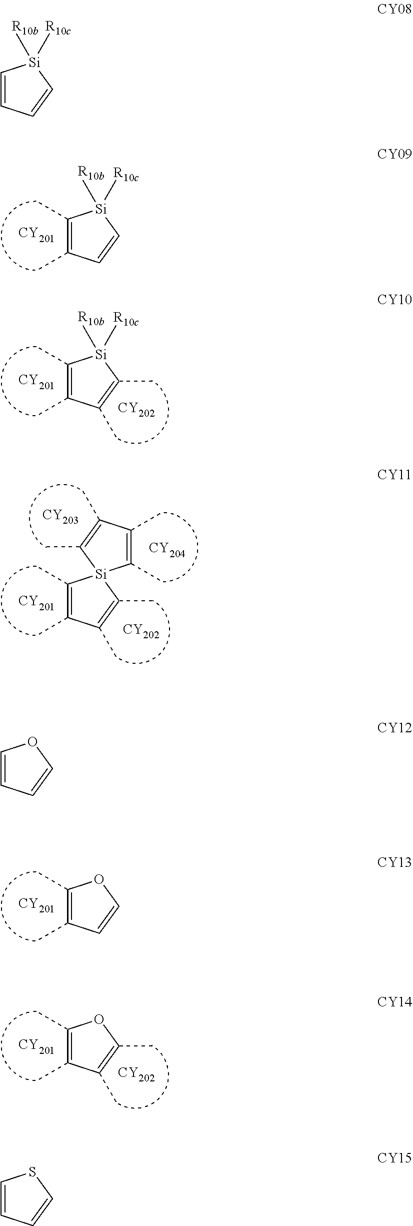

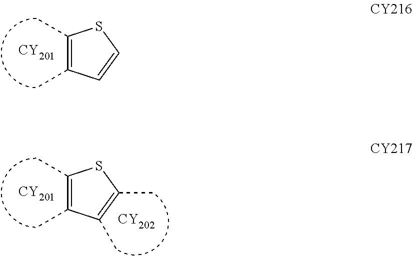

In Formulae CY201 to CY217, R_{10b} and R_{10c}, are each independently the same as described in connection with R₁₀ₐ, and ring CY₂₀₁ to ring CY₂₀₄ may each independently be a C₃-C₂₀ carbocyclic group or a C₁-C₂₀ heterocyclic group, and at least one hydrogen in Formulae CY201 to CY217 may be unsubstituted or substituted with at least one R₁₀ₐ as described in the present specification.

In an embodiment, in Formulae CY201 to CY217, ring CY₂₀₁ to ring CY₂₀₄7 may each independently be a benzene group, a naphthalene group, a phenanthrene group, or an anthracene group.

In one or more embodiments, each of Formulae 201 and 202 may include at least one of the groups represented by Formulae CY201 to CY203.

In one or more embodiments, Formula 201 may include at least one of groups represented by Formulae CY201 to CY203 and/or at least one of groups represented by Formulae CY204 to CY217.

In one or more embodiments, xa1 in Formula 201 is 1, R₂₀₁ is a group represented by one of Formulae CY201 to CY203, xa2 may be 0, and R₂₀₂ may be a group represented by one of Formulae CY204 to CY207.

In one or more embodiments, each of Formulae 201 and 202 may not include any of the groups represented by Formulae CY201 to CY203.

In one or more embodiments, each of Formulae 201 and 202 may not include any of the groups represented by Formulae CY201 to CY203, and may include at least one of groups represented by Formulae CY204 to CY217.

In an embodiment, each of Formulae 201 and 202 may not include any of the groups represented by Formulae CY201 to CY217.

In one or more embodiments, the hole transport region may include one of Compounds HT1 to HT46, m-MTDATA, TDATA, 2-TNATA, NPB(NPD), β-NPB, TPD, Spiro-TPD, Spiro-NPB, methylated-NPB, TAPC, HMTPD, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), or any combination thereof:

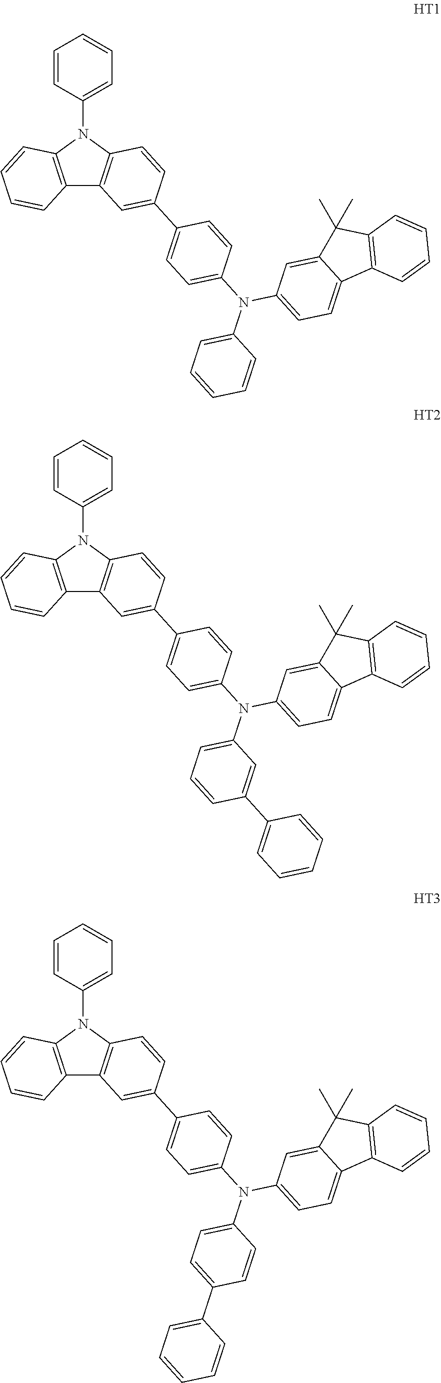 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21]

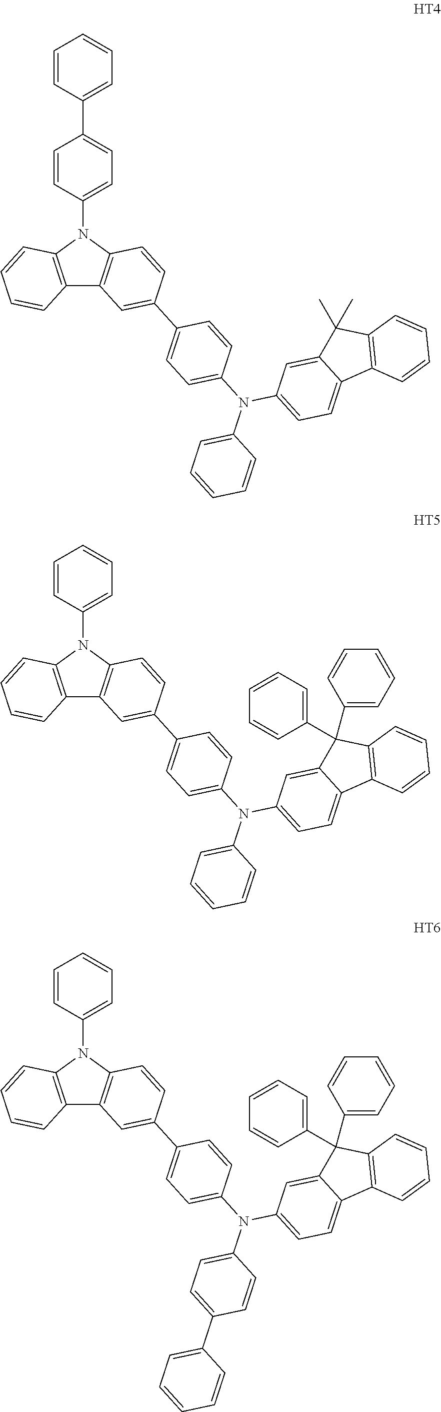 [SMILES: CC1(C)c2ccccc2-c2ccc(N(c3ccccc3)c3ccc(-c4ccc5c(c4)c4ccccc4n5-c4ccc(-c5ccccc5)cc4)cc3)cc21 ; c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

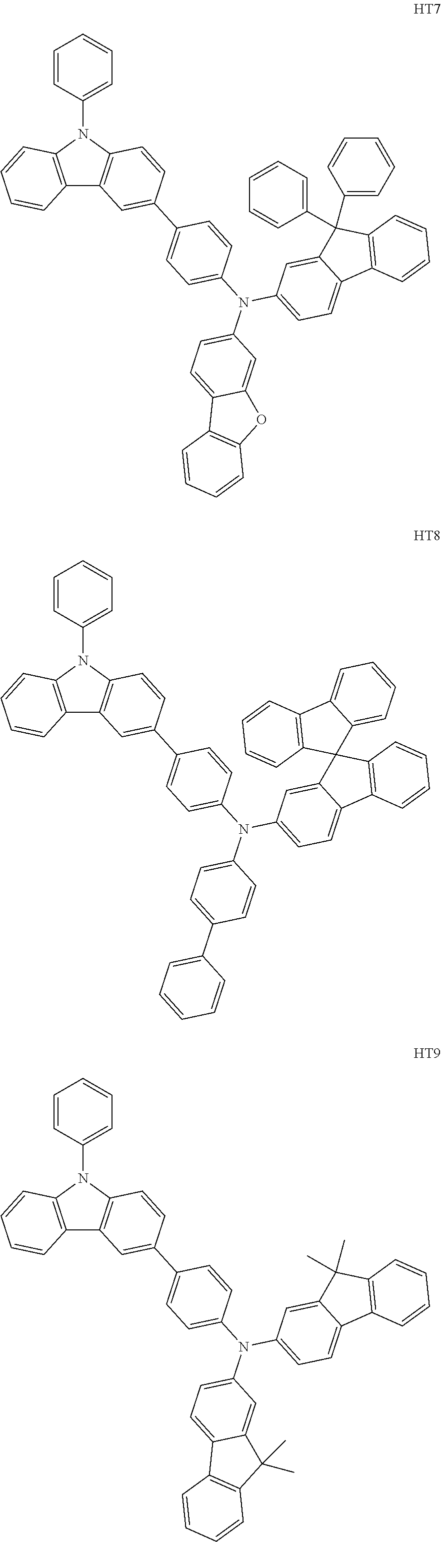 [SMILES: c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1 ; CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21]

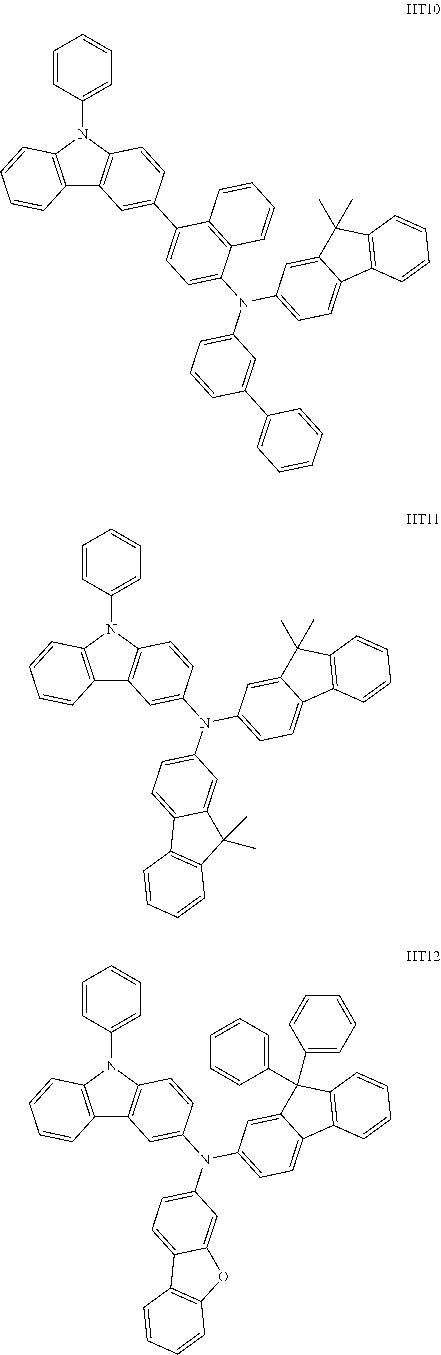 [SMILES: CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1]

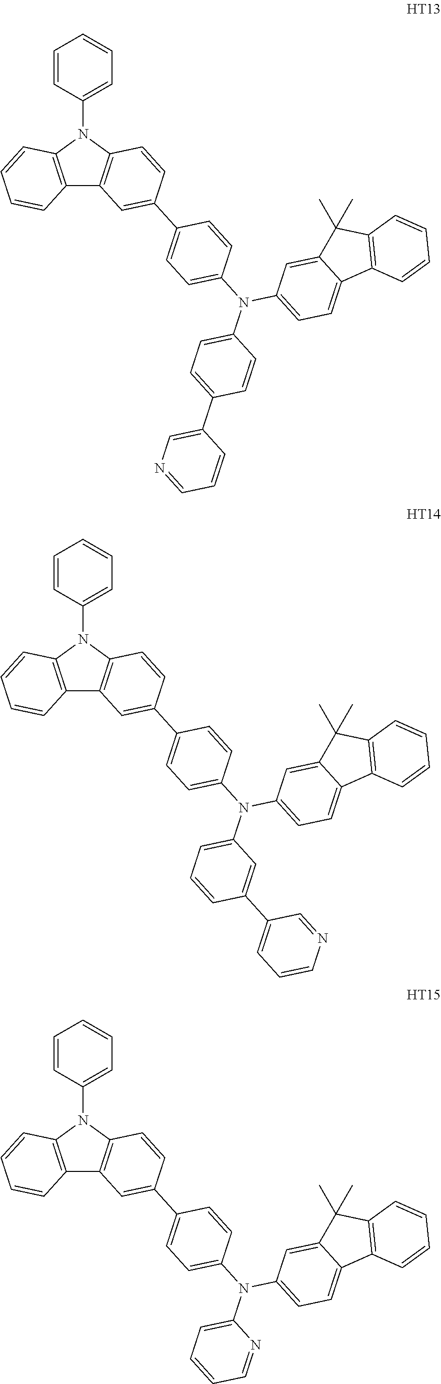 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21]

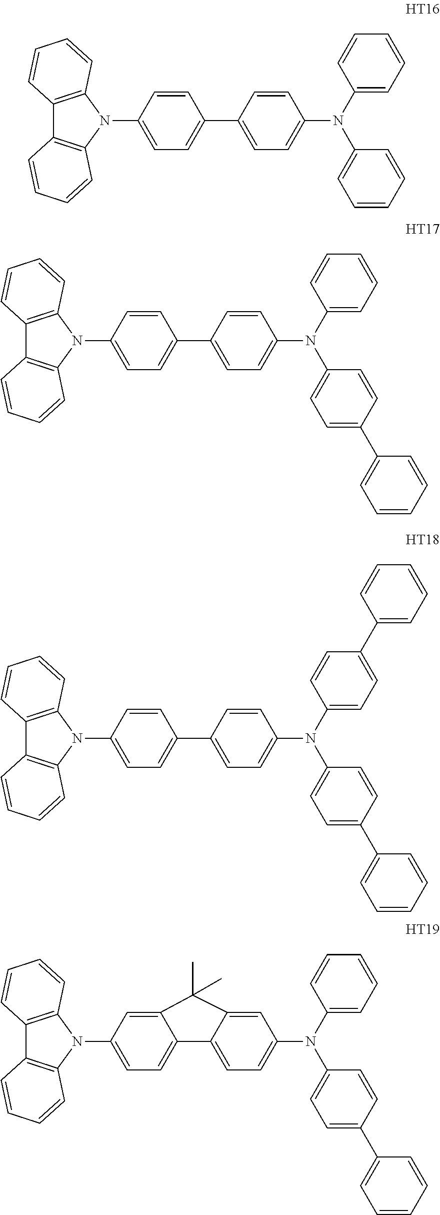

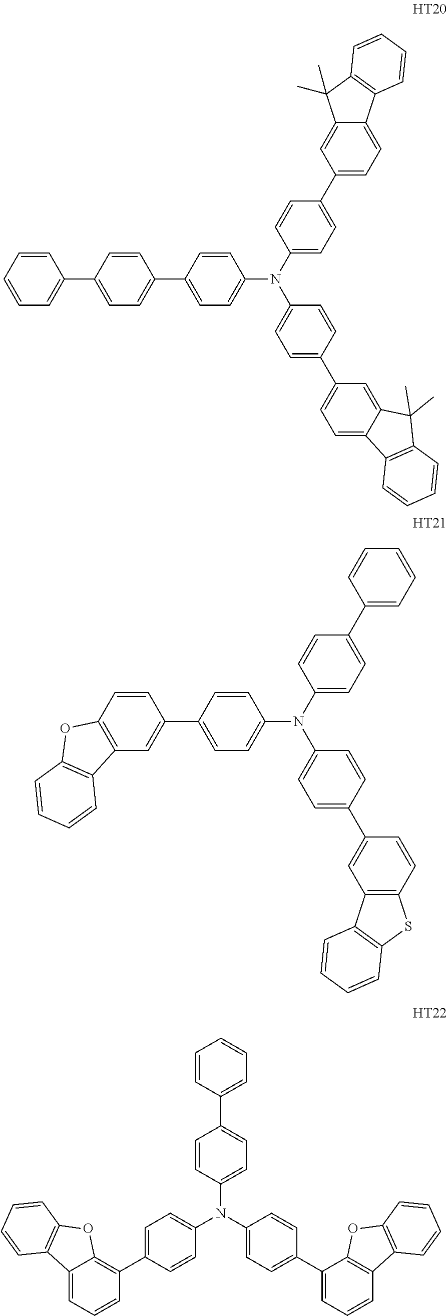 [SMILES: CC1(C)c2ccccc2-c2ccc(-c3ccc(N(c4ccc(-c5ccc(-c6ccccc6)cc5)cc4)c4ccc(-c5ccc6c(c5)C(C)(C)c5ccccc5-6)cc4)cc3)cc21 ; c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1]

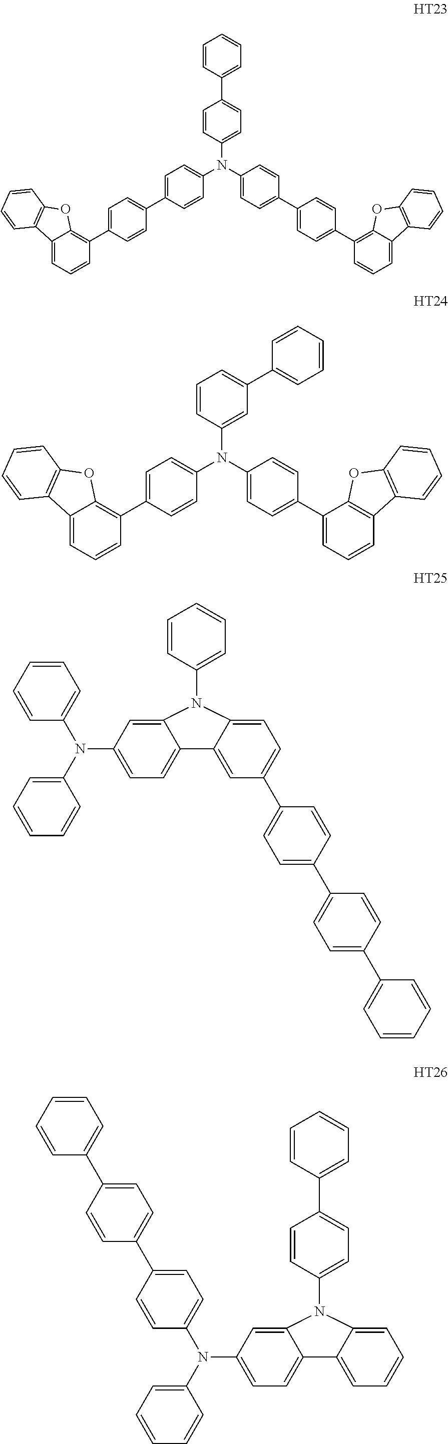 [SMILES: c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1 ; c1ccc(-c2cccc(N(c3ccc(-c4cccc5c4oc4ccccc45)cc3)c3ccc(-c4cccc5c4oc4ccccc45)cc3)c2)cc1 ; c1ccc(-c2ccc(-c3ccc(-c4ccc5c(c4)c4ccc(N(c6ccccc6)c6ccccc6)cc4n5-c4ccccc4)cc3)cc2)cc1 ; c1ccc(-c2ccc(-c3ccc(N(c4ccccc4)c4ccc5c6ccccc6n(-c6ccc(-c7ccccc7)cc6)c5c4)cc3)cc2)cc1]

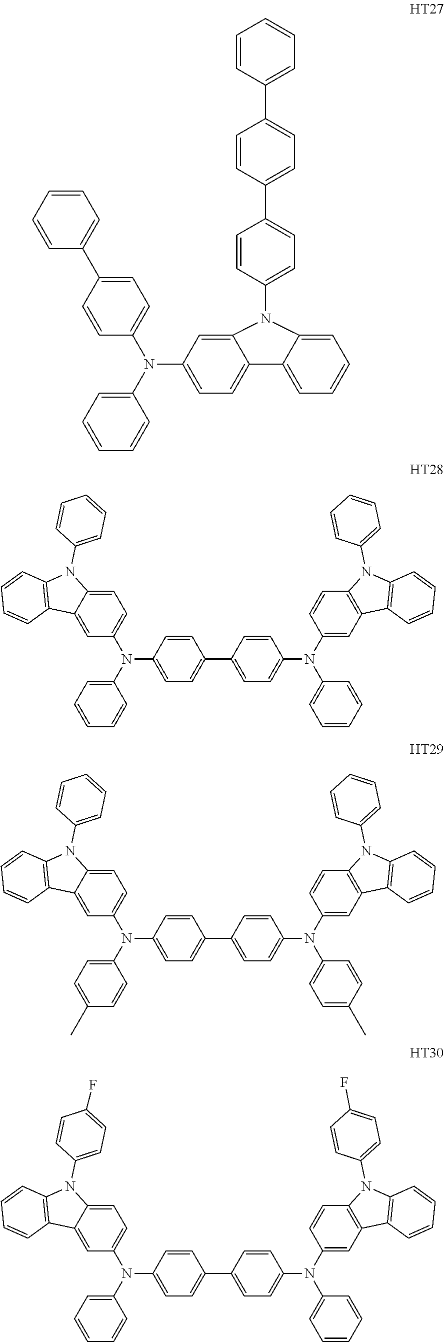 [SMILES: c1ccc(-c2ccc(-c3ccc(-n4c5ccccc5c5ccc(N(c6ccccc6)c6ccc(-c7ccccc7)cc6)cc54)cc3)cc2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; Cc1ccc(N(c2ccc(-c3ccc(N(c4ccc(C)cc4)c4ccc5c(c4)c4ccccc4n5-c4ccccc4)cc3)cc2)c2ccc3c(c2)c2ccccc2n3-c2ccccc2)cc1 ; Fc1ccc(-n2c3ccccc3c3cc(N(c4ccccc4)c4ccc(-c5ccc(N(c6ccccc6)c6ccc7c(c6)c6ccccc6n7-c6ccc(F)cc6)cc5)cc4)ccc32)cc1]

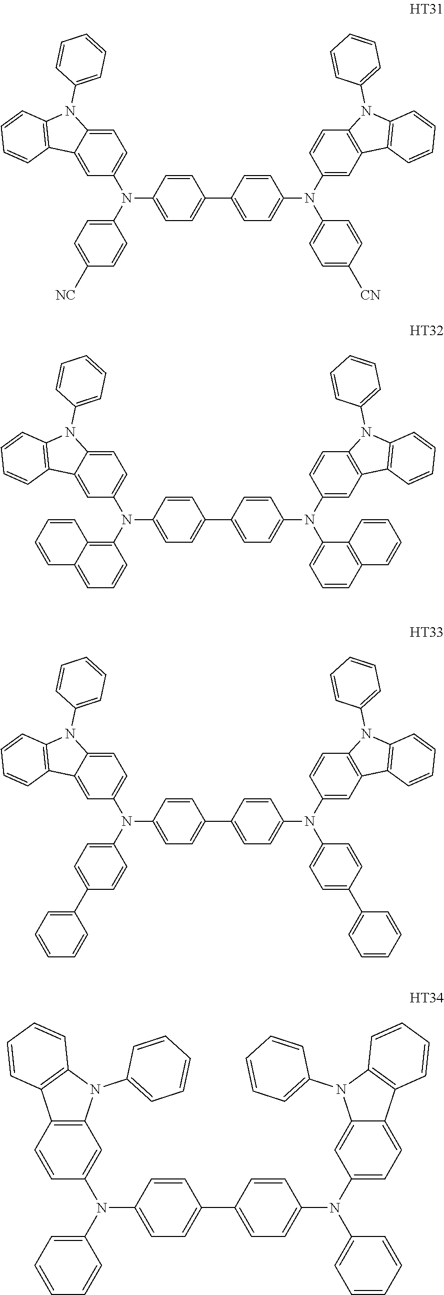 [SMILES: N#Cc1ccc(N(c2ccc(-c3ccc(N(c4ccc(C#N)cc4)c4ccc5c(c4)c4ccccc4n5-c4ccccc4)cc3)cc2)c2ccc3c(c2)c2ccccc2n3-c2ccccc2)cc1 ; c1ccc(-n2c3ccccc3c3cc(N(c4ccc(-c5ccc(N(c6ccc7c(c6)c6ccccc6n7-c6ccccc6)c6cccc7ccccc67)cc5)cc4)c4cccc5ccccc45)ccc32)cc1 ; c1ccc(-c2ccc(N(c3ccc(-c4ccc(N(c5ccc(-c6ccccc6)cc5)c5ccc6c(c5)c5ccccc5n6-c5ccccc5)cc4)cc3)c3ccc4c(c3)c3ccccc3n4-c3ccccc3)cc2)cc1 ; c1ccc(N(c2ccc(-c3ccc(N(c4ccccc4)c4ccc5c6ccccc6n(-c6ccccc6)c5c4)cc3)cc2)c2ccc3c4ccccc4n(-c4ccccc4)c3c2)cc1]

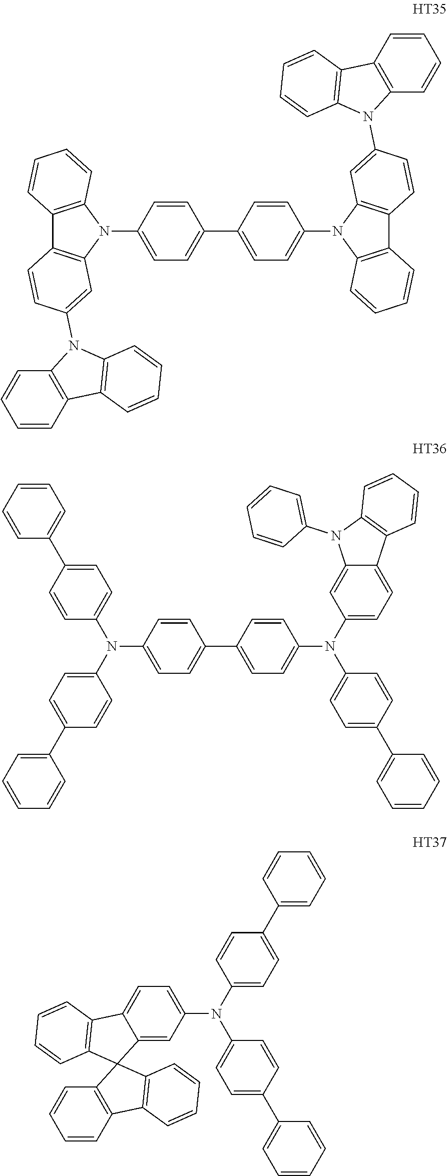 [SMILES: c1ccc2c(c1)c1ccccc1n2-c1ccc2c3ccccc3n(-c3ccc(-c4ccc(-n5c6ccccc6c6ccc(-n7c8ccccc8c8ccccc87)cc65)cc4)cc3)c2c1 ; c1ccc(-c2ccc(N(c3ccc(-c4ccccc4)cc3)c3ccc(-c4ccc(N(c5ccc(-c6ccccc6)cc5)c5ccc6c7ccccc7n(-c7ccccc7)c6c5)cc4)cc3)cc2)cc1 ; c1ccc(-c2ccc(N(c3ccc(-c4ccccc4)cc3)c3ccc4c(c3)C3(c5ccccc5-c5ccccc53)c3ccccc3-4)cc2)cc1]

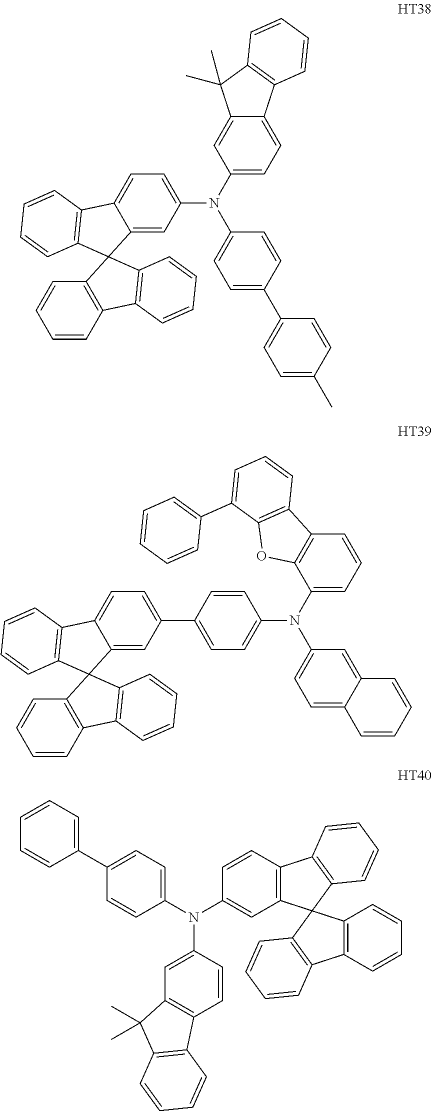

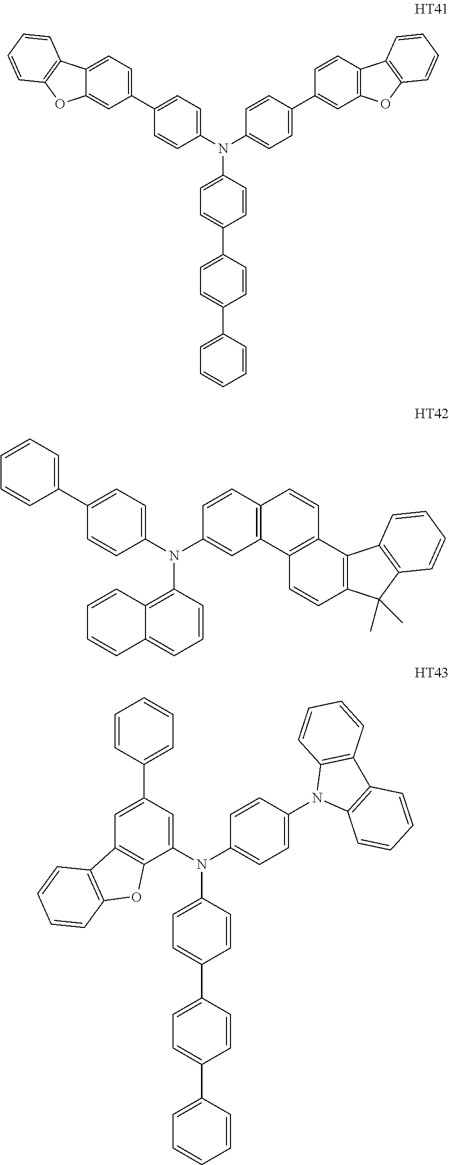 [SMILES: c1ccc(-c2ccc(-c3ccc(N(c4ccc(-c5ccc6c(c5)oc5ccccc56)cc4)c4ccc(-c5ccc6c(c5)oc5ccccc56)cc4)cc3)cc2)cc1 ; CC1(C)c2ccccc2-c2c1ccc1c2ccc2ccc(N(c3ccc(-c4ccccc4)cc3)c3cccc4ccccc34)cc21 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1]

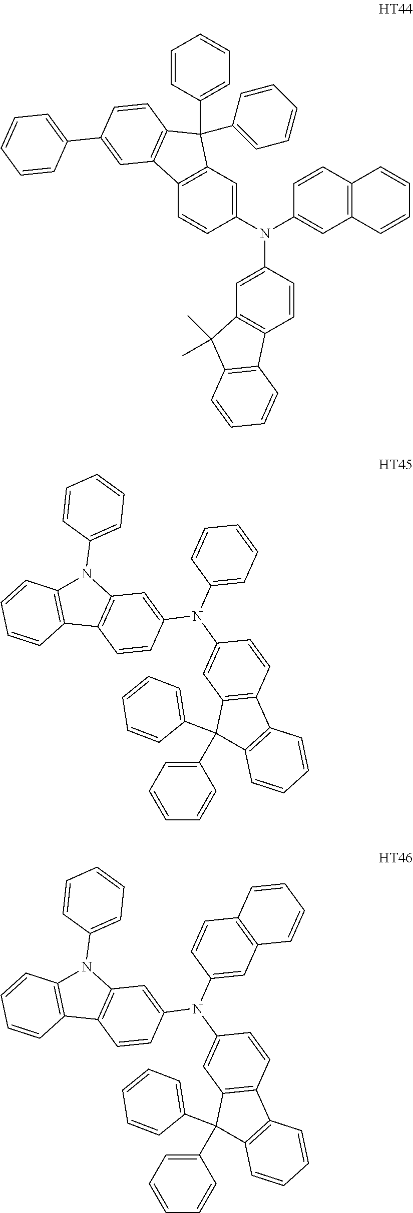 [SMILES: CC1(C)c2ccccc2-c2ccc(N(c3ccc4c(c3)C(c3ccccc3)(c3ccccc3)c3ccc(-c5ccccc5)cc3-4)c3ccc4ccccc4c3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; c1ccc(-n2c3ccccc3c3ccc(N(c4ccc5c(c4)C(c4ccccc4)(c4ccccc4)c4ccccc4-5)c4ccc5ccccc5c4)cc32)cc1]

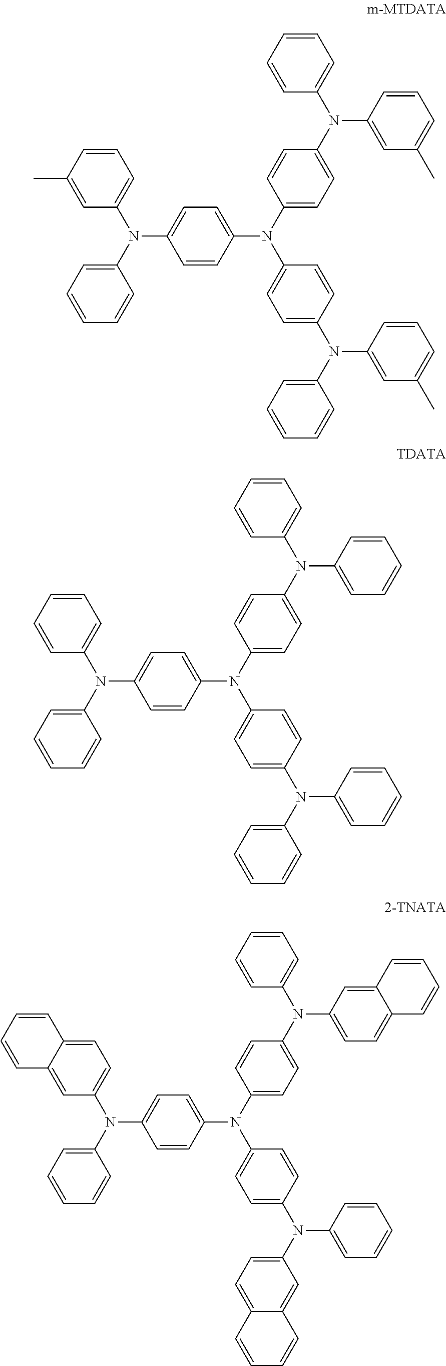

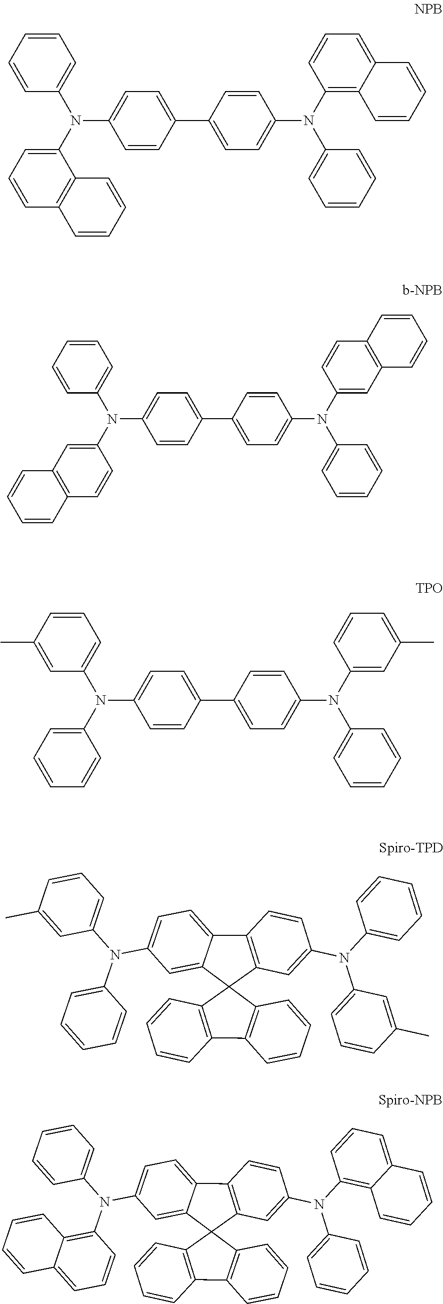

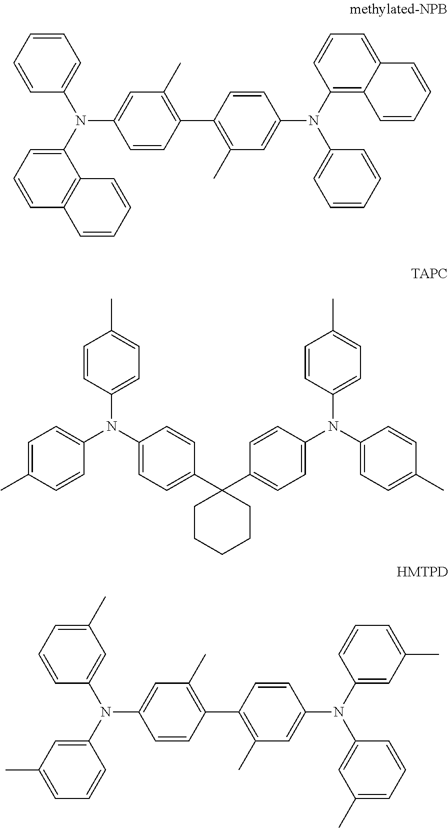

A thickness of the hole transport region may be in a range of about 50 Å to about 10,000 Å, for example, about 100 Å to about 4,000 Å. When the hole transport region includes a hole injection layer, a hole transport layer, or any combination thereof, a thickness of the hole injection layer may be in a range of about 100 Å to about 9,000 Å, for example, about 100 Å to about 1,000 Å, and a thickness of the hole transport layer may be in a range of about 50 Å to about 2,000 Å, for example, about 100 Å to about 1,500 Å. When the thicknesses of the hole transport region, the hole injection layer and the hole transport layer are within these ranges, satisfactory hole transporting characteristics may be obtained without a substantial increase in driving voltage.

The emission auxiliary layer may increase light-emission efficiency by compensating for an optical resonance distance according to the wavelength of light emitted by the emission layer, and the electron blocking layer may reduce or prevent leakage of electrons from the emission layer to an electron transport region. A material that may be included in the hole transport region as described above may be included in the emission auxiliary layer and the electron blocking layer.

p-Dopant

The hole transport region may further include, in addition to these materials, a charge-generation material for the improvement of conductive properties. The charge-generation material may be uniformly or non-uniformly dispersed in the hole transport region (for example, in the form of a single layer consisting of a charge-generation material).

The charge-generation material may be, for example, a p-dopant.

In an embodiment, the lowest unoccupied molecular orbital (LUMO) energy level of the p-dopant may be −3.5 eV or less.

In an embodiment, the p-dopant may include a quinone derivative, a cyano group-containing compound, a compound containing element EU and element EL2, or any combination thereof.

Non-limiting examples of the quinone derivative are TCNQ, F4-TCNQ, etc.

Non-limiting examples of the cyano group-containing compound are HAT-CN and a compound represented by Formula 221 below.

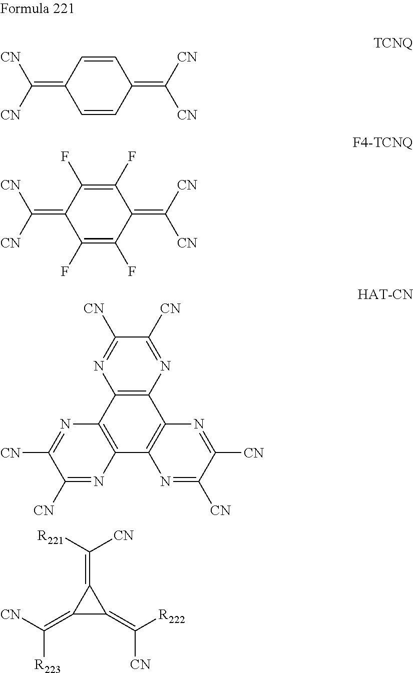

In Formula 221,

R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and

at least one of R₂₂₁ to R₂₂₃ may (e.g., may each) independently be a C₃-C₆₀ carbocyclic group; a C₁-C₆₀ heterocyclic group, each substituted with a cyano group; —F; —Cl; —Br; —I; a C₁-C₂₀ alkyl group substituted with a cyano group, —F, —Cl, —Br, —I, or any combination thereof; or any combination thereof.

In the compound containing element EL1 and element EL2, element EL1 may be a metal, a metalloid, or a combination thereof, and element EL2 may be a non-metal, a metalloid, or a combination thereof.

Non-limiting examples of the metal may be an alkali metal (for example, lithium (Li), sodium (Na), potassium (K), rubidium (Rb), cesium (Cs), etc.); an alkaline earth metal (for example, beryllium (Be), magnesium (Mg), calcium (Ca), strontium (Sr), barium (Ba), etc.); a transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), etc.); a post-transition metal (for example, zinc (Zn), indium (In), tin (Sn), etc.); and/or a lanthanide metal (for example, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), ruthenium (Lu), etc.).

Non-limiting examples of the metalloid are silicon (Si), antimony (Sb), and/or tellurium (Te).

Non-limiting examples of the non-metal are oxygen (O) and/or halogen (for example, F, Cl, Br, I, etc.).

In an embodiment, non-limiting examples of the compound containing element EL1 and element EL2 are a metal oxide, a metal halide (for example, metal fluoride, metal chloride, metal bromide, and/or metal iodide), a metalloid halide (for example, metalloid fluoride, metalloid chloride, metalloid bromide, or metalloid iodide), a metal telluride, or any combination thereof.

Non-limiting examples of the metal oxide are tungsten oxide (for example, WO, W₂O₃, WO₂, WO₃, W₂O₅, etc.), vanadium oxide (for example, VO, V₂O₃, VO₂, V₂O₅, etc.), molybdenum oxide (for example, MoO, Mo₂O₃, MoO₂, MoO₃, Mo₂O₅, etc.), and/or rhenium oxide (for example, ReO₃, etc.).

Non-limiting examples of the metal halide are alkali metal halide, alkaline earth metal halide, transition metal halide, post-transition metal halide, and/or lanthanide metal halide.

Non-limiting examples of the alkali metal halide are LiF, NaF, KF, RbF, CsF, LiCl, NaCl, KCl, RbCl, CsCl, LiBr, NaBr, KBr, RbBr, CsBr, LiI, NaI, KI, RbI, and/or CsI.

Non-limiting examples of the alkaline earth metal halide are BeF₂, MgF₂, CaF₂, SrF₂, BaF₂, BeCl₂, MgCl₂, CaCl₂, SrCl₂, BaCl₂, BeBr₂, MgBr₂, CaBr₂, SrBr₂, BaBr₂, BeI₂, MgI₂, CaI₂, SrI₂, and/or BaI₂.

Non-limiting examples of the transition metal halide are titanium halide (for example, TiF₄, TiCl₄, TiBr₄, TiI₄, etc.), zirconium halide (for example, ZrF₄, ZrCl₄, ZrBr₄, ZrI₄, etc.), hafnium halide (for example, HfF₄, HfCl₄, HfBr₄, HfI₄, etc.), vanadium halide (for example, VF₃, VCl₃, VBr₃, VI₃, etc.), niobium halide (for example, NbF₃, NbCl₃, NbBr₃, NbI₃, etc.), tantalum halide (for example, TaF₃, TaCl₃, TaBr₃, TaI₃, etc.), chromium halide (for example, CrF₃, CrCl₃, CrBr₃, CrI₃, etc.), molybdenum halide (for example, MoF₃, MoCl₃, MoBr₃, MoI₃, etc.), tungsten halide (for example, WF₃, WCl₃, WBr₃, WI₃, etc.), manganese halide (for example, MnF₂, MnCl₂, MnBr₂, MnI₂, etc.), technetium halide (for example, TcF₂, TcCl₂, TcBr₂, TcI₂, etc.), rhenium halide (for example, ReF₂, ReCl₂, ReBr₂, ReI₂, etc.), iron halide (for example, FeF₂, FeCl₂, FeBr₂, FeI₂, etc.), ruthenium halide (for example, RuF₂, RuCl₂, RuBr₂, RuI₂, etc.), osmium halide (for example, OsF₂, OsCl₂, OsBr₂, OsI₂, etc.), cobalt halide (for example, CoF₂, CoCl₂, CoBr₂, CoI₂, etc.), rhodium halide (for example, RhF₂, RhCl₂, RhBr₂, RhI₂, etc.), iridium halide (for example, IrF₂, IrCl₂, IrBr₂, IrI₂, etc.), nickel halide (for example, NiF₂, NiCl₂, NiBr₂, NiI₂, etc.), palladium halide (for example, PdF₂, PdCl₂, PdBr₂, PdI₂, etc.), platinum halide (for example, PtF₂, PtCl₂, PtBr₂, PtI₂, etc.), copper halide (for example, CuF, CuCl, CuBr, CuI, etc.), silver halide (for example, AgF, AgCl, AgBr, AgI, etc.), and/or gold halide (for example, AuF, AuCl, AuBr, AuI, etc.).

Non-limiting examples of the post-transition metal halide are zinc halide (for example, ZnF₂, ZnCl₂, ZnBr₂, ZnI₂, etc.), indium halide (for example, InI₃, etc.), and/or tin halide (for example, SnI₂, etc.).

Non-limiting examples of the lanthanide metal halide are YbF, YbF₂, YbF₃, SmF₃, YbCl, YbCl₂, YbCl₃ SmCl₃, YbBr, YbBr₂, YbBr₃ SmBr₃, YbI, YbI₂, YbI₃, and/or SmI₃.

An example of the metalloid halide is antimony halide (for example, SbCl₅, etc.).

Non-limiting examples of the metal telluride are alkali metal telluride (for example, Li₂Te, Na₂Te, K₂Te, Rb₂Te, Cs₂Te etc.), alkaline earth metal telluride (for example, BeTe, MgTe, CaTe, SrTe, BaTe, etc.), transition metal telluride (for example, TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, etc.), post-transition metal telluride (for example, ZnTe, etc.), and/or lanthanide metal telluride (for example, LaTe, CeTe, PrTe, NdTe, PmTe, EuTe, GdTe, TbTe, DyTe, HoTe, ErTe, TmTe, YbTe, LuTe, etc.).

### Emission Layer in Interlayer 130

When the light-emitting device 10 is a full-color light-emitting device, the emission layer may be patterned into a red emission layer, a green emission layer, and/or a blue emission layer, according to a sub-pixel. In one or more embodiments, the emission layer may have a stacked structure of two or more layers of a red emission layer, a green emission layer, and a blue emission layer, in which the two or more layers contact each other or are separated from each other. In one or more embodiments, the emission layer may include two or more materials of a red light-emitting material, a green light-emitting material, and a blue light-emitting material, in which the two or more materials are mixed with each other in a single layer to emit white light.

The emission layer may include a host and a dopant. The dopant may include a phosphorescent dopant, a fluorescent dopant, or any combination thereof.

The amount of the dopant in the emission layer may be from about 0.01 to about 15 parts by weight based on 100 parts by weight of the host.

In one or more embodiments, the emission layer may include a quantum dot.

In one embodiment, the emission layer may include a delayed fluorescence material. The delayed fluorescence material may act as (e.g., be) a host or a dopant in the emission layer.

A thickness of the emission layer may be in a range of about 100 Å to about 1,000 Å, for example, about 200 Å to about 600 Å. When the thickness of the emission layer is within these ranges, excellent light-emission characteristics may be obtained without a substantial increase in driving voltage.

### Host

In one or more embodiments, the host may include (e.g., be) a compound represented by Formula 301 below:

[Ar₃₀₁]_{xb11}-[(L₃₀₁)_{xb1}-R₃₀₁]_{xb21} Formula 301

In Formula 301,

Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xb11 may be 1, 2, or 3,

xb1 may be an integer selected from 0 to 5,

R₃₀₁ may be hydrogen, deuterium, —F, —Cl, —Br, —I, a hydroxyl group, a cyano group, a nitro group, a C₁-C₆₀alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),

xb21 may be an integer selected from 1 to 5, and

Q₃₀₁ to Q₃₀₃ may each independently be the same as described in connection with Q₁.

For example, xb11 in Formula 301 is 2 or more, two or more of Ar₃₀₁(s) may be linked to each other via a single bond.

In an embodiment, the host may include a compound represented by Formula 301-1, a compound represented by Formula 301-2, or any combination thereof:

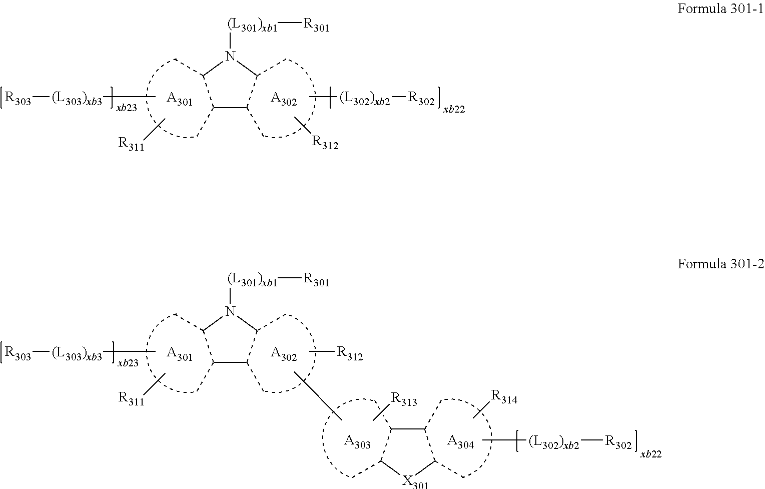

In Formulae 301-1 and 301-2,

ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

X₃₀₁ may be O, S, N—[(L₃₀₄)_{xb4}-R₃₀₄], C(R₃₀₄)(R₃₀₅), or Si(R₃₀₄)(R₃₀₅),

xb22 and xb23 may each independently be 0, 1, or 2,

L₃₀₁, xb1, and R₃₀₁ may each independently be the same as respectively described in the present specification,

L₃₀₂ to L₃₀₄ may each independently be the same as described in connection with L₃₀₁,

xb2 to xb4 may each independently be the same as described in connection with xb1, and

R₃₀₂ to R₃₀₅ and R₃₁₁ to R₃₁₄ may each independently be the same as described in connection with R₃₀₁.

In an embodiment, the host may include (e.g., be) an alkaline earth metal complex, a post-transition metal complex, or any combination thereof. In an embodiment, the host may include a Be complex (for example, Compound H55), a Mg complex, a Zn complex, or a combination thereof.

In an embodiment, the host may include (e.g., be) one of Compounds H1 to H124, 9,10-di(2-naphthyl)anthracene (ADN), 2-methyl-9,10-bis(naphthalen-2-yl)anthracene (MADN), 9,10-di-(2-naphthyl)-2-t-butyl-anthracene (TBADN), 4,4′-bis(N-carbazolyl)-1,1′-biphenyl (CBP), 1,3-di-9-carbazolylbenzene (mCP), 1,3,5-tri(carbazol-9-yl)benzene (TCP), or any combination thereof, but embodiments of the present disclosure are not limited thereto:

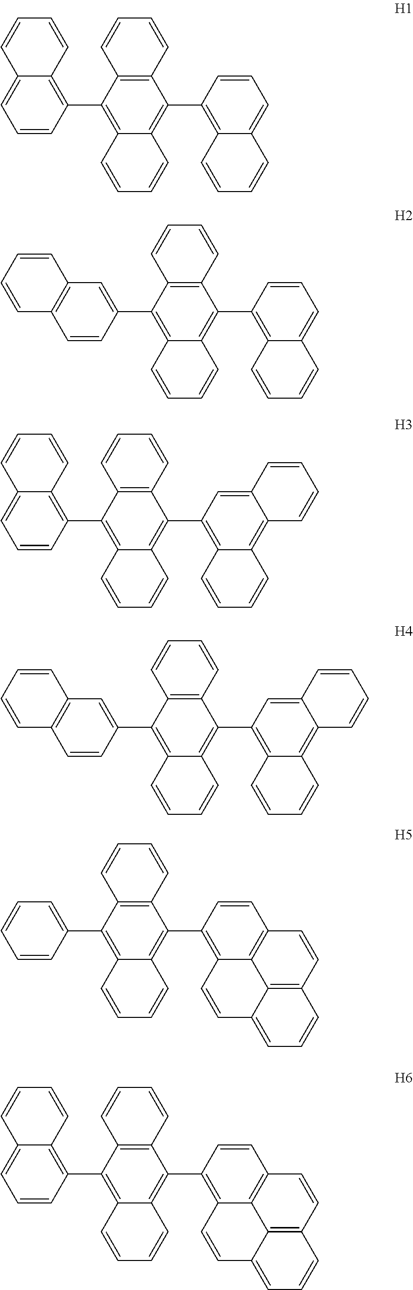

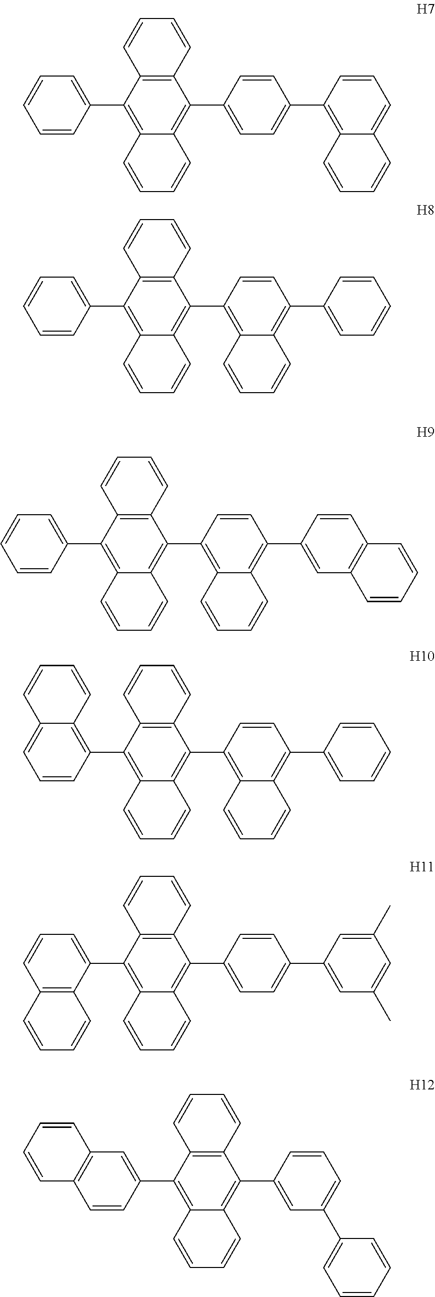

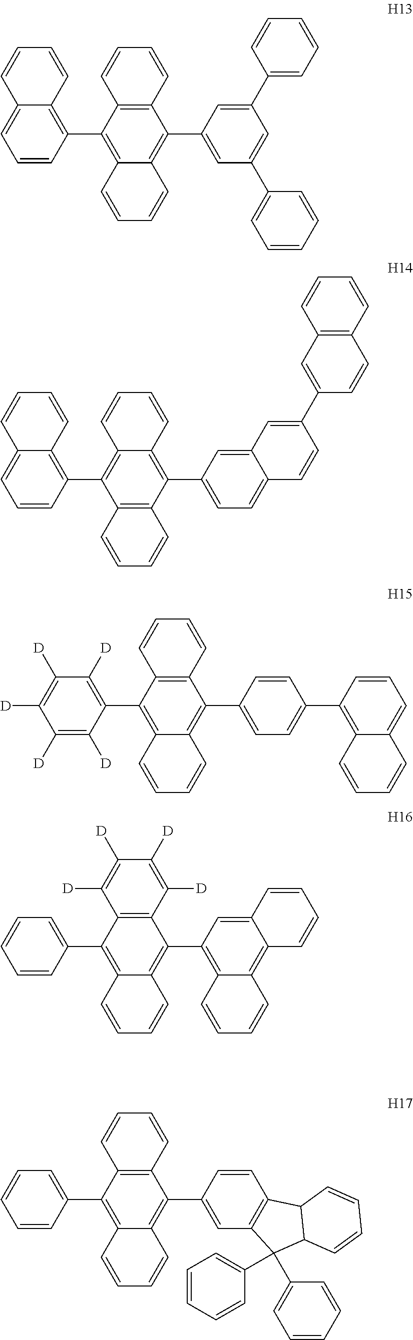

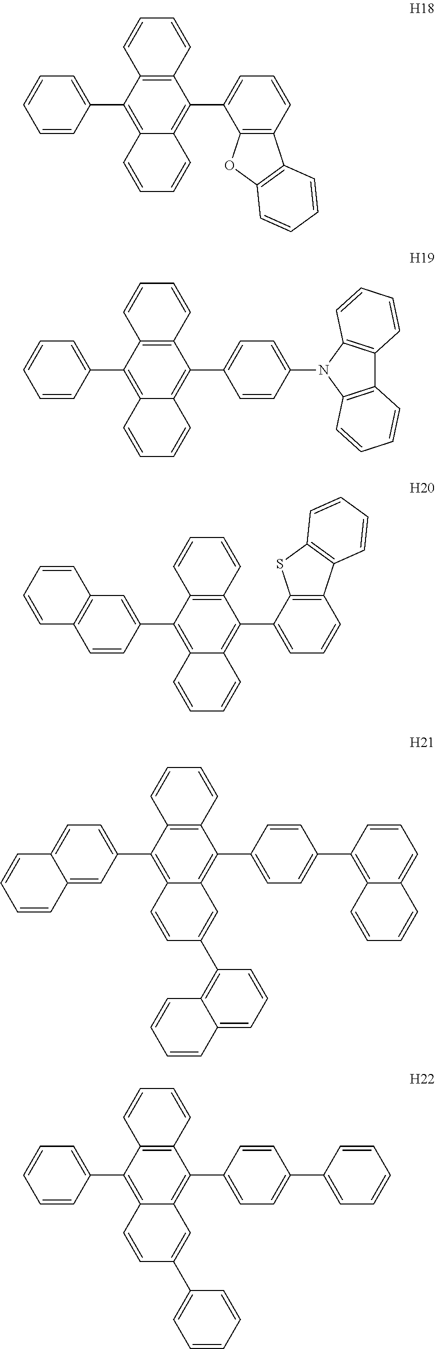

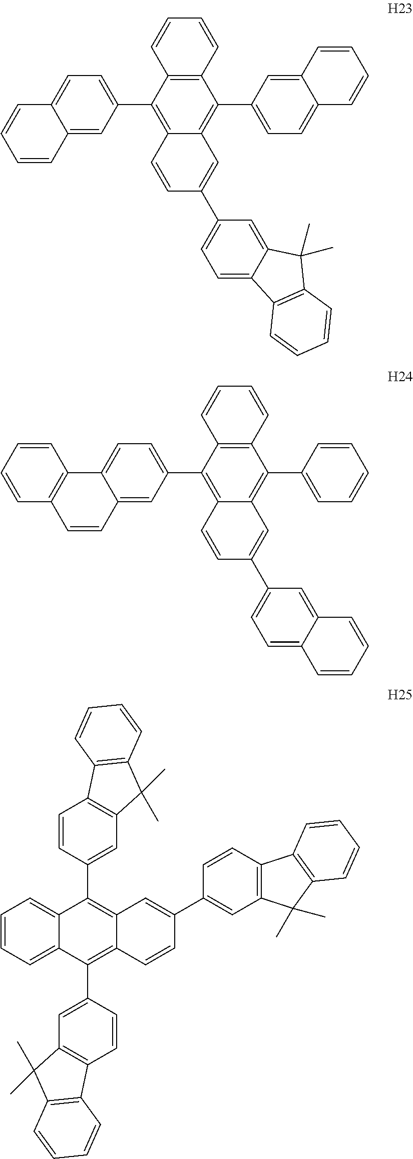

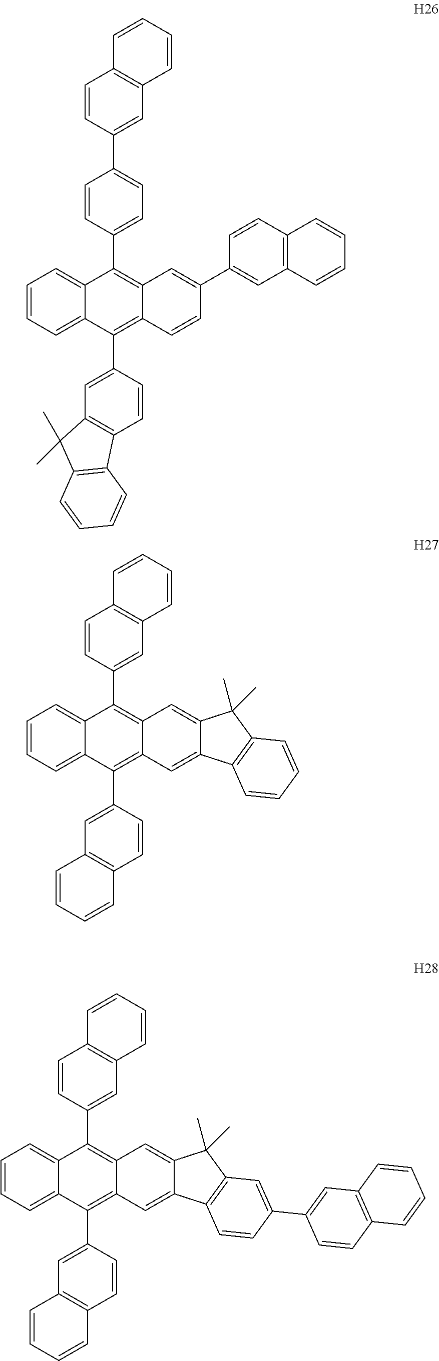

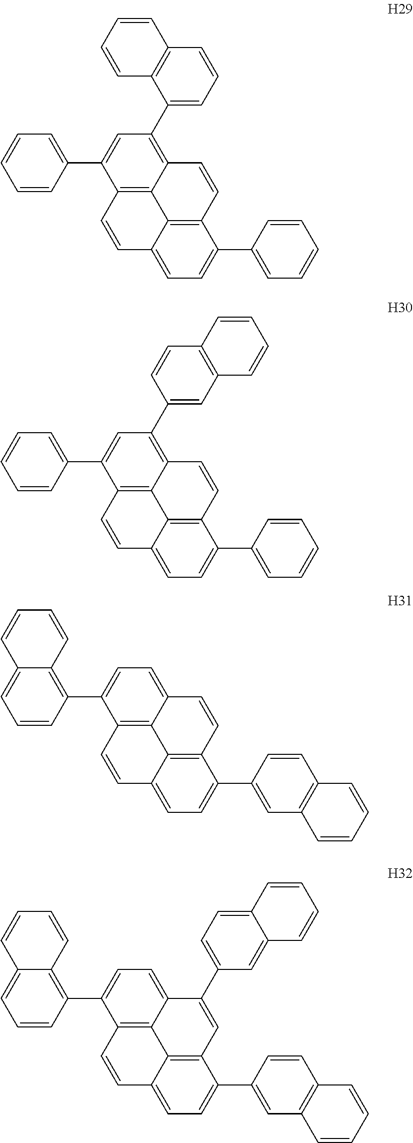

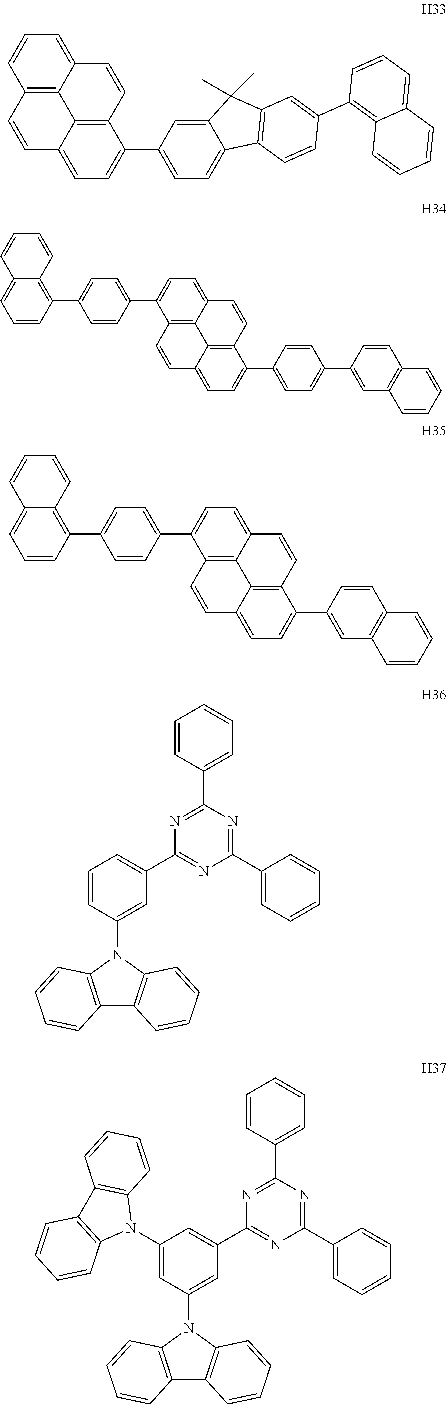

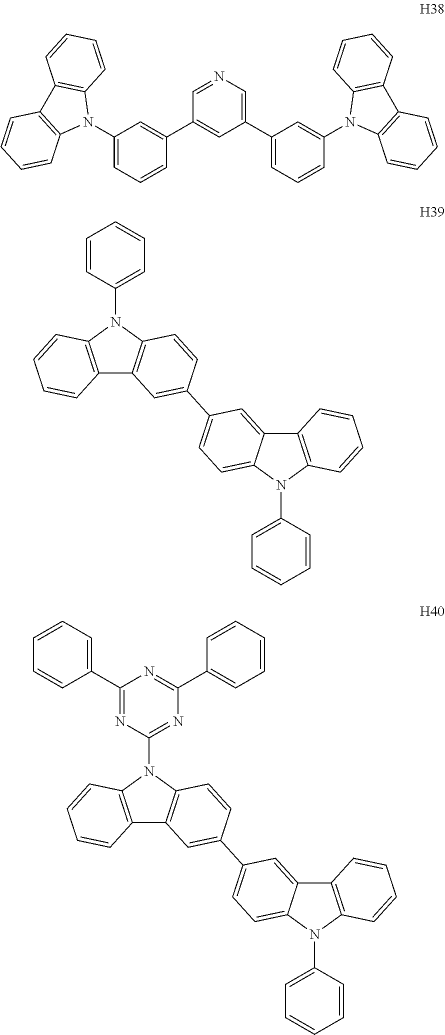

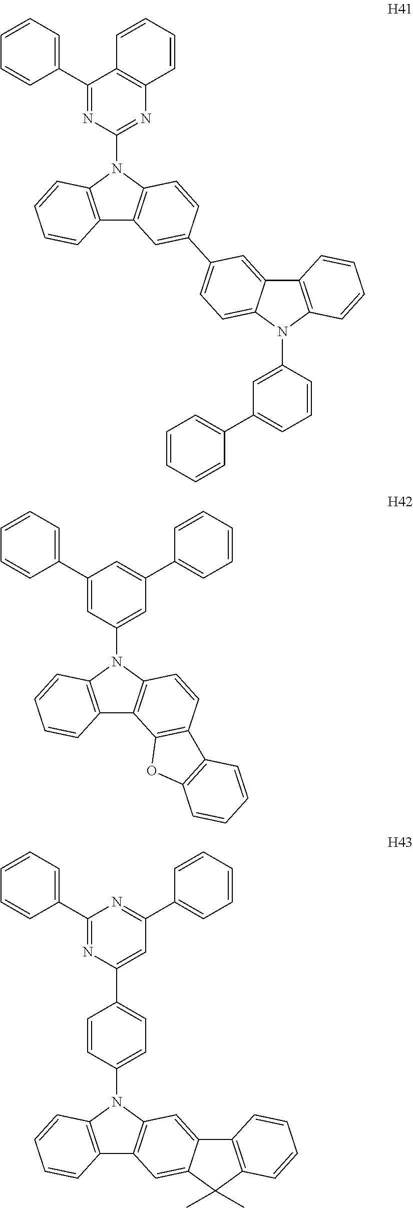

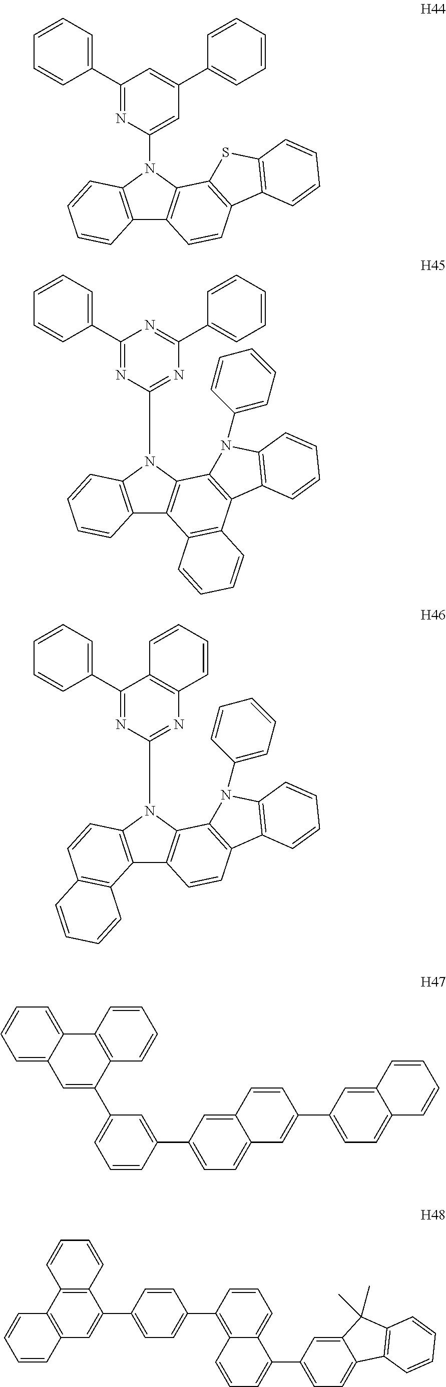

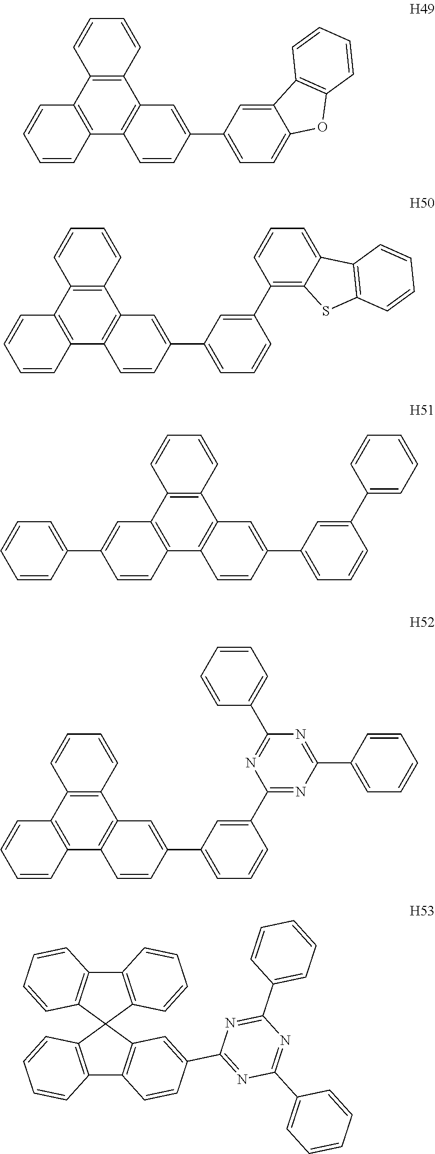

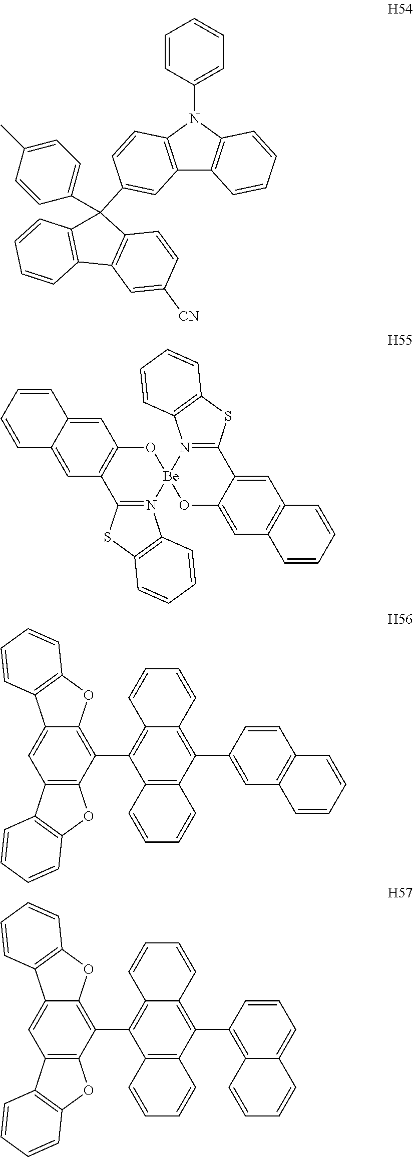

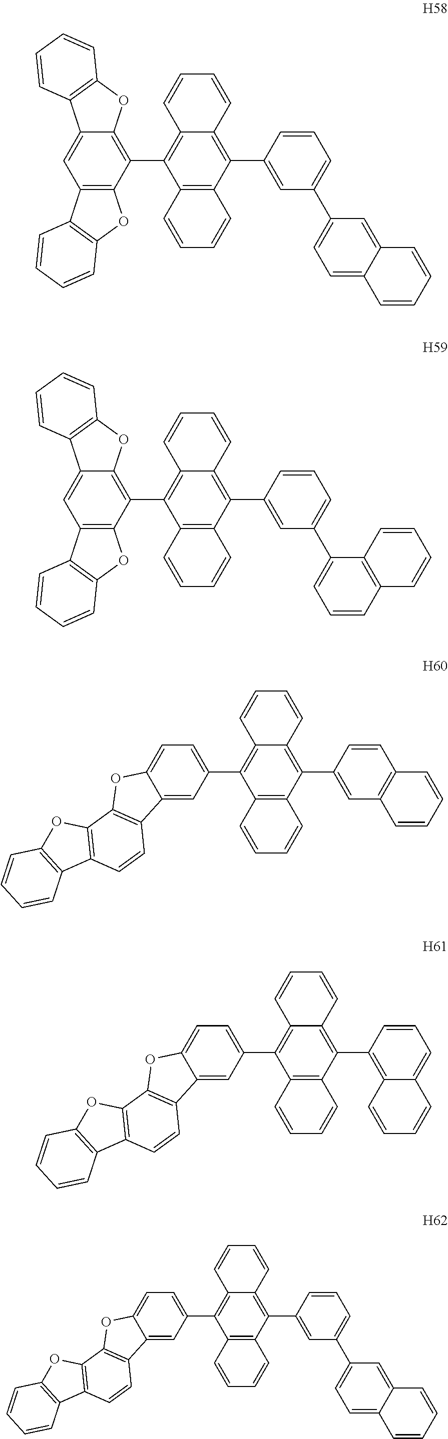 [SMILES: c1cc(-c2ccc3ccccc3c2)cc(-c2c3ccccc3c(-c3c4oc5ccccc5c4cc4c3oc3ccccc34)c3ccccc23)c1 ; c1cc(-c2cccc3ccccc23)cc(-c2c3ccccc3c(-c3c4oc5ccccc5c4cc4c3oc3ccccc34)c3ccccc23)c1 ; c1ccc2cc(-c3c4ccccc4c(-c4ccc5oc6c(ccc7c8ccccc8oc76)c5c4)c4ccccc34)ccc2c1 ; c1ccc2c(-c3c4ccccc4c(-c4ccc5oc6c(ccc7c8ccccc8oc76)c5c4)c4ccccc34)cccc2c1 ; c1cc(-c2ccc3ccccc3c2)cc(-c2c3ccccc3c(-c3ccc4oc5c(ccc6c7ccccc7oc65)c4c3)c3ccccc23)c1]

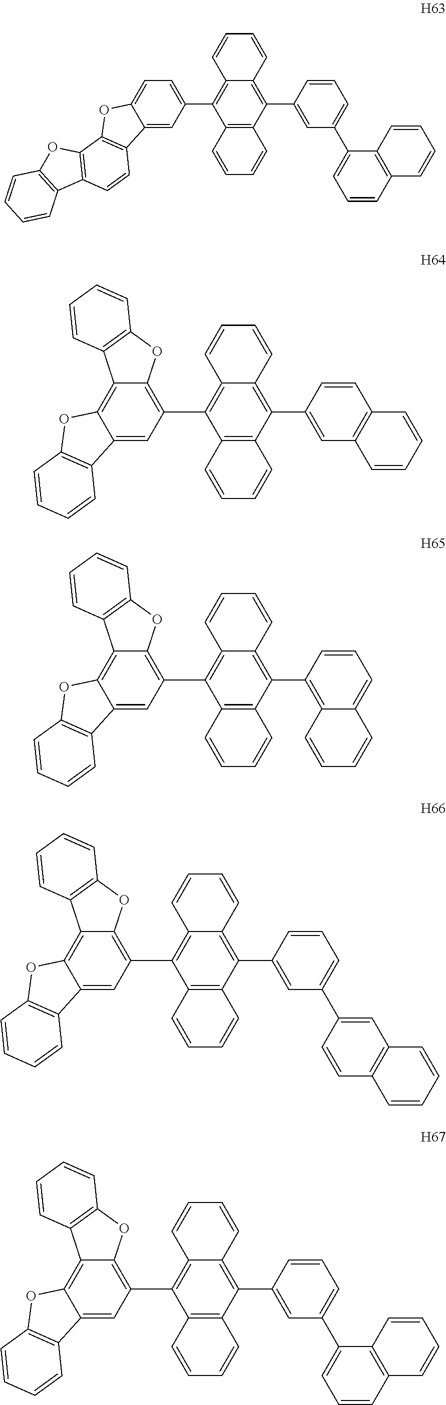

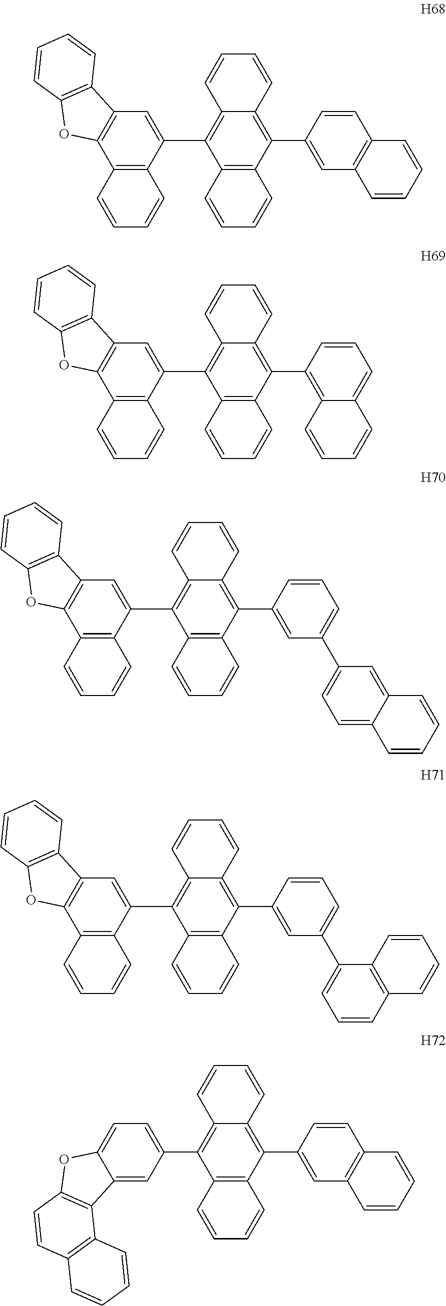 [SMILES: c1ccc2cc(-c3c4ccccc4c(-c4cc5c6ccccc6oc5c5ccccc45)c4ccccc34)ccc2c1 ; c1ccc2c(-c3c4ccccc4c(-c4cc5c6ccccc6oc5c5ccccc45)c4ccccc34)cccc2c1 ; c1cc(-c2ccc3ccccc3c2)cc(-c2c3ccccc3c(-c3cc4c5ccccc5oc4c4ccccc34)c3ccccc23)c1 ; c1cc(-c2cccc3ccccc23)cc(-c2c3ccccc3c(-c3cc4c5ccccc5oc4c4ccccc34)c3ccccc23)c1 ; c1ccc2cc(-c3c4ccccc4c(-c4ccc5oc6ccc7ccccc7c6c5c4)c4ccccc34)ccc2c1]

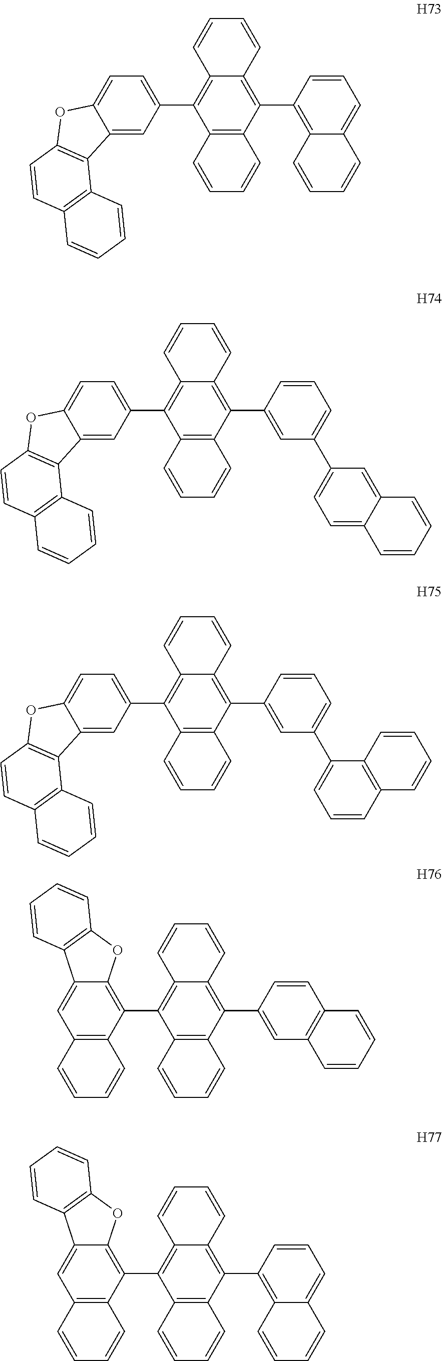

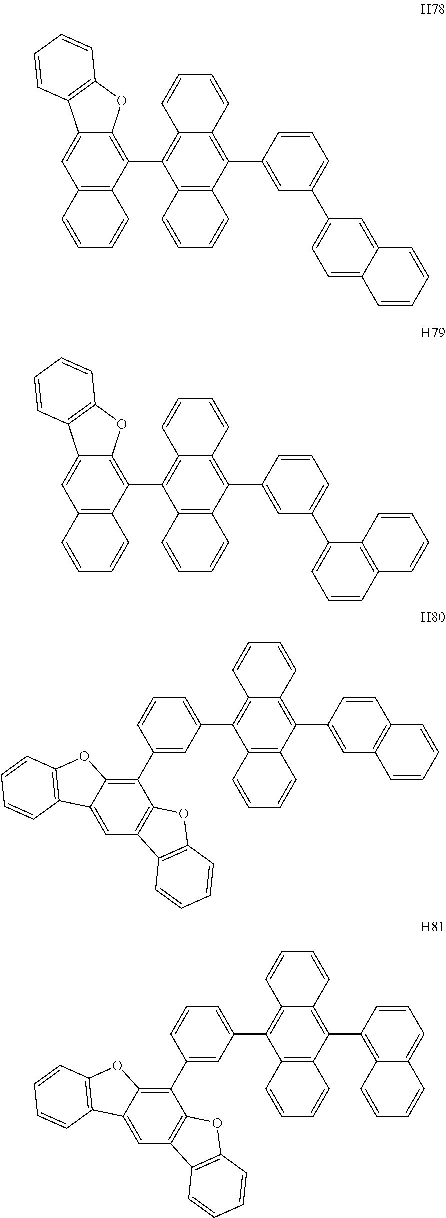 [SMILES: c1cc(-c2ccc3ccccc3c2)cc(-c2c3ccccc3c(-c3c4ccccc4cc4c3oc3ccccc34)c3ccccc23)c1 ; c1cc(-c2cccc3ccccc23)cc(-c2c3ccccc3c(-c3c4ccccc4cc4c3oc3ccccc34)c3ccccc23)c1 ; c1cc(-c2c3ccccc3c(-c3ccc4ccccc4c3)c3ccccc23)cc(-c2c3oc4ccccc4c3cc3c2oc2ccccc23)c1 ; c1cc(-c2c3ccccc3c(-c3cccc4ccccc34)c3ccccc23)cc(-c2c3oc4ccccc4c3cc3c2oc2ccccc23)c1]

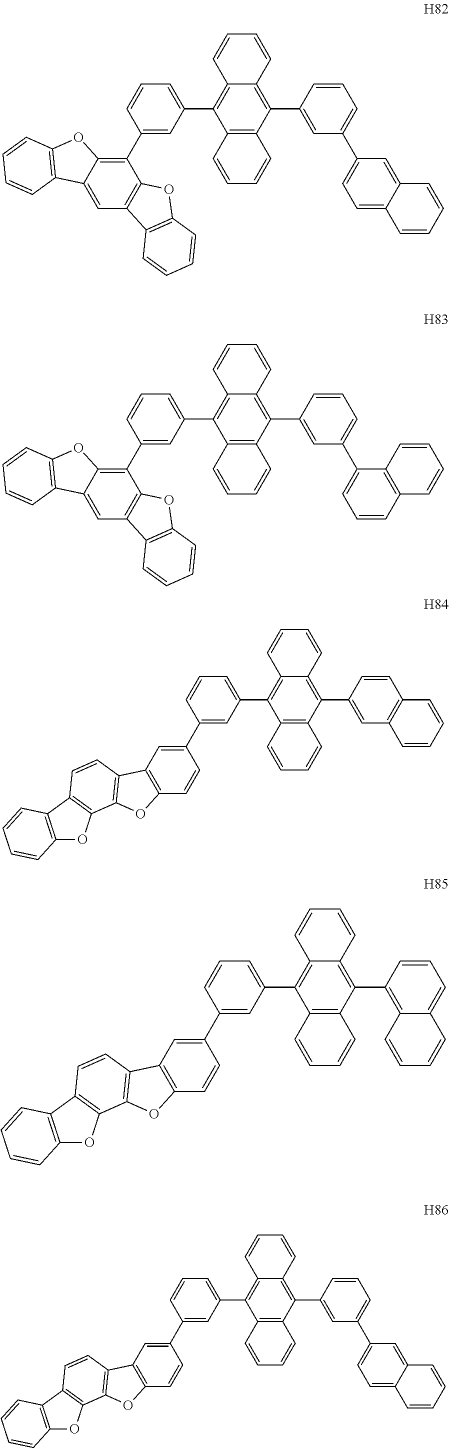 [SMILES: c1cc(-c2ccc3ccccc3c2)cc(-c2c3ccccc3c(-c3cccc(-c4c5oc6ccccc6c5cc5c4oc4ccccc45)c3)c3ccccc23)c1 ; c1cc(-c2cccc3ccccc23)cc(-c2c3ccccc3c(-c3cccc(-c4c5oc6ccccc6c5cc5c4oc4ccccc45)c3)c3ccccc23)c1 ; c1cc(-c2ccc3oc4c(ccc5c6ccccc6oc54)c3c2)cc(-c2c3ccccc3c(-c3ccc4ccccc4c3)c3ccccc23)c1 ; c1cc(-c2ccc3oc4c(ccc5c6ccccc6oc54)c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6c(ccc7c8ccccc8oc76)c5c4)c3)c3ccccc23)c1]

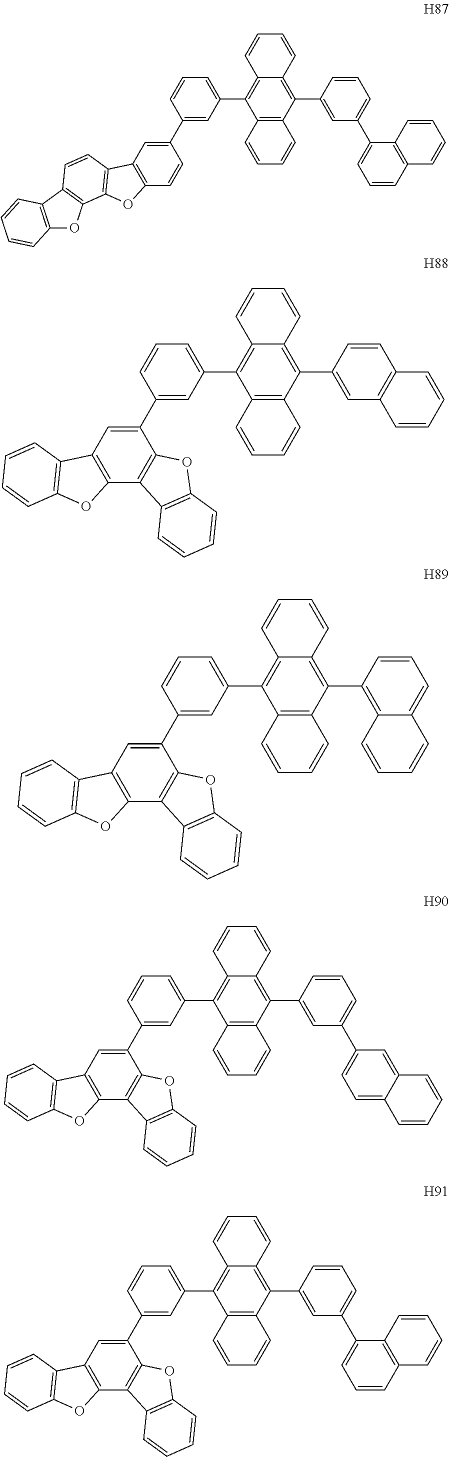 [SMILES: c1cc(-c2ccc3oc4c(ccc5c6ccccc6oc54)c3c2)cc(-c2c3ccccc3c(-c3cccc(-c4cccc5ccccc45)c3)c3ccccc23)c1 ; c1cc(-c2c3ccccc3c(-c3ccc4ccccc4c3)c3ccccc23)cc(-c2cc3c4ccccc4oc3c3c2oc2ccccc23)c1 ; c1cc(-c2c3ccccc3c(-c3cccc4ccccc34)c3ccccc23)cc(-c2cc3c4ccccc4oc3c3c2oc2ccccc23)c1 ; c1cc(-c2ccc3ccccc3c2)cc(-c2c3ccccc3c(-c3cccc(-c4cc5c6ccccc6oc5c5c4oc4ccccc45)c3)c3ccccc23)c1 ; c1cc(-c2cccc3ccccc23)cc(-c2c3ccccc3c(-c3cccc(-c4cc5c6ccccc6oc5c5c4oc4ccccc45)c3)c3ccccc23)c1]

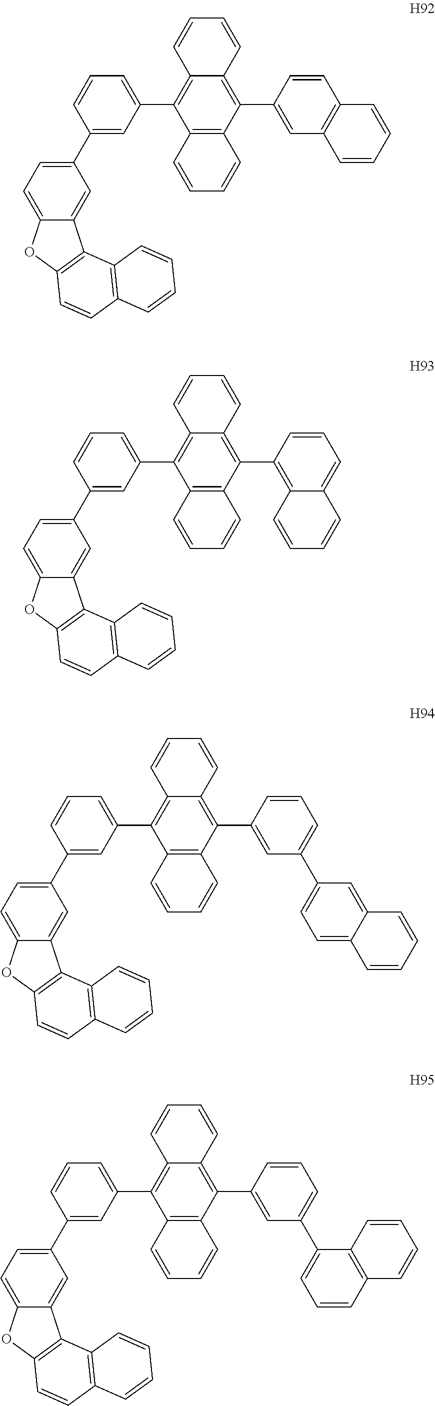 [SMILES: c1cc(-c2ccc3oc4ccc5ccccc5c4c3c2)cc(-c2c3ccccc3c(-c3ccc4ccccc4c3)c3ccccc23)c1 ; c1cc(-c2ccc3oc4ccc5ccccc5c4c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6ccc7ccccc7c6c5c4)c3)c3ccccc23)c1 ; c1cc(-c2ccc3oc4ccc5ccccc5c4c3c2)cc(-c2c3ccccc3c(-c3cccc(-c4cccc5ccccc45)c3)c3ccccc23)c1]

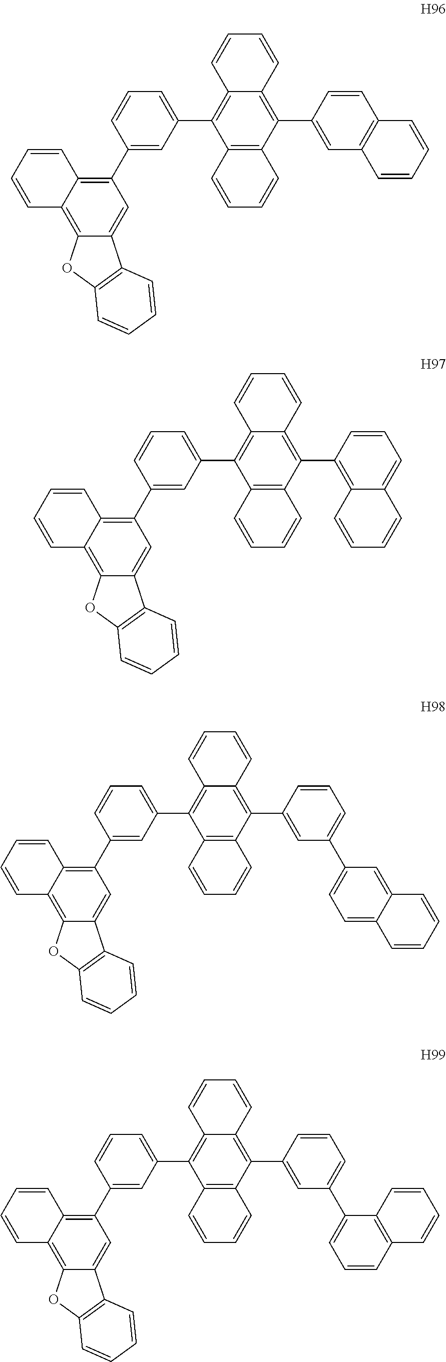 [SMILES: c1cc(-c2c3ccccc3c(-c3ccc4ccccc4c3)c3ccccc23)cc(-c2cc3c4ccccc4oc3c3ccccc23)c1 ; c1cc(-c2c3ccccc3c(-c3cccc4ccccc34)c3ccccc23)cc(-c2cc3c4ccccc4oc3c3ccccc23)c1 ; c1cc(-c2ccc3ccccc3c2)cc(-c2c3ccccc3c(-c3cccc(-c4cc5c6ccccc6oc5c5ccccc45)c3)c3ccccc23)c1 ; c1cc(-c2cccc3ccccc23)cc(-c2c3ccccc3c(-c3cccc(-c4cc5c6ccccc6oc5c5ccccc45)c3)c3ccccc23)c1]

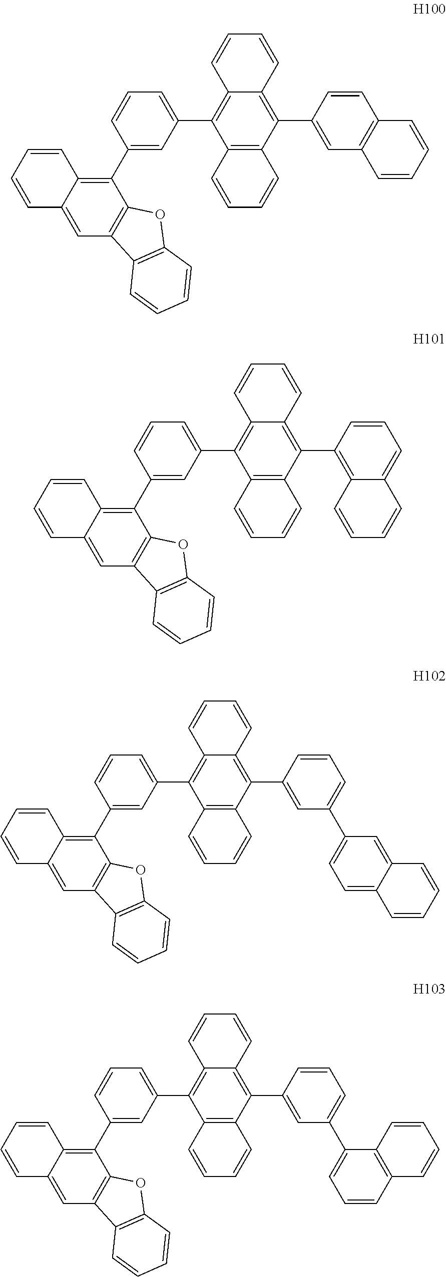 [SMILES: c1cc(-c2c3ccccc3c(-c3ccc4ccccc4c3)c3ccccc23)cc(-c2c3ccccc3cc3c2oc2ccccc23)c1 ; c1cc(-c2c3ccccc3c(-c3cccc4ccccc34)c3ccccc23)cc(-c2c3ccccc3cc3c2oc2ccccc23)c1 ; c1cc(-c2ccc3ccccc3c2)cc(-c2c3ccccc3c(-c3cccc(-c4c5ccccc5cc5c4oc4ccccc45)c3)c3ccccc23)c1 ; c1cc(-c2cccc3ccccc23)cc(-c2c3ccccc3c(-c3cccc(-c4c5ccccc5cc5c4oc4ccccc45)c3)c3ccccc23)c1]

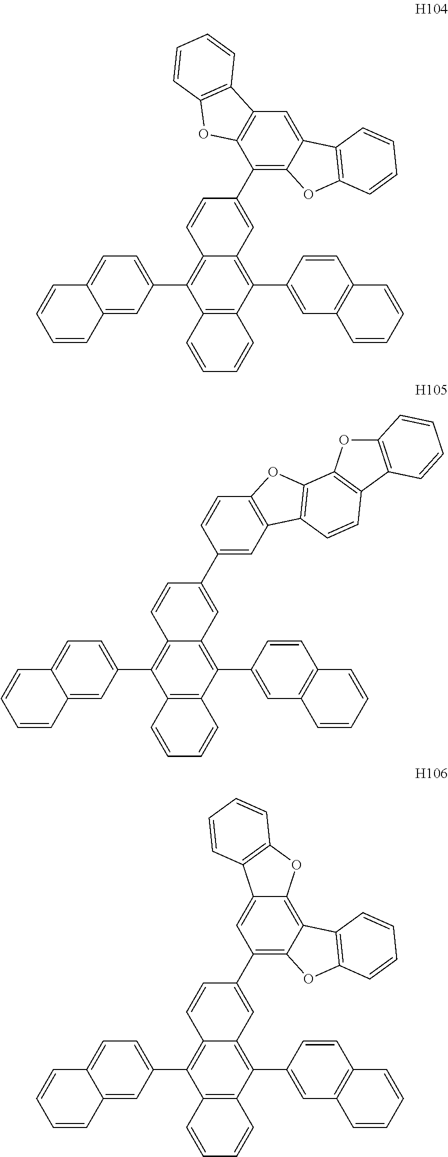

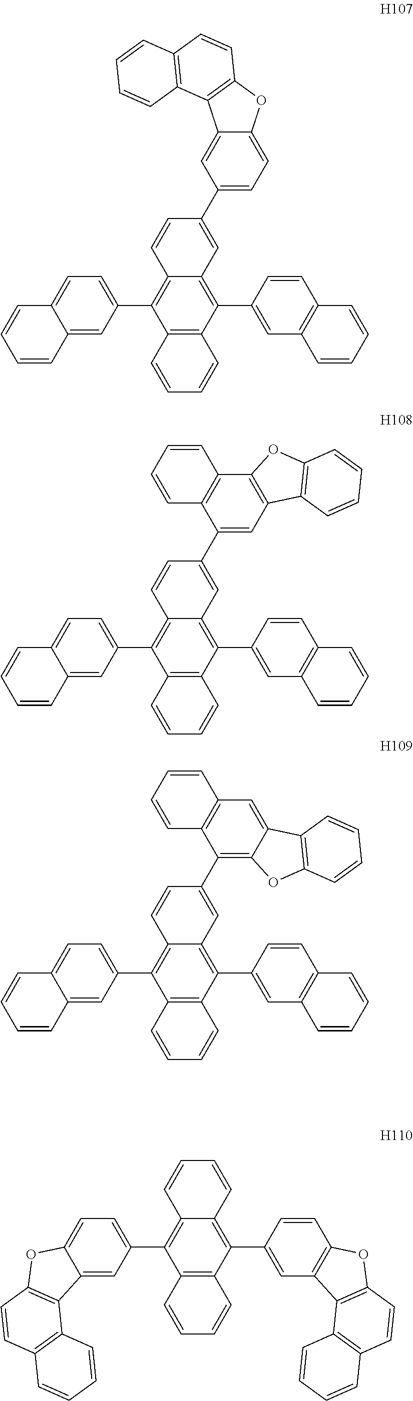

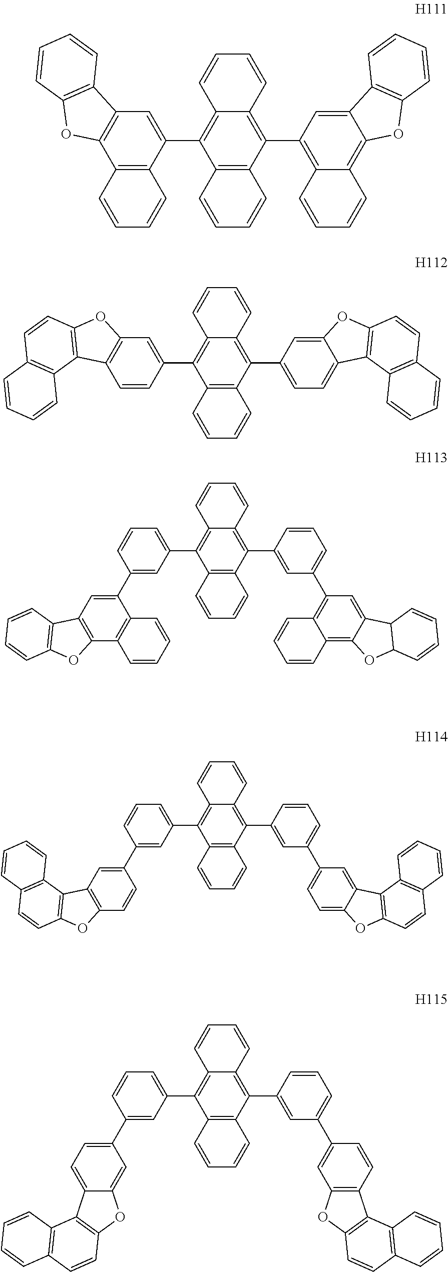 [SMILES: c1ccc2c(c1)oc1c3ccccc3c(-c3c4ccccc4c(-c4cc5c6ccccc6oc5c5ccccc45)c4ccccc34)cc21 ; c1ccc2c(c1)ccc1oc3cc(-c4c5ccccc5c(-c5ccc6c(c5)oc5ccc7ccccc7c56)c5ccccc45)ccc3c12 ; C1=CC2Oc3c(cc(-c4cccc(-c5c6ccccc6c(-c6cccc(-c7cc8c9ccccc9oc8c8ccccc78)c6)c6ccccc56)c4)c4ccccc34)C2C=C1 ; c1cc(-c2ccc3oc4ccc5ccccc5c4c3c2)cc(-c2c3ccccc3c(-c3cccc(-c4ccc5oc6ccc7ccccc7c6c5c4)c3)c3ccccc23)c1 ; c1cc(-c2ccc3c(c2)oc2ccc4ccccc4c23)cc(-c2c3ccccc3c(-c3cccc(-c4ccc5c(c4)oc4ccc6ccccc6c45)c3)c3ccccc23)c1]

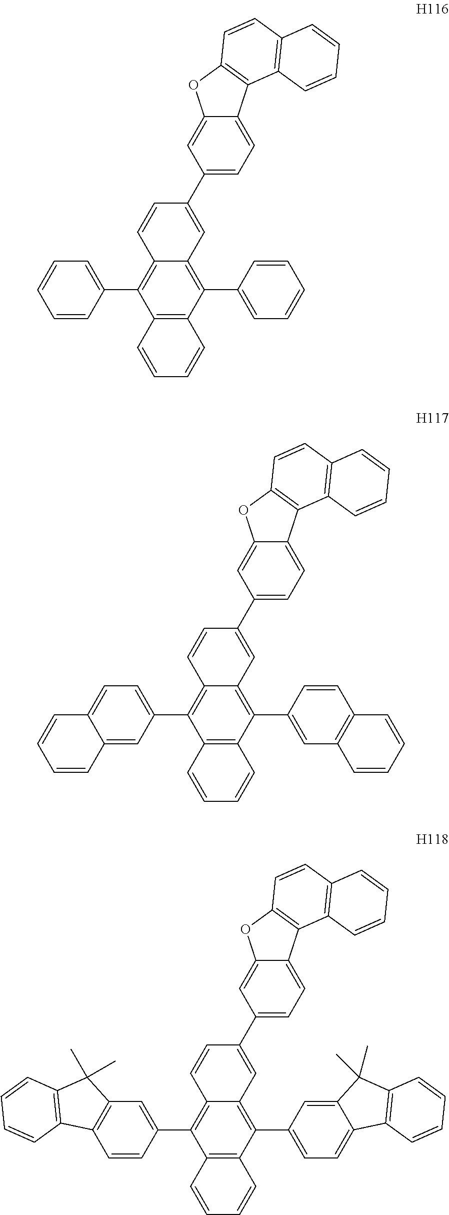 [SMILES: c1ccc(-c2c3ccccc3c(-c3ccccc3)c3cc(-c4ccc5c(c4)oc4ccc6ccccc6c45)ccc23)cc1 ; c1ccc2cc(-c3c4ccccc4c(-c4ccc5ccccc5c4)c4cc(-c5ccc6c(c5)oc5ccc7ccccc7c56)ccc34)ccc2c1 ; CC1(C)c2ccccc2-c2ccc(-c3c4ccccc4c(-c4ccc5c(c4)C(C)(C)c4ccccc4-5)c4cc(-c5ccc6c(c5)oc5ccc7ccccc7c56)ccc34)cc21]

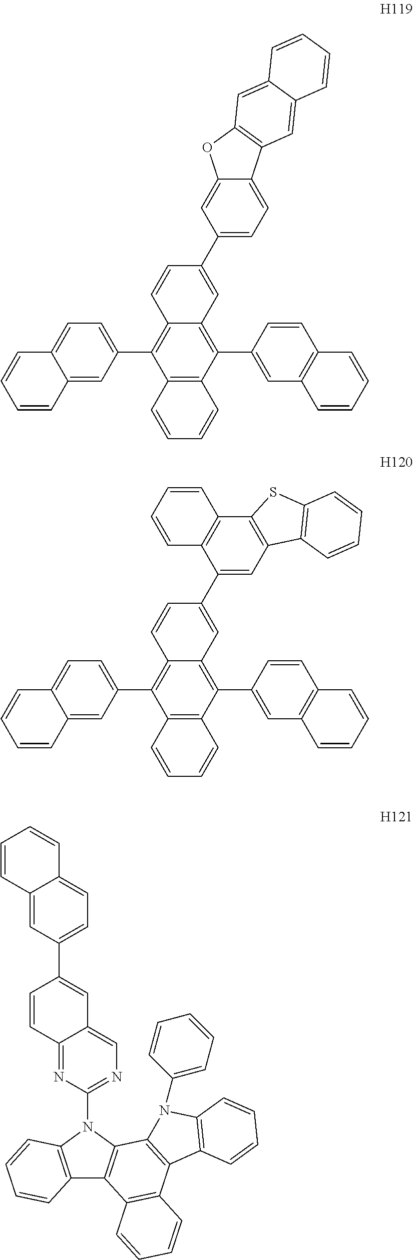 [SMILES: c1ccc2cc(-c3c4ccccc4c(-c4ccc5ccccc5c4)c4cc(-c5ccc6c(c5)oc5cc7ccccc7cc56)ccc34)ccc2c1 ; c1ccc2cc(-c3c4ccccc4c(-c4ccc5ccccc5c4)c4cc(-c5cc6c7ccccc7sc6c6ccccc56)ccc34)ccc2c1 ; c1ccc(-n2c3ccccc3c3c4ccccc4c4c5ccccc5n(-c5ncc6cc(-c7ccc8ccccc8c7)ccc6n5)c4c32)cc1]

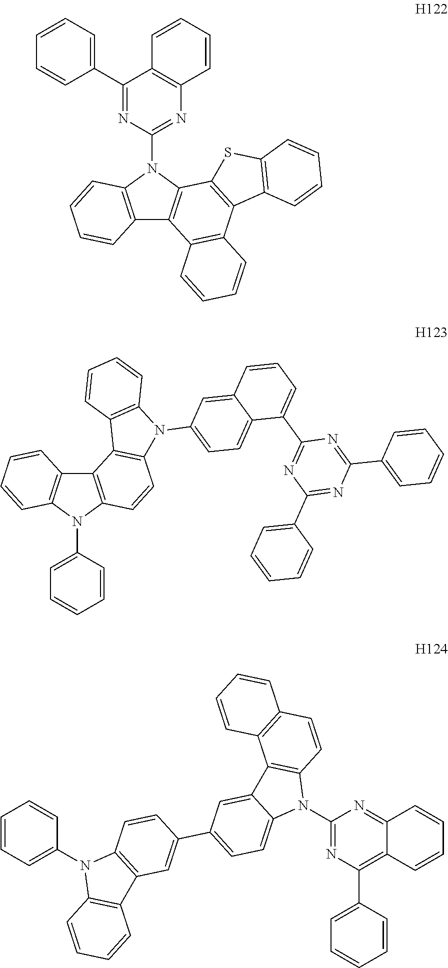 [SMILES: c1ccc(-c2nc(-n3c4ccccc4c4c5ccccc5c5c6ccccc6sc5c43)nc3ccccc23)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

### Phosphorescent Dopant

In an embodiment, the phosphorescent dopant may include (e.g., be) at least one transition metal as a central metal.

The phosphorescent dopant may include (e.g., be) a monodentate ligand, a bidentate ligand, a tridentate ligand, a tetradentate ligand, a pentadentate ligand, a hexadentate ligand, or any combination thereof.

The phosphorescent dopant may be electrically neutral.

For example, the phosphorescent dopant may include (e.g., be) an organometallic compound represented by Formula 401:

M(L₄₀₁)_{xc1}(L₄₀₂)_{xc2} Formula 401

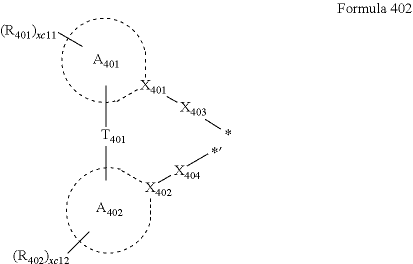

In Formulae 401 and 402,

M may be a transition metal (for example, iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),

L₄₀₁ may be a ligand represented by Formula 402, and xc1 may be 1, 2, or 3, wherein when xc1 is two or more, two or more of L₄₀₁(s) may be identical to or different from each other,

L₄₀₂ may be an organic ligand, and xc2 may be 0, 1, 2, 3, or 4, and when xc2 is 2 or more, two or more of L₄₀₂(s) may be identical to or different from each other,

X₄₀₁ and X₄₀₂ may each independently be nitrogen or carbon,

ring A₄₀₁ and ring A₄₀₂ may each independently be a C₃-C₆₀ carbocyclic group or a C₁-C₆₀ heterocyclic group,

T₄₀₁ may be a single bond, *—O—*′, *—S—*, *—C(═O)—*′, *—N(Q₄₁₁)-*′, *—C(Q₄₁₁)(Q₄₁₂)-*′, *—C(Q₄₁₁)=C(Q₄₁₂)-*′, *—C(Q₄₁₁)-*′, or *═C(Q₄₁₁)-*′,

X₄₀₃ and X₄₀₄ may each independently be a chemical bond (for example, a covalent bond or a coordination bond), O, S, N(Q₄₁₃), B(Q₄₁₃), P(Q₄₁₃), C(Q₄₁₃)(Q₄₁₄), or Si(Q₄₁₃)(Q₄₁₄),

Q₄₁₁ to Q₄₁₄ may each independently be the same as described in connection with Q₁,

R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),

Q₄₀₁ to Q₄₀₃ may each independently be the same as described in connection with Q₁,

xc11 and xc12 may each independently be an integer selected from 0 to 10, and

* and *′ in Formula 402 each indicate a binding site to M in Formula 401.

For example, in Formula 402, i) X₄₀₁ is nitrogen, and X₄₀₂ is carbon, or ii) each of X₄₀₁ and X₄₀₂ is nitrogen.

In an embodiment, when xc1 in Formula 402 is 2 or more, two ring A₄₀₁ in two or more of L₄₀₁(s) may be optionally linked to each other via T₄₀₂, which is a linking group, and/or two ring A₄₀₂ may optionally be linked to each other via T₄₀₃, which is a linking group (see Compounds PD1 to PD4 and PD7). T₄₀₂ and T₄₀₃ may each independently be the same as described in connection with T₄₀₁.

L₄₀₂ in Formula 401 may be an organic ligand. For example, L₄₀₂ may include a halogen group, a diketone group (for example, an acetylacetonate group), a carboxylic acid group (for example, a picolinate group), —C(═O), an isonitrile group, —CN group, a phosphorus group (for example, a phosphine group, a phosphite group, etc.), or any combination thereof.

The phosphorescent dopant may include, for example be, one of Compounds PD1 to PD25, or any combination thereof:

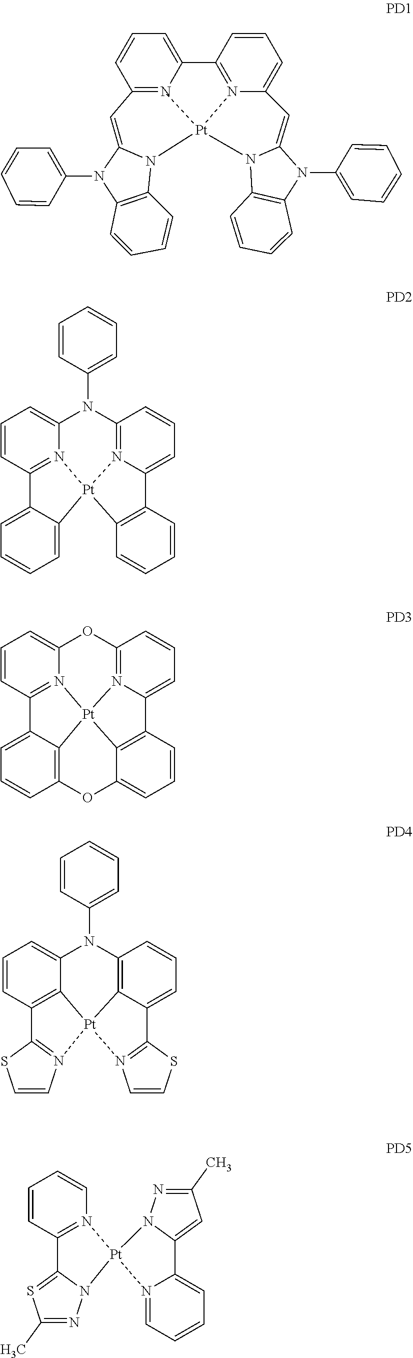

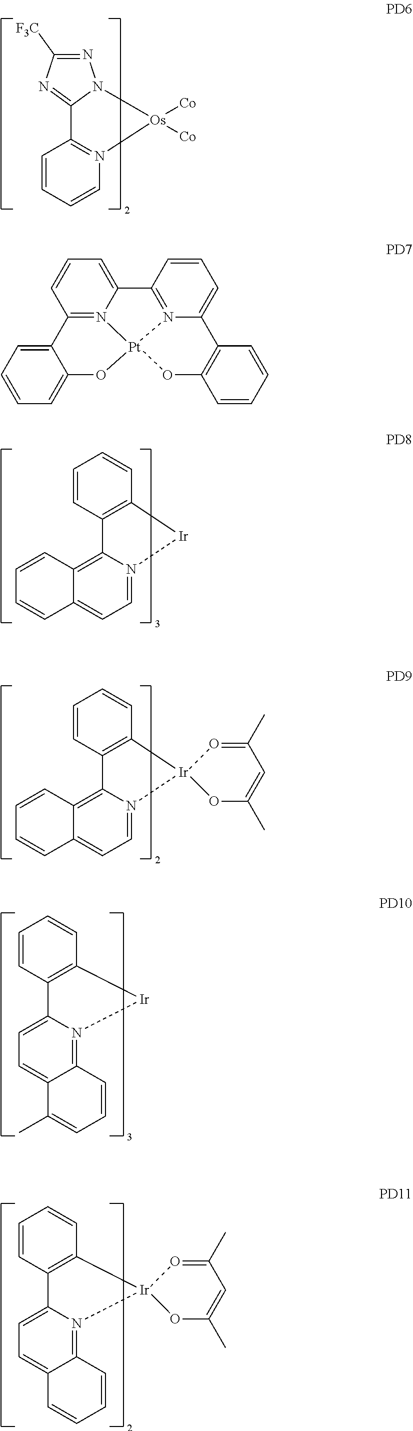

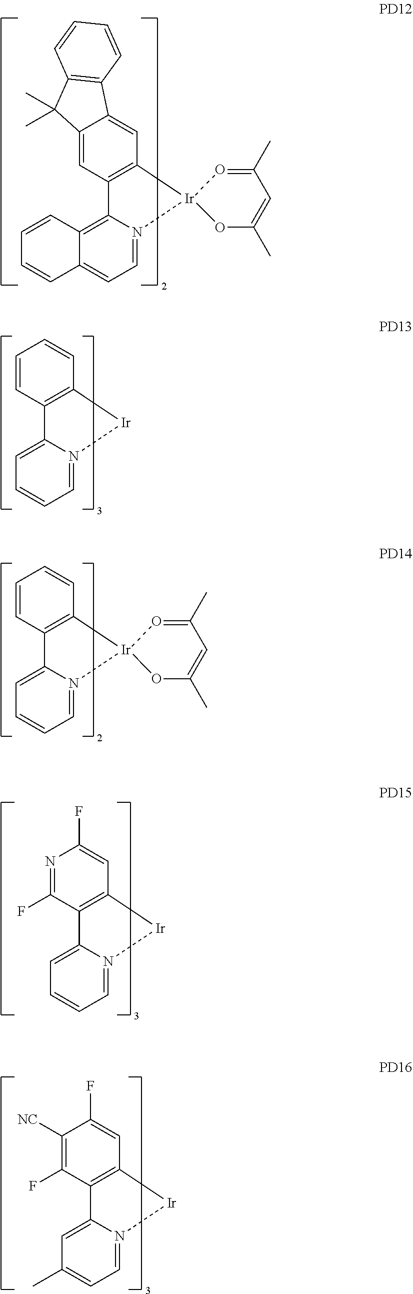

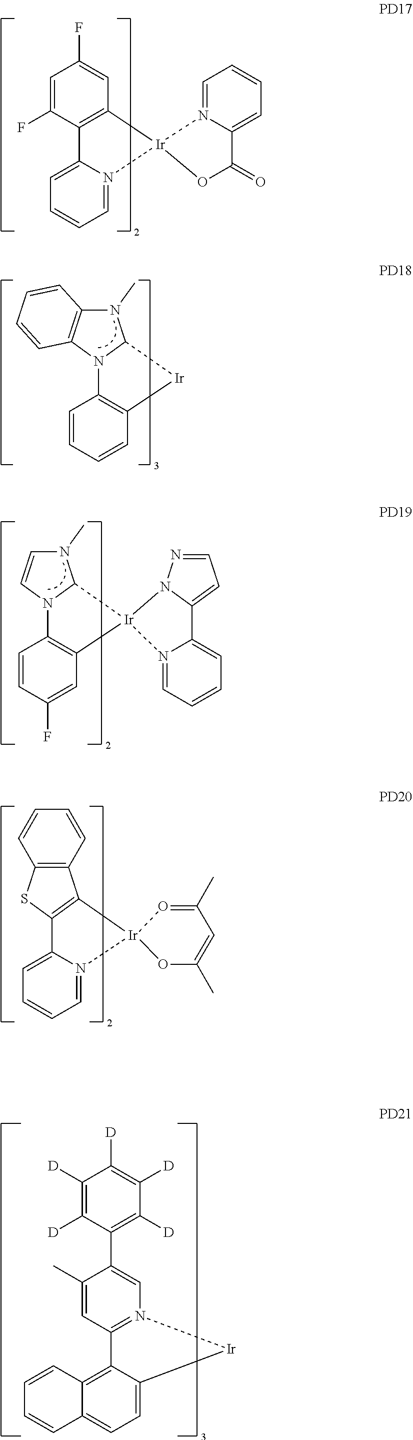

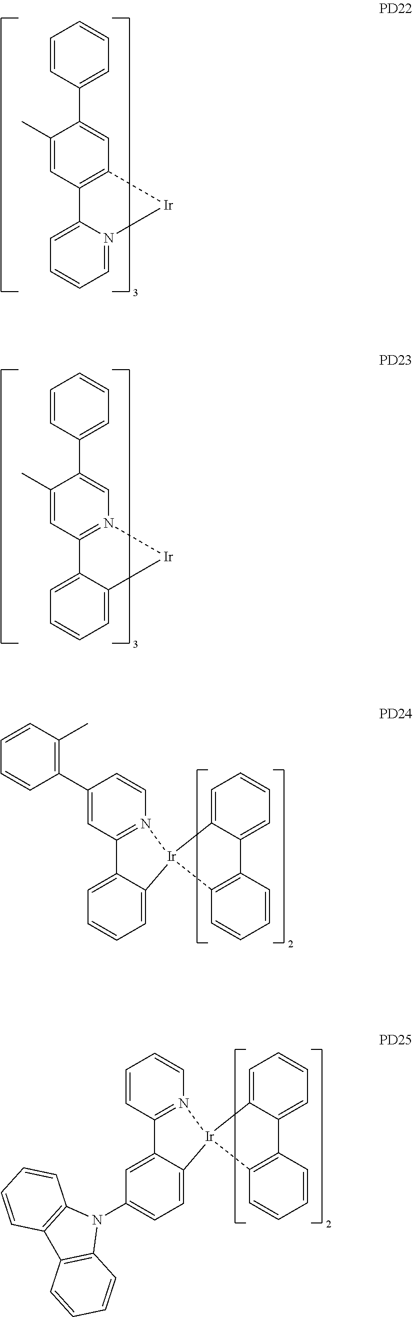

### Fluorescent Dopant

The fluorescent dopant may include (e.g., be) an amine group-containing compound, a styryl group-containing compound, or any combination thereof.

In an embodiment, the fluorescent dopant may include (e.g., be) a compound represented by Formula 501:

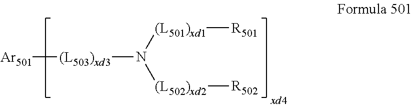

wherein, in Formula 501,

Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xd1 to xd3 may each independently be 0, 1, 2, or 3, and

xd4 may be 1, 2, 3, 4, 5, or 6.

In an embodiment, Ar₅₀₁ in Formula 501 may be a condensed cyclic group (for example, an anthracene group, a chrysene group, and/or a pyrene group) in which three or more monocyclic groups are condensed together.

In one or more embodiments, xd4 in Formula 501 may be 2.

In an embodiment, the fluorescent dopant may include (e.g., be): one of Compounds FD1 to FD36; DPVBi; DPAVBi; or any combination thereof:

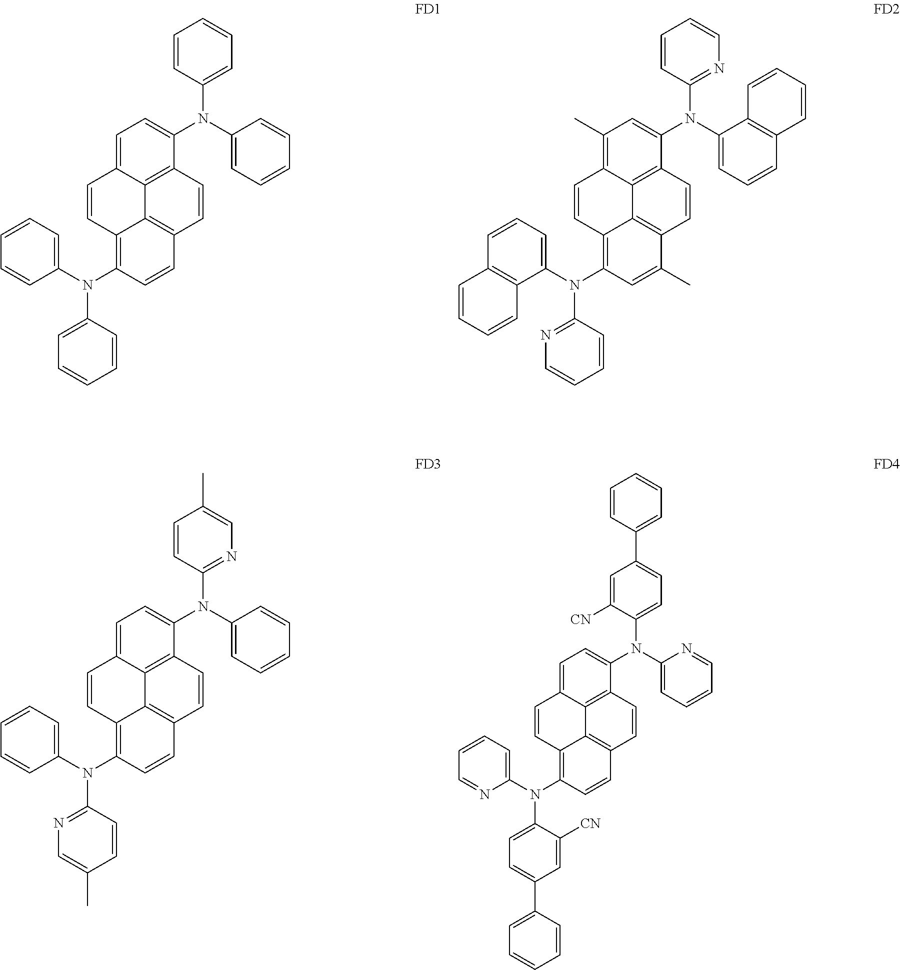

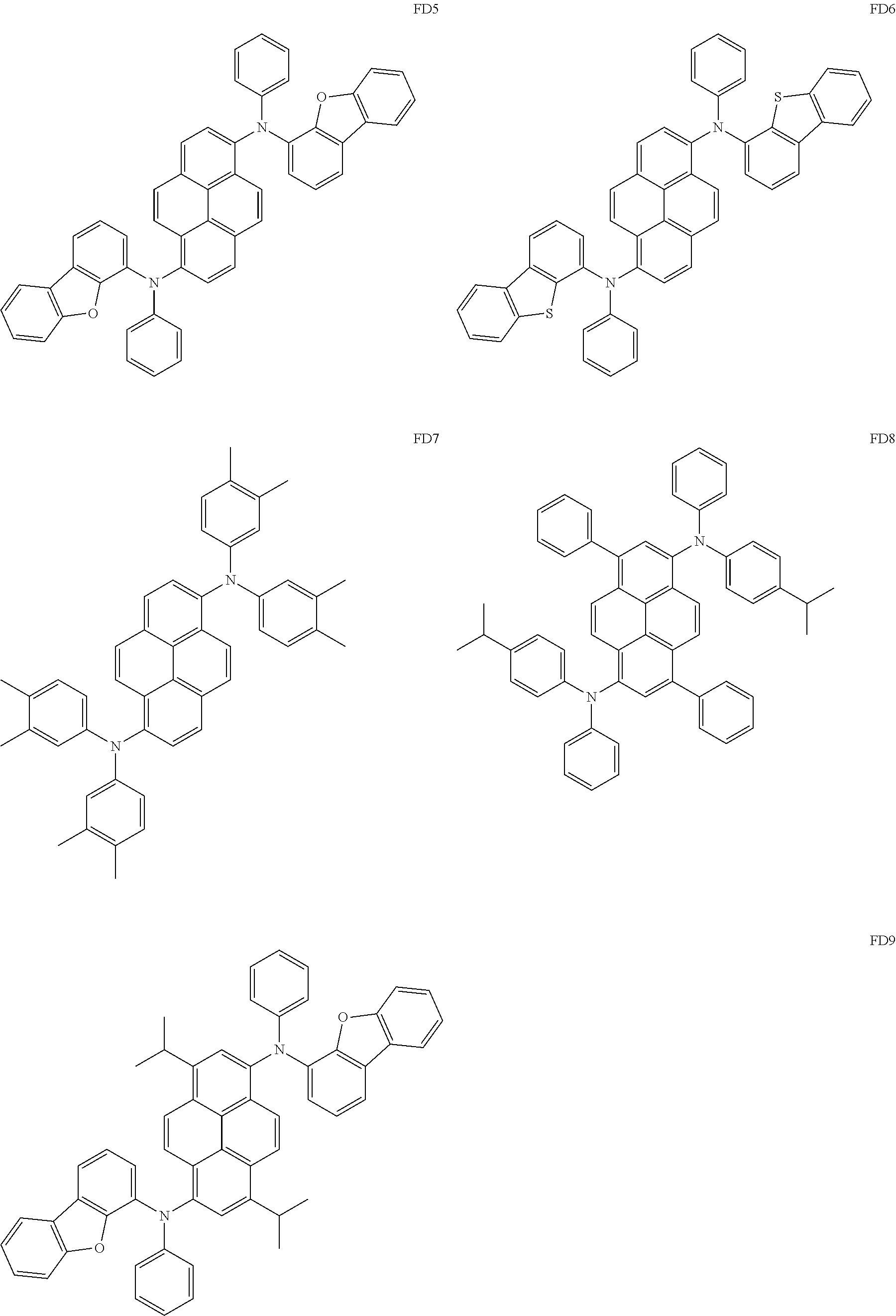

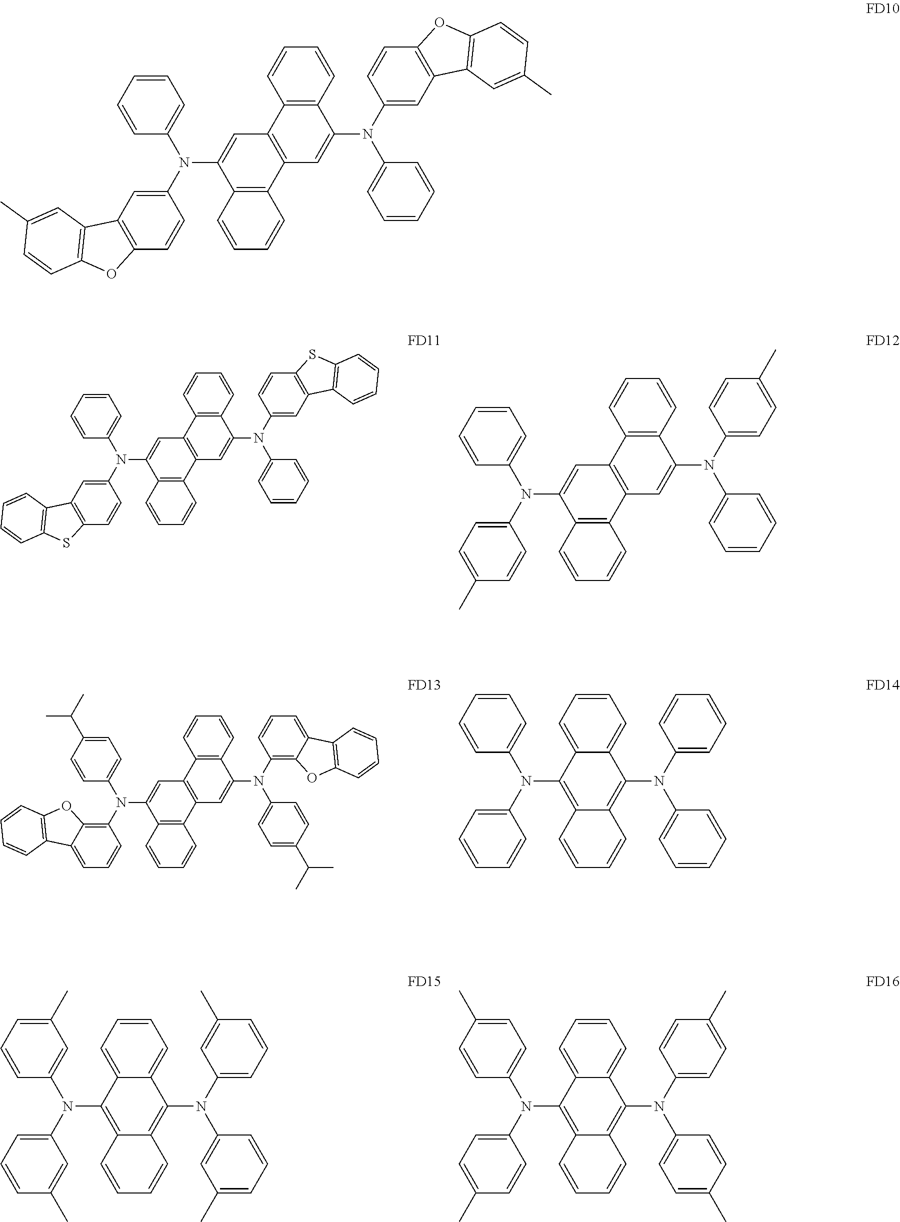

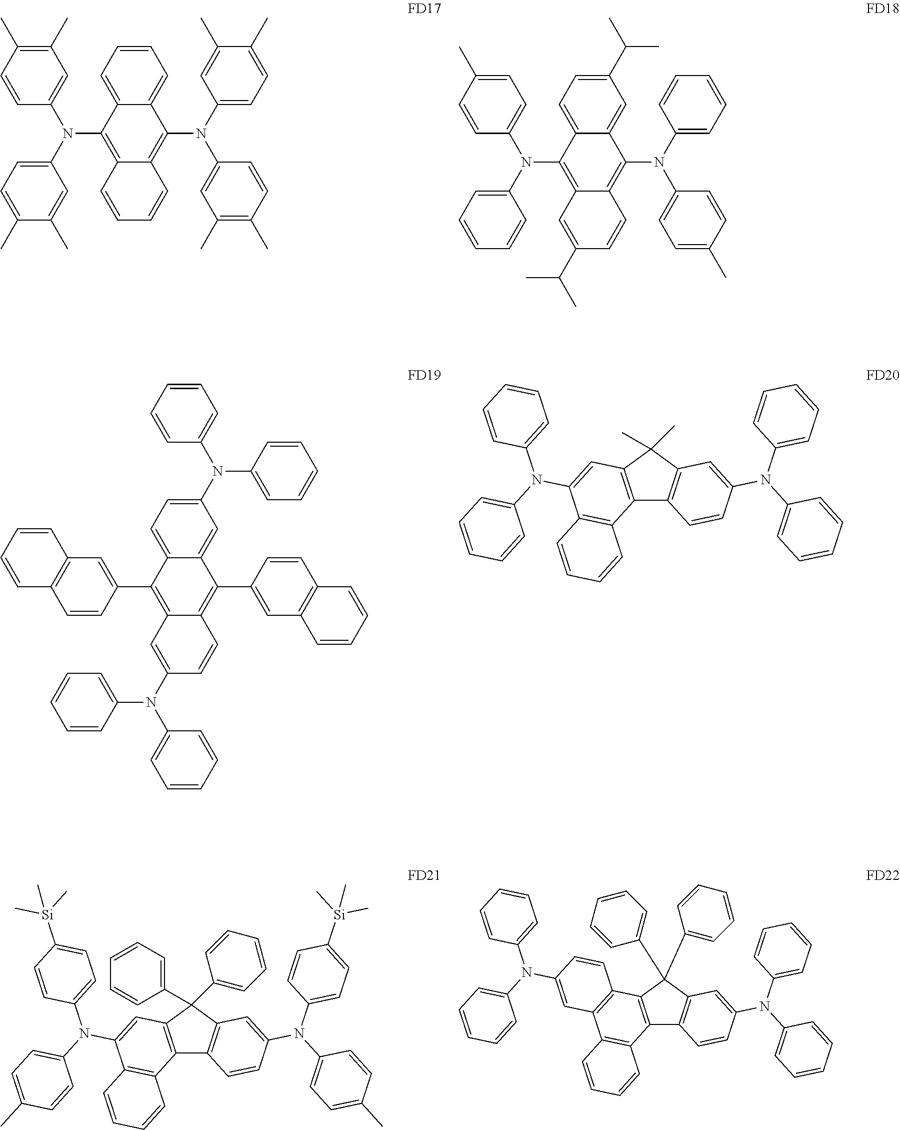

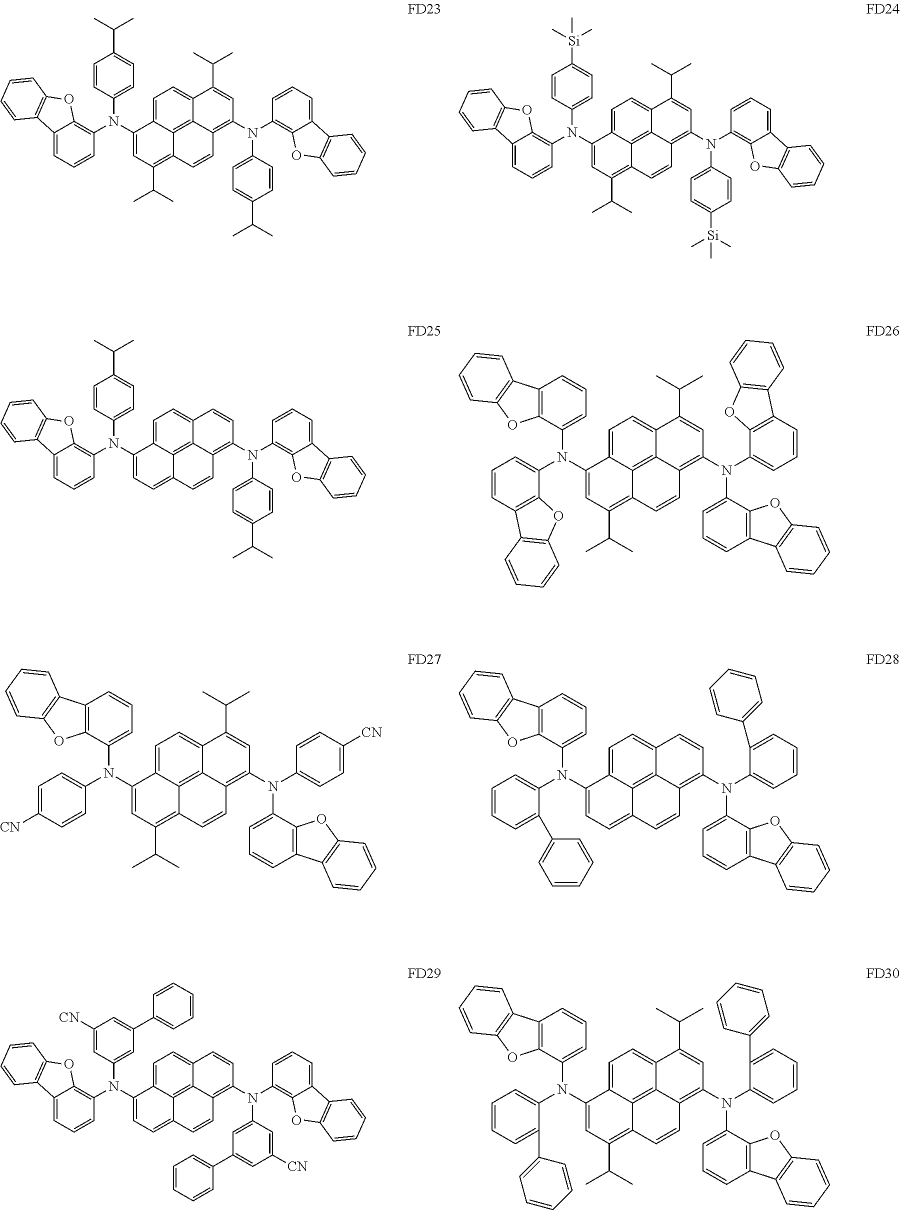

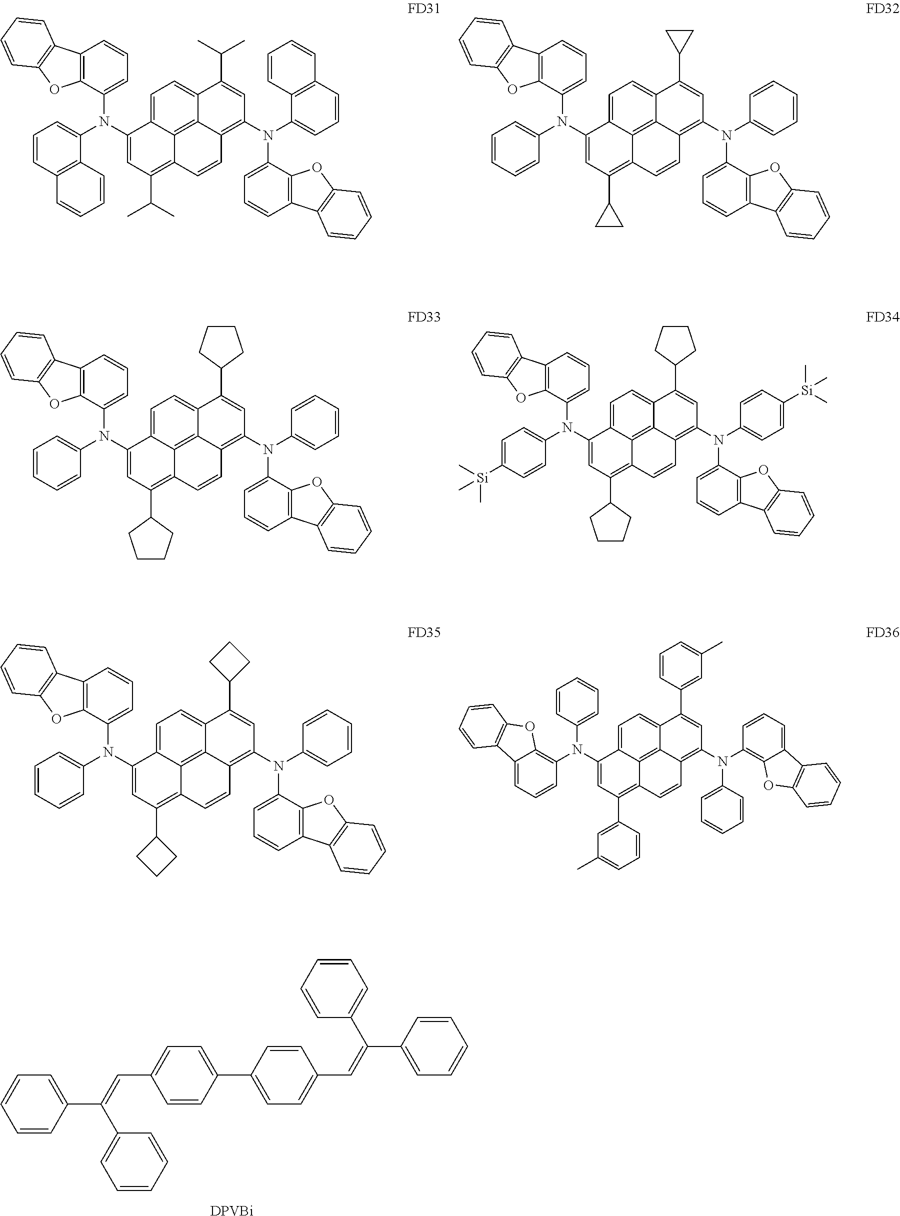

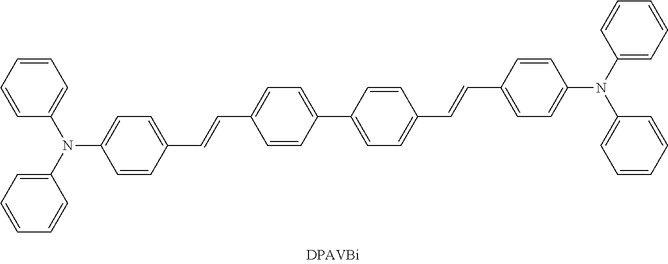 [SMILES: C(=C\c1ccc(N(c2ccccc2)c2ccccc2)cc1)/c1ccc(-c2ccc(/C=C/c3ccc(N(c4ccccc4)c4ccccc4)cc3)cc2)cc1]

### Delayed Fluorescence Material

In an embodiment, the emission layer may include a delayed fluorescence material.

The delayed fluorescence material may be selected from compounds capable of emitting delayed fluorescence based on a delayed fluorescence emission mechanism.

The delayed fluorescence material included in the emission layer may act as (e.g., be) a host or a dopant depending on the kind (e.g., type) of other materials included in the emission layer.

In an embodiment, the difference between the triplet energy level (eV) of the delayed fluorescence material and the singlet energy level (eV) of the delayed fluorescence material may be greater than or equal to 0 eV and less than or equal to 0.5 eV. When the difference between the triplet energy level (eV) of the delayed fluorescence material and the singlet energy level (eV) of the delayed fluorescence material satisfies the above-described range, up-conversion from the triplet state to the singlet state of the delayed fluorescence material may effectively occur, and thus, the emission efficiency of the light-emitting device 10 may be improved.

In an embodiment, the delayed fluorescence material may include i) a material including at least one electron donor (for example, a π electron-rich C₃-C₆₀ cyclic group, such as a carbazole group) and at least one electron acceptor (for example, a sulfoxide group, a cyano group, and/or a π electron-deficient nitrogen-containing C₁-C₆₀ cyclic group), and/or ii) a material including a C₈-C₆₀ polycyclic group in which two or more cyclic groups are condensed while sharing boron (B).

In an embodiment, the delayed fluorescence material may include at least one of the following Compounds DF1 to DF9:

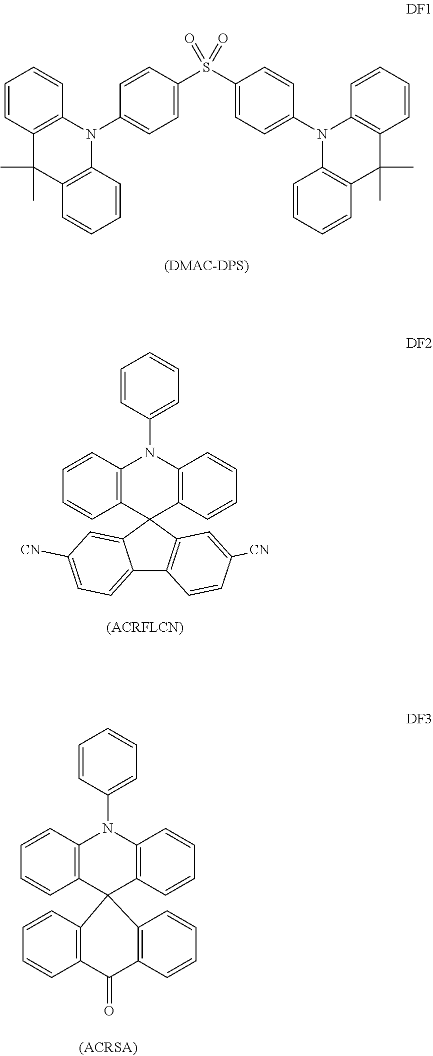

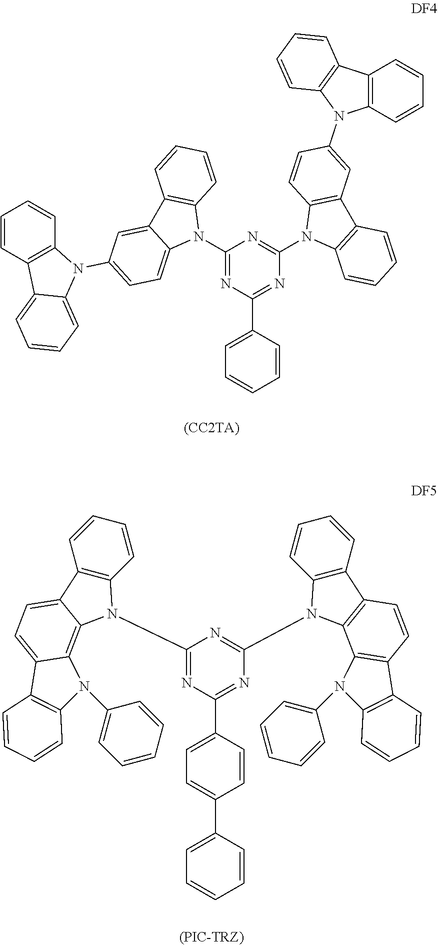 [SMILES: c1ccc(-c2nc(-n3c4ccccc4c4cc(-n5c6ccccc6c6ccccc65)ccc43)nc(-n3c4ccccc4c4cc(-n5c6ccccc6c6ccccc65)ccc43)n2)cc1 ; c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1]

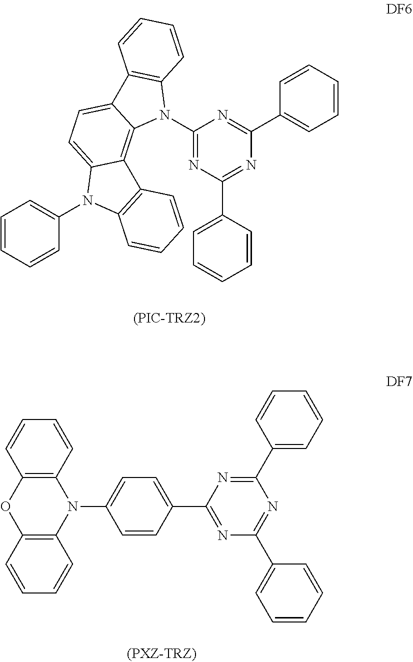 [SMILES: c1ccc(-c2nc(-c3ccccc3)nc(-n3c4ccccc4c4ccc5c(c6ccccc6n5-c5ccccc5)c43)n2)cc1 ; c1ccc(-c2nc(-c3ccccc3)nc(-c3ccc(N4c5ccccc5Oc5ccccc54)cc3)n2)cc1]

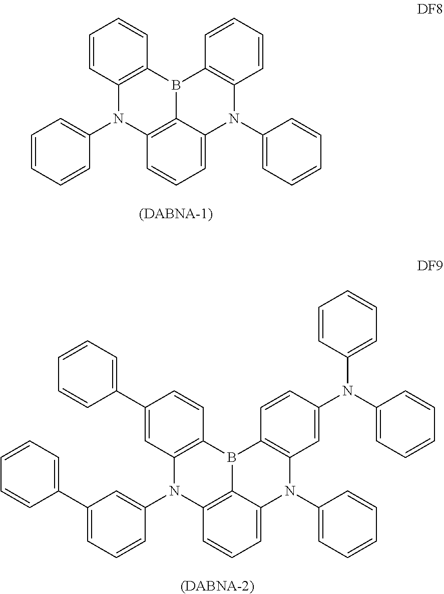

### Quantum Dot

The emission layer may include a quantum dot.

In the present specification, a quantum dot refers to a crystal of a semiconductor compound, and may include any material capable of emitting light of various emission wavelengths according to the size of the crystal.

A diameter of the quantum dot may be, for example, in a range of about 1 nm to about 10 nm.

The quantum dot may be synthesized by a wet chemical process, a metal organic (e.g., organometallic) chemical vapor deposition process, a molecular beam epitaxy process, or any suitable process similar thereto.

In the wet chemical process, a precursor material is mixed with an organic solvent to grow a quantum dot particle crystal. When the crystal grows, the organic solvent naturally acts as a dispersant coordinated on the surface of the quantum dot crystal and controls the growth of the crystal so that the growth of quantum dot particles can be controlled through a process which is more easily performed than vapor deposition methods, such as metal organic chemical vapor deposition (MOCVD) and/or molecular beam epitaxy (MBE), and which is a low cost process.

The quantum dot may include Groups II-VI semiconductor compounds, Groups III-V semiconductor compounds, Groups III-VI semiconductor compounds, Groups I-III-VI semiconductor compounds, Groups IV-VI semiconductor compounds, a Group IV element or compound; or any combination thereof.

Non-limiting examples of the Groups II-VI semiconductor compound are a binary compound, such as CdSe, CdTe, ZnS, ZnSe, ZnTe, ZnO, HgS, HgSe, HgTe, MgSe, and/or MgS; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, and/or MgZnS; a quaternary compound, such as CdZnSeS, CdZnSeTe, CdZnSTe, CdHgSeS, CdHgSeTe, CdHgSTe, HgZnSeS, HgZnSeTe, and/or HgZnSTe; or any combination thereof.

Non-limiting examples of the Groups III-V semiconductor compound are a binary compound, such as GaN, GaP, GaAs, GaSb, AlN, AIP, AlAs, AlSb, InN, InP, InAs, InSb, or the like; a ternary compound, such as GaNP, GaNAs, GaNSb, GaPAs, GaPSb, AlNP, AlNAs, AlNSb, AlPAs, AlPSb, InGaP, InNP, InAlP, InNAs, InNSb, InPAs, InPSb, and/or the like; a quaternary compound, such as GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, GaAlNP, and/or the like; or any combination thereof. The Groups III-V semiconductor compound may further include a Group II element. Non-limiting examples of the Groups III-V further including a Group II element are InZnP, InGaZnP, InAlZnP, etc.

Non-limiting examples of the Groups III-VI semiconductor compound are a binary compound, such as GaS, GaSe, Ga₂Se₃, GaTe, InS, InSe, In₂S₃, In₂Se₃, or InTe; a ternary compound, such as InGaS₃, and/or InGaSe₃; or any combination thereof.

Non-limiting examples of the Groups I-III-VI semiconductor compound are a ternary compound, such as AgInS, AgInS₂, CuInS, CuInS₂, CuGaO₂, AgGaO₂, and/or AgAlO₂; or any combination thereof.

Non-limiting examples of the Groups IV-VI semiconductor compound are a binary compound, such as SnS, SnSe, SnTe, PbS, PbSe, PbTe, and/or the like; a ternary compound, such as SnSeS, SnSeTe, SnSTe, PbSeS, PbSeTe, PbSTe, SnPbS, SnPbSe, SnPbTe, and/or the like; a quaternary compound, such as SnPbSSe, SnPbSeTe, SnPbSTe, and/or the like; or any combination thereof.

The Group IV element or compound may include a single element compound, such as Si and/or Ge; a binary compound, such as SiC and/or SiGe; or any combination thereof.

Each element included in a multi-element compound such as the binary compound, ternary compound and quaternary compound, may exist in a particle with a uniform concentration or non-uniform concentration.

In one embodiment, the quantum dot may have a single structure or a dual core-shell structure. In the case of the quantum dot having a single structure, the concentration of each element included in the corresponding quantum dot is uniform. In an embodiment, in the dual core-shell structure, the material contained in the core and the material contained in the shell may be different from each other.

The shell of the quantum dot may act as (e.g., be) a protective layer to reduce or prevent chemical degeneration of the core to maintain semiconductor characteristics and/or as a charging layer to impart electrophoretic characteristics to the quantum dot. The shell may be a single layer or a multi-layer. The interface between the core and the shell may have a concentration gradient in which the concentration of elements existing in the shell decreases toward the center of the of the core.

Non-limiting examples of the shell of the quantum dot may be an oxide of metal, metalloid, or non-metal; a semiconductor compound; or any combination thereof. Non-limiting examples of the oxide of metal, metalloid, or non-metal are a binary compound, such as SiO₂, Al₂O₃, TiO₂, ZnO, MnO, Mn₂O₃, Mn₃O₄, CuO, FeO, Fe₂O₃, Fe₃O₄, CoO, CO₃O₄, and/or NiO; a ternary compound, such as MgAl₂O₄, CoFe₂O₄, NiFe₂O₄, and/or CoMn₂O₄; or any combination thereof. Non-limiting examples of the semiconductor compound are, as described herein, Groups II-VI semiconductor compounds; Groups III-V semiconductor compounds; Groups III-VI semiconductor compounds; Groups I-III-VI semiconductor compounds; Groups IV-VI semiconductor compounds; or any combination thereof. In addition, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AIP, AlSb, or any combination thereof.

A full width at half maximum (FWHM) of an emission wavelength spectrum of the quantum dot may be about 45 nm or less, for example, about 40 nm or less, for example, about 30 nm or less, and within these ranges, color purity or color gamut may be increased. In addition, because the light emitted through the quantum dot is emitted in all directions, the wide viewing angle can be improved.

In addition, the quantum dot may be a spherical particle, a pyramidal particle, a multi-arm particle, a cubic nanoparticle, a nanotube particle, a nanowire particle, a nanofiber particle, or a nanoplate particle.

Because the energy band gap can be adjusted by controlling the size of the quantum dot, light having various wavelength bands can be obtained from the quantum dot emission layer. Therefore, by utilizing quantum dots of different sizes, a light-emitting display that emits light of various suitable wavelengths may be implemented. In an embodiment, the size of the quantum dot may be selected from to emit red, green and/or blue light. In addition, the size of the quantum dot may be configured to emit white light by combining light of various suitable colors.

### Electron Transport Region in Interlayer 130

The electron transport region may have: i) a single-layered structure consisting of a single layer consisting of a single material, ii) a single-layered structure consisting of a single layer consisting of a plurality of different materials, or iii) a multi-layered structure including a plurality of layers including different materials.

The electron transport region may include a buffer layer, a hole blocking layer, an electron control layer, an electron transport layer, an electron injection layer, or any combination thereof.

In an embodiment, the electron transport region may have an electron transport layer/electron injection layer structure, a hole blocking layer/electron transport layer/electron injection layer structure, an electron control layer/electron transport layer/electron injection layer structure, or a buffer layer/electron transport layer/electron injection layer structure, wherein, for each structure, constituting layers are sequentially stacked from an emission layer in the respective stated order.

In an embodiment, the electron transport region (for example, the buffer layer, the hole blocking layer, the electron control layer, or the electron transport layer in the electron transport region) may include a metal-free compound including at least one π electron-deficient nitrogen-containing C₁-C₆₀ cyclic group.

In an embodiment, the electron transport region may include a compound represented by Formula 601 below:

[Ar₆₀₁]ₓₑ₁₁-[(L₆₀₁)ₓₑ₁-R₆₀₁]ₓₑ₂₁ Formula 601

wherein, in Formula 601,

Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xe11 may be 1, 2, or 3,

xe1 may be 0, 1, 2, 3, 4, or 5,

R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),

Q₆₀₁ to Q₆₀₃ may each independently be the same as described in connection with Q₁,

xe21 may be 1, 2, 3, 4, or 5, and

at least one of Ar₆₀₁, L₆₀₁, or R₆₀₁ may each independently be air electron-deficient nitrogen-containing C₁-C₆₀ cyclic group unsubstituted or substituted with at least one R₁₀ₐ.

For example, when xe11 in Formula 601 is 2 or more, two or more of Ar₆₀₁(s) may be linked to each other via a single bond.

In an embodiment, Ar₆₀₁ in Formula 601 may be a substituted or unsubstituted anthracene group.

In an embodiment, the electron transport region may include a compound represented by Formula 601-1:

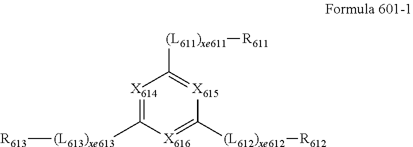

In Formula 601-1,

X₆₁₄ may be N or C(R₆₁₄), X₆₁₅ may be N or C(R₆₁₅), X₆₁₆ may be N or C(R₆₁₆), at least one of X₆₁₄ to X₆₁₆ may be N,

L₆₁₁ to L₆₁₃ may each independently be the same as described in connection with L₆₀₁,

xe611 to xe613 may each independently be the same as described in connection with xe1,

R₆₁₁ to R₆₁₃ may each independently be the same as described in connection with R₆₀₁,

R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

For example, xe1 and xe611 to xe613 in Formulae 601 and 601-1 may each independently be 0, 1, or 2.

The electron transport region may include one of Compounds ET1 to ET45, 2,9-dimethyl-4,7-diphenyl-1,10-phenanthroline (BCP), 4,7-diphenyl-1,10-phenanthroline (Bphen), Alq₃, BAlq, TAZ, NTAZ, or any combination thereof:

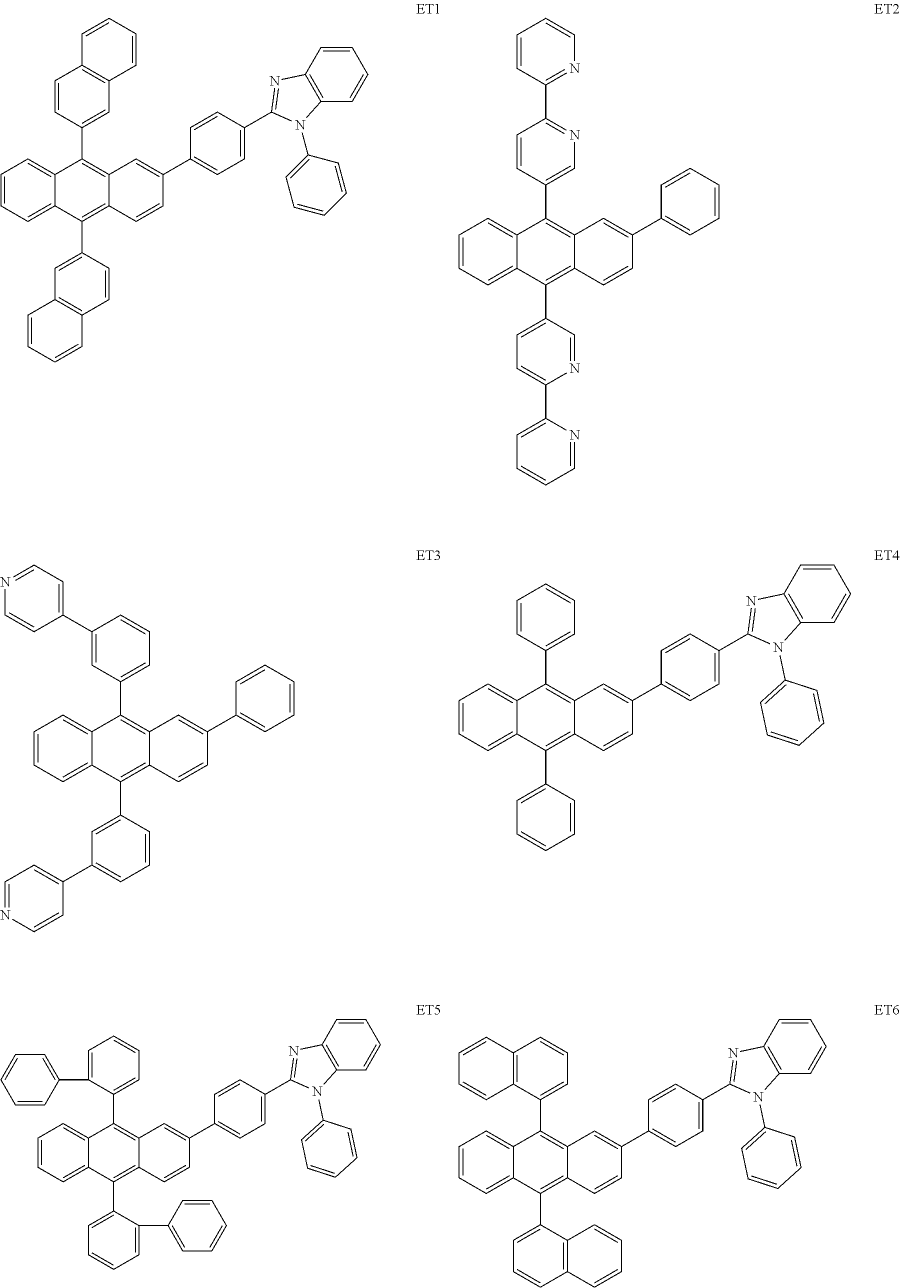

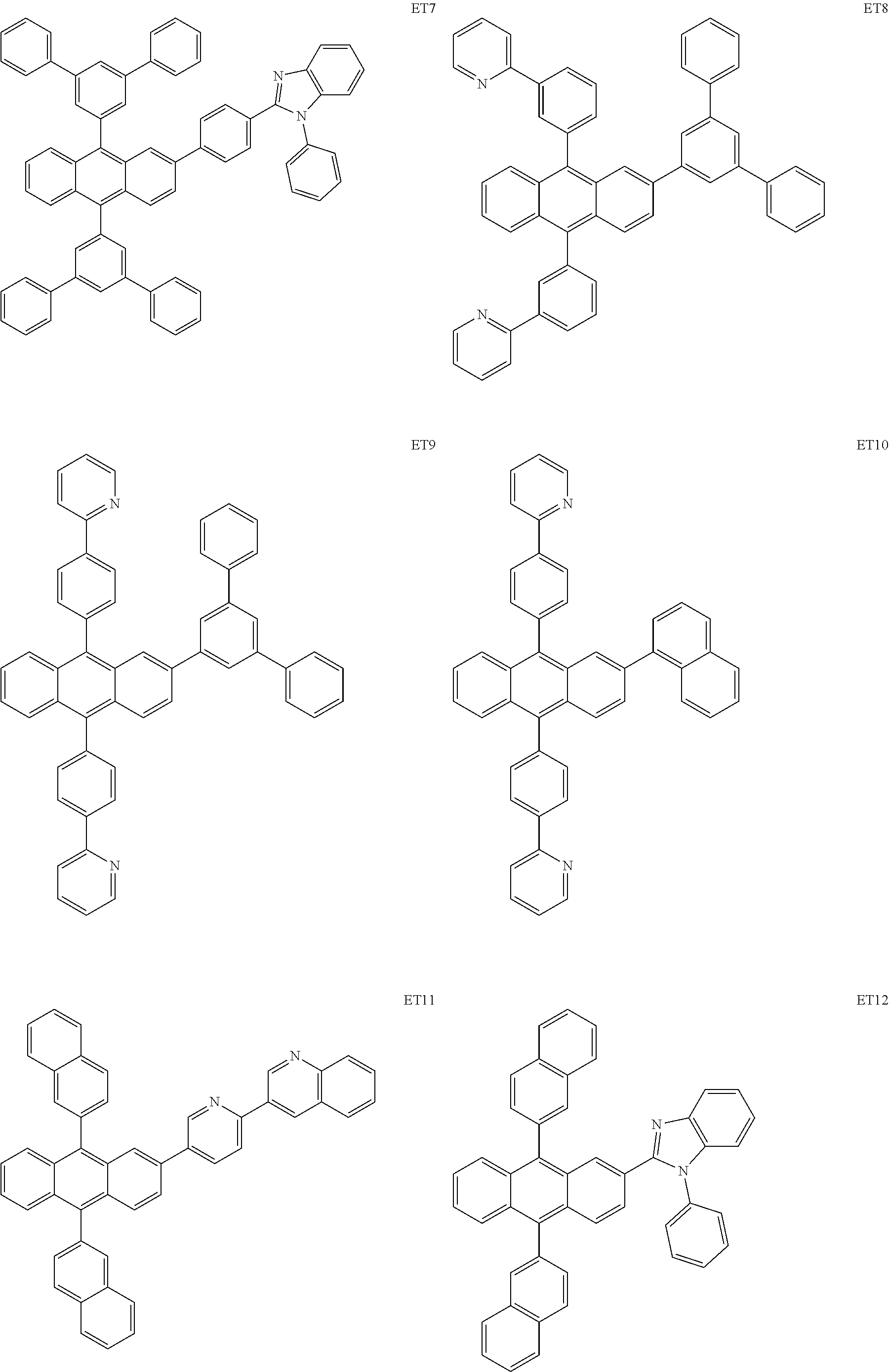

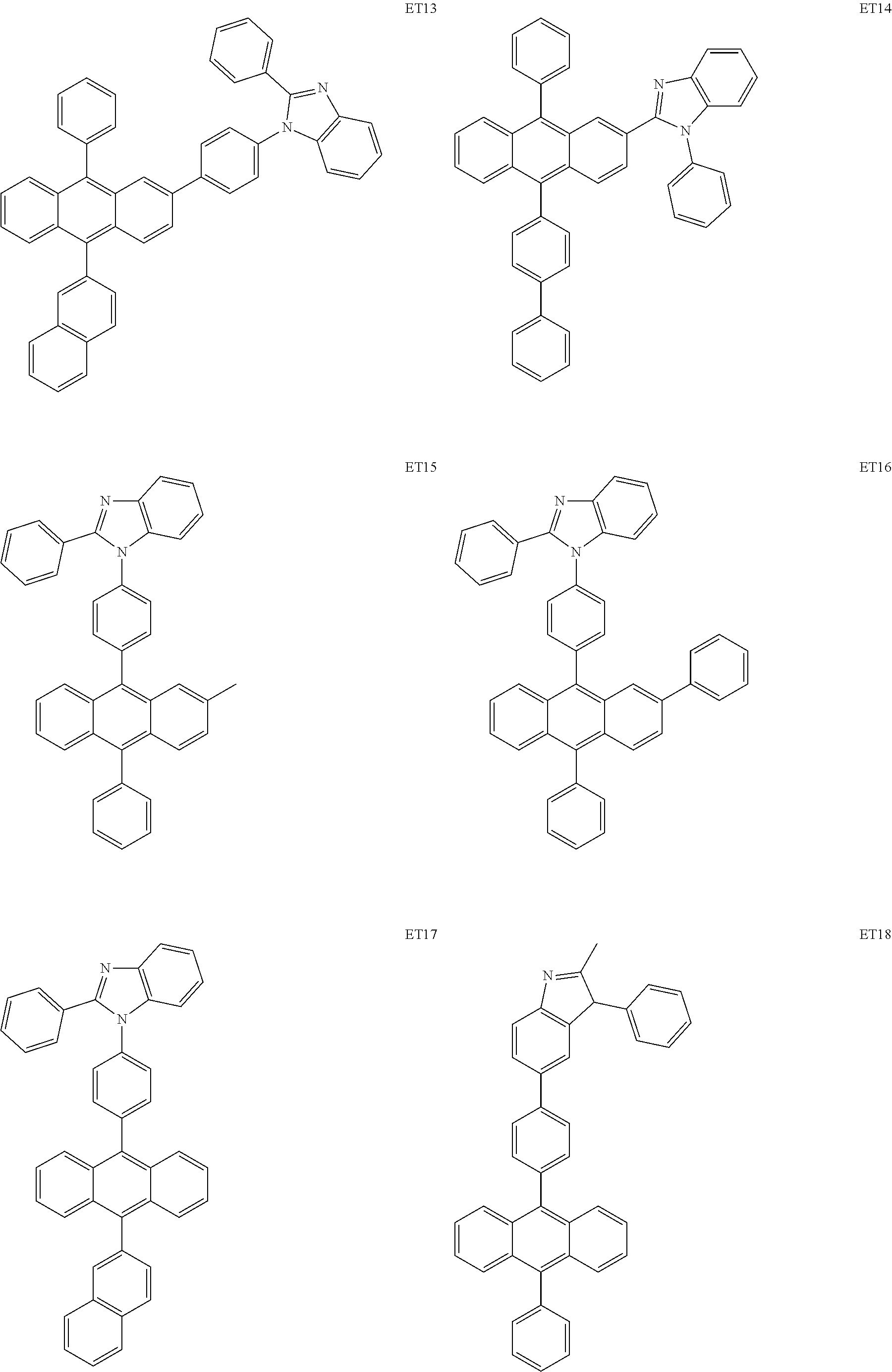 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; CC1=Nc2ccc(-c3ccc(-c4c5ccccc5c(-c5ccccc5)c5ccccc45)cc3)cc2C1c1ccccc1]

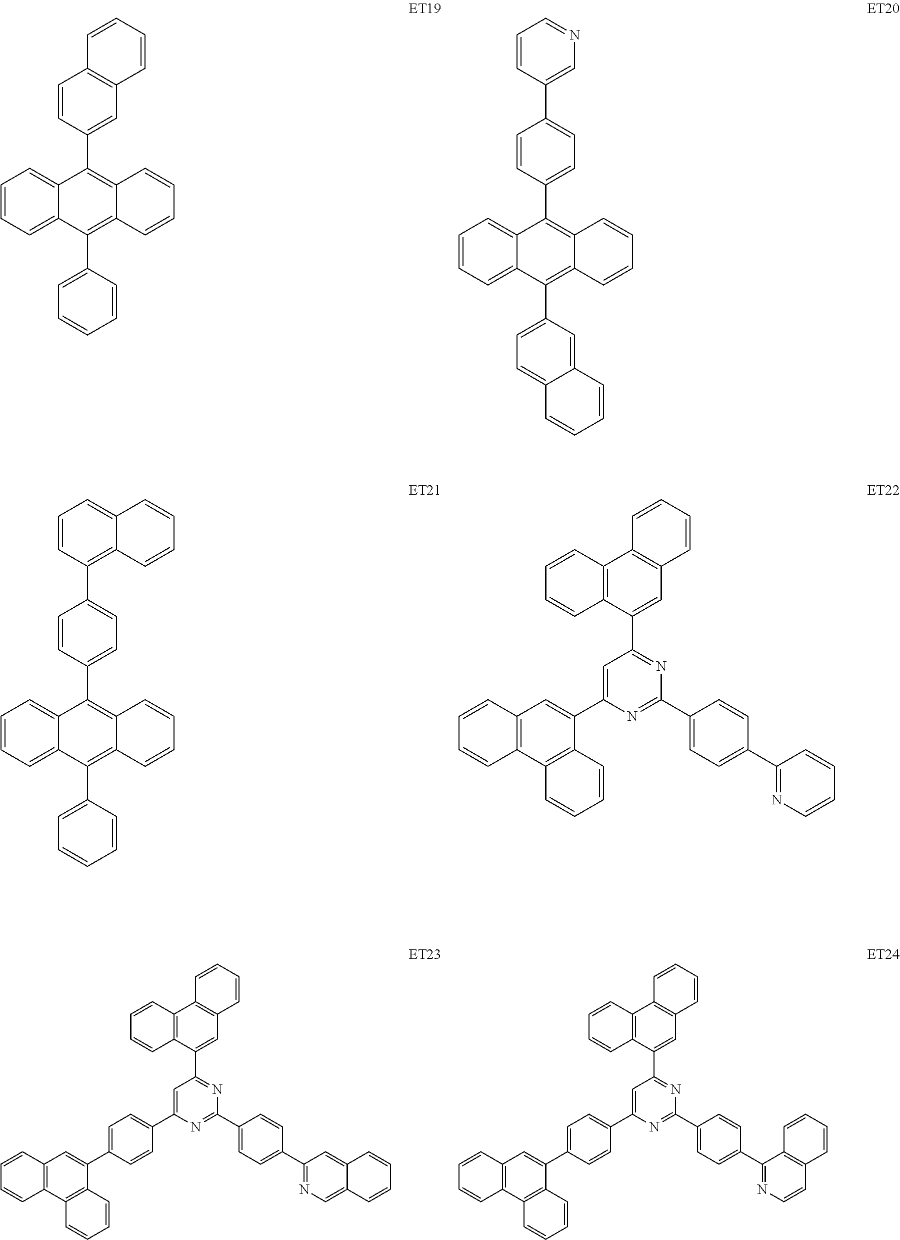

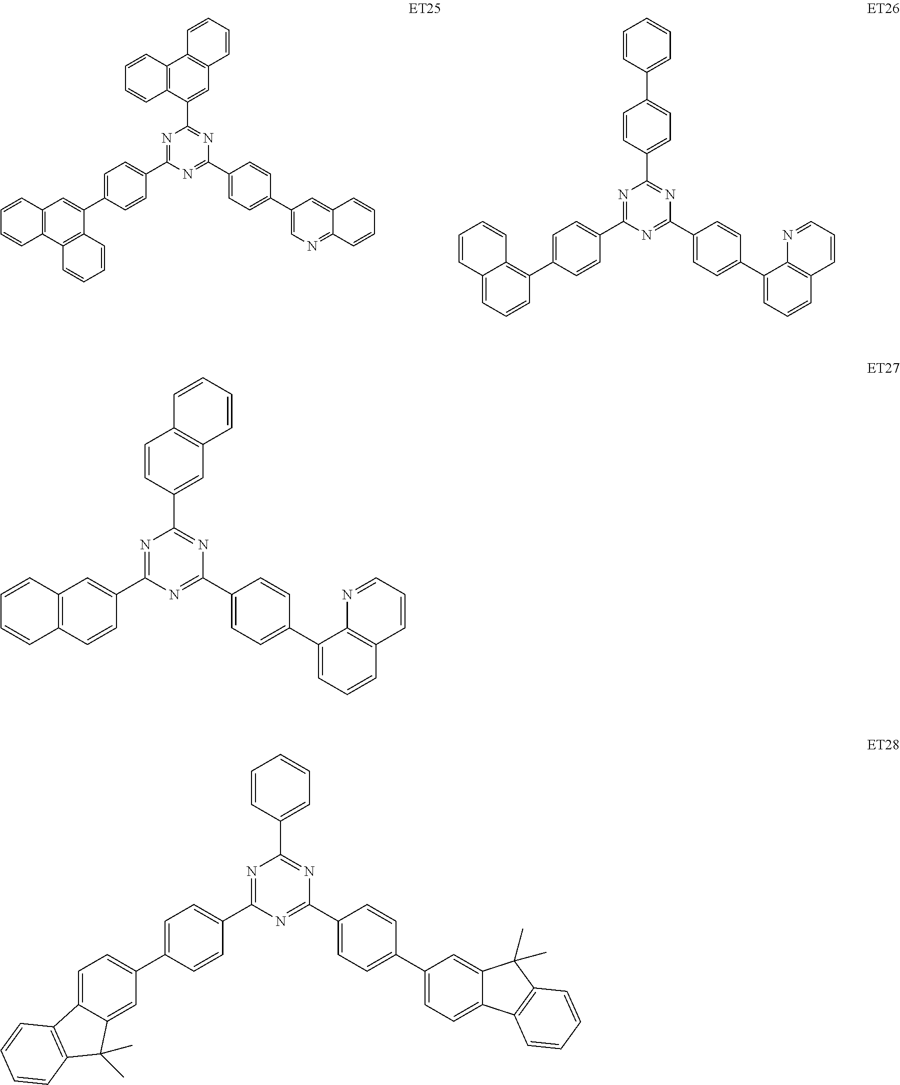 [SMILES: c1ccc2ncc(-c3ccc(-c4nc(-c5ccc(-c6cc7ccccc7c7ccccc67)cc5)nc(-c5cc6ccccc6c6ccccc56)n4)cc3)cc2c1 ; c1ccc(-c2ccc(-c3nc(-c4ccc(-c5cccc6ccccc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21]

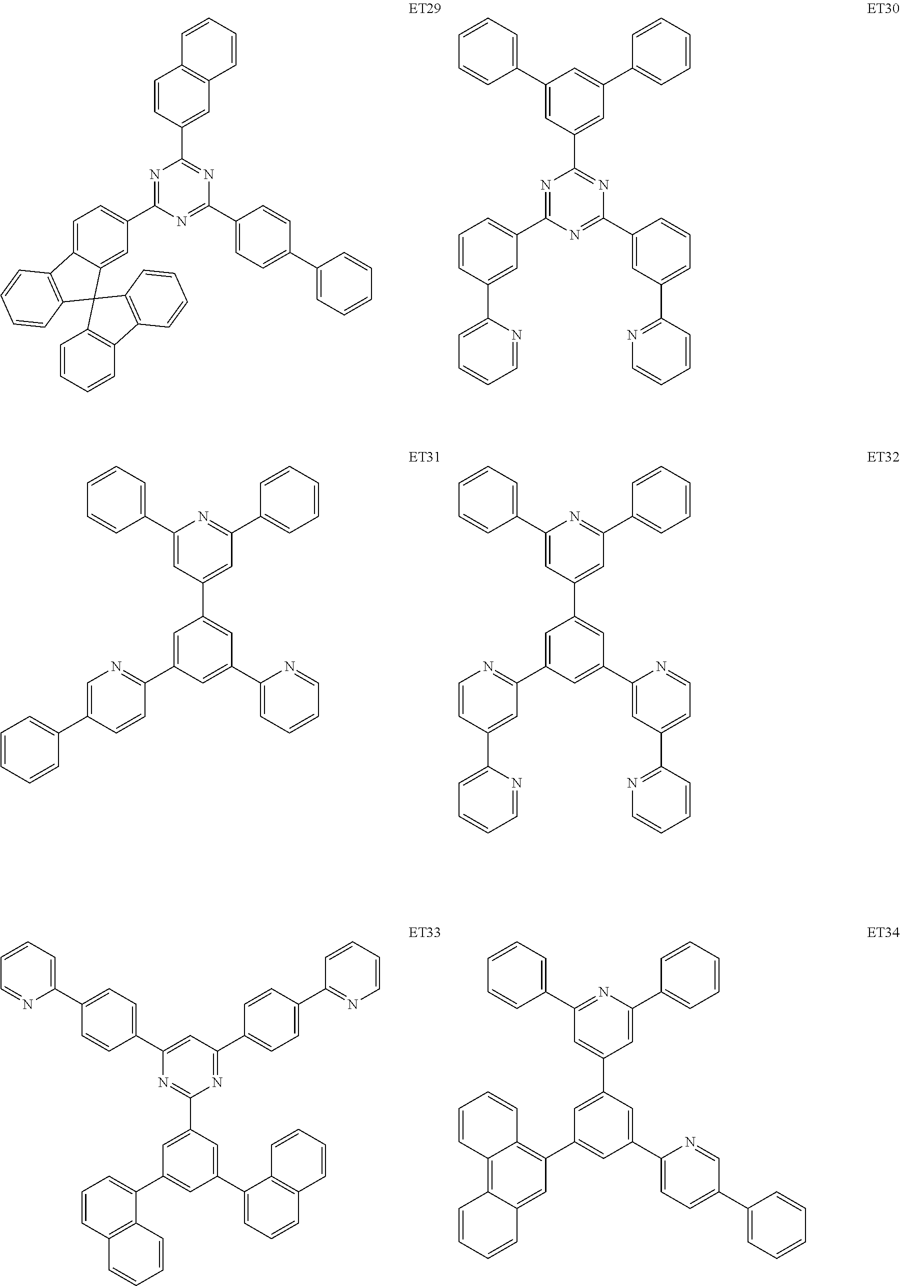

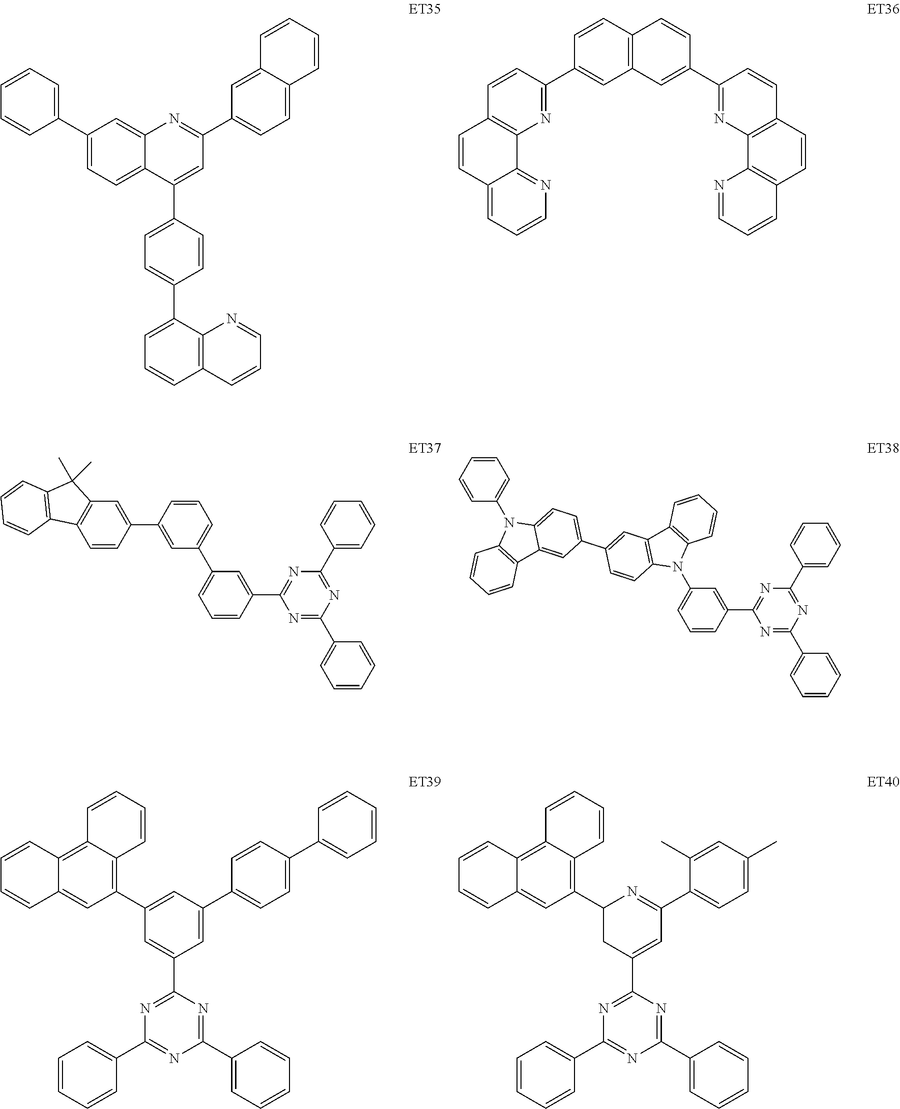 [SMILES: c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12 ; CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cc(-c4nc(-c5ccccc5)nc(-c5ccccc5)n4)cc(-c4cc5ccccc5c5ccccc45)c3)cc2)cc1 ; Cc1ccc(C2=NC(c3cc4ccccc4c4ccccc34)CC(c3nc(-c4ccccc4)nc(-c4ccccc4)n3)=C2)c(C)c1]

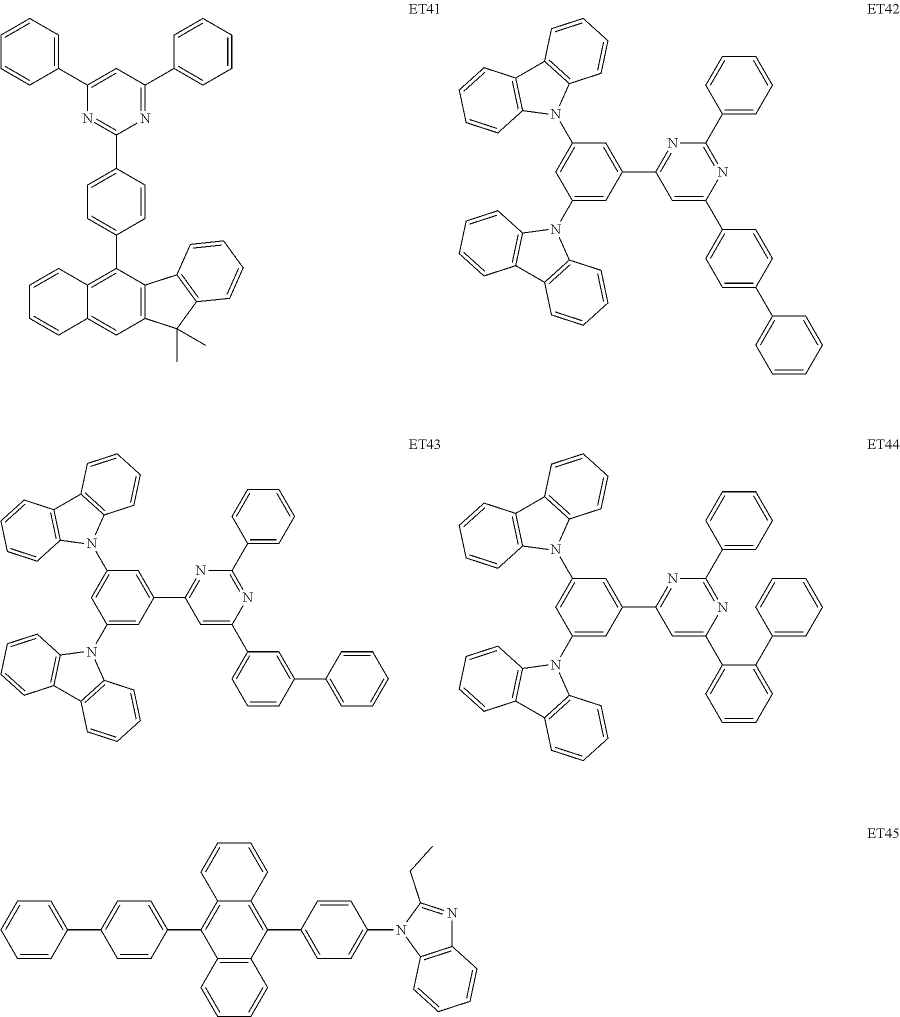 [SMILES: CC1(C)c2ccccc2-c2c1cc1ccccc1c2-c1ccc(-c2nc(-c3ccccc3)cc(-c3ccccc3)n2)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1 ; c1ccc(-c2nc(-c3cc(-n4c5ccccc5c5ccccc54)cc(-n4c5ccccc5c5ccccc54)c3)cc(-c3ccccc3-c3ccccc3)n2)cc1 ; CCc1nc2ccccc2n1-c1ccc(-c2c3ccccc3c(-c3ccc(-c4ccccc4)cc3)c3ccccc23)cc1]

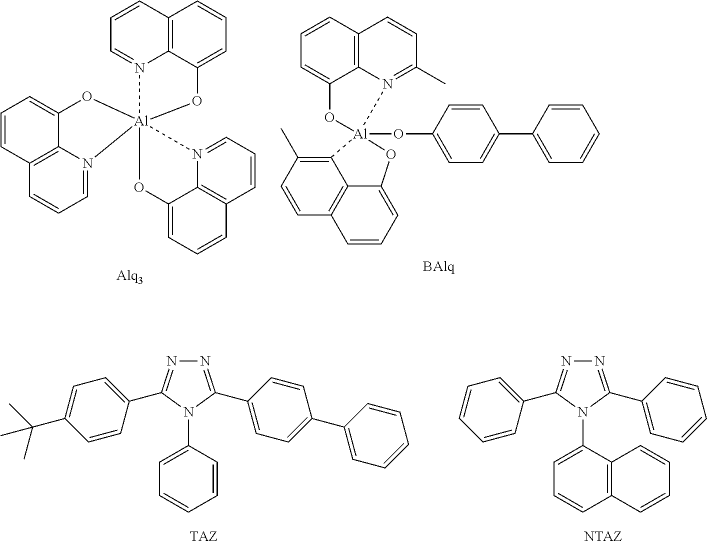

The thickness of the electron transport region may be from about 160 Å to about 5,000 Å, for example, from about 100 Å to about 4,000 Å. When the electron transport region includes a buffer layer, a hole blocking layer, an electron control layer, an electron transport layer, or any combination thereof, the thickness of the buffer layer, the hole blocking layer, or the electron control layer may each independently be from about 20 Å to about 1000 Å, for example, about 30 Å to about 300 Å, and the thickness of the electron transport layer may be from about 100 Å to about 1000 Å, for example, about 150 Å to about 500 Å. When the thicknesses of the buffer layer, hole blocking layer, electron control layer, and/or electron transport layer are within these ranges, satisfactory electron transporting characteristics may be obtained without a substantial increase in driving voltage.

The electron transport region (for example, the electron transport layer in the electron transport region) may further include, in addition to the materials described above, a metal-containing material.

The metal-containing material may include an alkali metal complex, alkaline earth metal complex, or any combination thereof. The metal ion of the alkali metal complex may be a Li ion, a Na ion, a K ion, a Rb ion, or a Cs ion, and the metal ion of the alkaline earth metal complex may be a Be ion, a Mg ion, a Ca ion, a Sr ion, or a Ba ion. A ligand coordinated with the metal ion of the alkali metal complex or the alkaline earth-metal complex may include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or any combination thereof.

For example, the metal-containing material may include a Li complex. The Li complex may include, for example, Compound ET-D1 (LiQ) or ET-D2:

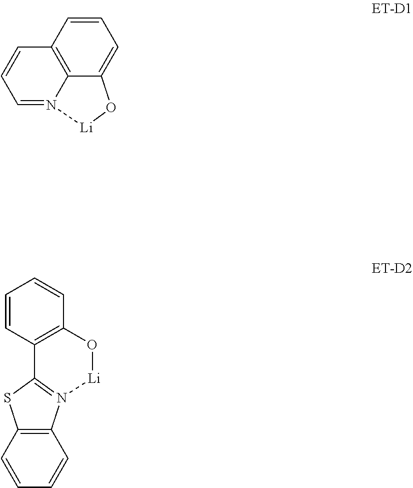

The electron transport region may include an electron injection layer that facilitates the injection of electrons from the second electrode 150. The electron injection layer may directly contact the second electrode 150.

The electron injection layer may have: i) a single-layered structure consisting of a single layer consisting of a single material, ii) a single-layered structure consisting of a single layer consisting of a plurality of different materials, or iii) a multi-layered structure including a plurality of layers including different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may be oxides, halides (for example, fluorides, chlorides, bromides, and/or iodides), and/or tellurides of the alkali metal, the alkaline earth metal, the rare earth metal, or any combination thereof.

The alkali metal-containing compound may include alkali metal oxides (such as Li₂O, Cs₂O, and/or K₂O), alkali metal halides (such as LiF, NaF, CsF, KF, LiI, NaI, CsI, and/or KI), or any combination thereof. The alkaline earth metal-containing compound may include an alkaline earth metal compound, such as BaO, SrO, CaO, BaₓSr₁₋ₓO (x is a real number satisfying the condition of 0<x<1), BaₓCa₁₋ₓO (x is a real number satisfying the condition of 0<x<1), and/or the like. The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In an embodiment, the rare earth metal-containing compound may include lanthanide metal telluride. Non-limiting examples of the lanthanide metal telluride are LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, and/or Lu₂Te₃.

The alkali metal complex, the alkaline earth-metal complex, and the rare earth metal complex may include i) one of ions of the alkali metal, the alkaline earth metal, and the rare earth metal and ii), as a ligand bonded to the metal ion, for example, hydroxyquinoline, hydroxyisoquinoline, hydroxybenzoquinoline, hydroxyacridine, hydroxyphenanthridine, hydroxyphenyloxazole, hydroxyphenylthiazole, hydroxydiphenyloxadiazole, hydroxydiphenylthiadiazole, hydroxyphenylpyridine, hydroxyphenyl benzimidazole, hydroxyphenylbenzothiazole, bipyridine, phenanthroline, cyclopentadiene, or any combination thereof.

The electron injection layer may include (e.g., consist of) an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In an embodiment, the electron injection layer may further include an organic material (for example, a compound represented by Formula 601).

In an embodiment, the electron injection layer may include (e.g., consist) of i) an alkali metal-containing compound (for example, an alkali metal halide), ii) a) an alkali metal-containing compound (for example, an alkali metal halide); and b) an alkali metal, an alkaline earth metal, a rare earth metal, or any combination thereof. In an embodiment, the electron injection layer may be a KI:Yb co-deposited layer, a RbI:Yb co-deposited layer, and/or the like.

When the electron injection layer further includes an organic material, the alkali metal, the alkaline earth metal, the rare earth metal, the alkali metal-containing compound, the alkaline earth metal-containing compound, the rare earth metal-containing compound, the alkali metal complex, the alkaline earth-metal complex, the rare earth metal complex, or any combination thereof may be homogeneously or non-homogeneously dispersed in a matrix including the organic material.

A thickness of the electron injection layer may be in a range of about 1 Å to about 100 Å, and, for example, about 3 Å to about 90 Å. When the thickness of the electron injection layer is within the ranges described above, the electron injection layer may have satisfactory electron injection characteristics without a substantial increase in driving voltage.

### Second Electrode 150

The second electrode 150 may be located on the interlayer 130 having such a structure. The second electrode 150 may be a cathode, which is an electron injection electrode, and as the material for forming the second electrode 150, a metal, an alloy, an electrically conductive compound, or any combination thereof, each having a low work function, may be utilized.

In an embodiment, the second electrode 150 may include at least one selected from lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or a combination thereof. The second electrode 150 may be a transmissive electrode, a semi-transmissive electrode, or a reflective electrode.

The second electrode 150 may have a single-layered structure or a multi-layered structure including two or more layers.

### Capping Layer

A first capping layer may be located outside the first electrode 110 (e.g., on the side opposite to the second electrode), and/or a second capping layer may be located outside the second electrode 150 (e.g., on the side opposite to the first electrode). In some embodiments, the light-emitting device 10 may have a structure in which the first capping layer, the first electrode 110, the interlayer 130, and the second electrode 150 are sequentially stacked in this stated order, a structure in which the first electrode 110, the interlayer 130, the second electrode 150, and the second capping layer are sequentially stacked in this stated order, or a structure in which the first capping layer, the first electrode 110, the interlayer 130, the second electrode 150, and the second capping layer are sequentially stacked in this stated order.

Light generated in the emission layer of the interlayer 130 of the light-emitting device 10 may be extracted (e.g., emitted) toward the outside through the first electrode 110, which is a semi-transmissive electrode or a transmissive electrode, and the first capping layer or light generated in the emission layer of the interlayer 130 of the light-emitting device 10 may be extracted (e.g., emitted) toward the outside through the second electrode 150, which is a semi-transmissive electrode or a transmissive electrode, and the second capping layer.

The first capping layer and the second capping layer may increase external emission efficiency according to the principle of constructive interference. Accordingly, the light extraction efficiency of the light-emitting device 10 is increased, so that the emission efficiency of the light-emitting device 10 may be improved.

Each of the first capping layer and second capping layer may include a material having a refractive index (at 589 nm) of 1.6 or more.

The first capping layer and the second capping layer may each independently be an organic capping layer including an organic material, an inorganic capping layer including an inorganic material, or an organic-inorganic composite capping layer including an organic material and an inorganic material.

At least one selected from the first capping layer and the second capping layer may each independently include a carbocyclic compound, a heterocyclic compound an amine group-containing compound, a porphyrin derivative, a phthalocyanine derivative, a naphthalocyanine derivative, an alkali metal complex, an alkaline earth-based complex, or any combination thereof. The carbocyclic compound, the heterocyclic compound, and/or the amine group-containing compound may be optionally substituted with a substituent containing O, N, S, Se, Si, F, Cl, Br, I, or any combination thereof.

In an embodiment, at least one of the first capping layer or the second capping layer may each independently include an amine group-containing compound.

In an embodiment, at least one of the first capping layer or the second capping layer may each independently include a compound represented by Formula 201, a compound represented by Formula 202, or any combination thereof.

In one or more embodiments, at least one of the first capping layer or the second capping layer may each independently include a compound selected from Compounds HT28 to HT33, Compounds CP1 to CP6, β-NPB, or any combination thereof, but embodiments of the present disclosure are not limited thereto:

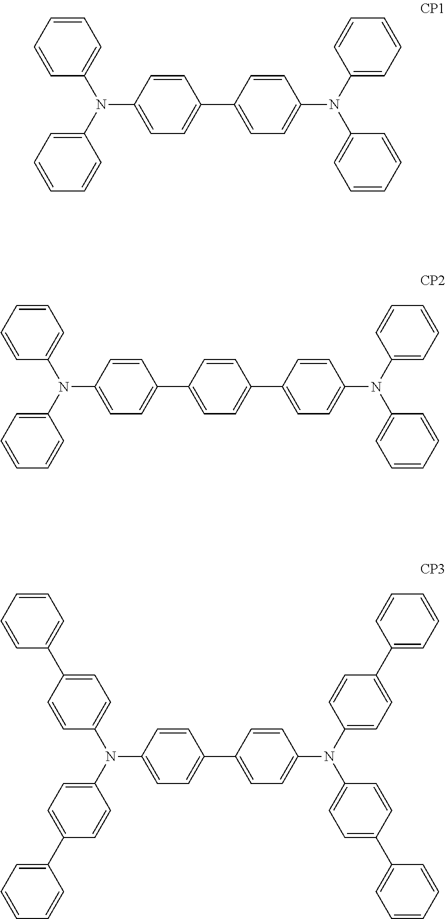 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1]

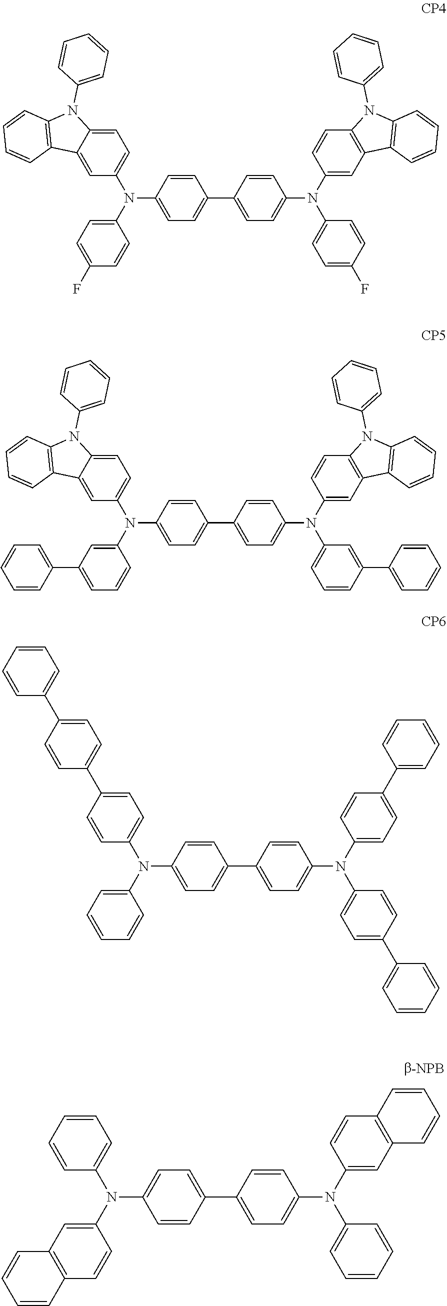 [SMILES: Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

### Film

The condensed cyclic compound represented by Formula 1 may be included in various films. Therefore, according to another embodiment, a film including the condensed cyclic compound represented by Formula 1 may be provided. The film may be, for example, an optical member (or light control means) (e.g., a color filter, a color conversion member, a capping layer, a light extraction efficiency enhancement layer, a selective light absorption layer, a polarizing layer, a quantum dot-containing layer, etc.), a light blocking member (eg, a light reflecting layer, a light absorbing layer, etc.), a protective member (eg, an insulating layer, a dielectric layer, etc.), and/or the like.

### Electronic Apparatus

The light-emitting device may be included in various suitable electronic apparatuses. In an embodiment, the electronic apparatus including the light-emitting device may be a light-emitting apparatus, an authentication apparatus, and/or the like.

The electronic apparatus (for example, light-emitting apparatus) may further include, in addition to the light-emitting device, i) a color filter, ii) a color conversion layer, or iii) a color filter and a color conversion layer. The color filter and/or the color conversion layer may be located in at least one traveling direction of light emitted from the light-emitting device. In an embodiment, the light emitted from the light-emitting device may be blue light or white light. The light-emitting device may be the same as described above. In an embodiment, the color conversion layer may include quantum dots. The quantum dot may be, for example, a quantum dot as described herein.

The electronic apparatus may include a first substrate. The first substrate may include a plurality of subpixel areas, the color filter may include a plurality of color filter areas respectively corresponding to the subpixel areas, and the color conversion layer may include a plurality of color conversion areas respectively corresponding to the subpixel areas.

A pixel-defining film may be located among the subpixel areas to define each of the subpixel areas.

The color filter may further include a plurality of color filter areas and light-blocking patterns located among the color filter areas, and the color conversion layer may include a plurality of color conversion areas and light-blocking patterns located among the color conversion areas.

The color filter areas (or the color conversion areas) may include a first area emitting first color light, a second area emitting second color light, and/or a third area emitting third color light, and the first color light, the second color light, and/or the third color light may have different maximum emission wavelengths from one another. In an embodiment, the first color light may be red light, the second color light may be green light, and the third color light may be blue light. In an embodiment, the color filter areas (or the color conversion areas) may include quantum dots. In some embodiments, the first area may include a red quantum dot, the second area may include a green quantum dot, and the third area may not include a quantum dot. The quantum dot is the same as described in the present specification. The first area, the second area, and/or the third area may each include a scatter.

In an embodiment, the light-emitting device may emit first light, the first area may absorb the first light to emit first first-color light, the second area may absorb the first light to emit second first-color light, and the third area may absorb the first light to emit third first-color light. In this regard, the first first-color light, the second first-color light, and the third first-color light may have different maximum emission wavelengths from one another. For example, the first light may be blue light, the first first-color light may be red light, the second first-color light may be green light, and the third first-color light may be blue light.

The electronic apparatus may further include a thin-film transistor in addition to the light-emitting device 10 as described above. The thin-film transistor may include a source electrode, a drain electrode, and an activation (e.g., active) layer, wherein one of the source electrode or the drain electrode may be electrically connected to one of the first electrode or the second electrode of the light-emitting device.

The thin-film transistor may further include a gate electrode, a gate insulating film, etc.

The activation layer may include crystalline silicon, amorphous silicon, organic semiconductor, oxide semiconductor, or the like.

The electronic apparatus may further include a sealing portion (e.g., a sealing layer) for sealing the light-emitting device. The sealing portion and/or the color conversion layer may be placed between the color filter and the light-emitting device.

The sealing portion allows light from the light-emitting device to be extracted to the outside, while simultaneously preventing ambient air and moisture from penetrating into the light-emitting device. The sealing portion may be a sealing substrate including a transparent glass or a plastic substrate. The sealing portion may be a thin-film encapsulation layer including at least one layer of an organic layer and/or an inorganic layer. When the sealing portion is a thin film encapsulation layer, the electronic apparatus may be flexible.

Various suitable functional layers may be additionally located on the sealing portion, in addition to the color filter and/or the color conversion layer, according to the application or utilization of the electronic apparatus. The functional layers may include a touch screen layer, a polarizing layer, and/or the like. The touch screen layer may be a pressure-sensitive touch screen layer, a capacitive touch screen layer, and/or an infrared touch screen layer. The authentication apparatus may be, for example, a biometric authentication apparatus that authenticates an individual by utilizing biometric information of a living body (for example, fingertips, pupils, etc.).

The authentication apparatus may further include, in addition to the light-emitting device, a biometric information collector.

The electronic apparatus may be applied to various suitable displays, light sources, lighting apparatuses, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, and/or endoscope displays), fish finders, various suitable measuring instruments, meters (for example, meters for a vehicle, an aircraft, and/or a vessel), projectors, and/or the like.

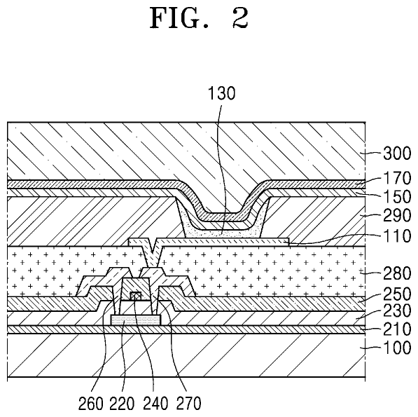

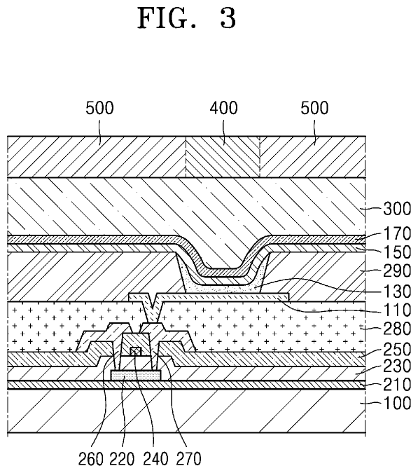

### Description of FIGS. 2 and 3

FIG. 2 is a schematic cross-sectional view showing a light-emitting apparatus according to an embodiment of the present disclosure.

The light-emitting apparatus of FIG. 2 includes a substrate 100, a thin-film transistor (TFT), a light-emitting device, and an encapsulation portion 300 that seals the light-emitting device.

The substrate 100 may be a flexible substrate, a glass substrate, or a metal substrate. A buffer layer 210 may be formed on the substrate 100. The buffer layer 210 may prevent or substantially prevent penetration of impurities through the substrate 100 and may provide a flat surface on the substrate 100.

A TFT may be located on the buffer layer 210. The TFT may include an activation (e.g., active) layer 220, a gate electrode 240, a source electrode 260, and a drain electrode 270.

The activation layer 220 may include an inorganic semiconductor such as silicon and/or polysilicon, an organic semiconductor, and/or an oxide semiconductor, and may include a source region, a drain region and a channel region.

A gate insulating film 230 for insulating the activation layer 220 from the gate electrode 240 may be located on the activation layer 220, and the gate electrode 240 may be located on the gate insulating film 230.

An interlayer insulating film 250 is located on the gate electrode 240. The interlayer insulating film 250 may be placed between the gate electrode 240 and the source electrode 260 to insulate the gate electrode 240 from the source electrode 260 and between the gate electrode 240 and the drain electrode 270 to insulate the gate electrode 240 from the drain electrode 270.

The source electrode 260 and the drain electrode 270 may be located on the interlayer insulating film 250. The interlayer insulating film 250 and the gate insulating film 230 may be formed to expose the source region and the drain region of the activation layer 220, and the source electrode 260 and the drain electrode 270 may be in contact with the exposed portions of the source region and the drain region of the activation layer 220.

The TFT is electrically connected to a light-emitting device to drive the light-emitting device, and is covered by a passivation layer 280. The passivation layer 280 may include an inorganic insulating film, an organic insulating film, or a combination thereof. A light-emitting device is provided on the passivation layer 280. The light-emitting device may include the first electrode 110, the interlayer 130, and the second electrode 150.

The first electrode 110 may be formed on the passivation layer 280. The passivation layer 280 does not completely cover the drain electrode 270 and exposes a portion of the drain electrode 270, and the first electrode 110 is connected to the exposed portion of the drain electrode 270.

A pixel defining layer 290 containing an insulating material may be located on the first electrode 110. The pixel defining layer 290 exposes a region of the first electrode 110, and the interlayer 130 may be formed in the exposed region of the first electrode 110. The pixel defining layer 290 may be a polyimide or polyacrylic organic film. In an embodiment, at least some layers of the interlayer 130 may extend beyond the upper portion of the pixel defining layer 290 to be located in the form of a common layer.

The second electrode 150 may be located on the interlayer 130, and a capping layer 170 may be additionally formed on the second electrode 150. The capping layer 170 may be formed to cover the second electrode 150.

The encapsulation portion 300 may be located on the capping layer 170. The encapsulation portion 300 may be located on the light-emitting device to protect the light-emitting device from moisture and/or oxygen. The encapsulation portion 300 may include: an inorganic film including silicon nitride (SiNx), silicon oxide (SiOx), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (for example, polymethyl methacrylate, polyacrylic acid, and/or the like), an epoxy-based resin (for example, aliphatic glycidyl ether (AGE), and/or the like), or a combination thereof; or a combination of the inorganic film and the organic film.

FIG. 3 shows a schematic cross-sectional view showing a light-emitting apparatus according to an embodiment of the present disclosure.

The light-emitting apparatus of FIG. 3 is the same as the light-emitting apparatus of FIG. 2, except that a light-blocking pattern 500 and a functional region 400 are additionally located on the encapsulation portion 300. The functional region 400 may be i) a color filter area, ii) a color conversion area, or iii) a combination of the color filter area and the color conversion area. In an embodiment, the light-emitting device included in the light-emitting apparatus of FIG. 3 may be a tandem light-emitting device.

### Manufacture Method

Respective layers included in the hole transport region, the emission layer, and respective layers included in the electron transport region may be formed in a certain region by utilizing one or more suitable methods selected from vacuum deposition, spin coating, casting, Langmuir-Blodgett (LB) deposition, ink-jet printing, laser-printing, and laser-induced thermal imaging.

When layers constituting the hole transport region, the emission layer, and layers constituting the electron transport region are formed by vacuum deposition, the deposition may be performed at a deposition temperature of about 100° C. to about 500° C., a vacuum degree of about 10⁻⁸ torr to about 10⁻³ torr, and a deposition speed of about 0.01 Å/sec to about 100 Å/sec, depending on a material to be included in the layer to be formed and the structure of the layer to be formed.

### Definition of Terms

The term “C₃-C₆₀ carbocyclic group” as used herein refers to a cyclic group consisting of only carbon as a ring-forming atom and having three to sixty carbon atoms, and the term “C₁-C₆₀ heterocyclic group” as used herein refers to a cyclic group that has one to sixty carbon atoms and further has, in addition to carbon, a heteroatom. The C₃-C₆₀ carbocyclic group and the C₁-C₆₀ heterocyclic group may each be a monocyclic group consisting of one ring or a polycyclic group in which two or more rings are condensed with each other. In an embodiment, the number of ring-forming atoms of the C₁-C₆₀ heterocyclic group may be from 3 to 61.

The “cyclic group” as used herein may include the C₃-C₆₀ carbocyclic group and the C₁-C₆₀ heterocyclic group.

The term “π electron-rich C₃-C₆₀ cyclic group” as used herein refers to a cyclic group that has three to sixty carbon atoms and does not include *—N═*′ as a ring-forming moiety, and the term “π electron-deficient nitrogen-containing C₁-C₆₀ cyclic group” as used herein refers to a heterocyclic group that has one to sixty carbon atoms and also includes *—N═*′ as a ring-forming moiety.

For example,

the C₃-C₆₀ carbocyclic group may be i) group T1 or ii) a condensed cyclic group in which two or more groups T1 are condensed with each other (for example, a cyclopentadiene group, an adamantane group, a norbornane group, a benzene group, a pentalene group, a naphthalene group, an azulene group, an indacene group, acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),

the C₁-C₆₀ heterocyclic group may be i) group T2, ii) a condensed cyclic group in which two or more groups T2 are condensed with each other, or iii) a condensed cyclic group in which at least one group T2 and at least one group T1 are condensed with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),

the π electron-rich C₃-C₆₀ cyclic group may be i) group T1, ii) a condensed cyclic group in which two or more groups T1 are condensed with each other, iii) group T3, iv) a condensed cyclic group in which two or more groups T3 are condensed with each other, or v) a condensed cyclic group in which at least one group T3 and at least one group T1 are condensed with each other (for example, the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, etc.),

the π electron-deficient nitrogen-containing C₁-C₆₀ cyclic group may be i) group T4, ii) a condensed cyclic group in which two or more group T4 are condensed with each other, iii) a condensed cyclic group in which at least one group T4 and at least one group T1 are condensed with each other, iv) a condensed cyclic group in which at least one group T4 and at least one group T3 are condensed with each other, or v) a condensed cyclic group in which at least one group T4, at least one group T1, and at least one group T3 are condensed with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),

group T1 may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane group (or a bicyclo[2.2.1]heptane group), a norbornene group, a bicyclo[1.1.1]pentane group, a bicyclo[2.1.1]hexane group, a bicyclo[2.2.2]octane group, or a benzene group,

group T2 may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group,

group T3 may be a furan group, a thiophene group, a 1H-pyrrole group, a silole group, or a borole group, and

group T4 may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “the cyclic group,” “the C₃-C₆₀ carbocyclic group,” “the C₁-C₆₀ heterocyclic group,” “the π electron-rich C₃-C₆₀ cyclic group,” or “the π electron-deficient nitrogen-containing C₁-C₆₀ cyclic group” as used herein each refer to a monovalent group or a polyvalent group (for example, a divalent group, a trivalent group, a tetravalent group, etc.) that is bonded to another group (e.g., a cyclic group), depending on the structure of a formula in connection with which the terms are used. In an embodiment, “a benzene group” may be a benzo group, a phenyl group, a phenylene group, or the like, which may be easily understand by one of ordinary skill in the art according to the structure of a formula including the “benzene group.”

Non-limiting examples of the monovalent C₃-C₆₀ carbocyclic group and the monovalent C₁-C₆₀ heterocyclic group are a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and/or a monovalent non-aromatic condensed heteropolycyclic group, and non-limiting examples of the divalent C₃-C₆₀ carbocyclic group and the divalent C₁-C₆₀ heterocyclic group are a C₃-C₁₀ cycloalkylene group, a C₁-C₁₀heterocycloalkylene group, a C₃-C₁₀ cycloalkenylene group, a C₁-C₁₀heterocycloalkenylene group, a C₆-C₆₀ arylene group, a C₁-C₆₀ heteroarylene group, a divalent non-aromatic condensed polycyclic group, and/or a substituted or unsubstituted divalent non-aromatic condensed heteropolycyclic group.

The term “C₁-C₆₀ alkyl group” as used herein refers to a linear or branched aliphatic hydrocarbon monovalent group that has one to sixty carbon atoms, and non-limiting examples thereof are a methyl group, an ethyl group, an n-propyl group, an isopropyl group, an n-butyl group, a sec-butyl group, an isobutyl group, a tert-butyl group, an n-pentyl group, a tert-pentyl group, neopentyl group, an isopentyl group, a sec-pentyl group, 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and/or a tert-decyl group. The term “C₁-C₆₀ alkylene group” as used herein refers to a divalent group having the same structure as the C₁-C₆₀ alkyl group.

The term “C₂-C₆₀ alkenyl group” as used herein refers to a monovalent hydrocarbon group having at least one carbon-carbon double bond in the middle or at a terminal end (e.g., the terminus) of the C₂-C₆₀ alkyl group, and non-limiting examples thereof are an ethenyl group, a propenyl group, and/or a butenyl group. The term “C₂-C₆₀ alkenylene group” as used herein refers to a divalent group having the same structure as the C₂-C₆₀ alkenyl group.

The term “C₂-C₆₀ alkynyl group” as used herein refers to a monovalent hydrocarbon group having at least one carbon-carbon triple bond in the middle or at a terminal end (e.g., the terminus) of the C₂-C₆₀ alkyl group, and non-limiting examples thereof include an ethynyl group, and/or a propynyl group. The term “C₁-C₆₀ alkynylene group” as used herein refers to a divalent group having the same structure as the C₁-C₆₀ alkynyl group.

The term “C₁-C₆₀ alkoxy group” as used herein refers to a monovalent group represented by —OA₁₀₁ (wherein A₁₀₁ is the C₁-C₆₀ alkyl group), and non-limiting examples thereof include a methoxy group, an ethoxy group, and an isopropyloxy group.

The term “C₃-C₁₀ cycloalkyl group” as used herein refers to a monovalent saturated hydrocarbon cyclic group having 3 to 10 carbon atoms, and on-limiting examples thereof are a cyclopropyl group, a cyclobutyl group, a cyclopentyl group, a cyclohexyl group, a cycloheptyl group, a cyclooctyl group, an adamantanyl group, a norbornanyl group (or bicyclo[2.2.1]heptyl group), a bicyclo[1.1.1]pentyl group, a bicyclo[2.1.1]hexyl group, and/or a bicyclo[2.2.2]octyl group. The term “C₃-C₁₀ cycloalkylene group” as used herein refers to a divalent group having the same structure as the C₃-C₁₀ cycloalkyl group.

The term “C₁-C₁₀ heterocycloalkyl group” as used herein refers to a monovalent cyclic group that further includes, in addition to 1 to 10 carbon atoms, at least one heteroatom as a ring-forming atom, and non-limiting examples thereof are a 1,2,3,4-oxatriazolidinyl group, a tetrahydrofuranyl group, and/or a tetrahydrothiophenyl group. The term “C₁-C₁₀ heterocycloalkylene group” as used herein refers to a divalent group having the same structure as the C₁-C₁₀ heterocycloalkyl group.

The term “C₃-C₁₀ cycloalkenyl group” as used herein refers to a monovalent monocyclic group that has three to ten carbon atoms and at least one carbon-carbon double bond in the ring thereof and no aromaticity, and non-limiting examples thereof are a cyclopentenyl group, a cyclohexenyl group, and/or a cycloheptenyl group. The term “C₃-C₁₀ cycloalkenylene group” as used herein refers to a divalent group having the same structure as the C₃-C₁₀ cycloalkenyl group.

The term “C₁-C₁₀ heterocycloalkenyl group” as used herein refers to a monovalent cyclic group that has, in addition to 1 to 10 carbon atoms, at least one heteroatom as a ring-forming atom, and at least one carbon-carbon double bond in the cyclic structure thereof. Non-limiting examples of the C₁-C₁₀ heterocycloalkenyl group include a 4,5-dihydro-1,2,3,4-oxatriazolyl group, a 2,3-dihydrofuranyl group, and/or a 2,3-dihydrothienyl group. The term “C₁-C₁₀ heterocycloalkenylene group” as used herein refers to a divalent group having the same structure as the C₁-C₁₀ heterocycloalkenyl group.

The term “C₆-C₆₀ aryl group” as used herein refers to a monovalent group having a carbocyclic aromatic system having six to sixty carbon atoms, and the term “C₆-C₆₀ arylene group” as used herein refers to a divalent group having a carbocyclic aromatic system having six to sixty carbon atoms. Non-limiting examples of the C₆-C₆₀ aryl group are a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, a fluorenyl group, and/or an ovalenyl group. When the C₆-C₆₀ aryl group and the C₆-C₆₀ arylene group, each include two or more rings, the two or more rings may be condensed with each other.

The term “C₁-C₆₀ heteroaryl group” as used herein refers to a monovalent group having a heterocyclic aromatic system that has, in addition to 1 to 60 carbon atoms, at least one heteroatom as a ring-forming atom. The term “C₁-C₆₀ heteroarylene group” as used herein refers to a divalent group having a heterocyclic aromatic system that has, in addition to 1 to 60 carbon atoms, at least one heteroatom as a ring-forming atom. Non-limiting examples of the C₁-C₆₀ heteroaryl group are a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, a carbazolyl group, a dibenzofuranyl group, a dibenzothiofuranyl group, and/or a naphthyridinyl group. When the C₁-C₆₀ heteroaryl group and the C₁-C₆₀ heteroarylene group, each include two or more rings, the two or more rings may be condensed with each other.

The term “monovalent non-aromatic condensed polycyclic group” as used herein refers to a monovalent group having two or more rings condensed to each other, only carbon atoms (for example, having 8 to 60 carbon atoms) as ring-forming atoms, and no aromaticity in its entire molecular structure (e.g., the entire molecular structure is not aromatic). Non-limiting examples of the monovalent non-aromatic condensed polycyclic group are an indenyl group, a fluorenyl group, a spiro-bifluorenyl group, a benzofluorenyl group, an indenophenanthrenyl group, an adamantyl group, and/or an indenon anthracenyl group. The term “divalent non-aromatic condensed polycyclic group” as used herein refers to a divalent group having the same structure as a monovalent non-aromatic condensed polycyclic group.

The term “monovalent non-aromatic condensed heteropolycyclic group” as used herein refers to a monovalent group having two or more rings condensed to each other, at least one heteroatom other than carbon atoms (for example, having 1 to 60 carbon atoms), as a ring-forming atom, and no aromaticity in its entire molecular structure (e.g., the entire molecular structure is not aromatic. Non-limiting examples of the monovalent non-aromatic condensed heteropolycyclic group are a pyrrolyl group, a thienyl group, a furanyl group, an indolyl group, a benzoindolyl group, a naphthoindolyl group, an isoindolyl group, a benzoisoindolyl group, a naphthoisoindolyl group, a benzosilolyl group, a benzothienyl group, a benzofuranyl group, a carbazolyl group, a dibenzosilolyl group, a dibenzothienyl group, a dibenzofuranyl group, an azacarbazolyl group, an azafluorenyl group, an azadibenzosilolyl group, an azadibenzothienyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indeno carbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothienyl group, a benzonaphtho silolyl group, a benzofurodibenzofuranyl group, a benzofurodibenzothienyl group, an azaadamantyl, and/or a benzothienodibenzothienyl group. The term “divalent non-aromatic condensed heteropolycyclic group” as used herein refers to a divalent group having the same structure as a monovalent non-aromatic condensed heteropolycyclic group.

The term “C₆-C₆₀ aryloxy group” as used herein refers to a monovalent group represented by —OA₁₀₂ (wherein A₁₀₂ is the C₆-C₆₀ aryl group), and the term “C₆-C₆₀ arylthio group” as used herein refers to a monovalent group represented by —SA₁₀₃ (wherein A₁₀₃ is the C₆-C₆₀ aryl group).

The term “C₇-C₆₀ arylalkyl group” as used herein refers to a monovalent group represented by -A₁₀₄A₁₀₅ (wherein A₁₀₄ is a C₁-C₅₄ alkylene group and A₁₀₅ is a C₆-C₅₉ aryl group), and the term “C₂-C₆₀ heteroarylalkyl group” as used herein refers to a monovalent group represented by -A₁₀₆A₁₀₇ (wherein A₁₀₆ is a C₁-C₅₉ alkylene group and A₁₀₇ is a C₁-C₅₉ heteroaryl group).

The term “R₁₀ₐ” as used herein refers to:

deuterium (-D), —F, —Cl, —Br, —I, a hydroxyl group, a cyano group, or a nitro group;

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;

a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —C(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),

wherein Q₁ to Q₃, Q₁₁ to Q₁₃, Q₂₁ to Q₂₃, and Q₃₁ to Q₃₃ may each independently be: hydrogen; deuterium (-D); —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof; a C₇-C₆₀ arylalkyl group; or a C₂-C₆₀ heteroarylalkyl group.

The term “hetero atom” as used herein refers to any atom other than a carbon atom. Non-limiting examples of the heteroatom are O, S, N, P, Si, B, Ge, Se, or any combination thereof.

The term “third-row transition metal” as used herein includes hafnium(Hf), tantalum(Ta), tungsten(W), rhenium(Re), osmium(Os), iridium(Ir), platinum(Pt), gold (Au), and/or the like.

The term “Ph” as used herein refers to a phenyl group, the term “Me” as used herein refers to a methyl group, the term “Et” as used herein refers to an ethyl group, the term “tert-Bu” or “But” as used herein refers to a tert-butyl group, and the term “OMe” as used herein refers to a methoxy group.

The term “biphenyl group” as used herein refers to “a phenyl group substituted with a phenyl group.” In other words, the “biphenyl group” is a substituted phenyl group having a C₆-C₆₀ aryl group as a substituent.

The term “terphenyl group” as used herein refers to “a phenyl group substituted with a biphenyl group”. In other words, the “terphenyl group” is a substituted phenyl group having, as a substituent, a C₆-C₆₀ aryl group substituted with a C₆-C₆₀ aryl group.

* and *′ as used herein, unless defined otherwise, each refer to a binding site to a neighboring atom in a corresponding formula or a moiety.

Hereinafter, a compound according to embodiments and a light-emitting device according to embodiments will be described in more detail with reference to Synthesis Examples and Examples. The wording “B was utilized instead of A” used in describing Synthesis Examples refers to that an identical molar equivalent of B was utilized in place of A.

### EXAMPLES

### Synthesis Example 1: Synthesis of Compound 1

The heterocyclic Compound 1 according to an embodiment may be synthesized by, for example, Reaction Scheme 1.

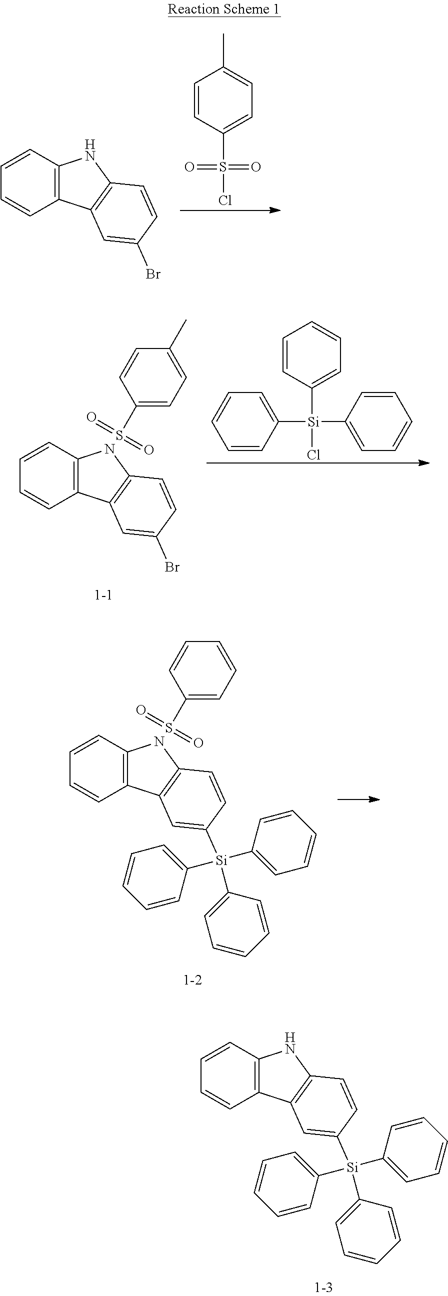

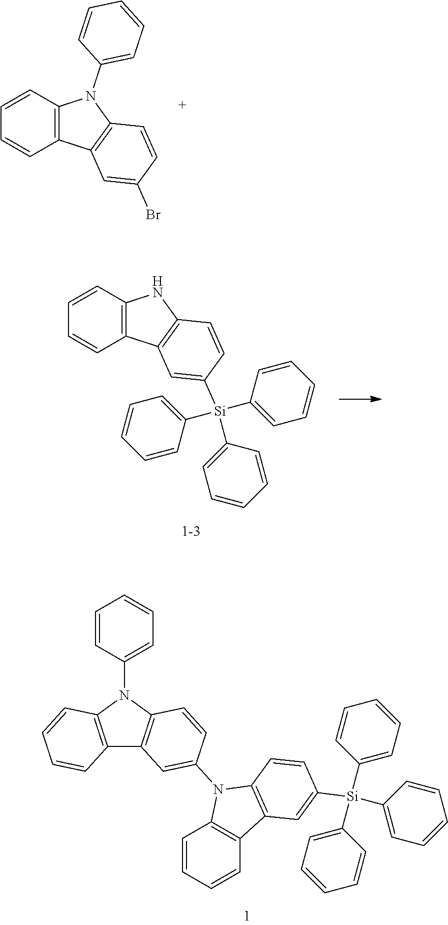 [SMILES: Brc1ccc2c(c1)c1ccccc1n2-c1ccccc1 ; c1ccc([Si](c2ccccc2)(c2ccccc2)c2ccc3[nH]c4ccccc4c3c2)cc1 ; c1ccc(-n2c3ccccc3c3cc(-n4c5ccccc5c5cc([Si](c6ccccc6)(c6ccccc6)c6ccccc6)ccc54)ccc32)cc1]

### Synthesis of Intermediate 1-1

3-bromo-9H-carbazole (CAS number=1592-95-6), potassium hydroxide, and 4-toluenesulfonylchloride were reacted to thereby obtain Intermediate 1-1. M+1 peak value of Intermediate 1-1 was confirmed by liquid chromatography mass spectrometry (LC-MS).

C₁₉H₁₄BrNO₂S: M+1 399.99

### Synthesis of Intermediate 1-2

Intermediate 1-1 was reacted with n-BuLi and then reacted with chlorotriphenylsilane (CAS number=76-86-8) to thereby obtain Intermediate 1-2. M+1 peak value of Intermediate 1-2 was confirmed by liquid chromatography mass spectrometry (LC-MS).

C₃₇H₂₉NO₂SSi: M+1 580.12

### Synthesis of Intermediate 1-3

Intermediate 1-2 was reacted with sodium hydroxide to thereby obtain Intermediate 1-3. M+1 peak value of Intermediate 1-3 was confirmed by liquid chromatography mass spectrometry (LC-MS).

C₃₀H₂₃NSi: M+1 426.20

### Synthesis of Compound 1

5 g of 3-bromo-9-phenylphenylcarbazole (CAS number=1153-85-1), 6.6 g of Intermediate 1-3, 2.2 g of sodiumtert-butoxide, 0.57 g of tris(dibenzylidene acetone)dipalladium(0), 0.5 ml of tritert-butylphosphine, and 75 ml of toluene were put in a reaction vessel and refluxed for 24 hours. After the reaction was completed, the reaction solution was extracted with ethylacetate, the collected organic layer was dried with magnesium sulfate, and a solvent was evaporated therefrom. The obtained residue was separated and purified by silica gel column chromatography to obtain 8.3 g (yield: 80%) of Compound 1. Compound 1 was confirmed by LC-MS and ¹H-NMR.

### Synthesis Example 2: Synthesis of Compound 21

The heterocyclic Compound 21 according to an embodiment may be synthesized by, for example, Reaction Scheme 1.

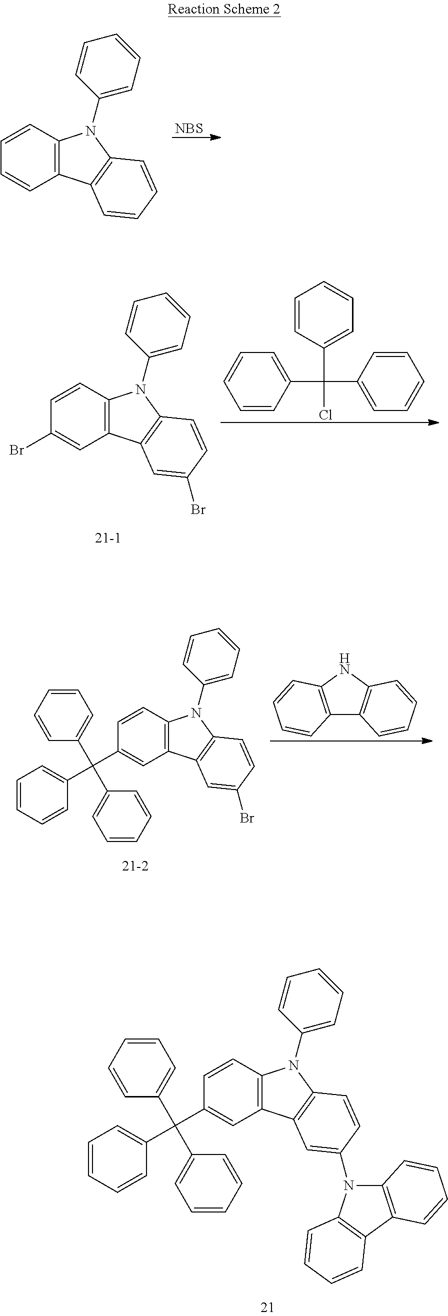

### Synthesis of Intermediate 21-1

9-phenyl-9H-carbazole (CAS number=1150-62-5) and N-bromosuccinimide were reacted to thereby obtain Intermediate 21-1. M+1 peak value of Intermediate 21-1 was confirmed by liquid chromatography mass spectrometry (LC-MS).

C₁₈H₁₁Br₂N: M+1 399.97

### Synthesis of Intermediate 21-2

Intermediate 21-1 was reacted with n-BuLi and then reacted with chlorotriphenylmethane (CAS number=76-83-5) to thereby obtain Intermediate 21-2. M+1 peak value of Intermediate 21-1 was confirmed by liquid chromatography mass spectrometry (LC-MS).

C₃₇H₂₆BrN: M+1 564.11

### Synthesis of Compound 21

4.5 g of Intermediate 21-2, 1.4 g of 9H-carbazole (CAS number=86-74-8), 1.15 g of sodiumtert-butoxide, 0.3 g of tris(dibenzylidene acetone)dipalladium(0), 0.25 ml of tritert-butylphosphine, and 40 ml of toluene were put in a reaction vessel and refluxed for 24 hours. After the reaction was completed, the reaction solution was extracted with ethylacetate, the collected organic layer was dried with magnesium sulfate, and a solvent was evaporated therefrom. The obtained residue was separated and purified by silica gel column chromatography to obtain 4.05 g (yield: 78%) of Compound 21. Compound 21 was confirmed by LC-MS and ¹H-NMR.

### Synthesis Example 3: Synthesis of Compound 28

The heterocyclic Compound 28 according to an embodiment may be synthesized by, for example, Reaction Scheme 3.

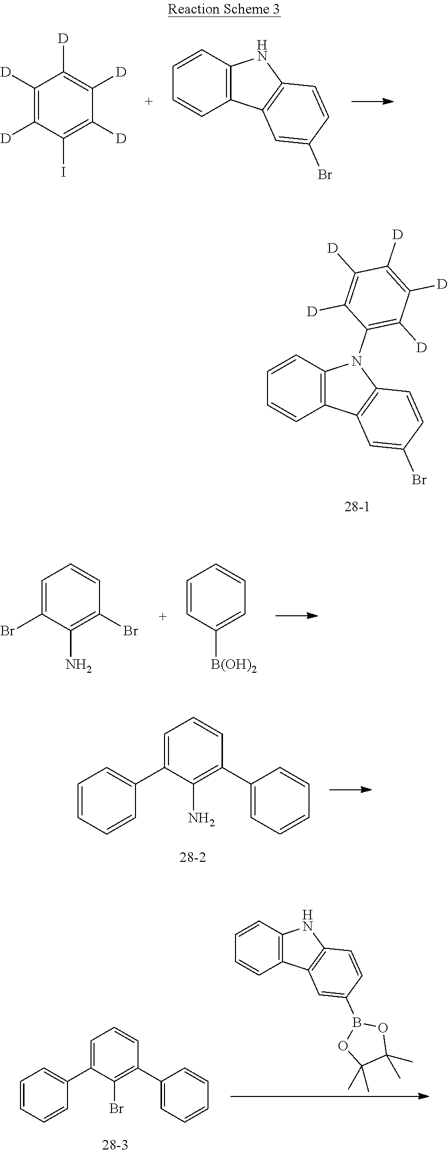

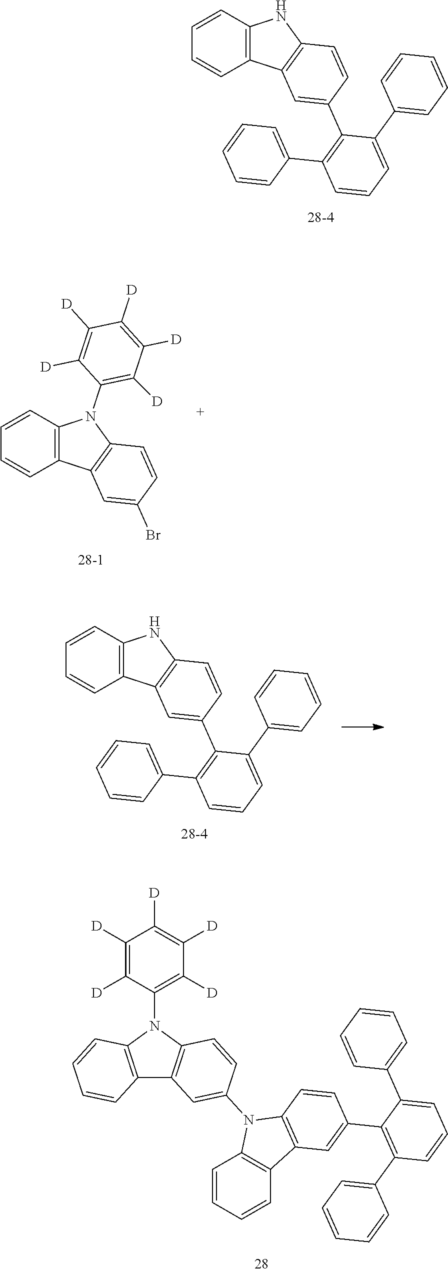 [SMILES: c1ccc(-c2cccc(-c3ccccc3)c2-c2ccc3[nH]c4ccccc4c3c2)cc1 ; [2H]c1c([2H])c([2H])c(-n2c3ccccc3c3cc(Br)ccc32)c([2H])c1[2H] ; c1ccc(-c2cccc(-c3ccccc3)c2-c2ccc3[nH]c4ccccc4c3c2)cc1 ; [2H]c1c([2H])c([2H])c(-n2c3ccccc3c3cc(-n4c5ccccc5c5cc(-c6c(-c7ccccc7)cccc6-c6ccccc6)ccc54)ccc32)c([2H])c1[2H]]

### Synthesis of Intermediate 28-1

Iodobenzene-d₅ (CAS number=7379-67-1) and 3-bromo-9H-carbazole (CAS number=1592-95-6) were reacted under a Cu catalyst condition to thereby obtain Intermediate 28-1. M+1 peak value of Intermediate 28-1 was confirmed by liquid chromatography mass spectrometry (LC-MS).

C₁₈H₇D₅BrN: M+1 327.11

### Synthesis of Intermediate 28-2

2,6-dibromoaniline (CAS number=608-30-0) and phenylboronic acid (CAS number=98-80-6) were reacted under a Pd catalyst condition to thereby obtain Intermediate 28-2. M+1 peak value of Intermediate 28-2 was confirmed by liquid chromatography mass spectrometry (LC-MS).

C₁₈H₁₅N: M+1 246.21

### Synthesis of Intermediate 28-3

Intermediate 28-2, hydrogen bromide, and sodium nitrite were reacted under a Cu catalyst condition to thereby obtain Intermediate 28-3. M+1 peak value of Intermediate 28-3 was confirmed by liquid chromatography mass spectrometry (LC-MS).

C₁₈H₁₃Br: M+1 309.09

### Synthesis of Intermediate 28-4

Intermediate 28-3 and 3-(4,4,5,5-tetramethyl-1,3,2-dioxaborolane-2-yl)-9H-carbazole (CAS number=855738-89-5) were reacted under a Pd catalyst condition to thereby obtain Intermediate 28-4. M+1 peak value of Intermediate 28-4 was confirmed by liquid chromatography mass spectrometry (LC-MS).

C₃₀H₂₁N: M+1 396.23

### Synthesis of Compound 28

5 g of Intermediate 28-1, 6 g of Intermediate 28-4, 2.2 g of sodiumtert-butoxide, 0.56 g of tris(dibenzylidene acetone)dipalladium(0), 0.5 ml of tritert-butylphosphine, and 75 ml of toluene were put in a reaction vessel and refluxed for 24 hours. After the reaction was completed, the reaction solution was extracted with ethylacetate, the collected organic layer was dried with magnesium sulfate, and a solvent was evaporated therefrom. The obtained residue was separated and purified by silica gel column chromatography to obtain 7.4 g (yield: 75%) of Compound 28. Compound 28 was confirmed by LC-MS and ¹H-NMR.

### Synthesis Example 4: Synthesis of Compound 56

The heterocyclic Compound 56 according to an embodiment may be synthesized by, for example, Reaction Scheme 4.

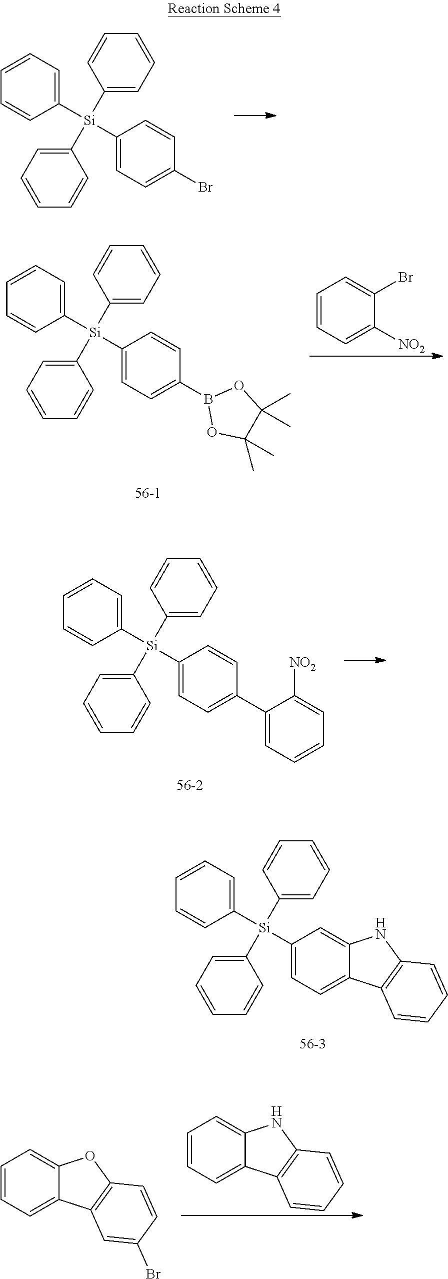

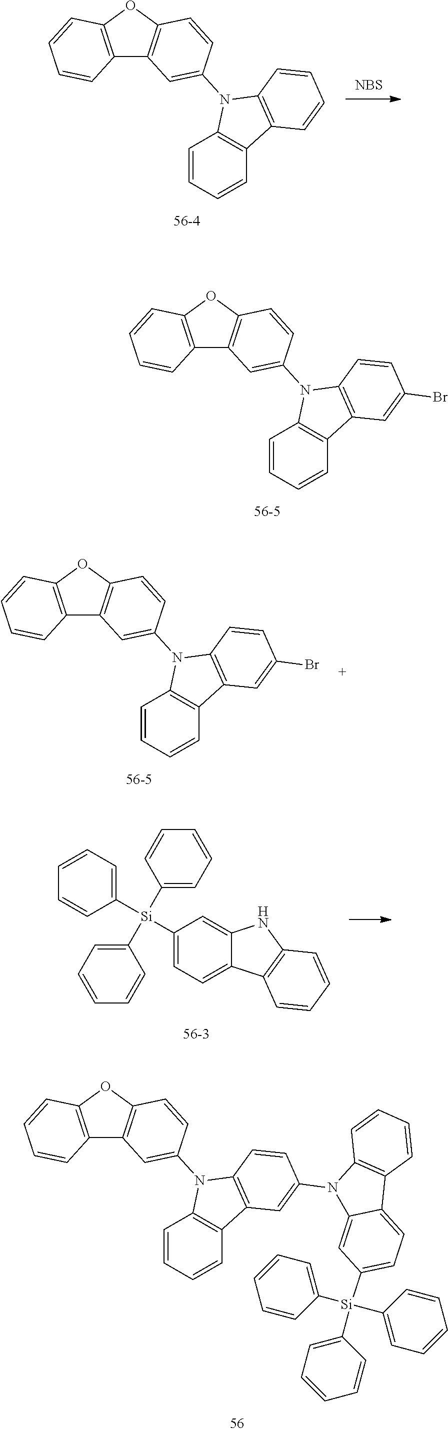

### Synthesis of Intermediate 56-1

(4-bromophenyl)triphenylsilane (CAS number=18737-40-1) and bis(pinacolato)diborone were reacted in a Pd catalyst condition to thereby obtain Intermediate 56-1. M+1 peak value of Intermediate 53-1 was confirmed by liquid chromatography mass spectrometry (LC-MS).

C₃₀H₃₁BO₂Si: M+1 463.24

### Synthesis of Intermediate 56-2

Intermediate 56-1 and 1-bromo-2-nitrobenzene (CAS number=577-19-5) were reacted under a Pd catalyst condition to thereby obtain Intermediate 56-2. M+1 peak value of Intermediate 56-2 was confirmed by liquid chromatography mass spectrometry (LC-MS).

C₃₀H₂₃NO₂Si: M+1 458.03

### Synthesis of Intermediate 56-3

Intermediate 56-2 and triphenylphosphine (CAS number=603-35-0) were reacted to thereby obtain Intermediate 56-3. M+1 peak value of Intermediate 56-3 was confirmed by liquid chromatography mass spectrometry (LC-MS).

C₃₀H₂₃NSi: M+1 426.26

### Synthesis of Intermediate 56-4

2-bromodibenzofuran (CAS number=86-76-0) and 9H-carbazole (CAS number=86-74-8) were reacted under a Pd catalyst condition to thereby obtain Intermediate 56-4. M+1 peak value of Intermediate 56-4 was confirmed by liquid chromatography mass spectrometry (LC-MS).

C₂₄H₁₅NO: M+1 334.05

### Synthesis of Intermediate 56-5

Intermediate 56-4 and N-bromosuccinimide were reacted to thereby obtain Intermediate 56-5. M+1 peak value of Intermediate 56-5 was confirmed by liquid chromatography mass spectrometry (LC-MS).

C₂₄H₁₄BrNO: M+1 412.13

### Synthesis of Compound 56

5 g of Intermediate 56-5, 6 g of Intermediate 56-6, 1.75 g of sodiumtert-butoxide, 0.44 g of tris(dibenzylidene acetone)dipalladium(0), 0.4 ml of tritert-butylphosphine, and 60 ml of toluene were put in a reaction vessel and refluxed for 24 hours. After the reaction was completed, the reaction solution was extracted with ethylacetate, the collected organic layer was dried with magnesium sulfate, and a solvent was evaporated therefrom. The obtained residue was separated and purified by silica gel column chromatography to obtain 7.8 g (yield: 85%) of Compound 56. Compound 56 was confirmed by LC-MS and ¹H-NMR.

### Synthesis Example 5: Synthesis of Compound 77

The heterocyclic Compound 77 according to an embodiment may be synthesized by, for example, Reaction Scheme 5.

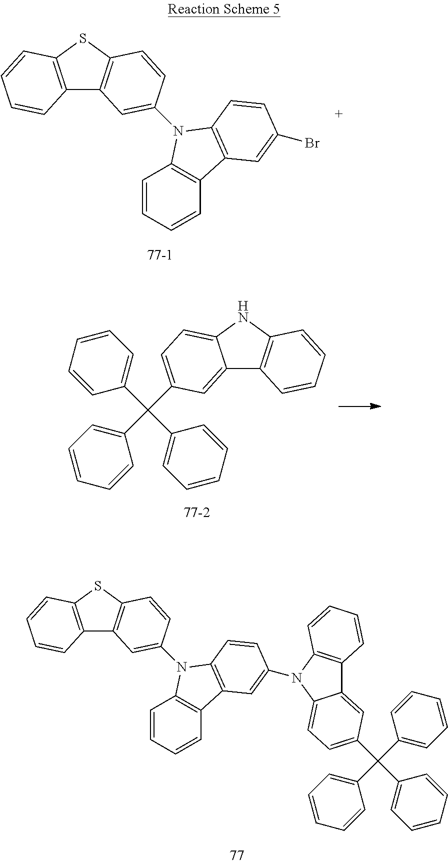 [SMILES: Brc1ccc2c(c1)c1ccccc1n2-c1ccc2sc3ccccc3c2c1 ; c1ccc(C(c2ccccc2)(c2ccccc2)c2ccc3[nH]c4ccccc4c3c2)cc1 ; c1ccc(C(c2ccccc2)(c2ccccc2)c2ccc3c(c2)c2ccccc2n3-c2ccc3c(c2)c2ccccc2n3-c2ccc3sc4ccccc4c3c2)cc1]

### Synthesis of Intermediate 77-1

Intermediate 77-1 was synthesized in the same manner as Intermediate 56-5, except that, in the synthesis process, 2-bromodibenzothiophene (CAS number=22439-61-8) was utilized instead of 2-bromodibenzofuran (CAS number=86-76-0). M+1 peak value of Intermediate 77-1 was confirmed by liquid chromatography mass spectrometry (LC-MS).

C₂₄H₁₄BrNS: M+1 428.01

### Synthesis of Intermediate 77-2

Intermediate 77-2 was synthesized in the same manner as Intermediate 1-3, except that, in the synthesis process, chlorotriphenylmethane (CAS number=76-83-5) was utilized instead of chlorotriphenylsilane (CAS number=76-86-8). M+1 peak value of Intermediate 77-2 was confirmed by liquid chromatography mass spectrometry (LC-MS).

C₃₁H₂₃N: M+1 410.19

### Synthesis of Compound 77

4 g of Intermediate 77-1, 3.9 g of Intermediate 77-2, 1.35 g of sodiumtert-butoxide, 0.34 g of tris(dibenzylidene acetone)dipalladium(0), 0.3 ml of tritert-butylphosphine, and 50 ml of toluene put in a reaction vessel and refluxed for 24 hours. After the reaction was completed, the reaction solution was extracted with ethylacetate, the collected organic layer was dried with magnesium sulfate, and a solvent was evaporated therefrom. The obtained residue was separated and purified by silica gel column chromatography to obtain 5.8 g (yield: 82%) of Compound 77. Compound 77 was confirmed by LC-MS and ¹H-NMR.

### Synthesis Example 6: Synthesis of Compound 98

The heterocyclic Compound 98 according to an embodiment may be synthesized by, for example, Reaction Scheme 6.

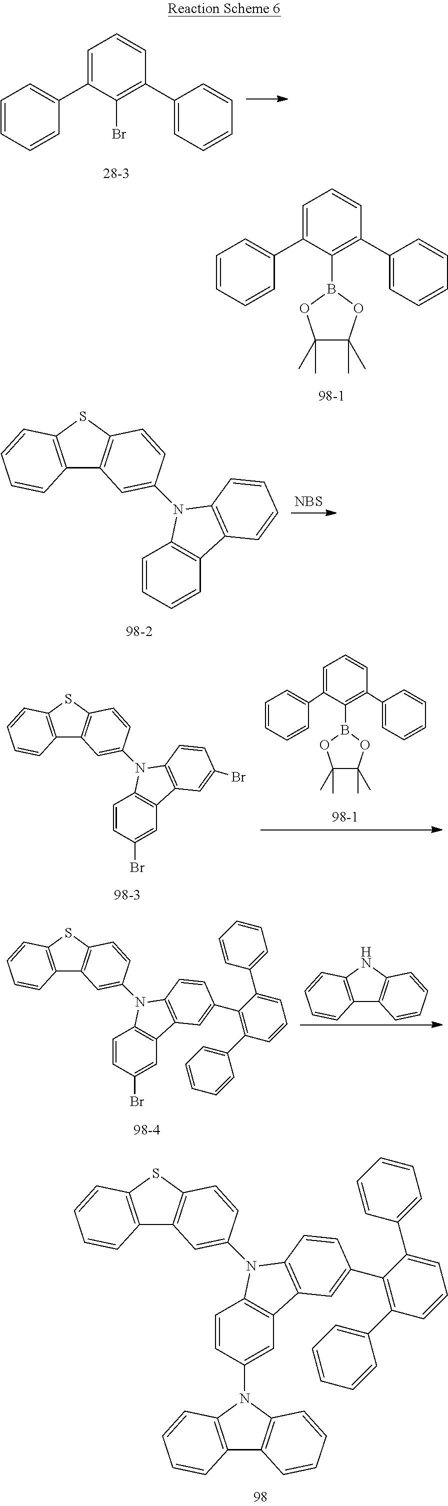

### Synthesis of Intermediate 98-1

Intermediate 28-3 and bis(pinacolato)diborone were reacted under a Pd catalyst condition to thereby obtain Intermediate 98-1. M+1 peak value of Intermediate 98-1 was confirmed by liquid chromatography mass spectrometry (LC-MS).

C₂₄H₂₅BO₂: M+1 357.22

### Synthesis of Intermediate 98-2

Intermediate 98-2 was synthesized in the same manner as Intermediate 56-4, except that, in the synthesis process, 2-bromodibenzothiophene (CAS number=22439-61-8) was utilized instead of 2-bromodibenzofuran (CAS number=86-76-0). M+1 peak value of Intermediate 98-2 was confirmed by liquid chromatography mass spectrometry (LC-MS).

C₂₄H₁₅NS: M+1 350.02

### Synthesis of Intermediate 98-3

Intermediate 98-2 and N-bromosuccinimide were reacted to thereby obtain Intermediate 98-3. M+1 peak value of Intermediate 98-3 was confirmed by liquid chromatography mass spectrometry (LC-MS).

C₂₄H₁₃Br₂NS: M+1 505.92

### Synthesis of Intermediate 98-4

Intermediate 98-3 and Intermediate 98-1 were reacted under a Pd catalyst condition to thereby obtain Intermediate 98-4. M+1 peak value of Intermediate 98-4 was confirmed by liquid chromatography mass spectrometry (LC-MS).

C₄₂H₂₆BrNS: M+1 656.13

### Synthesis of Compound 98

Compound 98 was synthesized in the same manner as Compound 21, except that, in the synthesis process, Intermediate 98-4 was utilized instead of Intermediate 21-2. 4.4 g (yield: 77%) of Compound 98 was obtained. Compound 98 was confirmed by LC-MS and ¹H-NMR.

### Synthesis Example 7: Synthesis of Compound 102

The polycyclic Compound 102 according to an embodiment may be synthesized by, for example, Reaction Scheme 7.

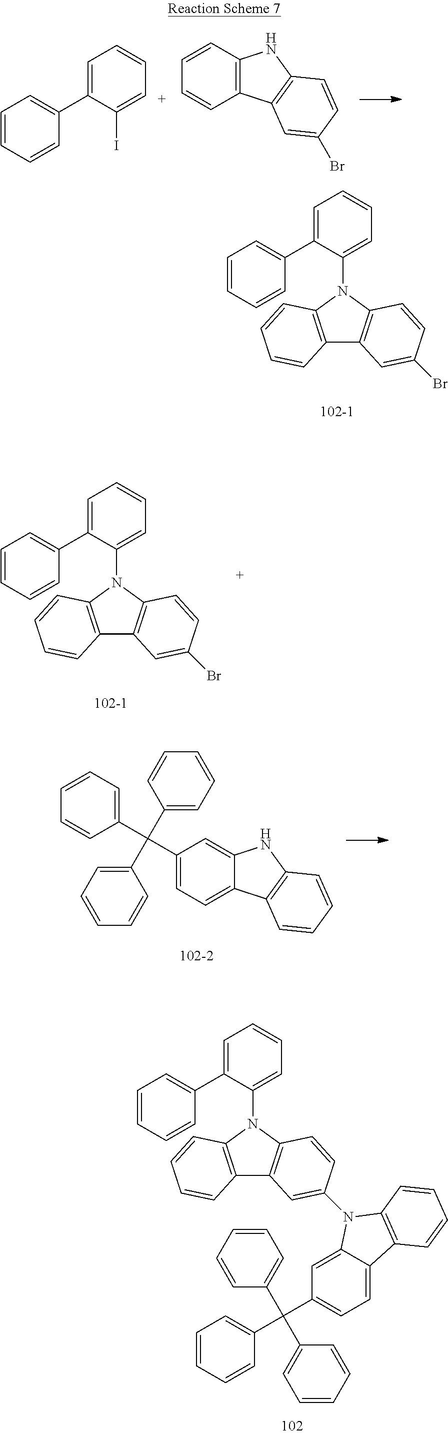

### Synthesis of Intermediate 102-1

2-iodobiphenyl (CAS number=2113-51-1) and 3-bromo-9H-carbazole (CAS number=1592-95-6) were reacted under a Cu catalyst condition to thereby obtain Intermediate 102-1. M+1 peak value of Intermediate 102-1 was confirmed by liquid chromatography mass spectrometry (LC-MS).

C24H16BrN: M+1 398.27

### Synthesis of Intermediate 102-2

Intermediate 102-2 was synthesized in the same manner as Intermediate 56-3, except that, in the synthesis process, chlorotriphenylmethane (CAS number=76-83-5) was utilized instead of chlorotriphenylsilane (CAS number=76-86-8). M+1 peak value of Intermediate 102-2 was confirmed by liquid chromatography mass spectrometry (LC-MS).

C₃₁H₂₃N: M+1 410.18

### Synthesis of Compound 102

4 g of Intermediate 102-1, 6 g of Intermediate 102-2, 1.63 g of sodiumtert-butoxide, 0.41 g of tris(dibenzylidene acetone)dipalladium(0), 0.36 ml of tritert-butylphosphine, and 55 ml of toluene were put in a reaction vessel and refluxed for 24 hours. After the reaction was completed, the reaction solution was extracted with ethylacetate, the collected organic layer was dried with magnesium sulfate, and a solvent was evaporated therefrom. The obtained residue was separated and purified by silica gel column chromatography to obtain 6.6 g (yield: 80%) of Compound 102. Compound 102 was confirmed by LC-MS and ¹H-NMR.

### Synthesis Example 8: Synthesis of Compound 113

The polycyclic Compound 113 according to an embodiment may be synthesized by, for example, Reaction Scheme 8.

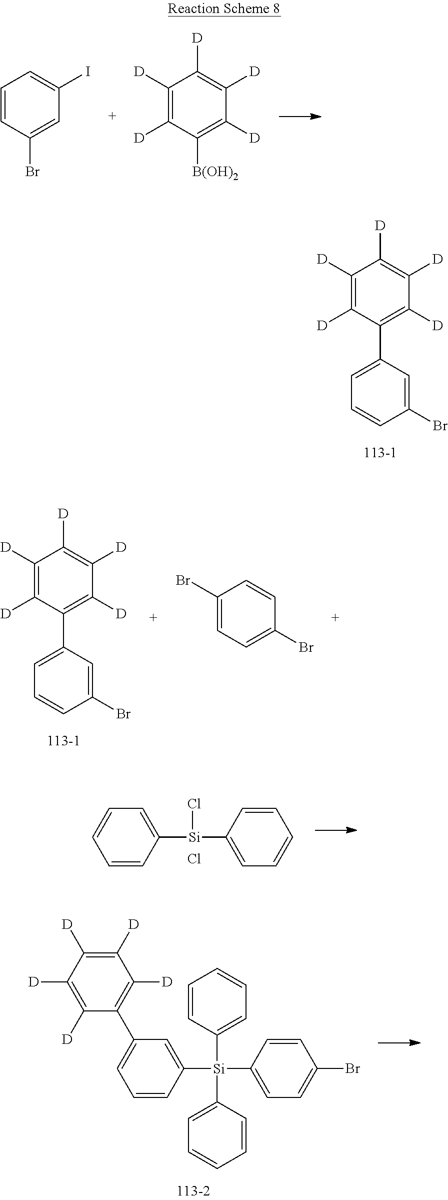

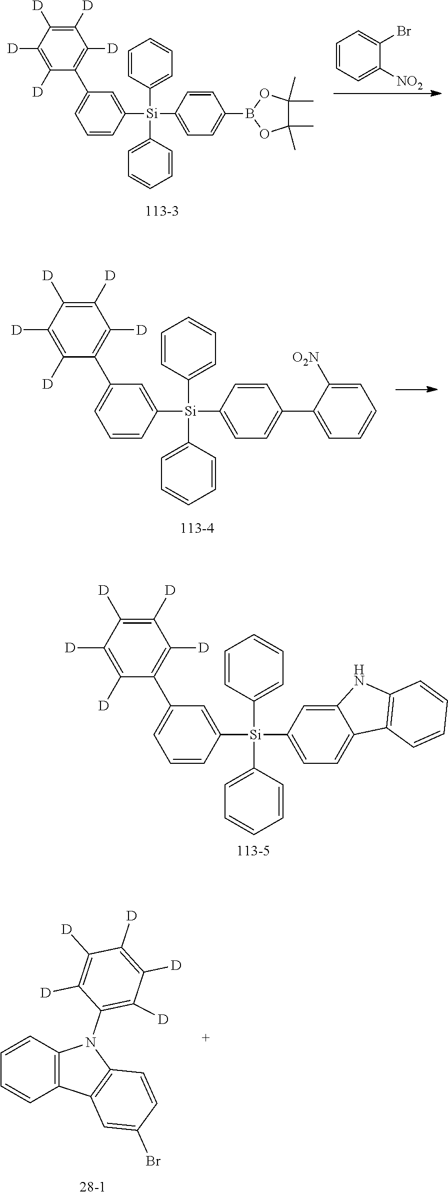

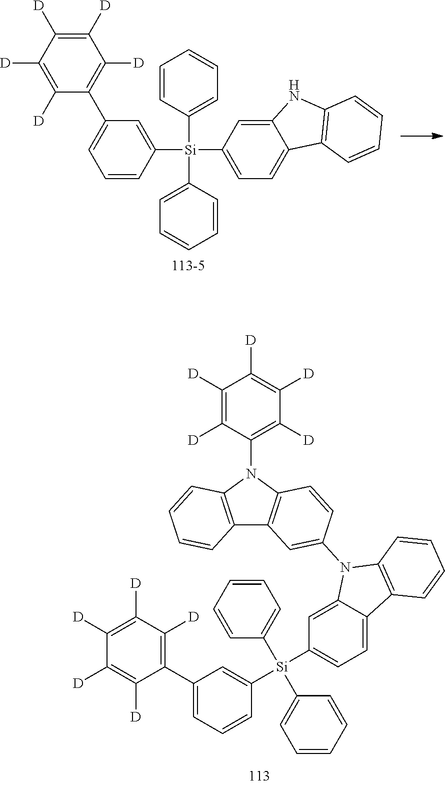 [SMILES: [2H]c1c([2H])c([2H])c(-c2cccc([Si](c3ccccc3)(c3ccccc3)c3ccc4c(c3)[nH]c3ccccc34)c2)c([2H])c1[2H] ; [2H]c1c([2H])c([2H])c(-c2cccc([Si](c3ccccc3)(c3ccccc3)c3ccc4c5ccccc5n(-c5ccc6c(c5)c5ccccc5n6-c5c([2H])c([2H])c([2H])c([2H])c5[2H])c4c3)c2)c([2H])c1[2H]]

### Synthesis of Intermediate 113-1

1-bromo-3-iodobenzene (CAS number=591-18-4) and (phenyl-d₅)boronic acid (CAS number=215527-70-1) were reacted under a Pd catalyst condition to thereby obtain Intermediate 113-1. M+1 peak value of Intermediate 113-1 was confirmed by liquid chromatography mass spectrometry (LC-MS).

C₁₂H₄D₅Br: M+1 238.12

### Synthesis of Intermediate 113-2

Intermediate 113-1 and 1,4-dibromobenzene (CAS number=106-37-6) were each reacted with n-BuLi, and then reacted with dichlorodiphenylsilane (CAS number=80-10-4) to thereby obtain Intermediate 113-2. M+1 peak value of Intermediate 113-2 was confirmed by liquid chromatography mass spectrometry (LC-MS).

C₃₀H₁₈D₅BrSi: M+1 496.12

### Synthesis of Intermediate 113-3

Intermediate 113-2 and bis(pinacolato)diborone were reacted under a Pd catalyst condition to thereby obtain Intermediate 113-3. M+1 peak value of Intermediate 113-3 was confirmed by liquid chromatography mass spectrometry (LC-MS).

C₃₆H₃₀D₅BO₂Si: M+1 544.31

### Synthesis of Intermediate 113-4

Intermediate 113-3 and 1-bromo-2-nitrobenzene (CAS number=577-19-5) were reacted under a Pd catalyst condition to thereby obtain Intermediate 113-4. M+1 peak value of Intermediate 113-4 was confirmed by liquid chromatography mass spectrometry (LC-MS).

C₃₆H₂₂D₅NO₂Si: M+1 539.28

### Synthesis of Intermediate 113-5

Intermediate 113-4 and triphenylphosphine (CAS number=603-35-0) were reacted to thereby obtain Intermediate 113-5. M+1 peak value of Intermediate 113-5 was confirmed by liquid chromatography mass spectrometry (LC-MS).

C₃₆H₂₂D₅NSi: M+1 507.21

### Synthesis of Compound 113

Compound 113 was synthesized in the same manner as Compound 28, except that, in the synthesis process, Intermediate 113-5 was utilized instead of Intermediate 28-4. 6.9 g (yield: 75%) of Compound 113 was obtained. Compound 113 was confirmed by LC-MS and ¹H-NMR.

### Synthesis Example 9: Synthesis of Compound 123

The heterocyclic Compound 123 according to an embodiment may be synthesized by, for example, Reaction Scheme 9.

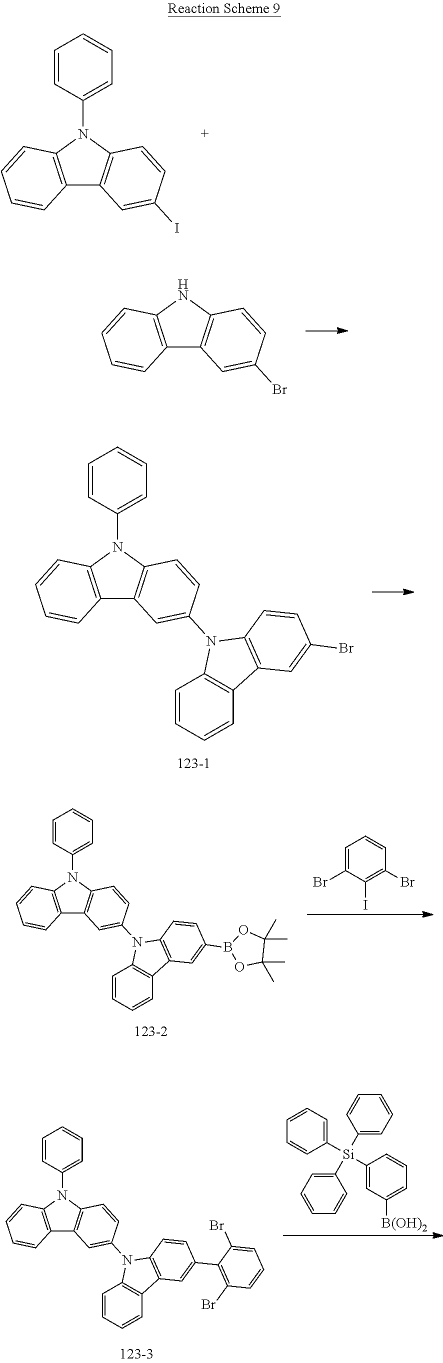

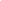

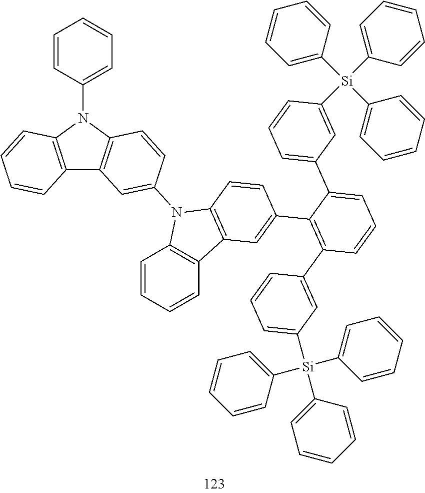 [SMILES: c1ccc(-n2c3ccccc3c3cc(-n4c5ccccc5c5cc(-c6c(-c7cccc([Si](c8ccccc8)(c8ccccc8)c8ccccc8)c7)cccc6-c6cccc([Si](c7ccccc7)(c7ccccc7)c7ccccc7)c6)ccc54)ccc32)cc1]

### Synthesis of Intermediate 123-1

3-iodo-9-phenyl-9H-carbazole (CAS number=502161-03-7) and 3-bromo-9H-carbazole (CAS number=1592-95-6) were reacted under a Cu catalyst condition to thereby obtain Intermediate 123-1. M+1 peak value of Intermediate 123-1 was confirmed by liquid chromatography mass spectrometry (LC-MS).

C₃₀H₁₉BrN₂: M+1 487.11

### Synthesis of Intermediate 123-2

Intermediate 123-1 and bis(pinacolato)diborone were reacted under a Pd catalyst condition to thereby obtain Intermediate 123-2. M+1 peak value of Intermediate 123-2 was confirmed by liquid chromatography mass spectrometry (LC-MS).

C₃₆H₃₁BN₂O₂: M+1 535.26

### Synthesis of Intermediate 123-3

Intermediate 123-2 and 1,3-dibromo-2-iodobenzene (CAS number=19821-80-8) were reacted under a Cu catalyst condition to thereby obtain Intermediate 123-3. M+1 peak value of Intermediate 123-3 was confirmed by liquid chromatography mass spectrometry (LC-MS).

C₃₆H₂₂Br₂N₂: M+1 641.01

### Synthesis of Compound 123

5 g of Intermediate 123-3, 6.2 g of 3-(triphenylsilyl)phenylboronic acid (CAS number=1253912-58-1), 0.45 g of tetrakis(triphenylphosphine)palladium, and 2.7 g of potassium carbonate were put in a reaction vessel, dissolved in 40 ml of toluene, 10 ml of ethanol, and 10 ml of distilled water, and refluxed for 24 hours. After the reaction was completed, the reaction solution was extracted with ethylacetate, the collected organic layer was dried with magnesium sulfate, and a solvent was evaporated therefrom. The obtained residue was separated and purified by silica gel column chromatography to obtain 5.8 g (yield: 65%) of Compound 123. Compound 123 was confirmed by LC-MS and ¹H-NMR.

¹H NMR and MS/FAB of the compounds synthesized according to Synthesis Examples 1 to 9 are shown in Table 1 below. Synthesis methods for other compounds than the compounds shown in Table 1 may be easily recognized by those skilled in the technical field by referring to the synthesis paths and source material materials described above.

TABLE 1

| | | MS/FAB | |
|---|---|---|---|
| | | [M + 1] | |
| Compound | H NMR (δ) | Calc | found |
| 1 | 8.55 (2H, d), 7.94 (2H, d), 7.83 (1H, s), 7.72-7.58 (6H, | 666.25 | 667.21 |
| | m), 7.50-7.35 (21H, m), 7.16 (2H, t) | | |
| 21 | 8.82 (1H, s), 8.55 (1H, s), 8.19 (1H, s), 7.94 (2H, m), | 650.27 | 651.34 |
| | 7.72-7.50 (9H, m), 7.38-7.16 (19H, m), 6.98 (1H, d) | | |
| 28 | 8.55 (2H, d), 8.06-7.67 (14H, m), 7.46-7.35 (9H, m), | 641.29 | 642.22 |
| | 7.16 (2H, t) | | |
| 56 | 8.55 (2H, d), 8.22 (1H, d), 7.98-7.94 (3H, m), 7.74- | 756.26 | 756.31 |
| | 7.61 (4H, m), 7.54-7.31 (24H, m), 7.16 (2H, t) | | |
| 77 | 8.55 (2H, d), 8.45 (1H, d), 8.10 (1H, d), 8.05 (1H, s), | 756.26 | 756.30 |
| | 7.94-7.90 (4H, m), 7.72 (1H, d), 7.67 (1H, s), 7.56- | | |
| | 7.16 (25H, m) | | |
| 98 | 8.55 (1H, d), 8.45 (1H, d), 8.30 (1H, d), 8.19-8.06 (5H, | 742.24 | 743.23 |
| | m), 7.93-7.67 (11H, m), 7.56-7.35 (13H, m), 7.20-7.16 | | |
| | (2H, m) | | |
| 102 | 8.55 (2H, d), 8.11 (1H, d), 7.94-7.91 (4H, m), 7.80 | 726.30 | 727.34 |
| | (1H, t), 7.72 (1H, d), 7.67 (1H, s), 7.41-7.16 (28H, m) | | |
| 113 | 8.55 (2H, d), 7.94 (2H, d), 7.88 (1H, s), 7.83 (1H, s), | 752.34 | 753.28 |
| | 7.72-7.56 (6H, m), 7.46-7.35 (14H, m), 7.16 (2H, t) | | |
| 123 | 8.55 (2H, d), 8.06 (2H, d), 7.99-7.83 (6H, m), 7.77- | 1152.43 | 1153.45 |
| | 7.35 (48H, m), 7.16 (2H, t) | | |

### Example 1

As an anode, a Corning 15 Ω/cm² (1,200 Å) ITO glass substrate was cut to a size of 50 mm×50 mm×0.7 mm, sonicated with isopropyl alcohol and pure water each for 5 minutes, and then cleaned by exposure to ultraviolet rays and ozone for 30 minutes. The ITO glass substrate was provided to a vacuum deposition apparatus.

N,N′-di(1-naphthyl)-N,N′-diphenylbenzidine (NPB) was vacuum deposited on the ITO anode formed on the glass substrate to form a hole injection layer having a thickness of 300 Å.

mCP was vacuum-deposited on the hole injection layer to form a hole transport layer having a thickness of 200 Å.

Compound 1 (host) and Ir(pmp)₃(dopant) were co-deposited at the weight ratio of 92:8 on the hole transport layer to form an emission layer having a thickness of 250 Å.

Then, TAZ was deposited on the emission layer to form a electron transport layer having a thickness of 200 Å.

LiF, which is an alkali metal halide, was deposited on the electron transport layer to form an electron injection layer having a thickness of 10 Å, and an Al was vacuum-deposited thereon to a thickness of 100 Å to form a LiF/Al electrode, thereby completing the manufacture of a light-emitting device.

Materials utilized in the above-described organic light-emitting device may be represented by the formula below.

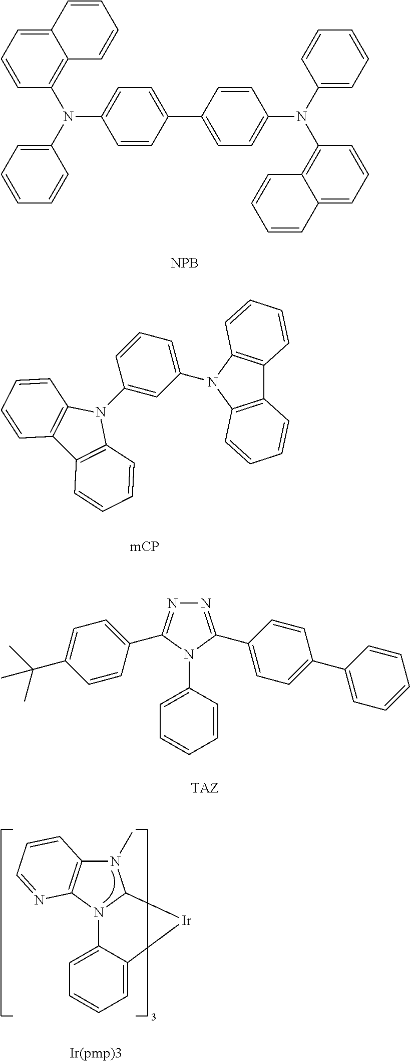

### Examples 2 to 9

Light-emitting devices were manufactured in the same manner as in Example 1, except that, in forming the emission layer, the compounds shown in Table 2 were each utilized instead of Compound 1.

### Comparative Examples 1 to 3

Light-emitting devices were manufactured in the same manner as in Example 1, except that Compounds A to C were each utilized to form emission layers.

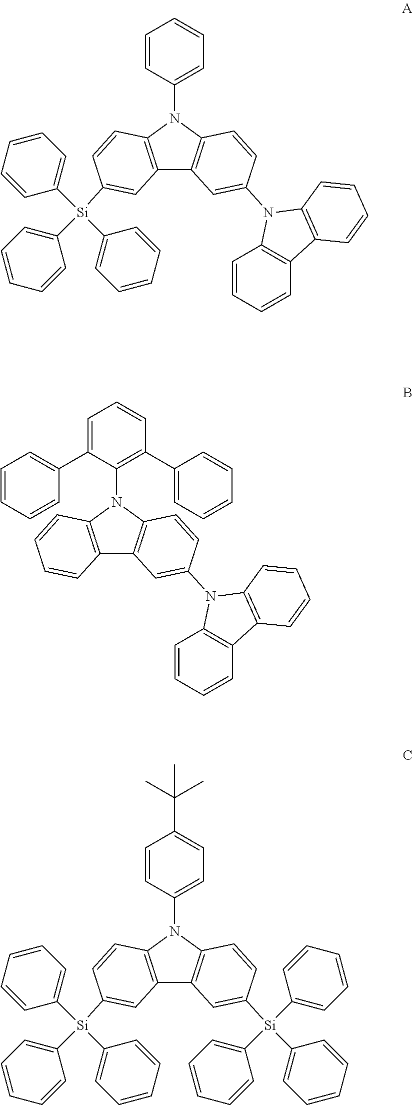 [SMILES: c1ccc(-n2c3ccc(-n4c5ccccc5c5ccccc54)cc3c3cc([Si](c4ccccc4)(c4ccccc4)c4ccccc4)ccc32)cc1 ; c1ccc(-c2cccc(-c3ccccc3)c2-n2c3ccccc3c3cc(-n4c5ccccc5c5ccccc54)ccc32)cc1 ; CC(C)(C)c1ccc(-n2c3ccc([Si](c4ccccc4)(c4ccccc4)c4ccccc4)cc3c3cc([Si](c4ccccc4)(c4ccccc4)c4ccccc4)ccc32)cc1]

### Evaluation Example 1

To evaluate characteristics of the light-emitting devices manufactured according to Examples 1 to 9 and Comparative Examples 1 to 3, the driving voltage at the current density of 10 mA/cm², luminescence efficiency, and maximum quantum efficiency thereof were measured. The driving voltage and current density of each of the organic light-emitting devices were each measured utilizing a source meter (Keithley Instrument, 2400 series), and the maximum quantum efficiency was measured utilizing the external quantum efficiency measurement device C9920-2-12 of Hamamatsu Photonics Inc In evaluating the maximum quantum efficiency, the luminance/current density was measured utilizing a luminance meter that was calibrated for wavelength sensitivity, and the maximum quantum efficiency was converted by assuming an angular luminance distribution (Lambertian) which introduced a perfect reflecting diffuser. Table 2 below shows the evaluation results of the characteristics of each of the organic light-emitting devices.

TABLE 2

| | | | | | Maximum | |
|---|---|---|---|---|---|---|
| | | Driving | Current | Emission | quantum | |
| | | voltage | density | efficiency | efficiency | Emission |
| No. | Host | (V) | (mA/cm²) | (cd/A) | (%) | color |
| Example 1 | Compound 1 | 4.8 | 2.3 | 22.8 | 22.4 | Blue |
| Example 2 | Compound 21 | 4.7 | 2.3 | 20.9 | 20.3 | Blue |
| Example 3 | Compound 28 | 4.6 | 2.3 | 22.3 | 21.7 | Blue |
| Example 4 | Compound 56 | 4.8 | 2.3 | 21.8 | 21.9 | Blue |
| Example 5 | Compound 77 | 4.7 | 2.3 | 20.7 | 20.4 | Blue |
| Example 6 | Compound 98 | 4.8 | 2.3 | 20.3 | 19.8 | Blue |
| Example 7 | Compound 102 | 4.6 | 2.3 | 20.8 | 20.4 | Blue |
| Example 8 | Compound 113 | 4.7 | 2.3 | 21.4 | 21.8 | Blue |
| Example 9 | Compound 123 | 5.0 | 2.3 | 19.9 | 19.9 | Blue |
| Comparative | Compound A | 5.1 | 2.3 | 18.7 | 18.3 | Blue |
| Example 1 | | | | | | |
| Comparative | Compound B | 4.8 | 2.3 | 17.8 | 18.1 | Blue |
| Example 2 | | | | | | |
| Comparative | Compound C | 4.9 | 2.3 | 18.5 | 17.9 | Blue |
| Example 3 | | | | | | |

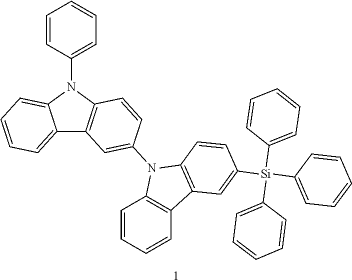

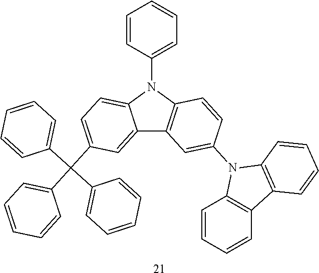 [SMILES: c1ccc(-n2c3ccc(-n4c5ccccc5c5ccccc54)cc3c3cc(C(c4ccccc4)(c4ccccc4)c4ccccc4)ccc32)cc1 ; "21"]

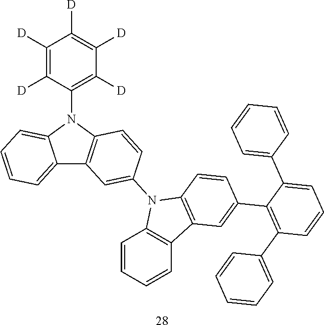

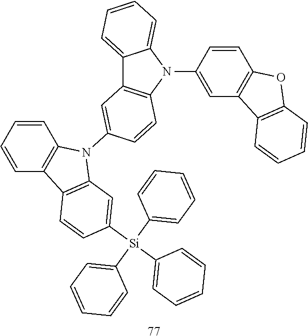

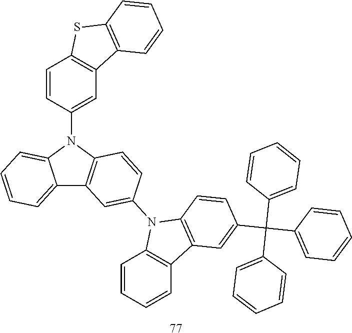

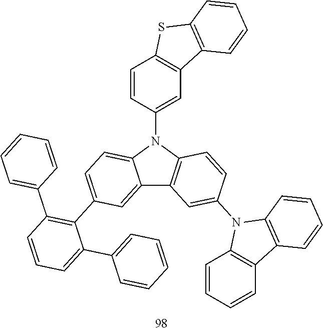

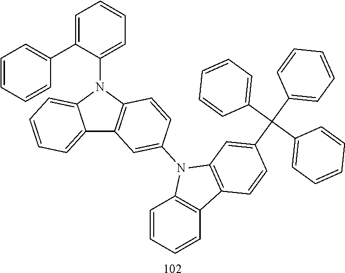

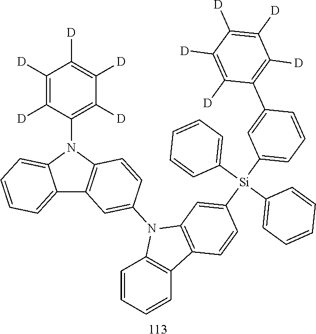

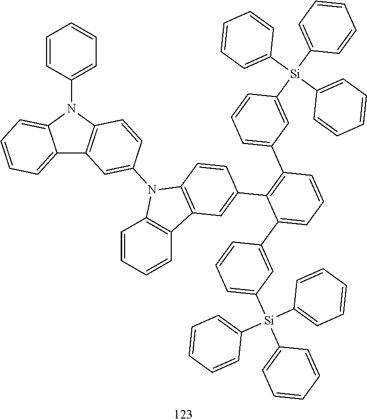 [SMILES: c1ccc(-n2c3ccccc3c3cc(-n4c5ccccc5c5cc(-c6c(-c7cccc([Si](c8ccccc8)(c8ccccc8)c8ccccc8)c7)cccc6-c6cccc([Si](c7ccccc7)(c7ccccc7)c7ccccc7)c6)ccc54)ccc32)cc1 ; "123"]

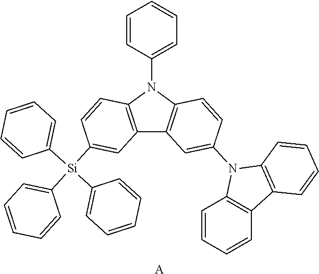

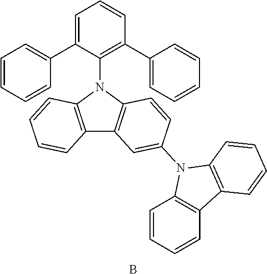

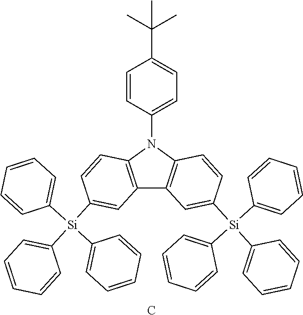

From Table 2, it can be seen that the light-emitting devices of Examples 1 to 9 have lower driving voltage, higher luminescence efficiency, and higher maximum quantum efficiency compared to the light-emitting devices of Comparative Examples 1 to 3.

The heterocyclic compound can be utilized in manufacturing a light-emitting device having high efficiency and low driving voltage, and the light-emitting device can be utilized in manufacturing a high-quality electronic apparatus having excellent luminescence efficiency and a long lifespan.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims, and equivalents thereof.

What is claimed is:

1. A heterocyclic compound represented by Formula 1: 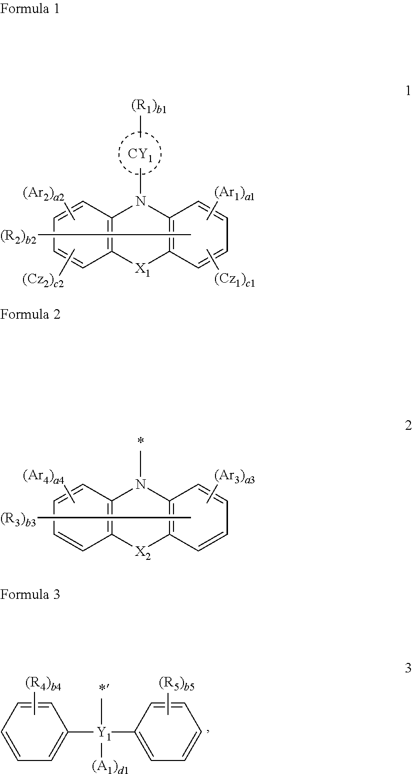
wherein, in Formula 1,
X₁ is a single bond, O, S, N(Z₁₁), C(Z₁₁)(Z₁₂), or Si(Z₁₁)(Z₁₂),
ring CY₁ is a C₃-C₆₀ carbocyclic group or a C₁-C₆₀ heterocyclic group,
Cz₁ and Cz₂ are each independently a group represented by Formula 2,
c1 and c2 are each independently an integer selected from 0 to 4, and a sum of c1 and c2 is 1 or more, and
b1 is an integer selected from 0 to 10, and b2 is an integer selected from 0 to 7,
wherein in Formula 2,
X₂ is a single bond, O, S, N(Z₂₁), C(Z₂₁)(Z₂₂), or Si(Z₂₁)(Z₂₂),
b3 is an integer selected from 0 to 7, and
* indicates a binding site to a neighboring atom of Formula 1,
wherein in Formulae 1 and 2,
Ar₁ to Ar₄ are each independently a group represented by Formula 3,
a1 and a2 are each independently an integer selected from 0 to 3, a3 and a4 are each independently an integer selected from 0 to 4, and a sum of a1 to a4 is 1 or more, and
when a2 and c1 are each 1, and a1, a3, a4 and c2 are each 0, Ar₂ is not a triphenylsilane,
wherein in Formula 3,
Y₁ is C, Si, or a phenylene group,
b4 and b5 are each independently an integer selected from 0 to 5,
A₁ is hydrogen, deuterium, —F, —Cl, —Br, —I, a hydroxyl group, a cyano group, a nitro group, a C₁-C₆₀ alkyl group unsubstituted or substituted with at least one R₁₀ₐ, or a C₆-C₆₀ aryl group unsubstituted or substituted with at least one R₁₀ₐ,
d1 is an integer selected from 1 to 3, and
*′ indicates a binding site to a neighboring atom of Formula 2 or 3,
wherein in Formulae 1 to 3,
R₁ to R₅, Z₁₁, Z₁₂, Z₂₁, and Z₂₂ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —C(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂), and
R₁₀ₐ is:
deuterium (-D), —F, —Cl, —Br, —I, a hydroxyl group, a cyano group, or a nitro group; a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —C(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —C(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —C(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
wherein Q₁ to Q₃, Q₁₁ to Q₁₃, Q₂₁ to Q₂₃, and Q₃₁ to Q₃₃ are each independently hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

2. The heterocyclic compound of claim 1, wherein, when ring CY₁ in Formula 1 is a benzene group, R₁ is:
hydrogen, deuterium, —F, —Cl, —Br, —I, a hydroxyl group, a cyano group, or a nitro group;
a C₁-C₂₀ alkyl group, a C₂-C₂₀ alkenyl group, a C₂-C₂₀ alkynyl group, a C₁-C₂₀ alkoxy group, or a phenyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a C₂-C₂₀ alkenyl group, a C₂-C₂₀ alkynyl group, a C₁-C₂₀ alkoxy group, or any combination thereof; or
a naphthalene group, a fluorenyl group, a dibenzofuran group, or a dibenzothiophene group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, C₂-C₂₀ alkenyl group, C₂-C₂₀ alkynyl group, C₁-C₂₀ alkoxy group, a phenyl group, or any combination thereof, and
when R₁ is a phenyl group, b1 is 1.

3. The heterocyclic compound of claim 1, wherein, in Formulae 1 and 2, a1 to a4, c1, and c2 are each independently 0 or 1.

4. The heterocyclic compound of claim 1, wherein, in Formula 1, when c1 is 1 and c2 is 0, i) a1 and a2 are each independently 0 or 1, or ii) a3 and a4 are each independently 0 or 1.

5. The heterocyclic compound of claim 1, wherein, in Formula 1, when c1 and c2 are each 1, a1 and a2 are each 0, and a3 and a4 are each independently 0 or 1.

6. The heterocyclic compound of claim 1, wherein, in Formulae 1 and 2, R₂, R₃, Z₁₁, Z₁₂, Z₂₁, and Z₂₂ are each independently:
hydrogen, deuterium, —F, —Cl, —Br, —I, a hydroxyl group, a cyano group, or a nitro group; or
a C₁-C₂₀ alkyl group, a C₂-C₂₀ alkenyl group, a C₂-C₂₀ alkynyl group, a C₁-C₂₀ alkoxy group, or a phenyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a C₂-C₂₀ alkenyl group, a C₂-C₂₀ alkynyl group, a C₁-C₂₀ alkoxy group, a phenyl group, or any combination thereof.

7. The heterocyclic compound of claim 1, wherein, in Formula 3, R₄ and R₅ are each independently:
hydrogen, deuterium, —F, —Cl, —Br, —I, a hydroxyl group, a cyano group, or a nitro group;
a C₁-C₂₀ alkyl group, a C₂-C₂₀ alkenyl group, a C₂-C₂₀ alkynyl group, or a C₁-C₂₀ alkoxy group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, —Si(Q₃₁)(Q₃₂)(Q₃₃), —C(Q₃₁)(Q₃₂)(Q₃₃), or any combination thereof;
a phenyl group, a fluorenyl group, a dibenzofuranyl group, or a dibenzothiophenyl group, each unsubstituted or substituted with deuterium, —F, —Cl, —Br, —CD₃, —CD₂H, —CDH₂, —CF₃, —CF₂H, —CFH₂, a hydroxyl group, a cyano group, a nitro group, a C₁-C₂₀ alkyl group, a C₂-C₂₀ alkenyl group, a C₂-C₂₀ alkynyl group, a C₁-C₂₀ alkoxy group, a phenyl group, a fluorenyl group, a dibenzofuranyl group, a dibenzothiophenyl group, —Si(Q₃₁)(Q₃₂)(Q₃₃), —C(Q₃₁)(Q₃₂)(Q₃₃), or any combination thereof; or
—Si(Q₁)(Q₂)(Q₃) or —C(Q₁)(Q₂)(Q₃).

8. The heterocyclic compound of claim 1, wherein the heterocyclic compound represented by Formula 1 is represented by one of Formulae 1-1 to 1-6: 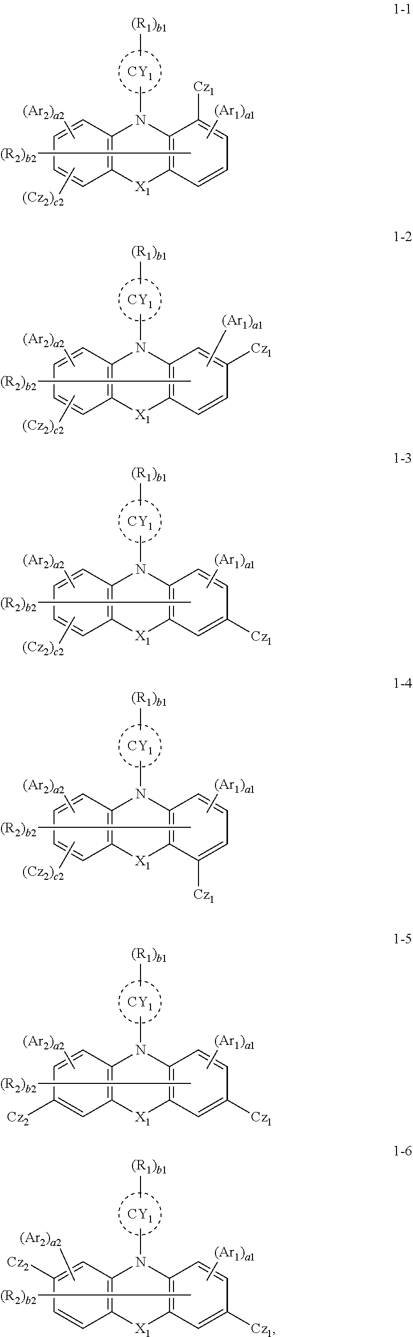
and
wherein, in Formulae 1-1 to 1-6,
X₁, CY₁, Ar₁, Ar₂, a1, a2, Cz₁, Cz₂, c2, R₁, R₂, b1, and b2 are each as defined in connection with Formula 1.

9. The heterocyclic compound of claim 1, wherein the heterocyclic compound represented by Formula 1 is represented by one of Formulae 1(1) to 1(4): 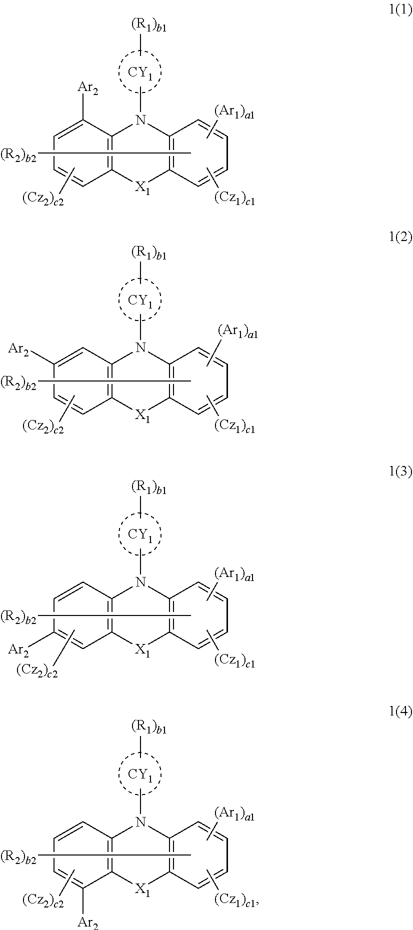
and
wherein, in Formulae 1(1) to 1(4),
X₁, CY₁, Ar₁, Ar₂, a1, Cz₁, Cz₂, c1, c2 R₁, R₂, b1, and b2 are each as defined in connection with Formula 1.

10. The heterocyclic compound of claim 1, wherein the group represented by Formula 2 is represented by one of Formulae 2-1 to 2-4: 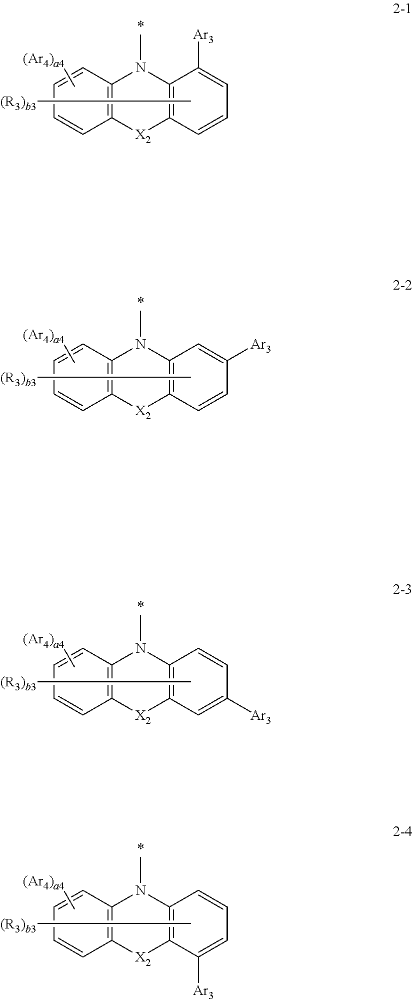
and
wherein, in Formulae 2-1 to 2-4,
X₂, Ar₃, Ar₄, a4, R₃, and b3 are each as defined in connection with Formula 2, and
* is a binding site to a neighboring atom in Formula 1.

11. The heterocyclic compound of claim 1, wherein the group represented by Formula 2 is represented by one of Formulae 2(1) to 2(5): 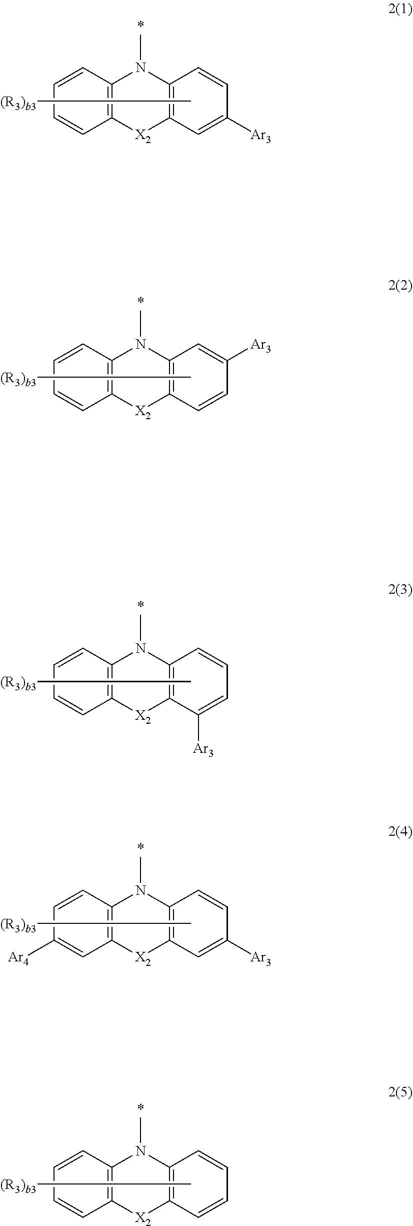
and
wherein, in Formulae 2(1) to 2(5),
X₂, Ar₃, Ar₄, R₃, and b3 are each as defined in connection with Formula 2, and
* is a binding site to a neighboring atom in Formula 1.

12. The heterocylic compound of claim 1, wherein the heterocylic compound represented by Formula 1 is represented by one selected from Formulae 1-2a, 1-2b, 1-3a to 1-3e, and 1-4a to 1-4d: 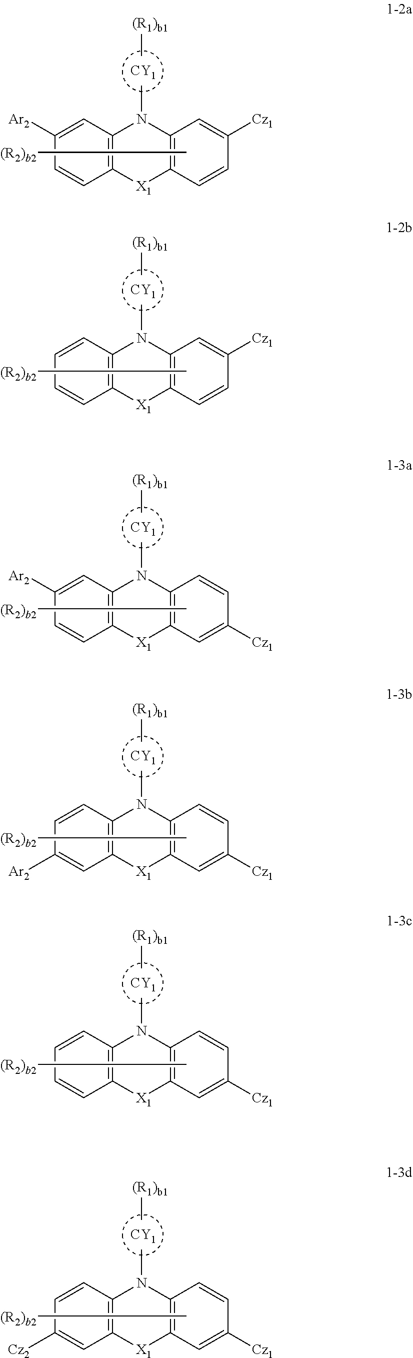 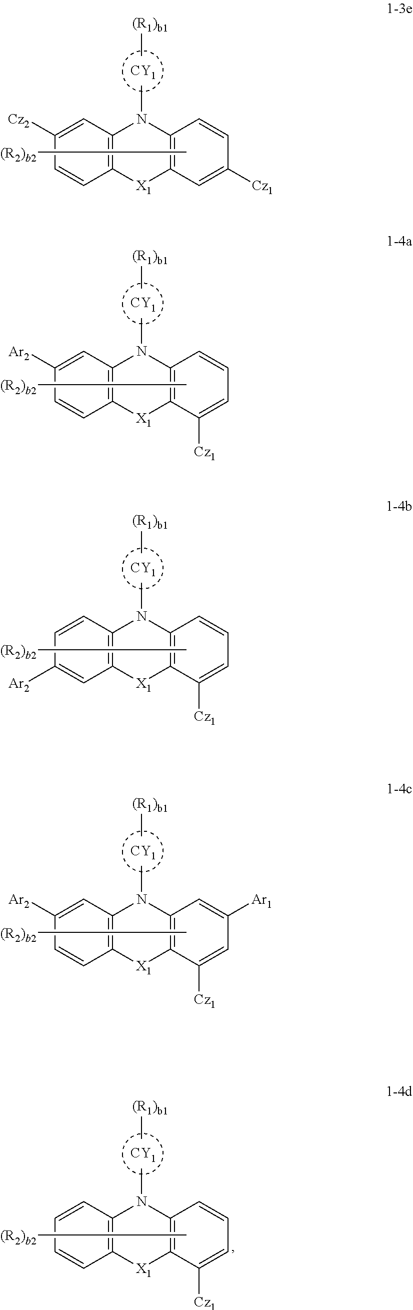
and
wherein, in Formulae 1-2a, 1-2b, 1-3a to 1-3e, and 1-4a to 1-4d,
X₁, CY₁, Ar₁, Ar₂, Cz₁, Cz₂, R₁, R₂, b1, and b2 are each as defined in connection with Formula 1.

13. The heterocylic compound of claim 1, wherein the heterocyclic compound is selected from Compounds 1 to 189:

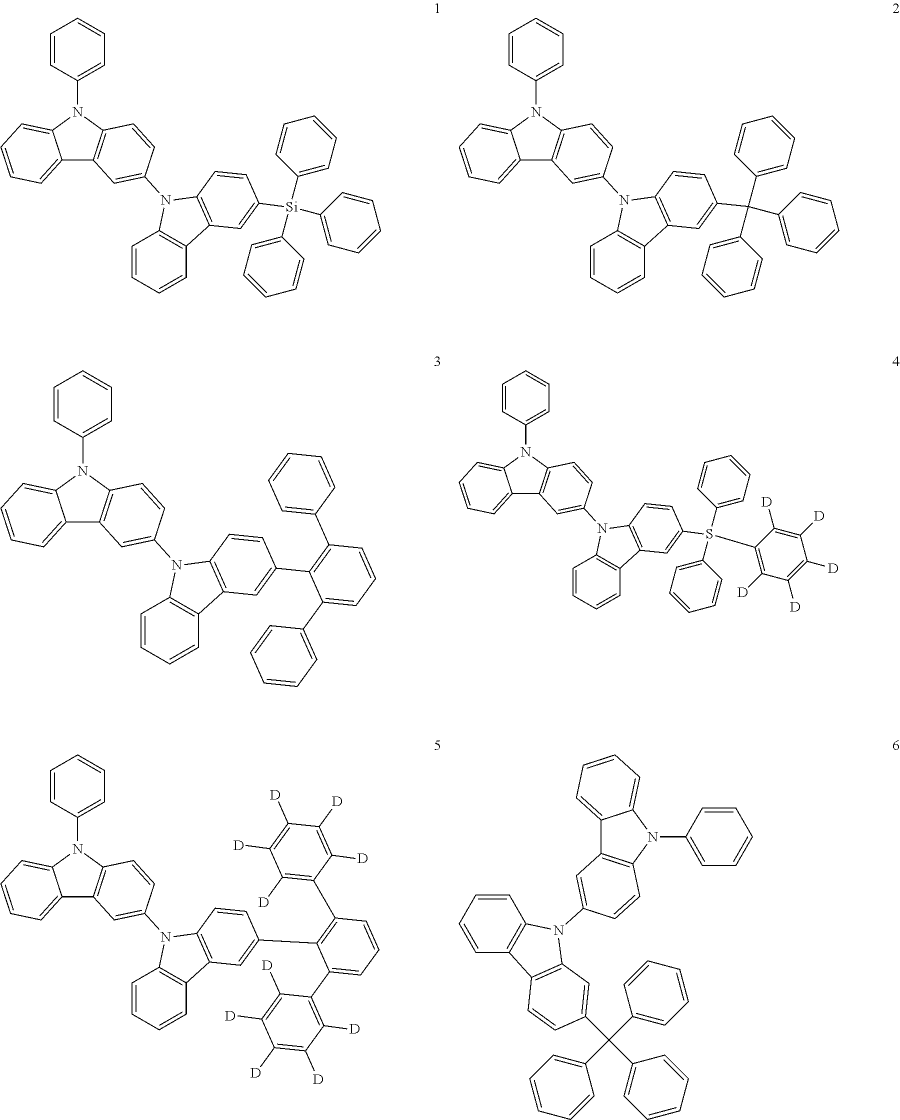

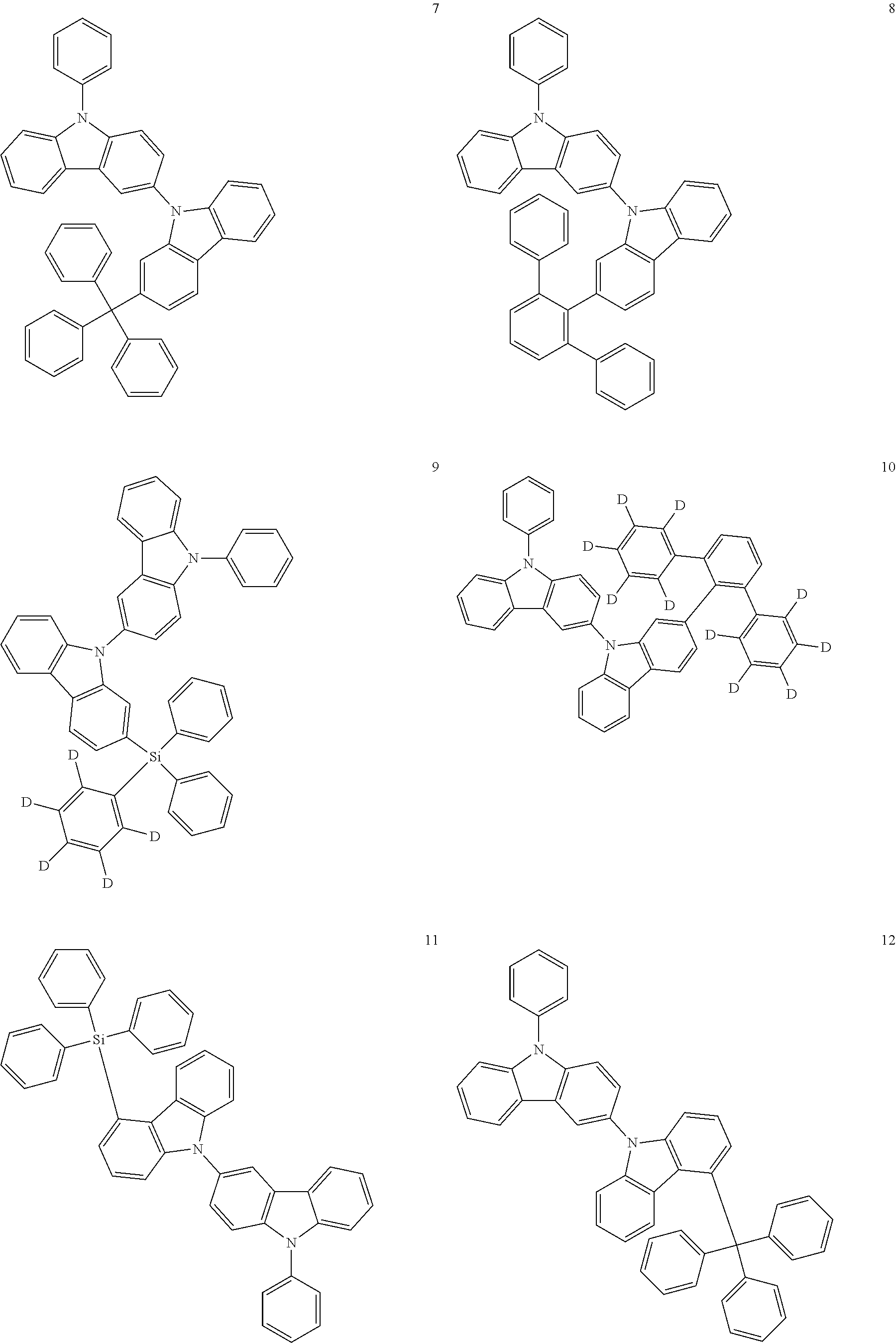

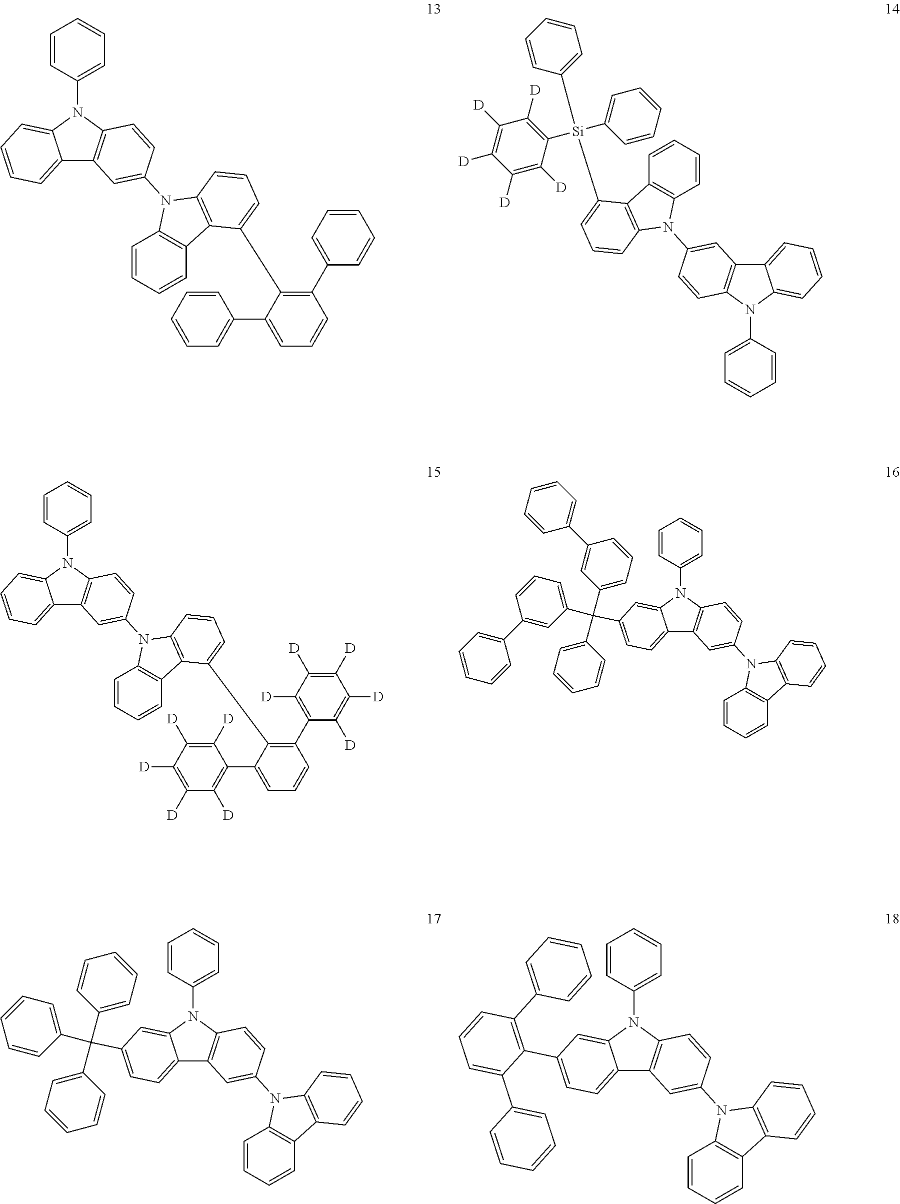

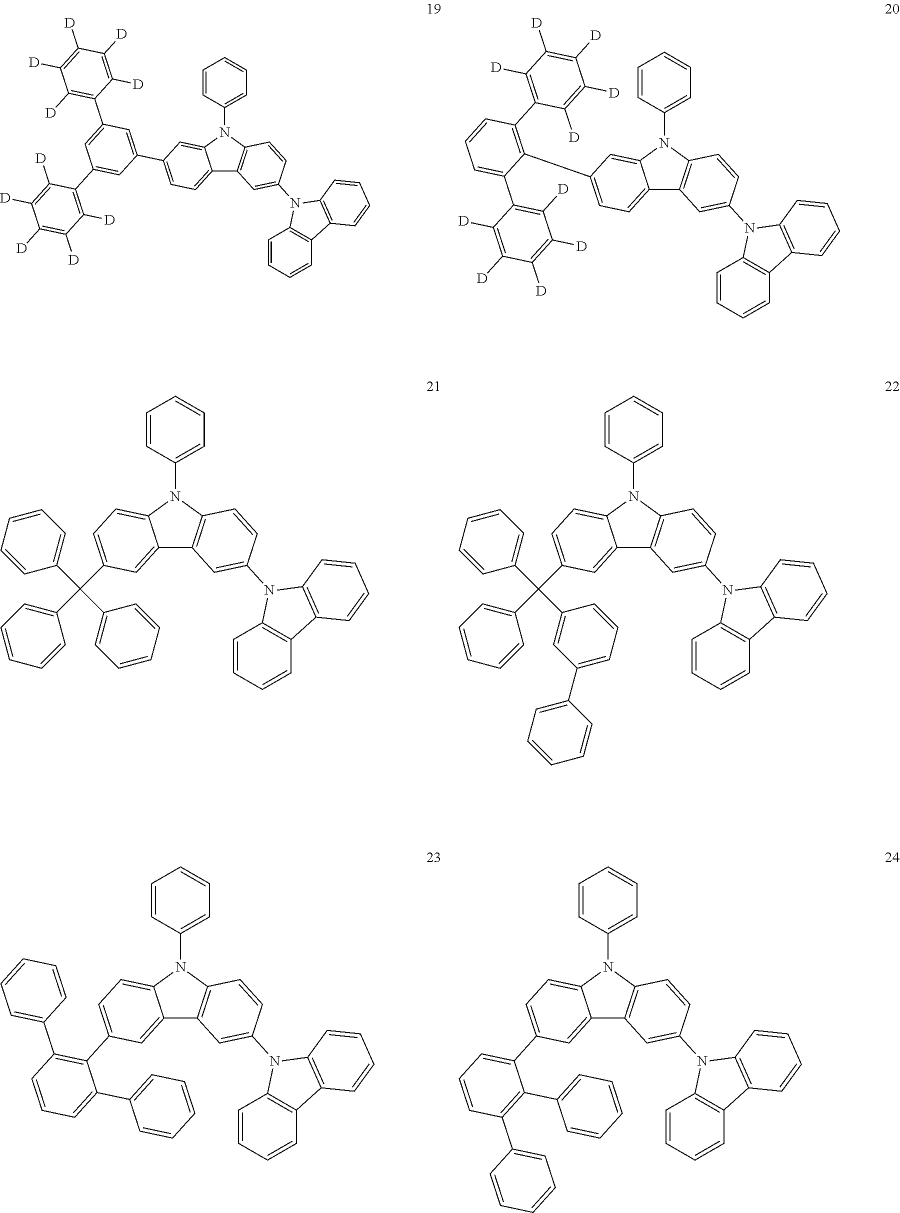

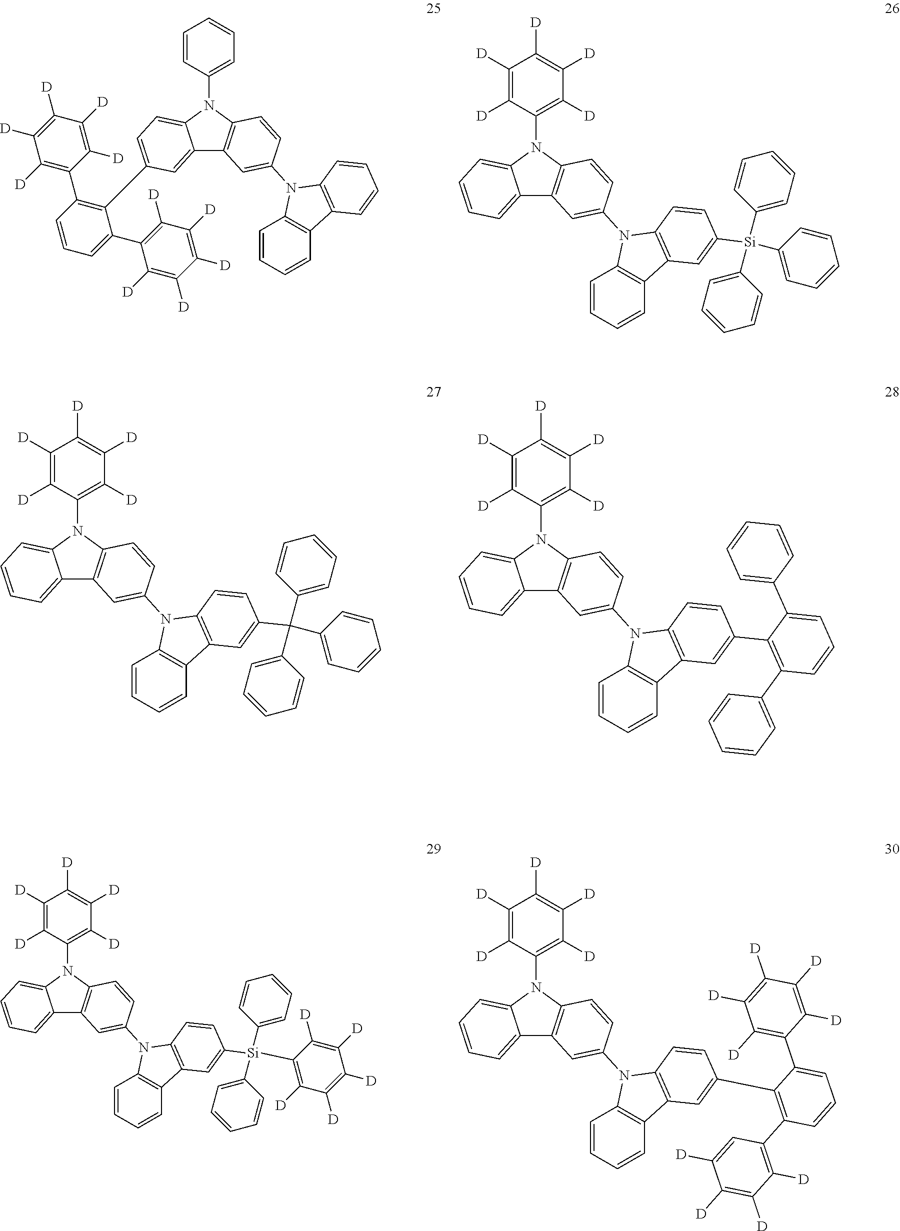

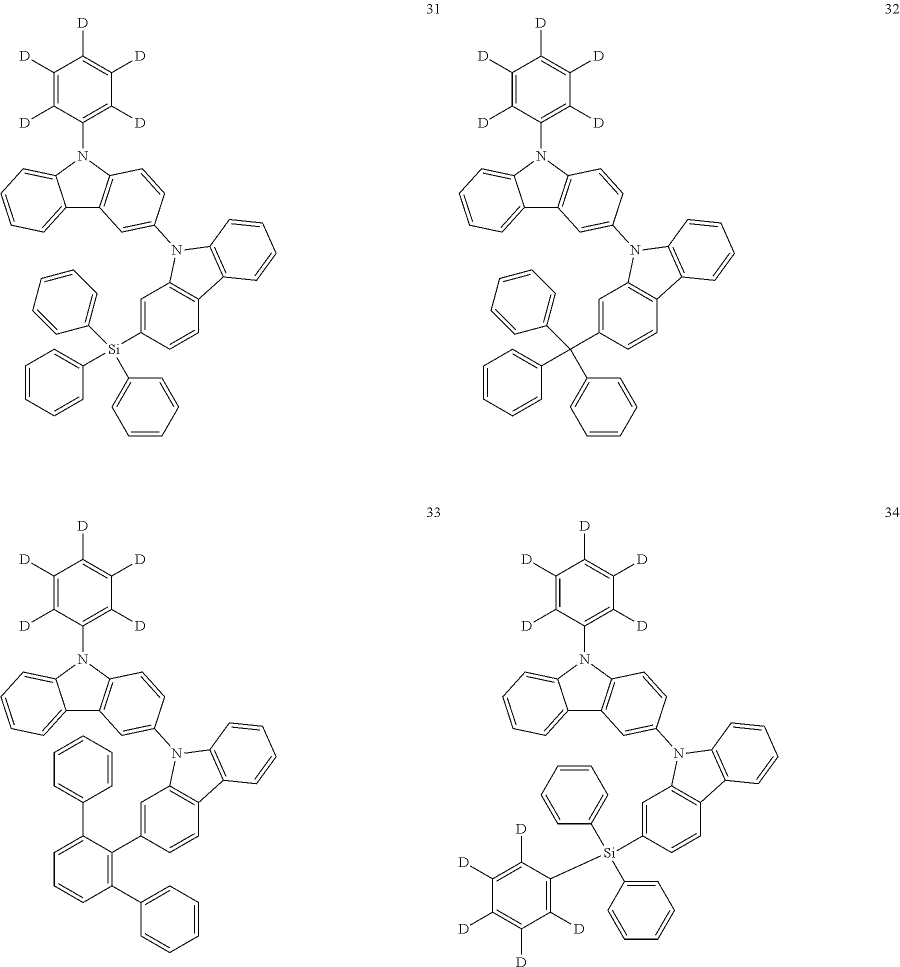 [SMILES: [2H]c1c([2H])c([2H])c(-n2c3ccccc3c3cc(-n4c5ccccc5c5ccc([Si](c6ccccc6)(c6ccccc6)c6ccccc6)cc54)ccc32)c([2H])c1[2H] ; [2H]c1c([2H])c([2H])c(-n2c3ccccc3c3cc(-n4c5ccccc5c5ccc(C(c6ccccc6)(c6ccccc6)c6ccccc6)cc54)ccc32)c([2H])c1[2H] ; [2H]c1c([2H])c([2H])c(-n2c3ccccc3c3cc(-n4c5ccccc5c5ccc(-c6c(-c7ccccc7)cccc6-c6ccccc6)cc54)ccc32)c([2H])c1[2H] ; [2H]c1c([2H])c([2H])c(-n2c3ccccc3c3cc(-n4c5ccccc5c5ccc([Si](c6ccccc6)(c6ccccc6)c6c([2H])c([2H])c([2H])c([2H])c6[2H])cc54)ccc32)c([2H])c1[2H]]

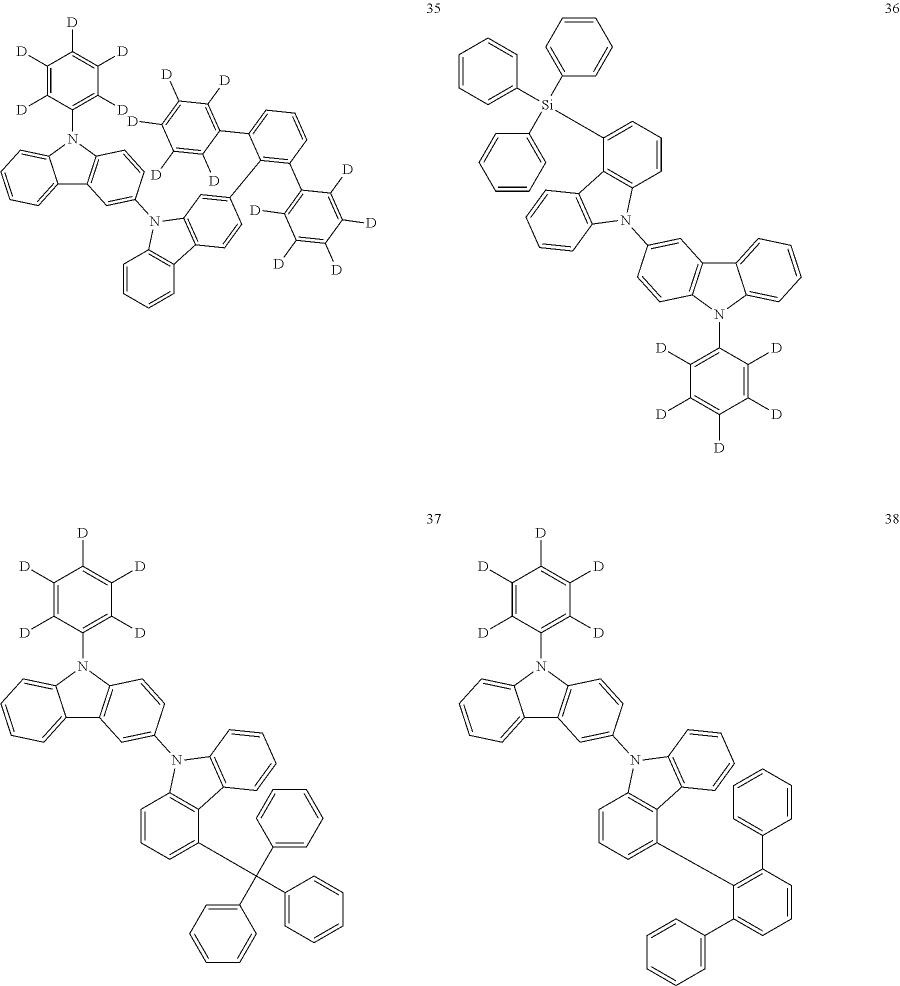

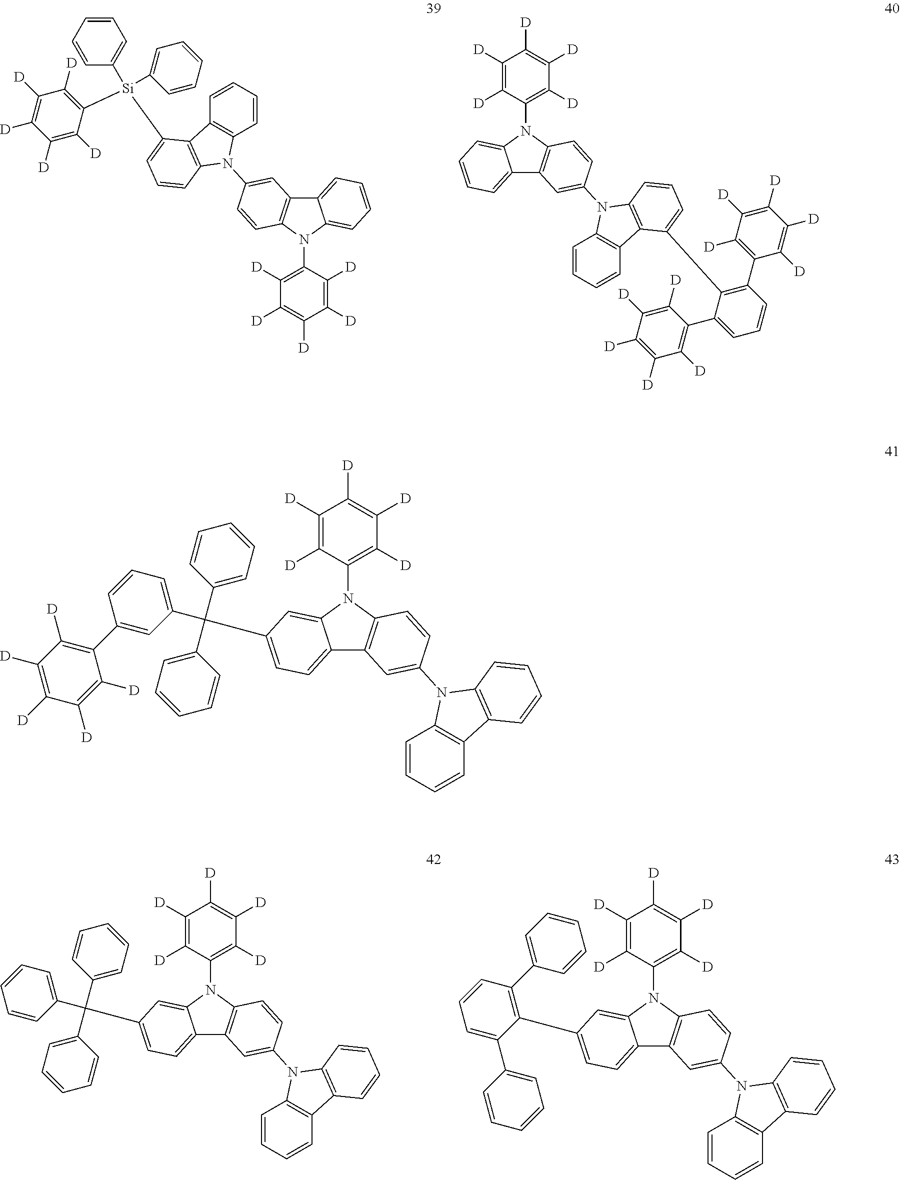

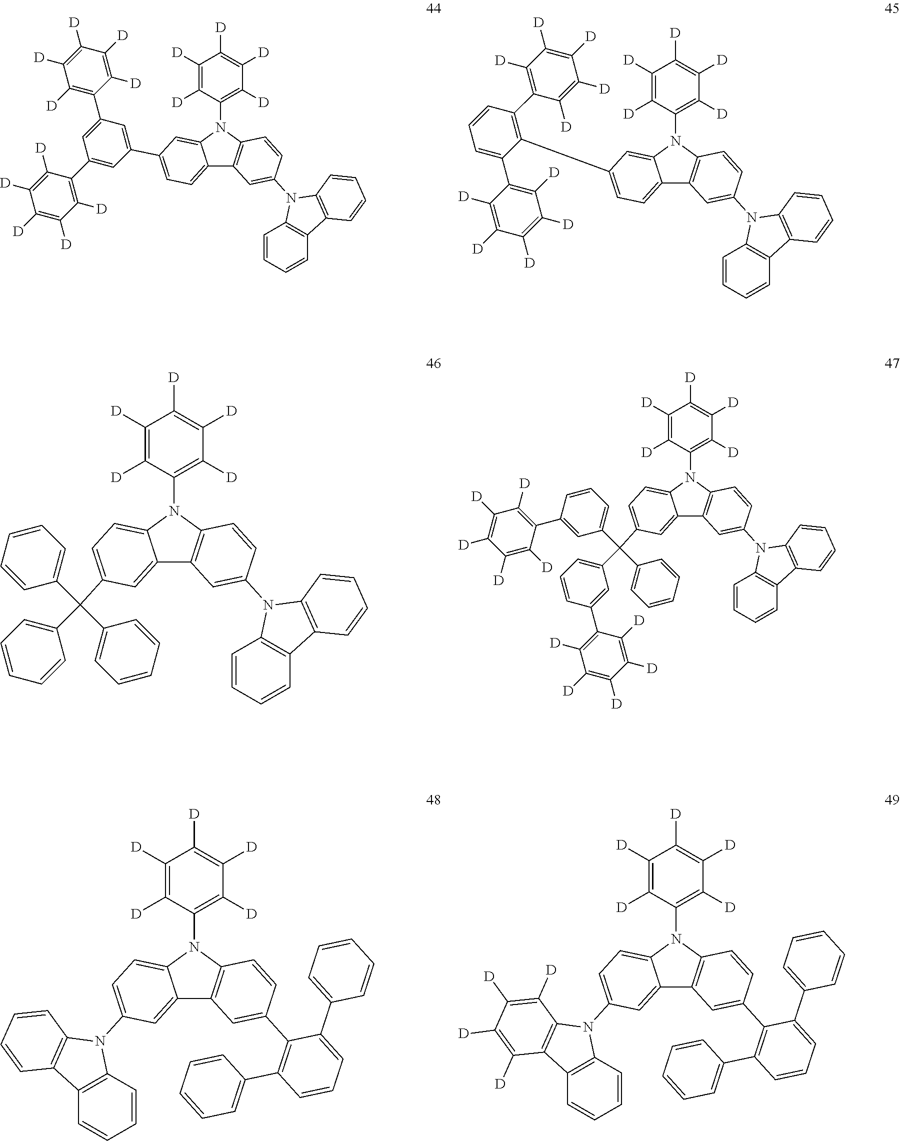

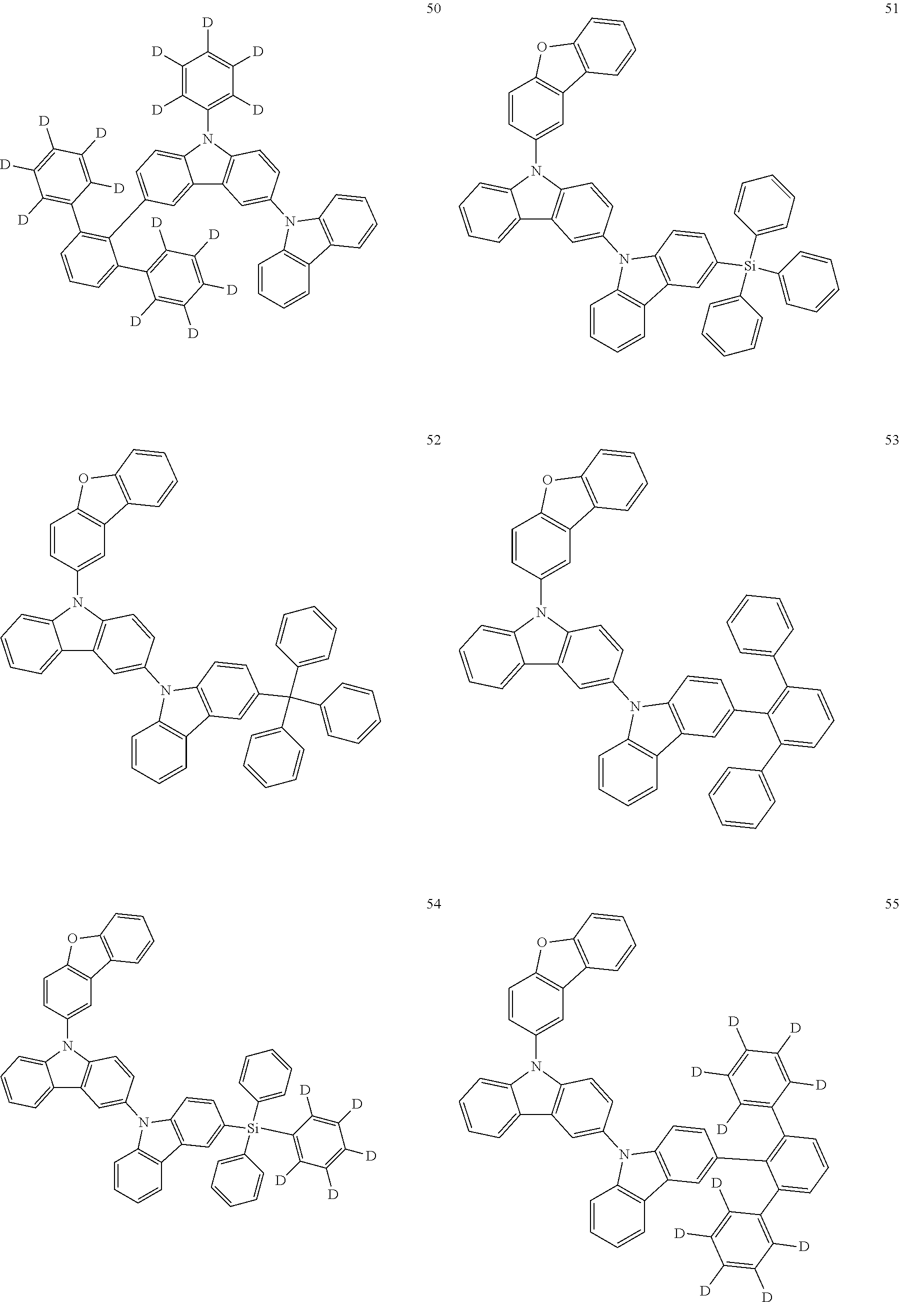

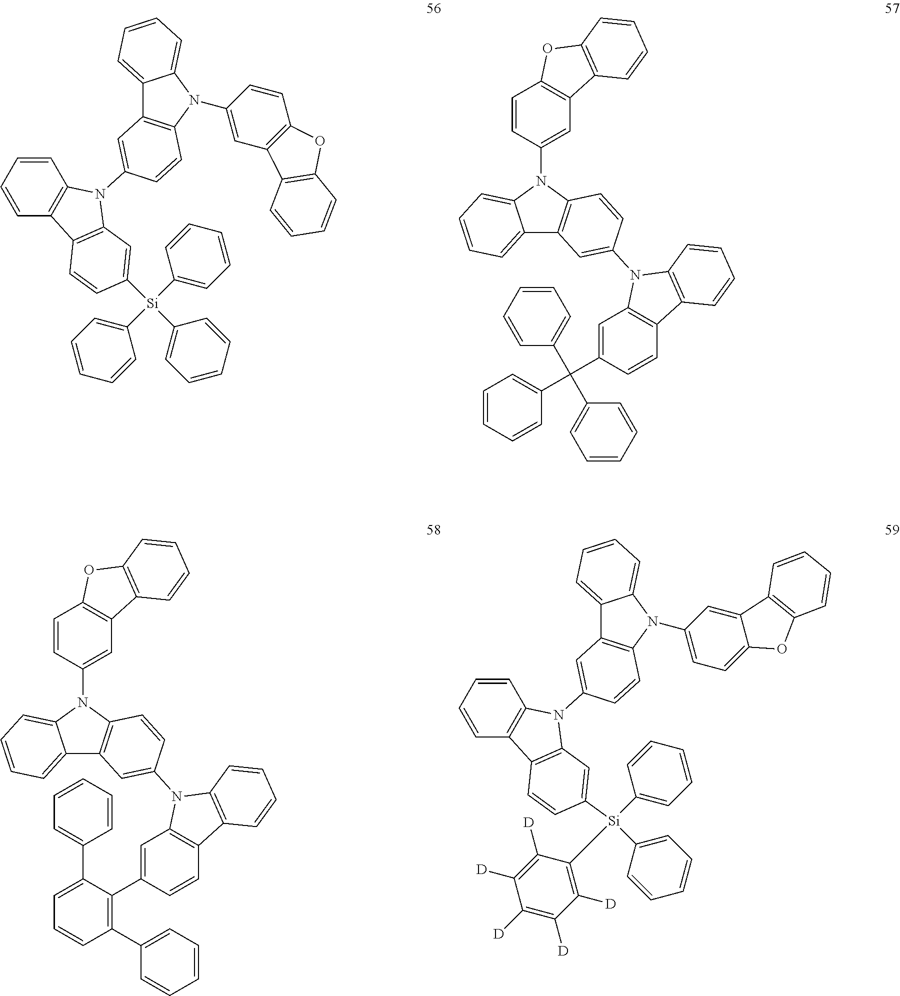 [SMILES: c1ccc([Si](c2ccccc2)(c2ccccc2)c2ccc3c4ccccc4n(-c4ccc5c(c4)c4ccccc4n5-c4ccc5oc6ccccc6c5c4)c3c2)cc1 ; c1ccc(C(c2ccccc2)(c2ccccc2)c2ccc3c4ccccc4n(-c4ccc5c(c4)c4ccccc4n5-c4ccc5oc6ccccc6c5c4)c3c2)cc1 ; c1ccc(-c2cccc(-c3ccccc3)c2-c2ccc3c4ccccc4n(-c4ccc5c(c4)c4ccccc4n5-c4ccc5oc6ccccc6c5c4)c3c2)cc1 ; [2H]c1c([2H])c([2H])c([Si](c2ccccc2)(c2ccccc2)c2ccc3c4ccccc4n(-c4ccc5c(c4)c4ccccc4n5-c4ccc5oc6ccccc6c5c4)c3c2)c([2H])c1[2H]]

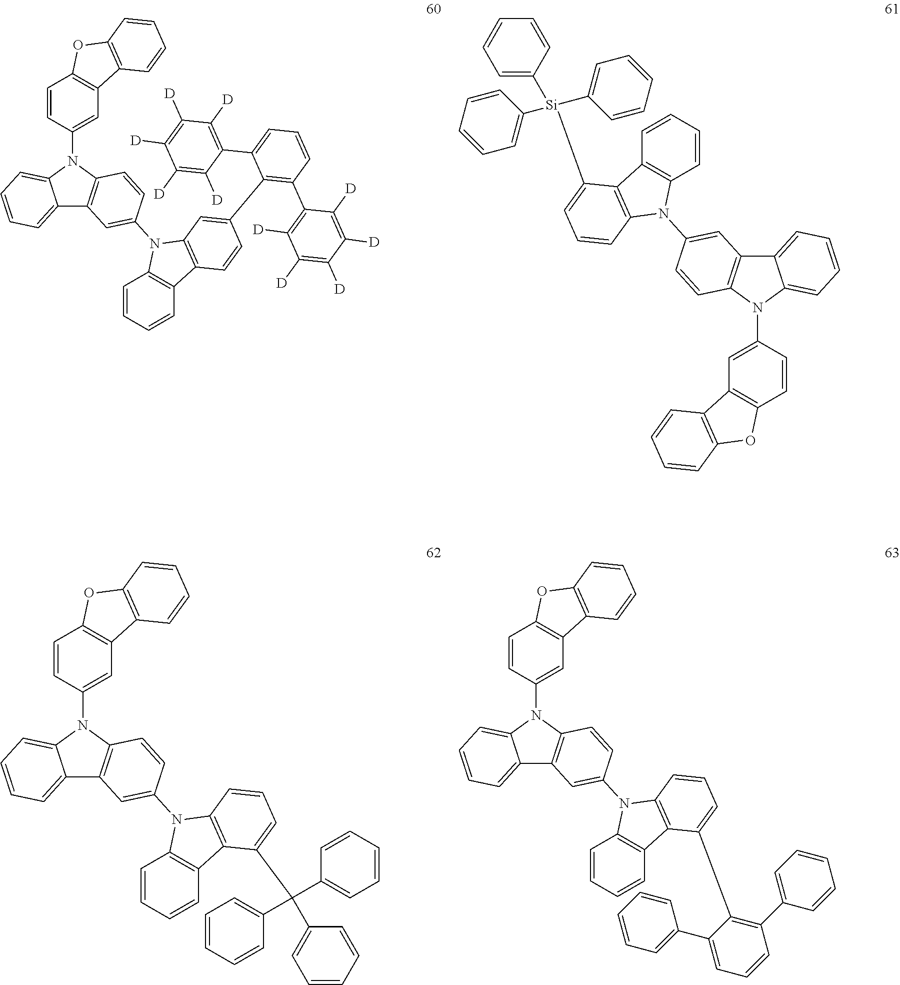 [SMILES: [2H]c1c([2H])c([2H])c(-c2cccc(-c3c([2H])c([2H])c([2H])c([2H])c3[2H])c2-c2ccc3c4ccccc4n(-c4ccc5c(c4)c4ccccc4n5-c4ccc5oc6ccccc6c5c4)c3c2)c([2H])c1[2H] ; c1ccc([Si](c2ccccc2)(c2ccccc2)c2cccc3c2c2ccccc2n3-c2ccc3c(c2)c2ccccc2n3-c2ccc3oc4ccccc4c3c2)cc1 ; c1ccc(C(c2ccccc2)(c2ccccc2)c2cccc3c2c2ccccc2n3-c2ccc3c(c2)c2ccccc2n3-c2ccc3oc4ccccc4c3c2)cc1 ; c1ccc(-c2cccc(-c3ccccc3)c2-c2cccc3c2c2ccccc2n3-c2ccc3c(c2)c2ccccc2n3-c2ccc3oc4ccccc4c3c2)cc1]

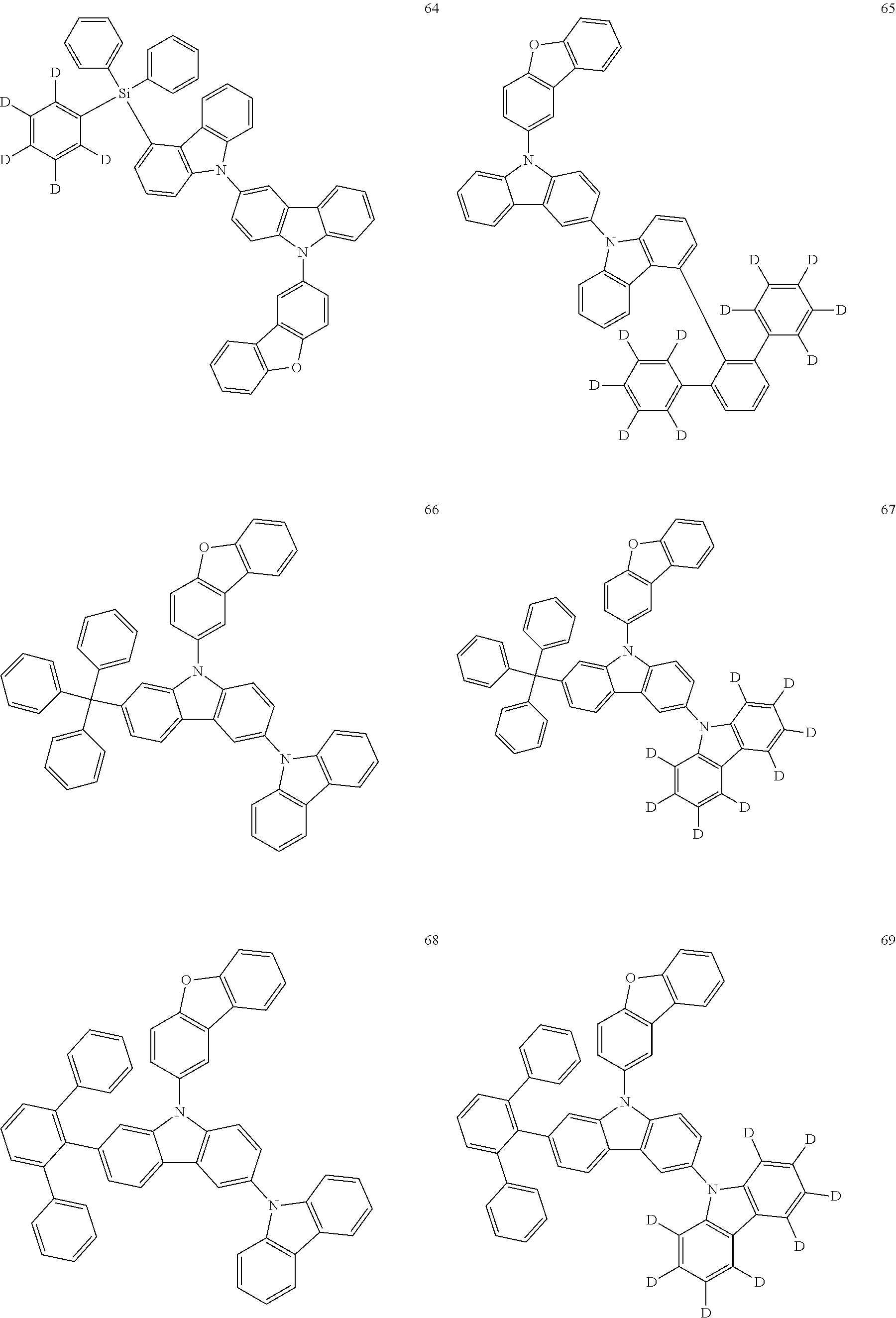

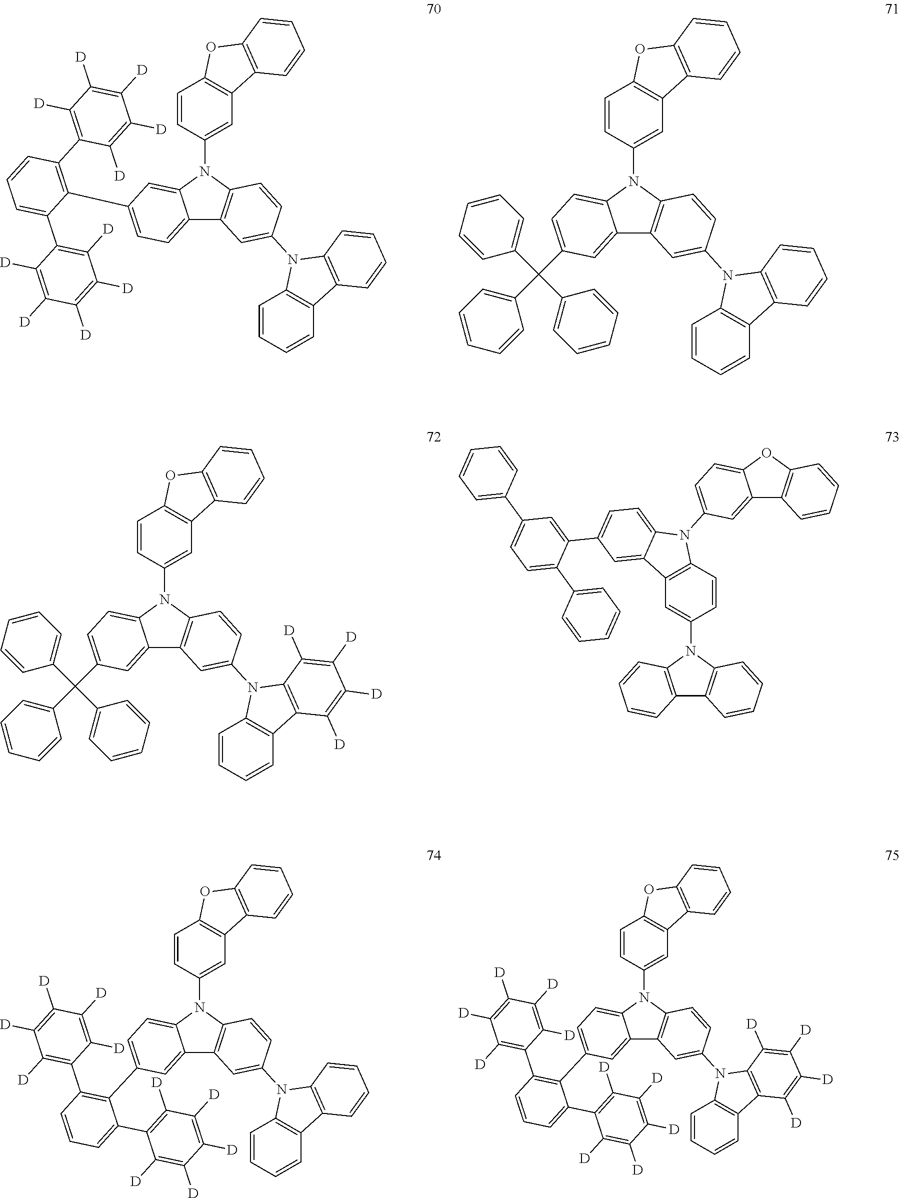

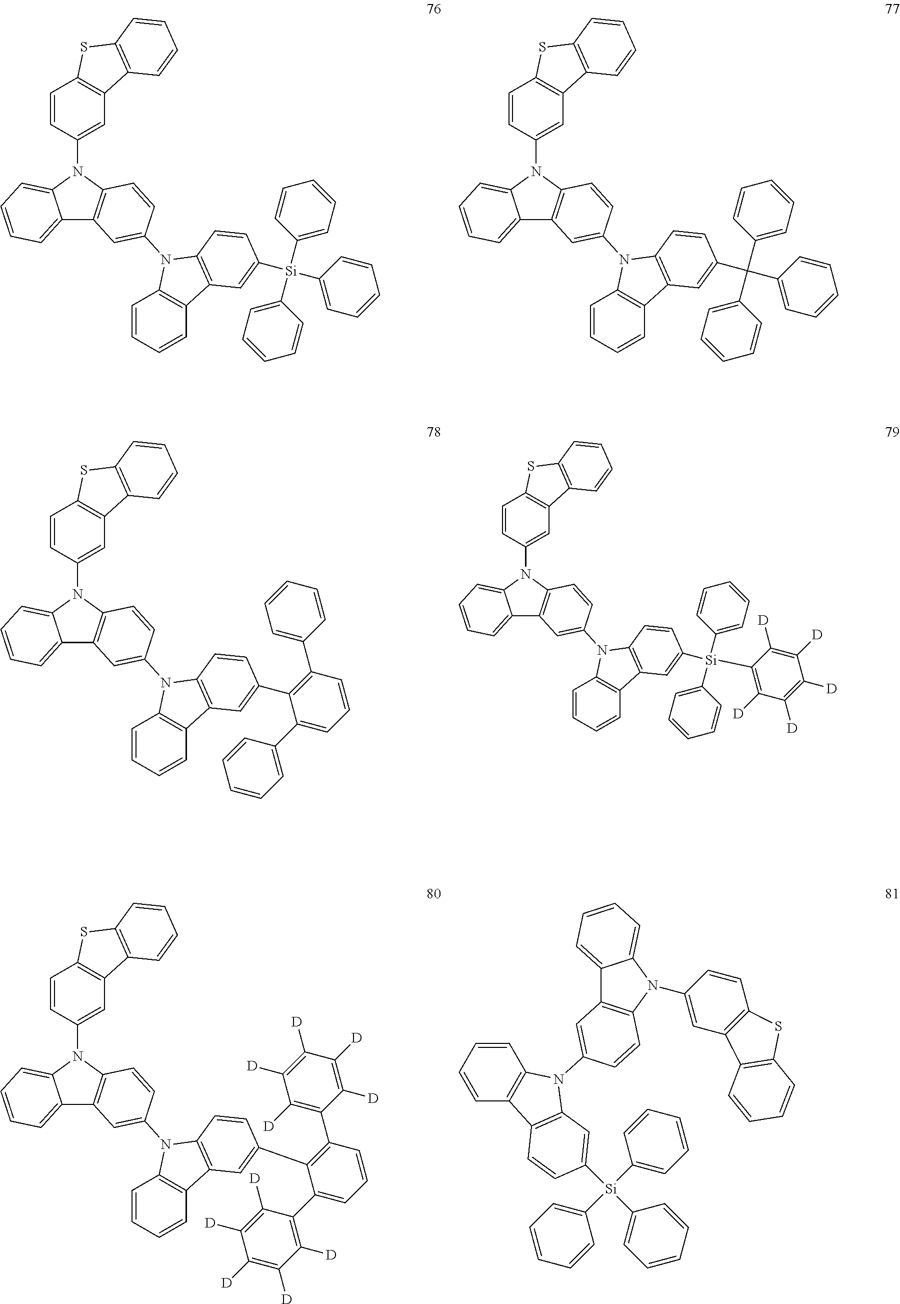

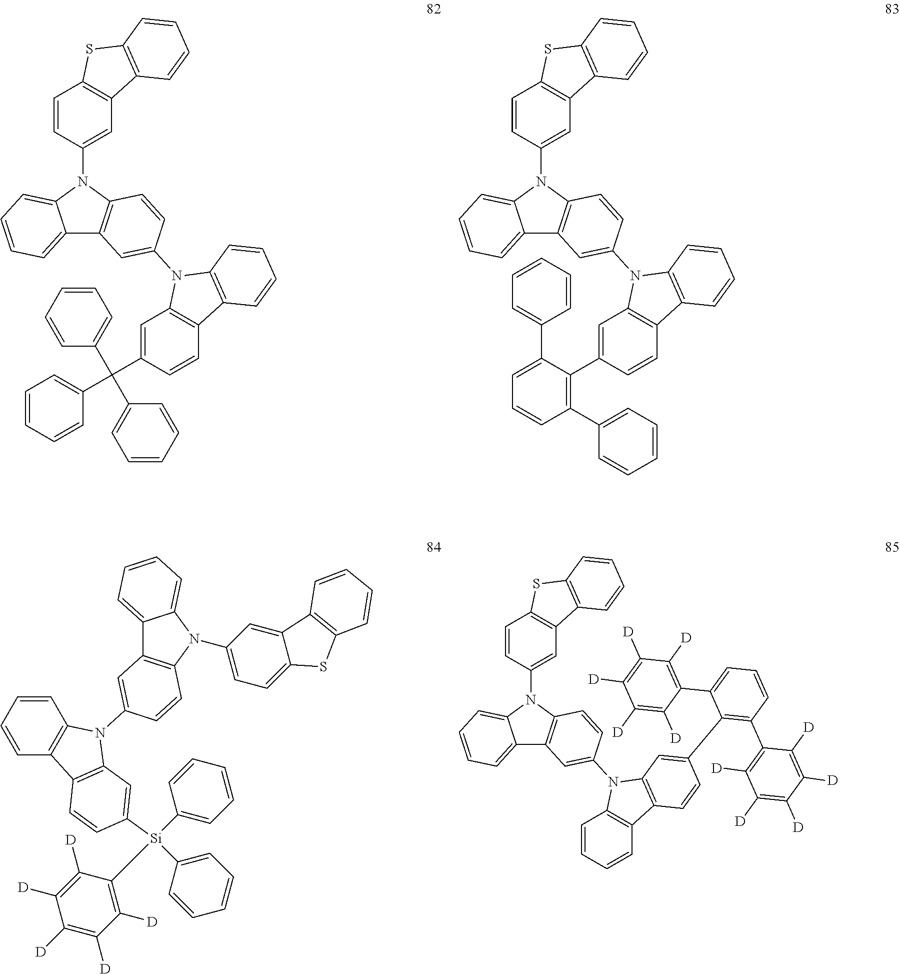 [SMILES: c1ccc(C(c2ccccc2)(c2ccccc2)c2ccc3c4ccccc4n(-c4ccc5c(c4)c4ccccc4n5-c4ccc5sc6ccccc6c5c4)c3c2)cc1 ; c1ccc(-c2cccc(-c3ccccc3)c2-c2ccc3c4ccccc4n(-c4ccc5c(c4)c4ccccc4n5-c4ccc5sc6ccccc6c5c4)c3c2)cc1 ; [2H]c1c([2H])c([2H])c([Si](c2ccccc2)(c2ccccc2)c2ccc3c4ccccc4n(-c4ccc5c(c4)c4ccccc4n5-c4ccc5sc6ccccc6c5c4)c3c2)c([2H])c1[2H] ; [2H]c1c([2H])c([2H])c(-c2cccc(-c3c([2H])c([2H])c([2H])c([2H])c3[2H])c2-c2ccc3c4ccccc4n(-c4ccc5c(c4)c4ccccc4n5-c4ccc5sc6ccccc6c5c4)c3c2)c([2H])c1[2H]]

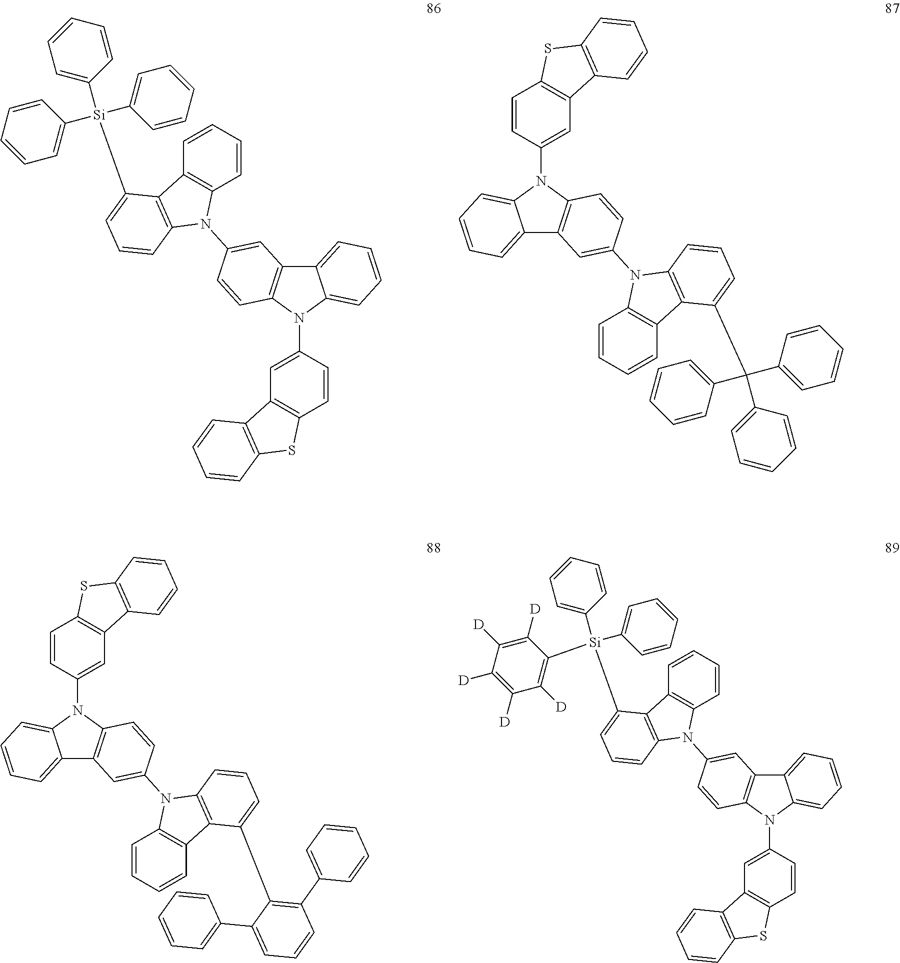 [SMILES: c1ccc([Si](c2ccccc2)(c2ccccc2)c2cccc3c2c2ccccc2n3-c2ccc3c(c2)c2ccccc2n3-c2ccc3sc4ccccc4c3c2)cc1 ; c1ccc(C(c2ccccc2)(c2ccccc2)c2cccc3c2c2ccccc2n3-c2ccc3c(c2)c2ccccc2n3-c2ccc3sc4ccccc4c3c2)cc1 ; c1ccc(-c2cccc(-c3ccccc3)c2-c2cccc3c2c2ccccc2n3-c2ccc3c(c2)c2ccccc2n3-c2ccc3sc4ccccc4c3c2)cc1 ; [2H]c1c([2H])c([2H])c([Si](c2ccccc2)(c2ccccc2)c2cccc3c2c2ccccc2n3-c2ccc3c(c2)c2ccccc2n3-c2ccc3sc4ccccc4c3c2)c([2H])c1[2H]]

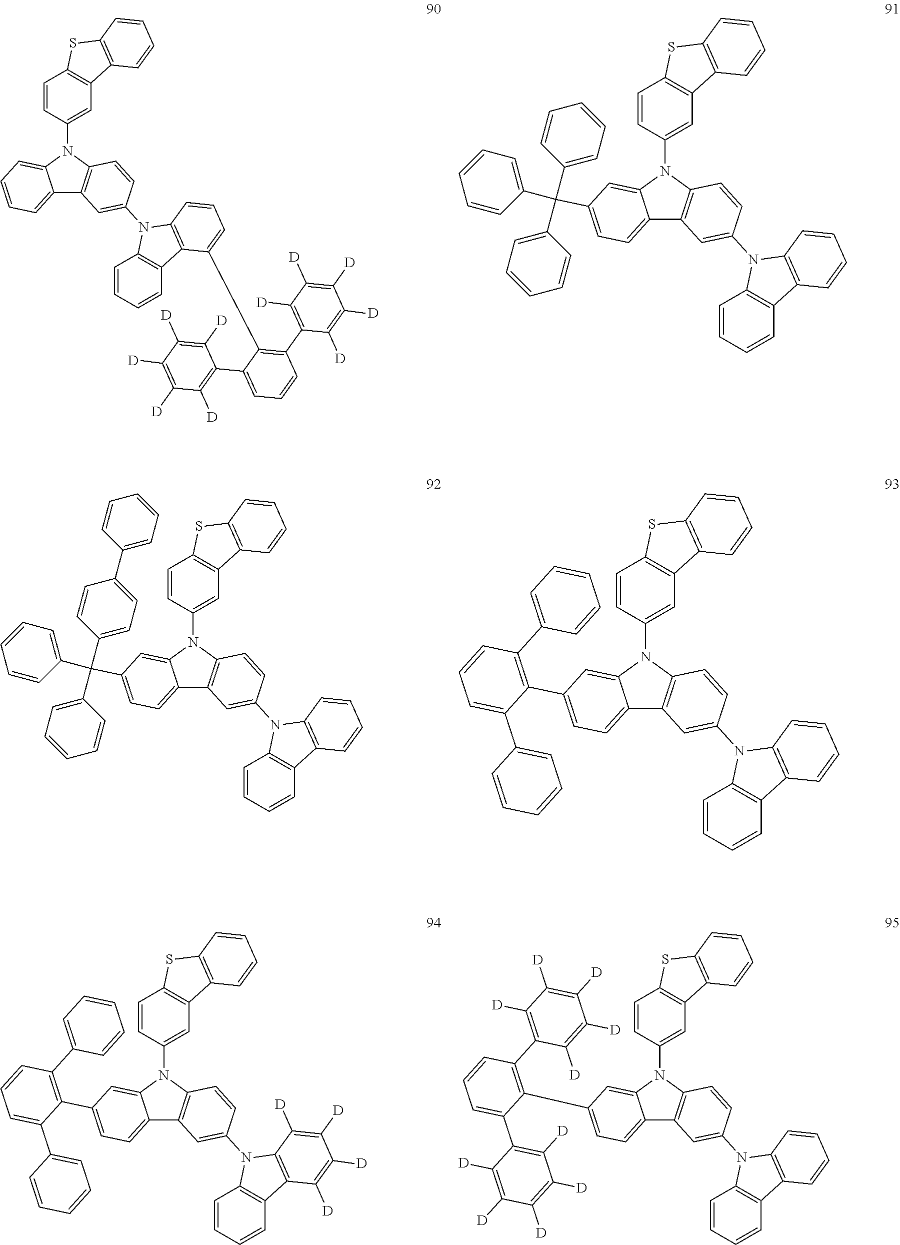

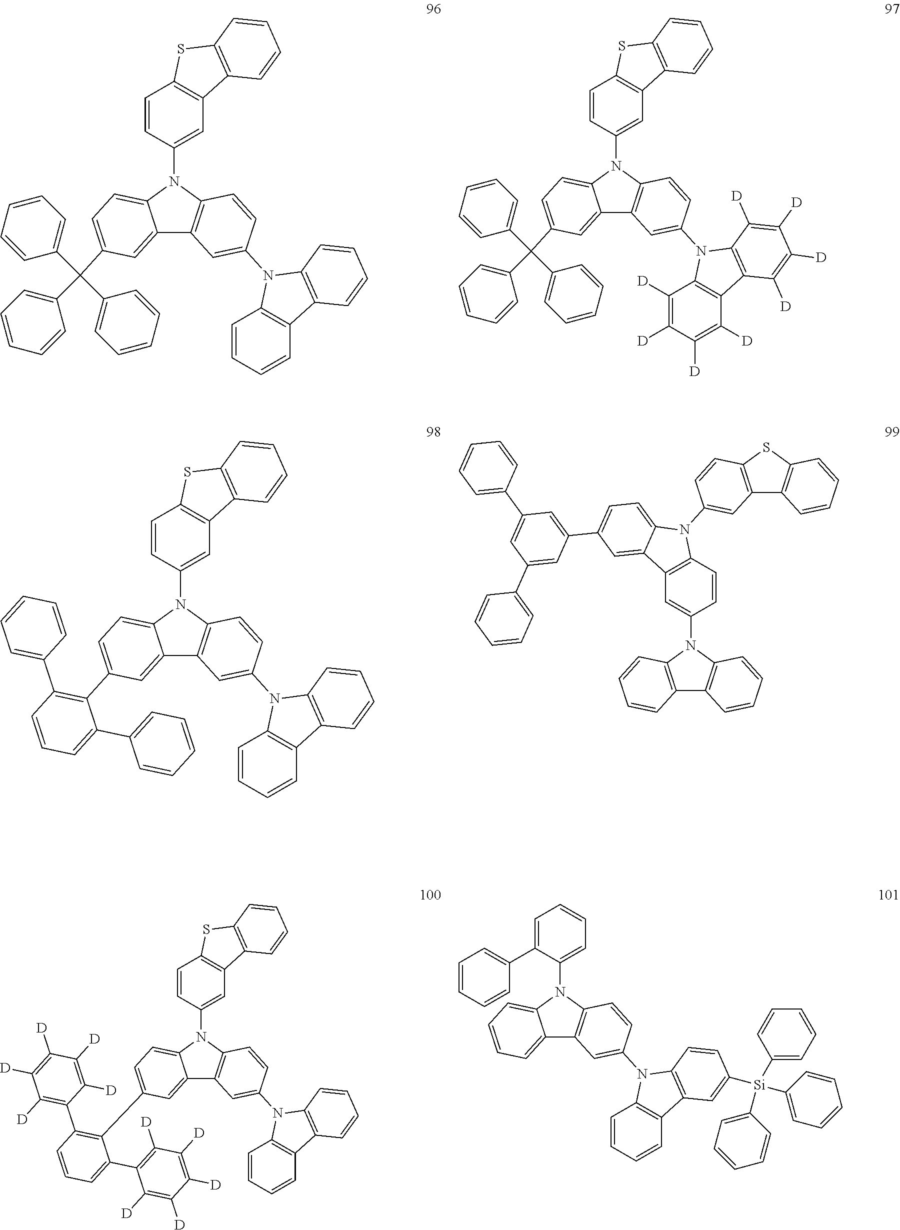

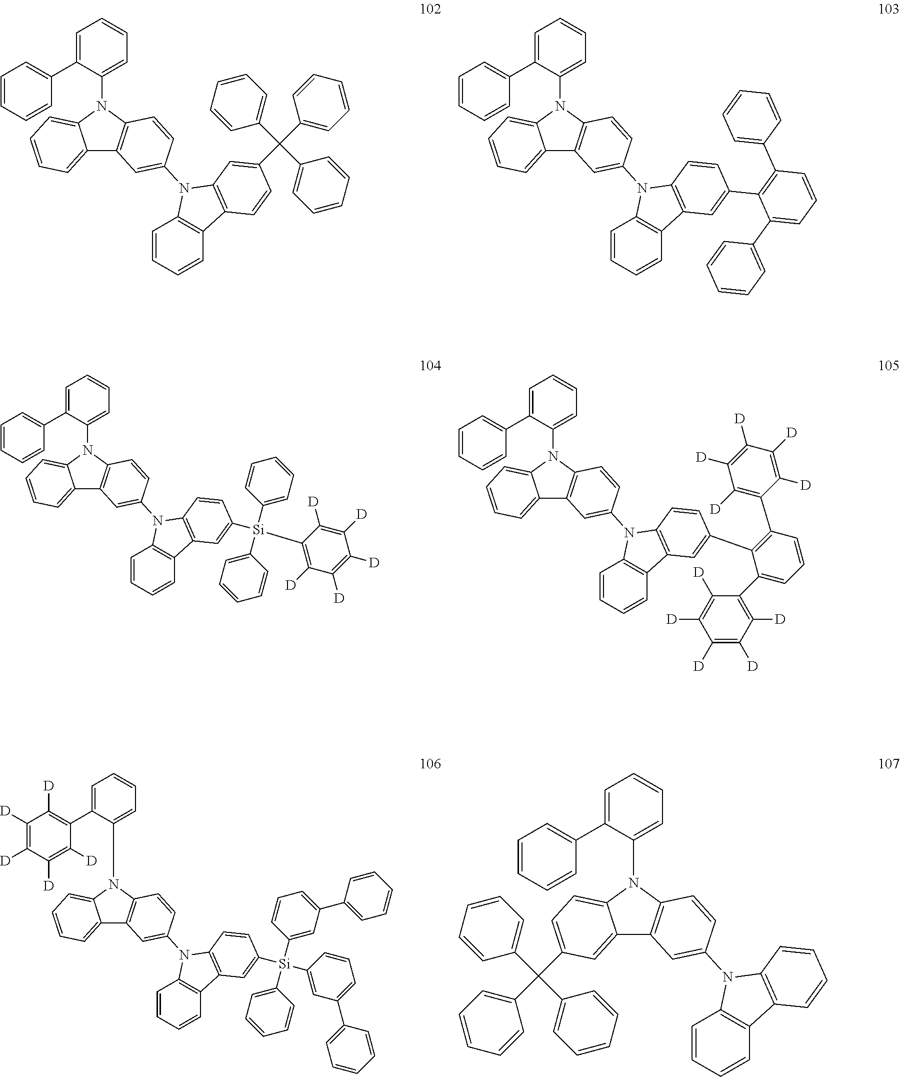

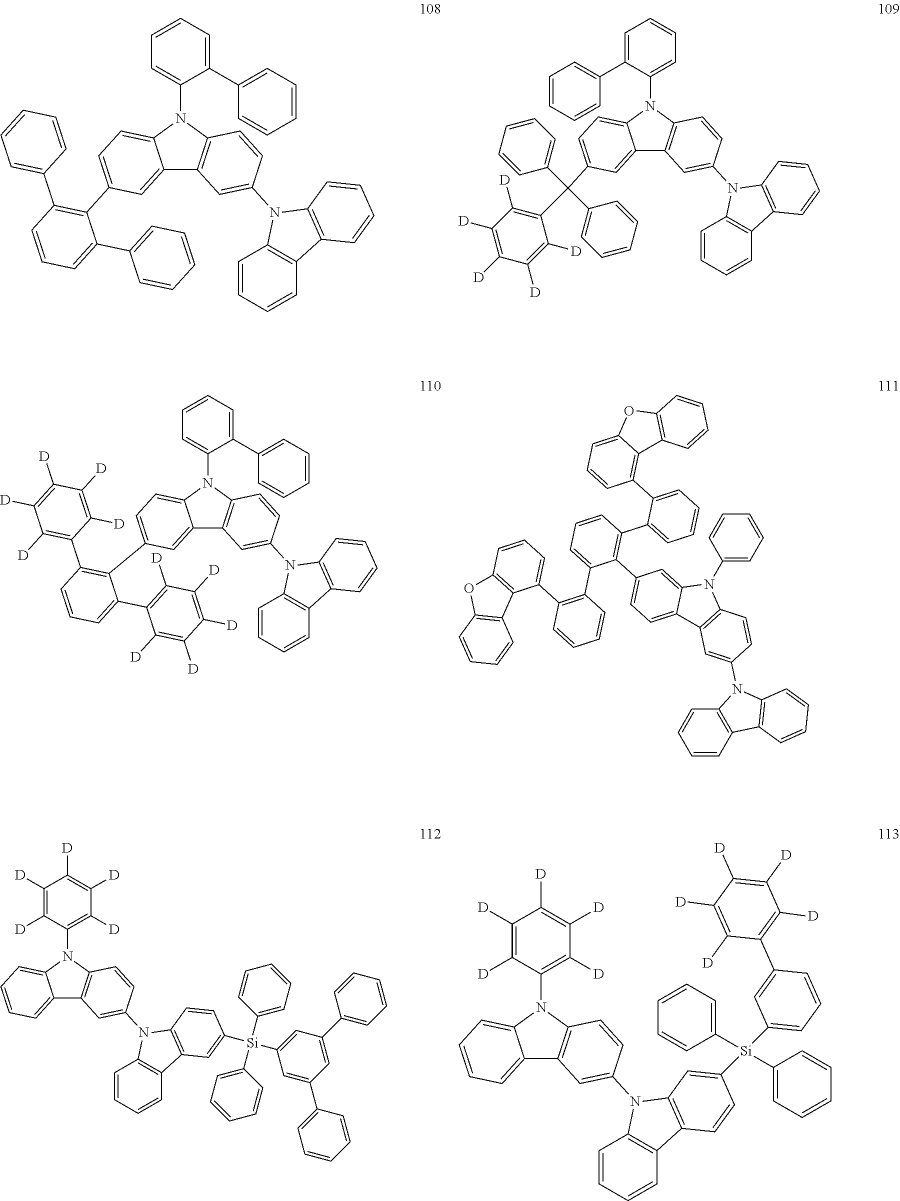

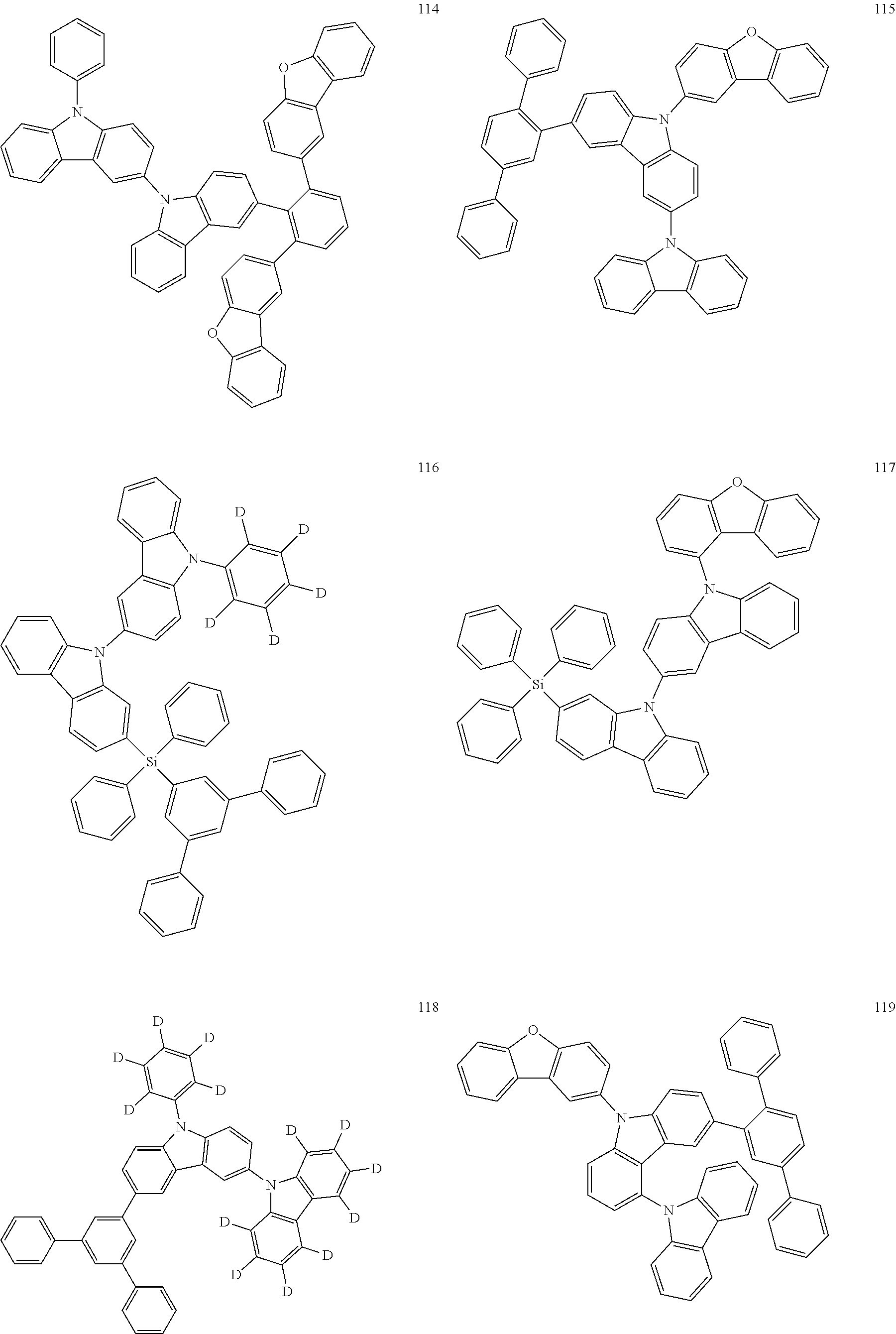

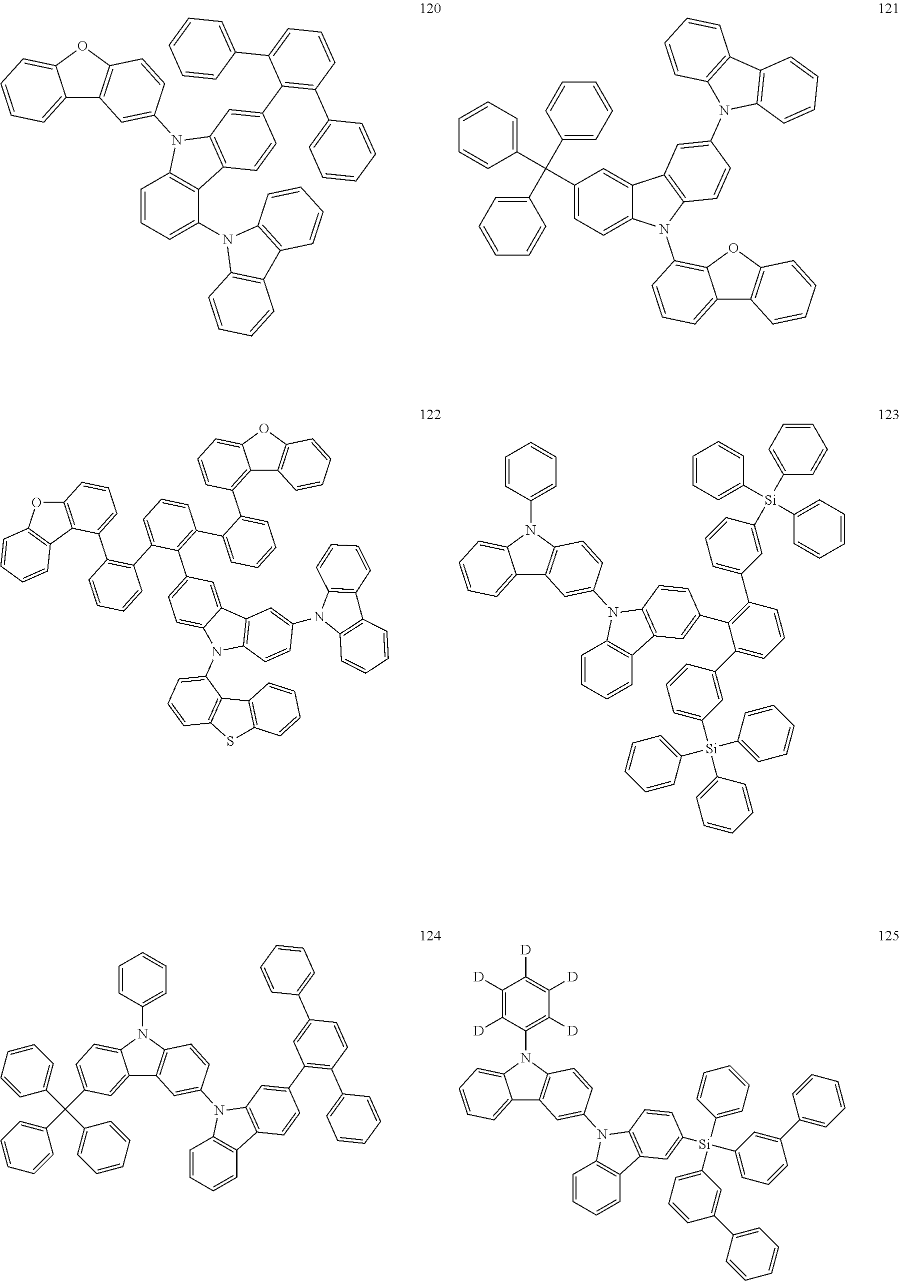

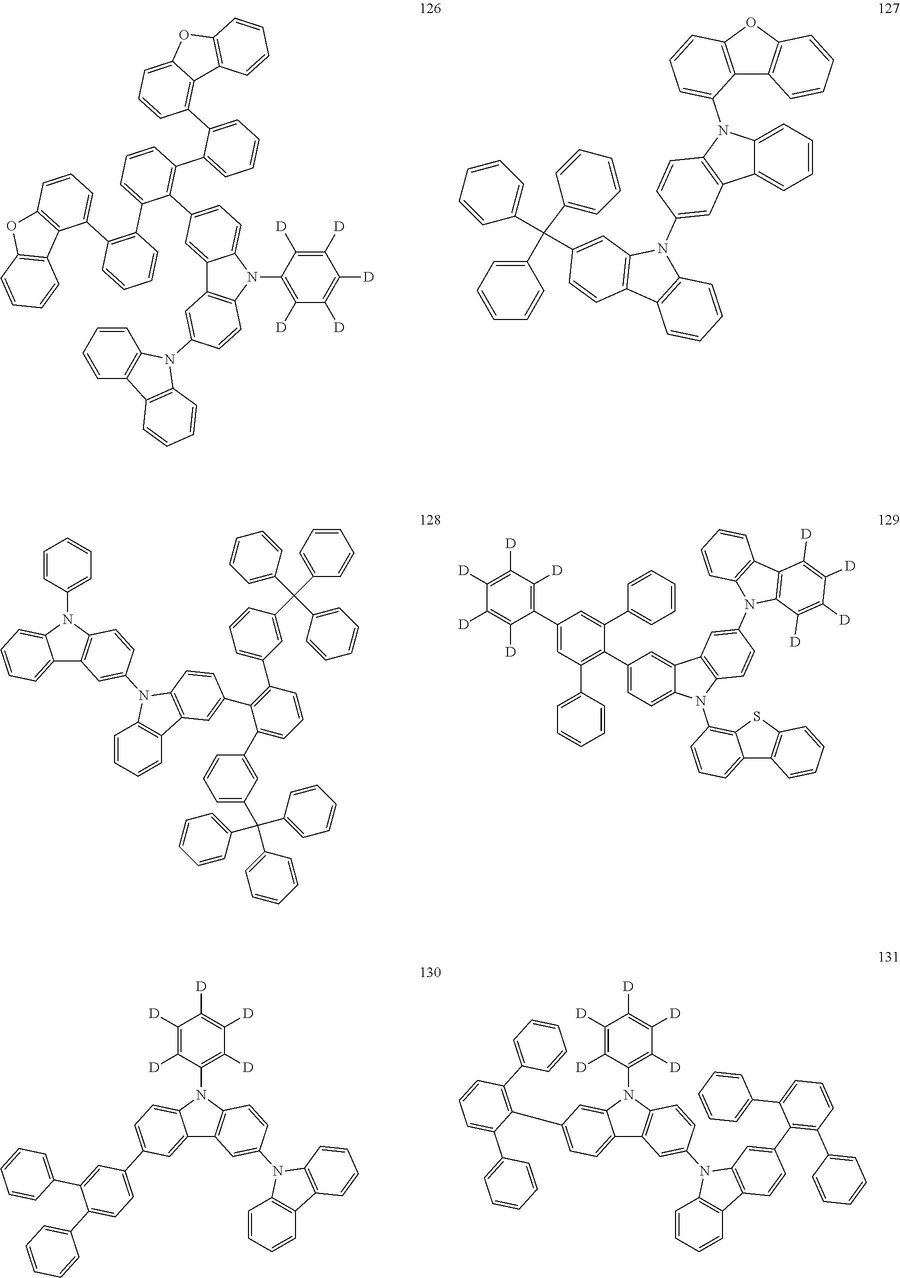

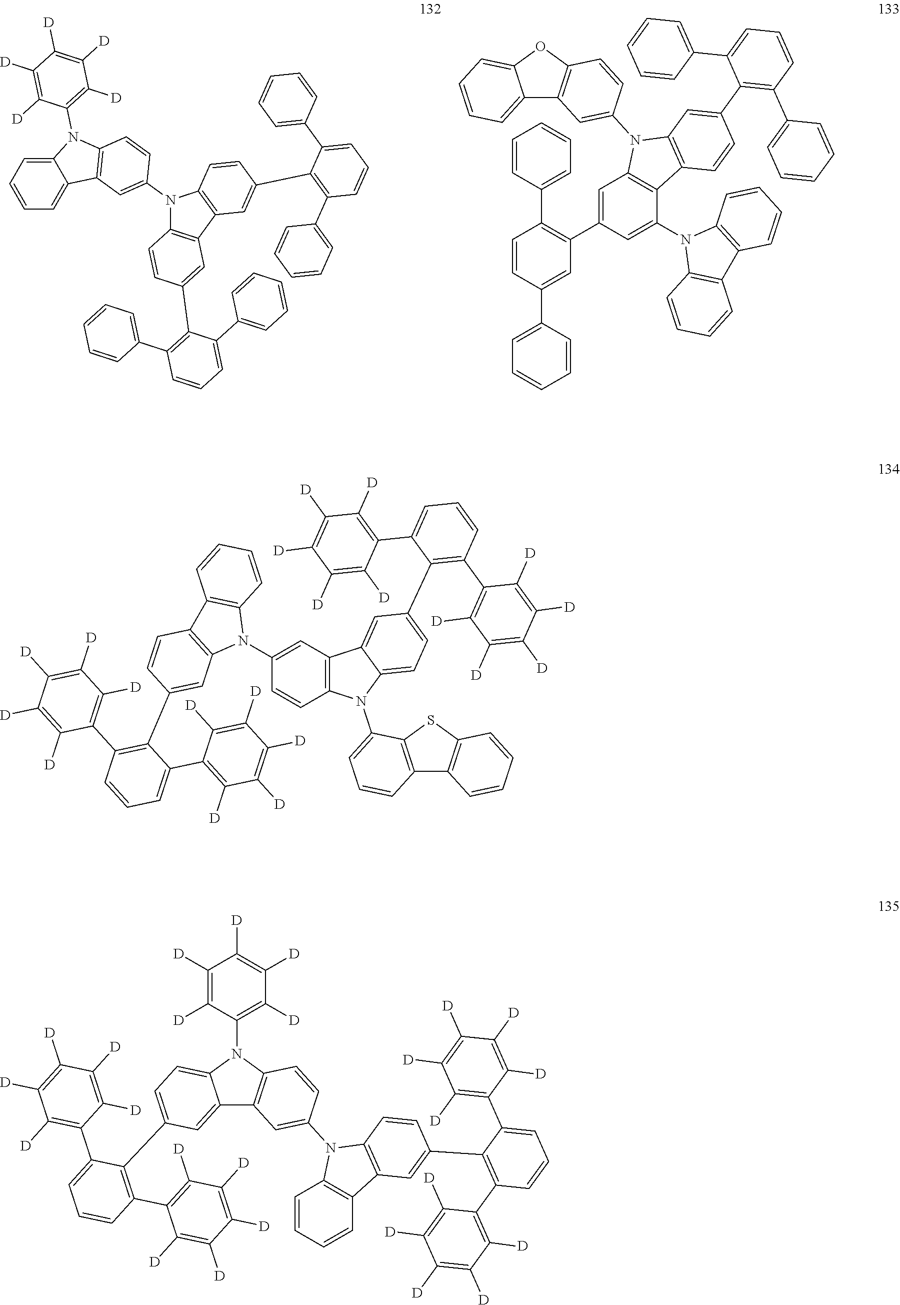

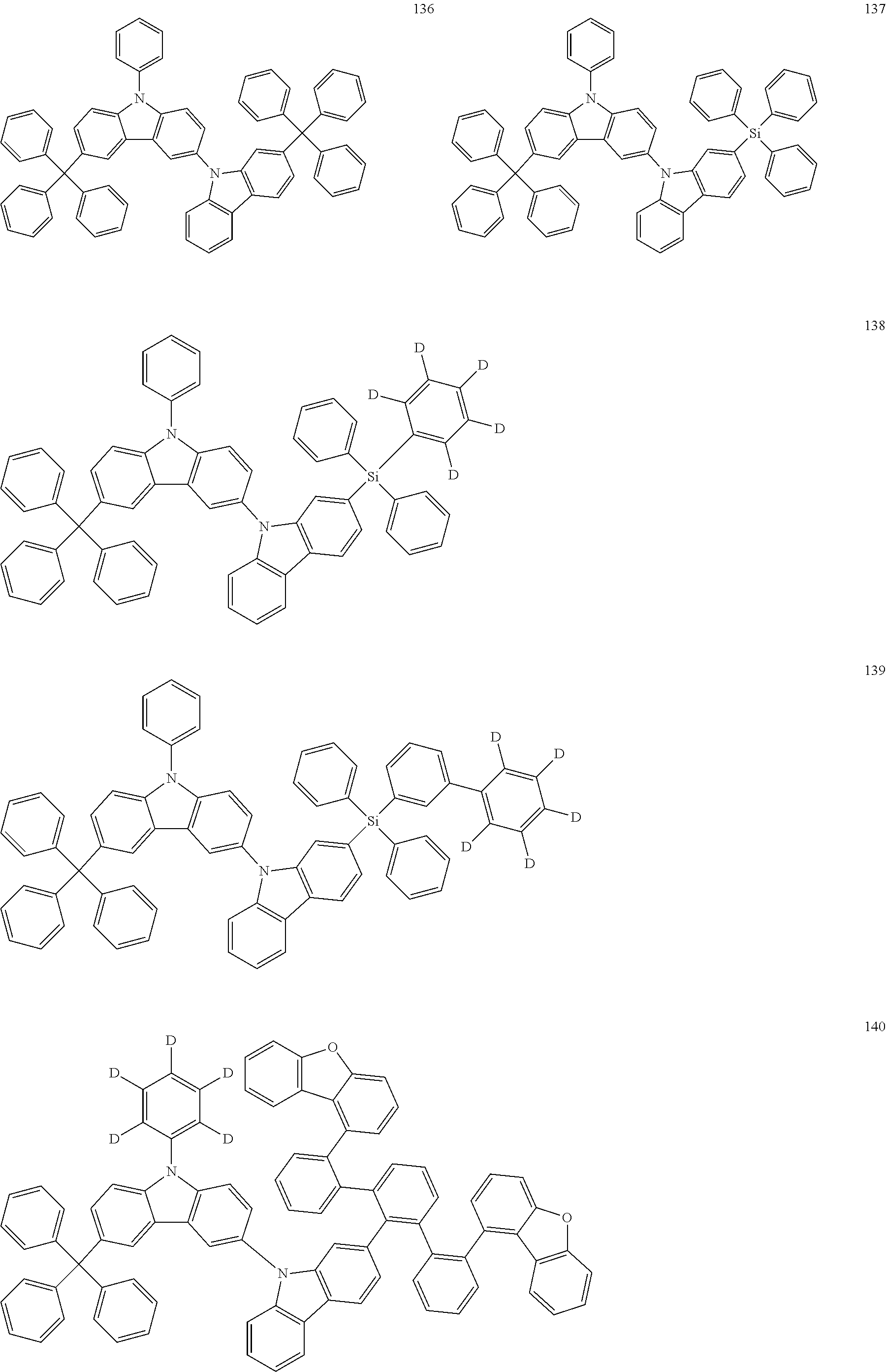

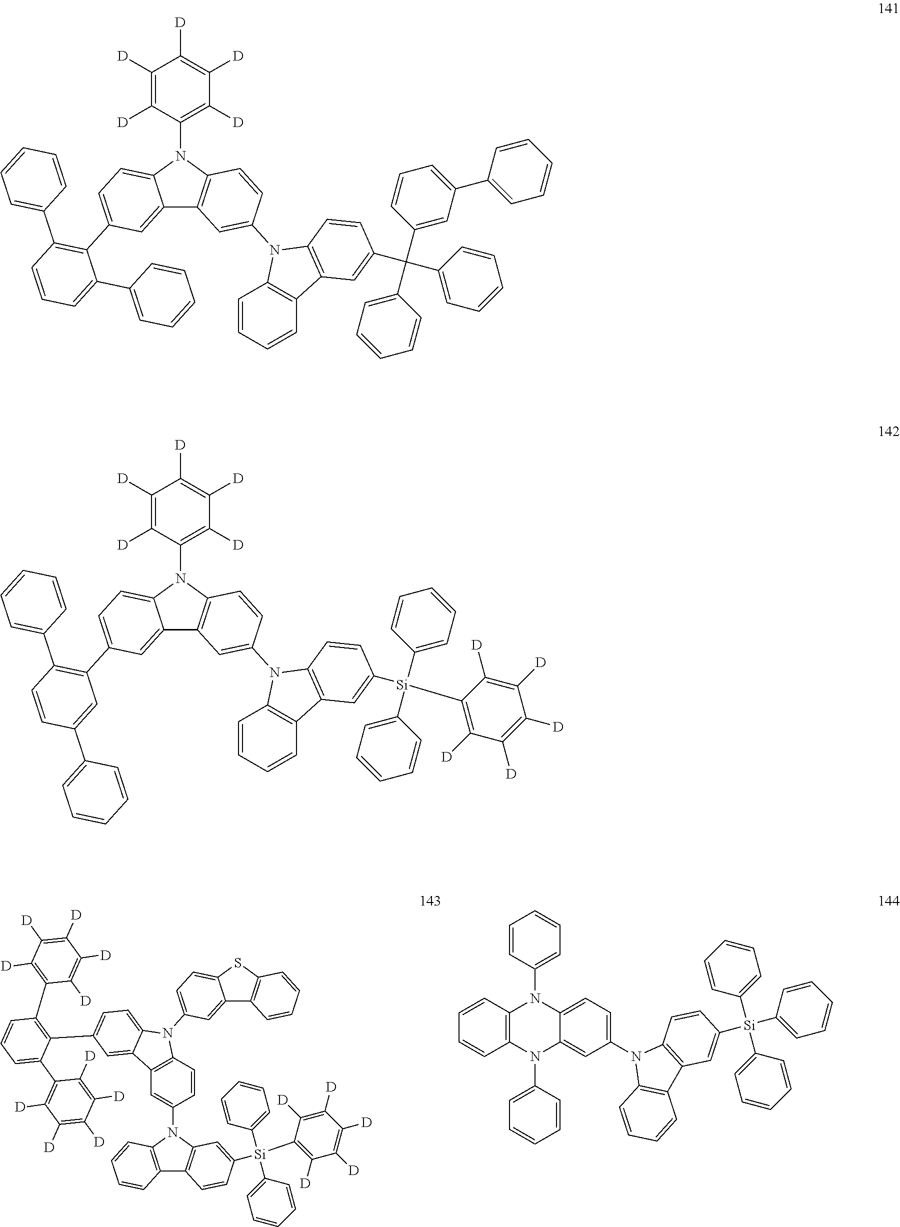

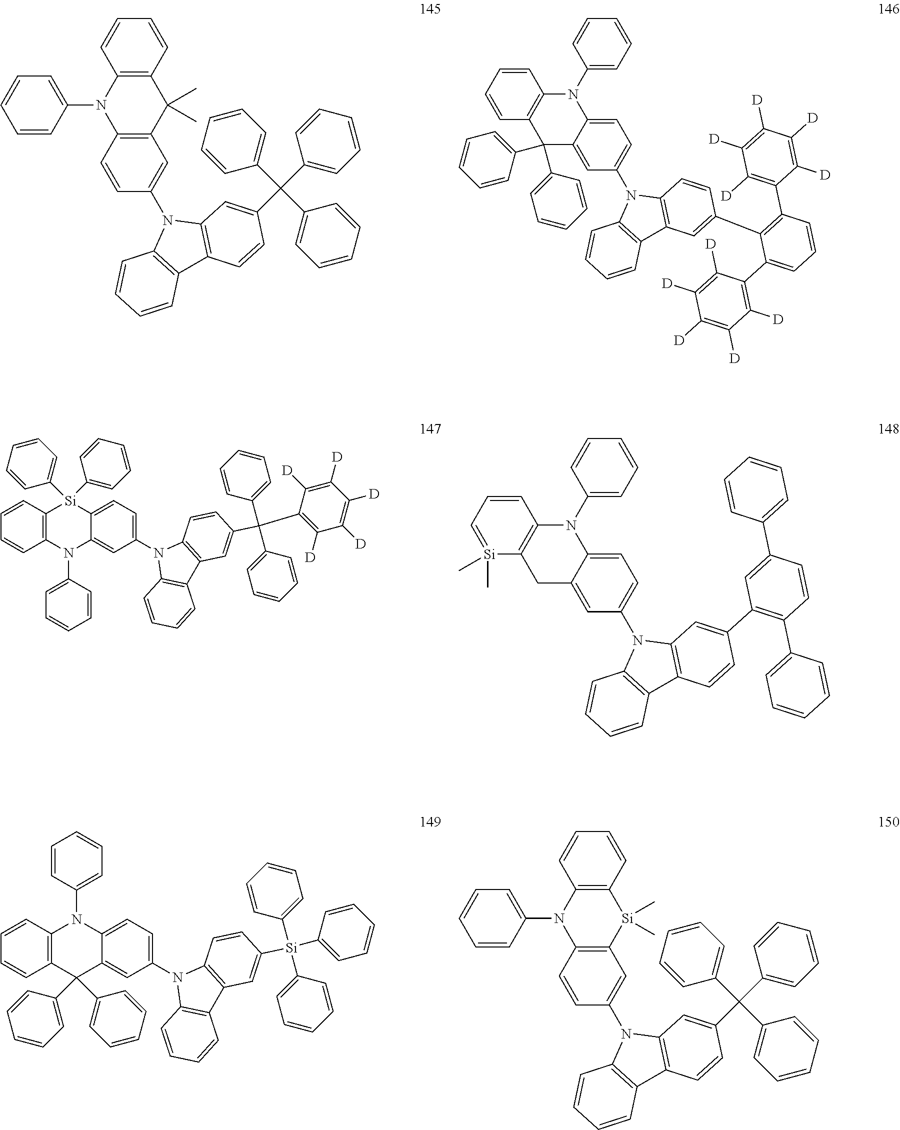

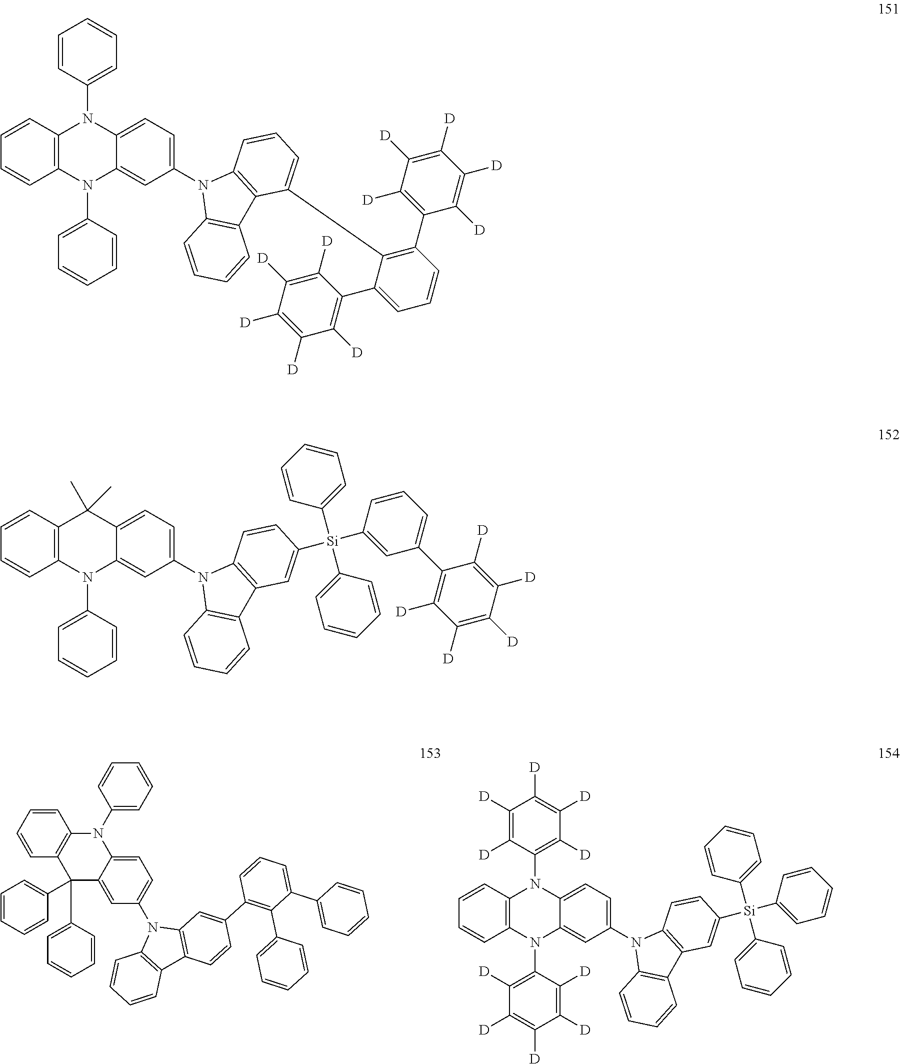

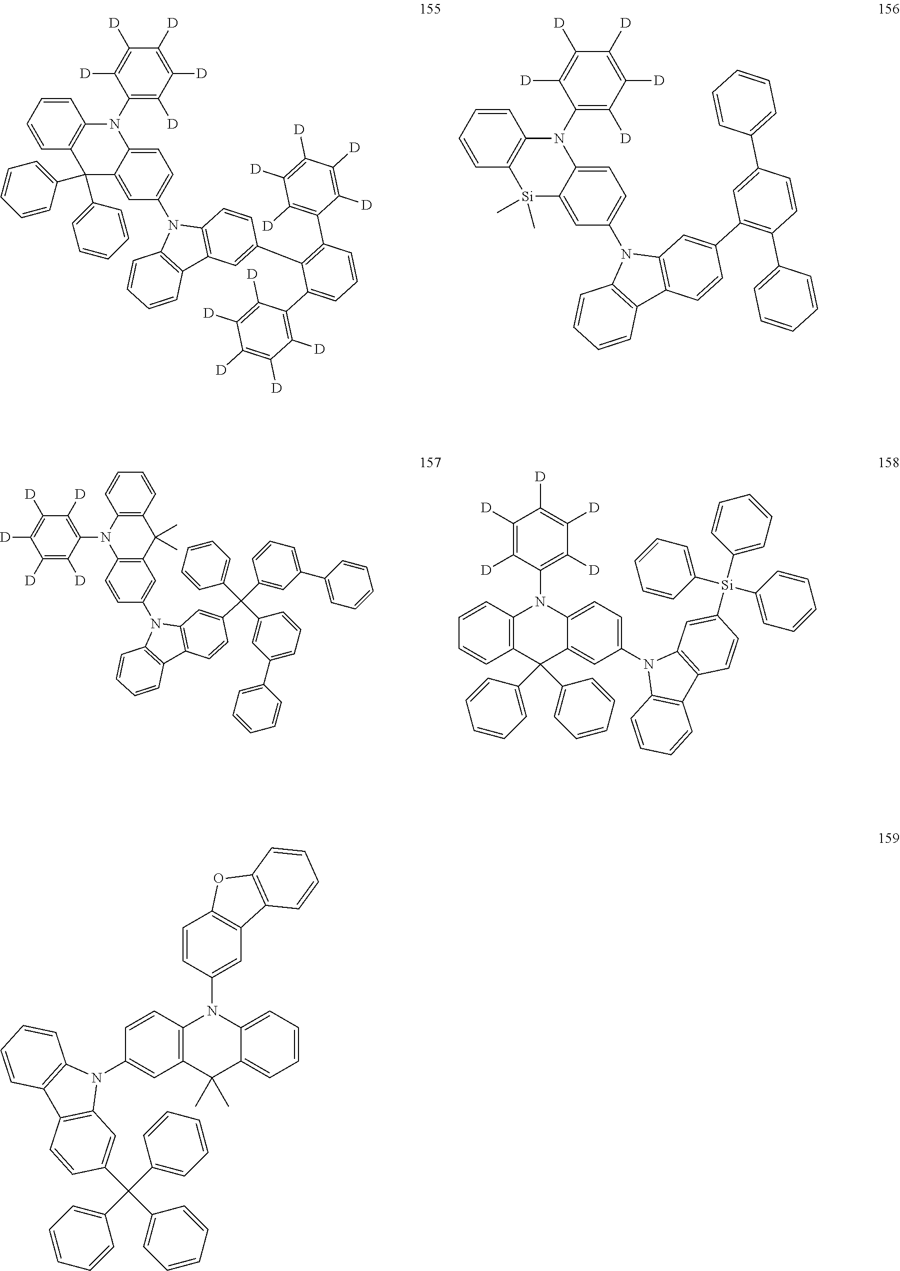

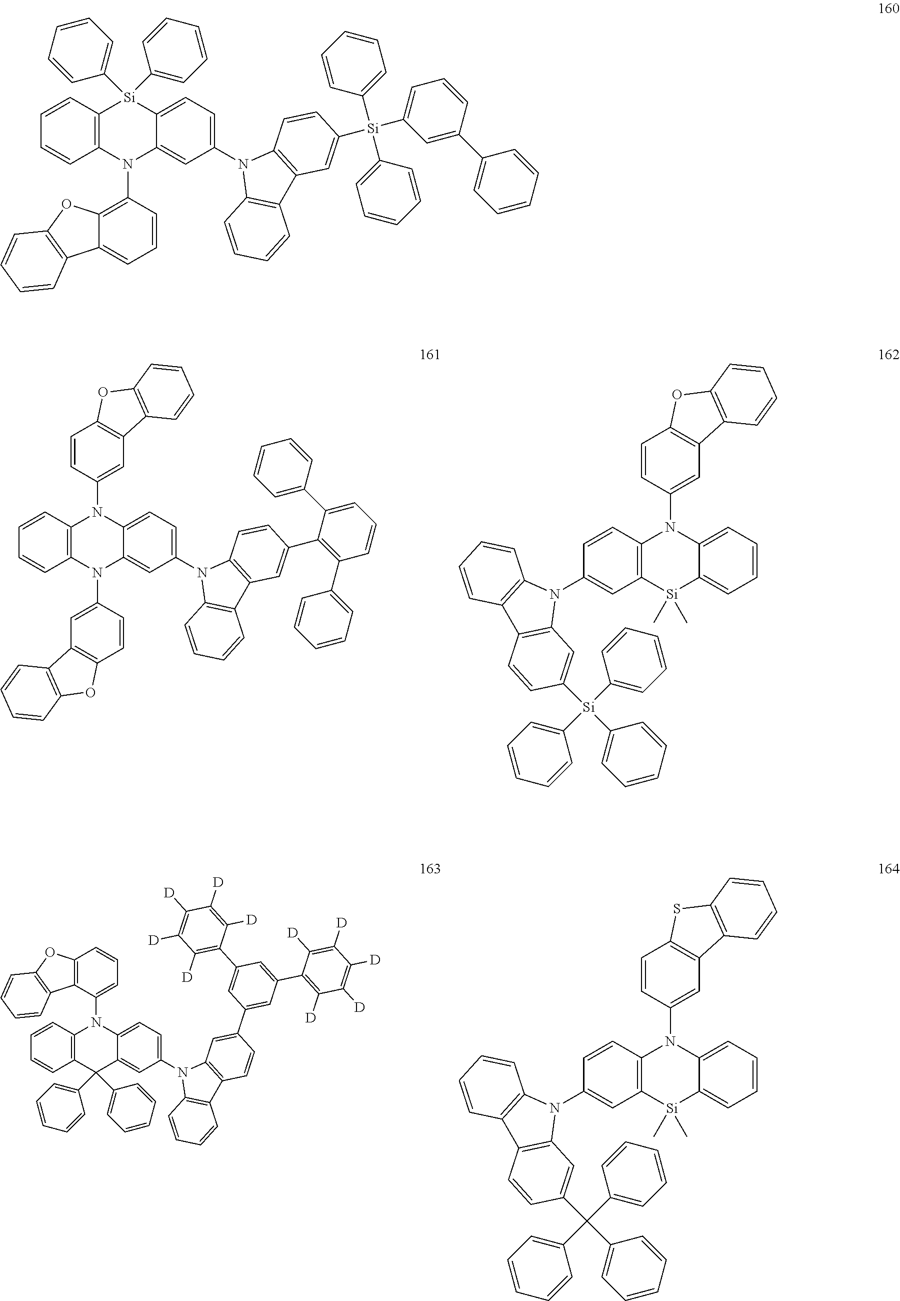

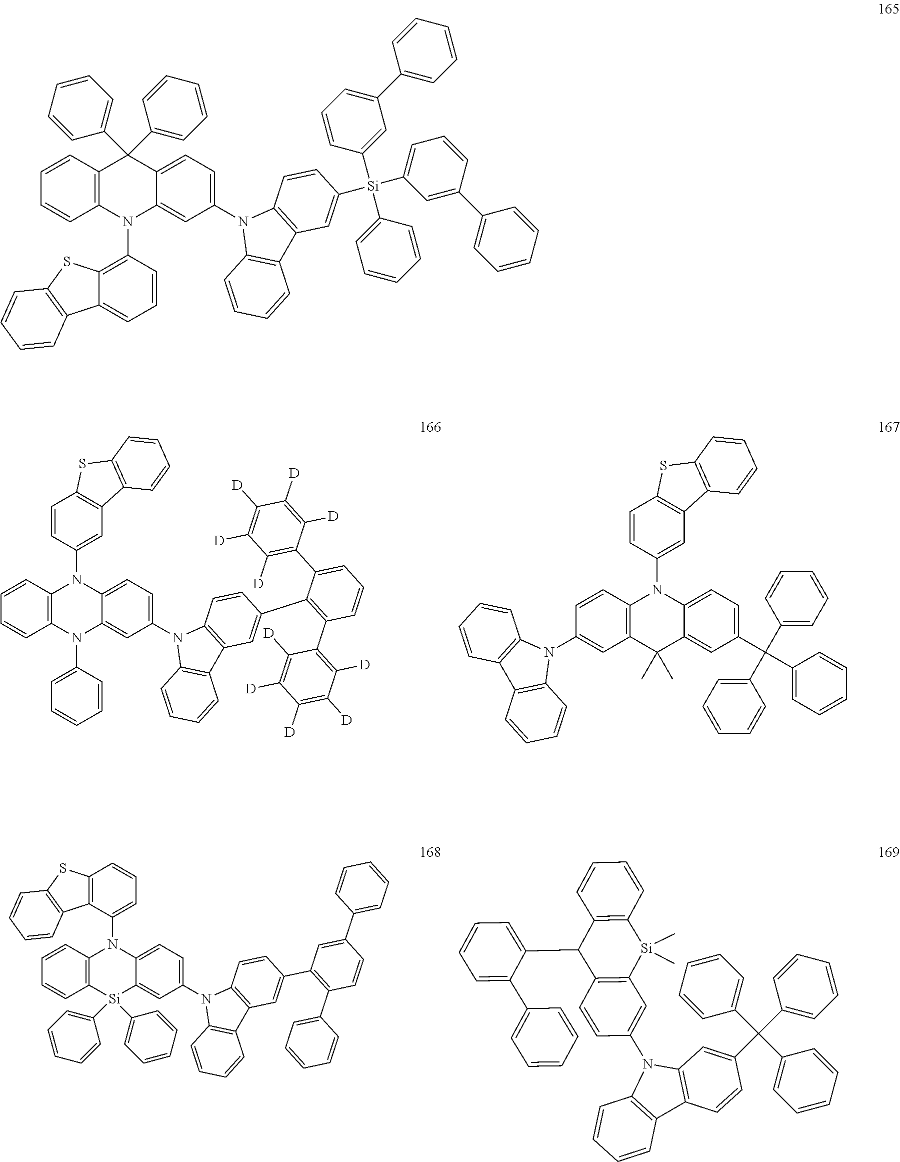

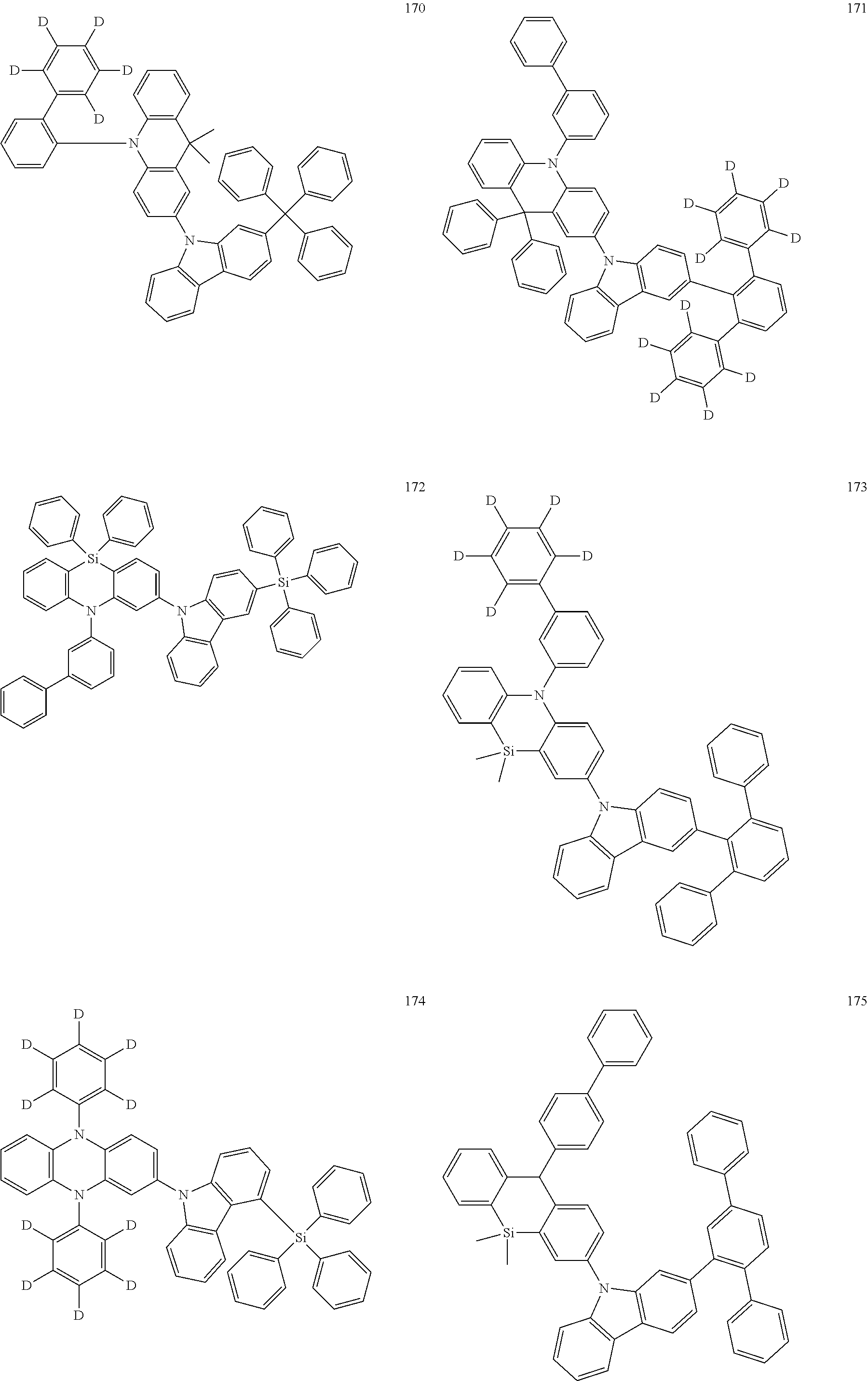

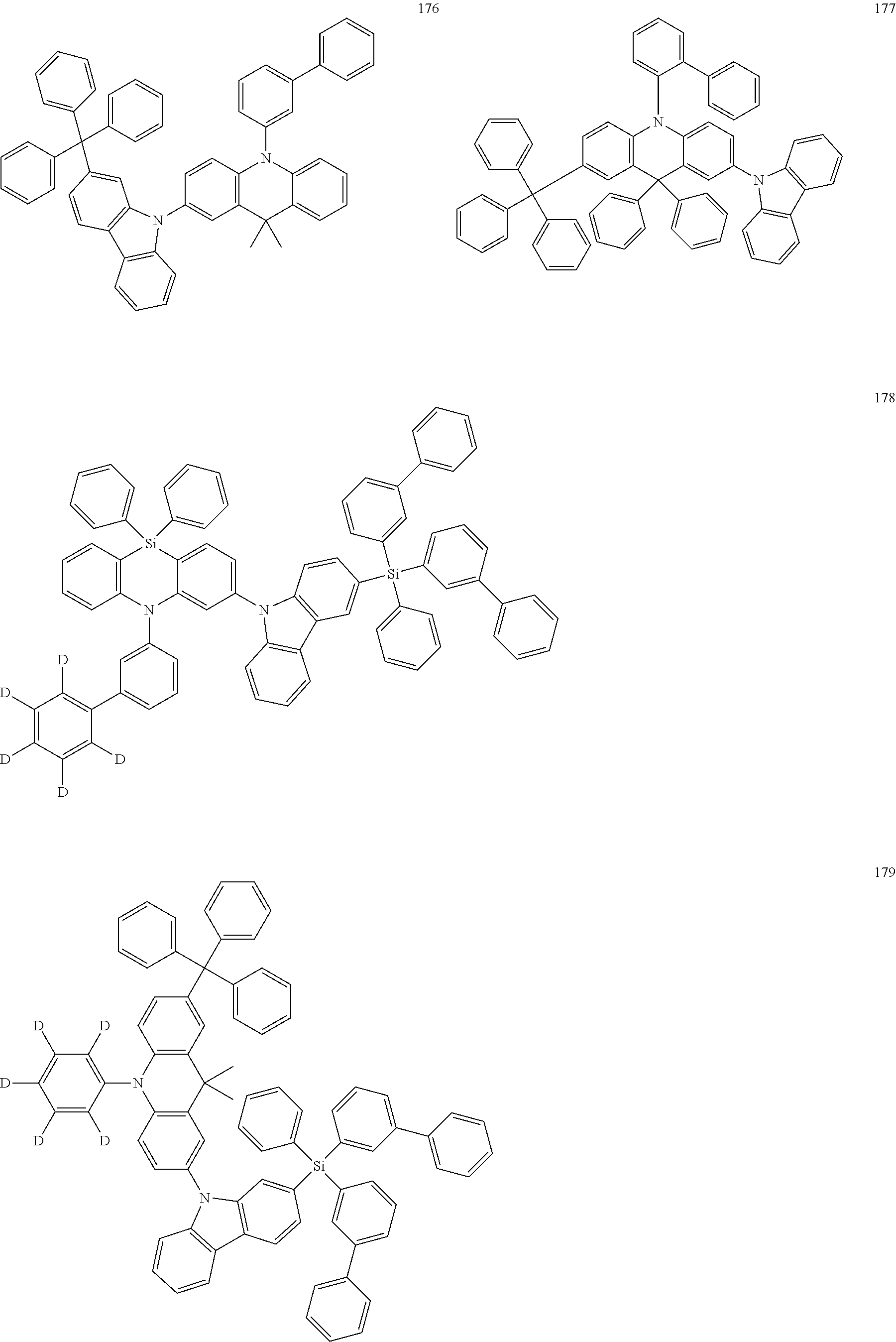

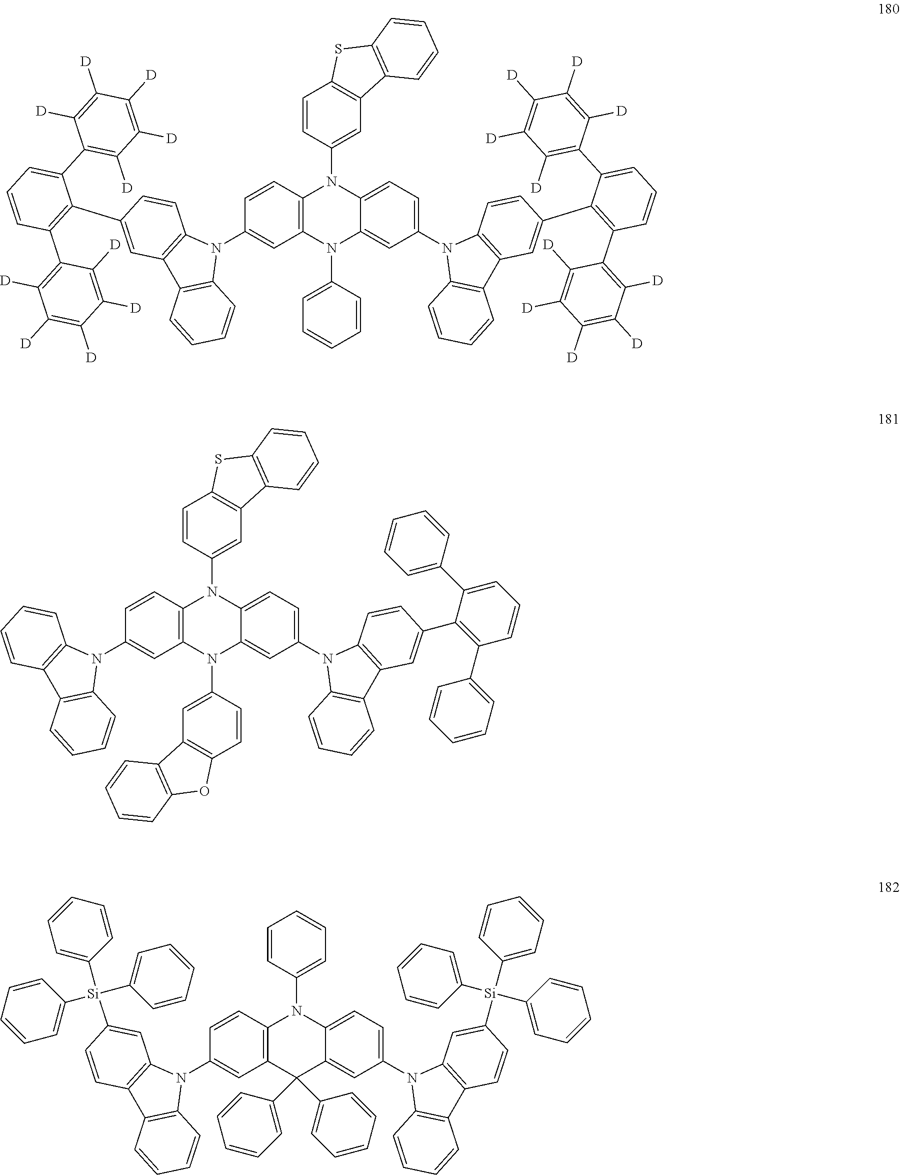

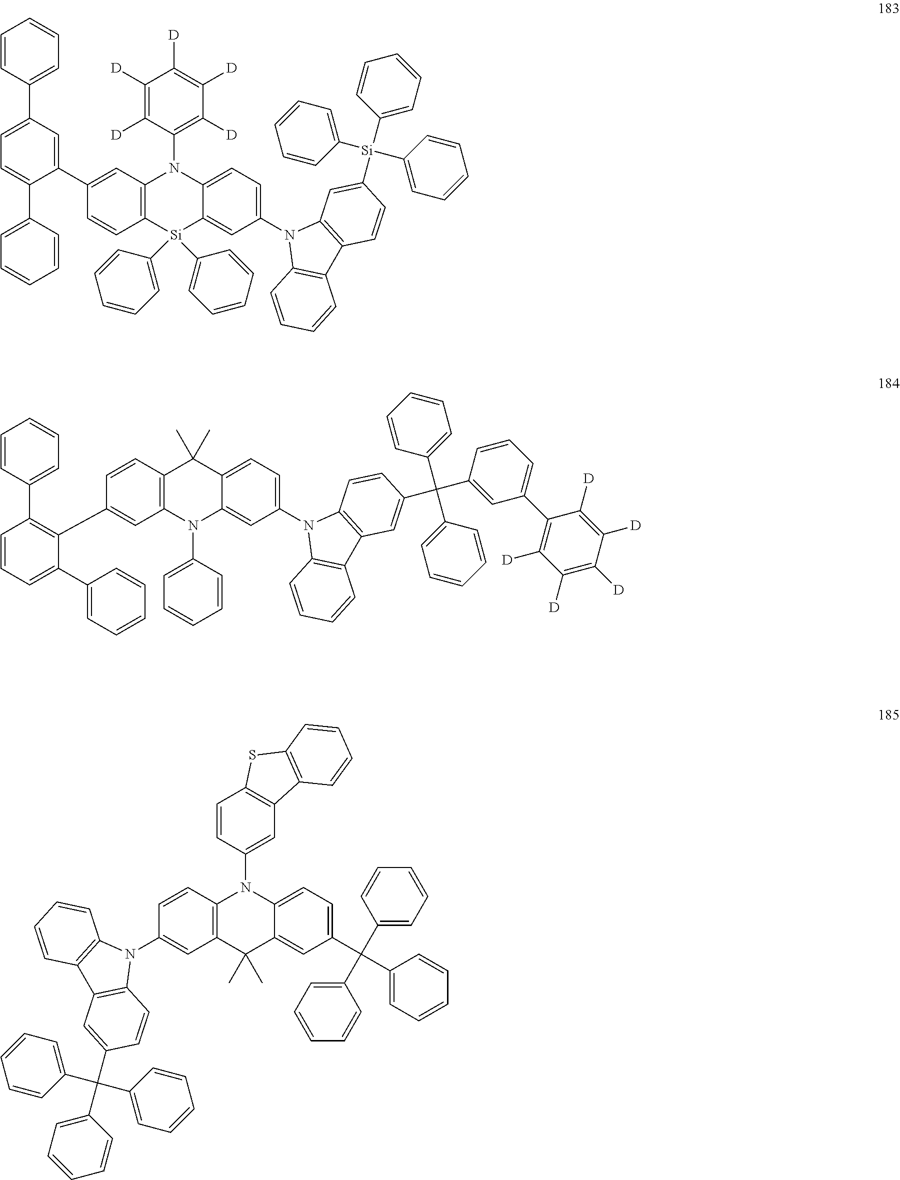 [SMILES: [2H]c1c([2H])c([2H])c(N2c3cc(-c4cc(-c5ccccc5)ccc4-c4ccccc4)ccc3[Si](c3ccccc3)(c3ccccc3)c3cc(-n4c5ccccc5c5ccc([Si](c6ccccc6)(c6ccccc6)c6ccccc6)cc54)ccc32)c([2H])c1[2H] ; [2H]c1c([2H])c([2H])c(-c2cccc(C(c3ccccc3)(c3ccccc3)c3ccc4c(c3)c3ccccc3n4-c3ccc4c(c3)N(c3ccccc3)c3cc(-c5c(-c6ccccc6)cccc5-c5ccccc5)ccc3C4(C)C)c2)c([2H])c1[2H] ; CC1(C)c2cc(-n3c4ccccc4c4cc(C(c5ccccc5)(c5ccccc5)c5ccccc5)ccc43)ccc2N(c2ccc3sc4ccccc4c3c2)c2ccc(C(c3ccccc3)(c3ccccc3)c3ccccc3)cc21]

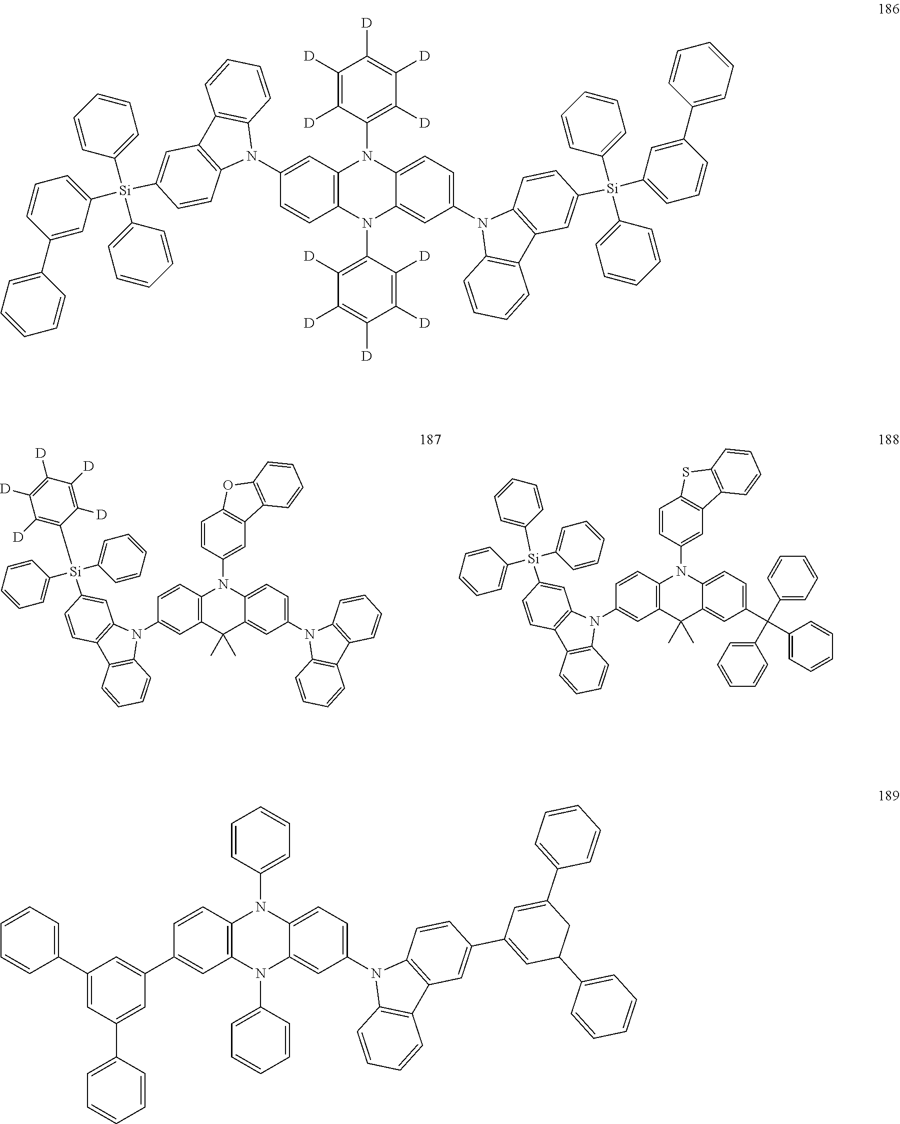

14. The heterocylic compound of claim 1, wherein
the heterocyclic compound is to emit blue light having a maximum emission wavelength of 390 nm or more and 440 nm or less, and
a difference between a singlet (S₁) energy level and a triplet (T₁) energy level is 0.6 eV or less.

15. A light-emitting device comprising:
a first electrode;
a second electrode facing the first electrode;
an interlayer located between the first electrode and the second electrode and comprising an emission layer; and
the heterocyclic compound of claim 1.

16. The light-emitting device of claim 15, wherein the emission layer comprises the heterocyclic compound.

17. The light-emitting device of claim 15, wherein the heterocyclic compound is a host.

18. An electronic apparatus comprising the light-emitting device of claim 15.

19. The electronic apparatus of claim 18, further comprising a thin-film transistor,
wherein the thin-film transistor comprises a source electrode and a drain electrode, and
the first electrode of the light-emitting device is electrically connected to at least one selected from the source electrode and the drain electrode of the thin-film transistor.

20. The electronic apparatus of claim 18, further comprising a color filter, a color conversion layer, a touch screen layer, a polarizing layer, or any combination thereof.
